(19) United States
(12) Patent Application Publication
(10) Pub. No.: US 2023/0094911 A1
(43) Pub. Date: Mar. 30, 2023

(54) Solid Supports and Methods for Depleting and/or Enriching Library Fragments Prepared from Biosamples

(71) Applicant: Illumina, Inc., San Diego, CA (US)

(72) Inventors: Robert Scott Kuersten, Madison, WI (US); Jeffrey Koble, San Diego, CA (US)

(73) Assignee: ILLUMINA, INC., San Diego, CA (US)

(21) Appl. No.: 17/937,021

(22) Filed: Sep. 30, 2022

Related U.S. Application Data
(63) Continuation of application No. PCT/US2022/077221, filed on Sep. 29, 2022.
(60) Provisional application No. 63/351,170, filed on Jun. 10, 2022.
(60) Provisional application No. 63/250,563, filed on Sep. 30, 2021.

(51) Int. Cl. C12Q 1/689; C12N 15/10

(52) U.S. Cl. CPC C12Q 1/689; C12N 15/1096; C12N 15/1072; C12Q 1/6844

(57) ABSTRACT

Described herein are solid supports and methods for depleting library fragments prepared from unwanted RNA sequences and/or enriching library fragments prepared from desired RNA sequences. These methods may incorporate microfluidics and flowcells for greater ease of use. Libraries enriched or depleted with the present methods may be used for sequencing. Also described are probes and methods for enzymatic depletion of ribosomal RNA from human microbiome samples.

### CROSS-REFERENCE TO RELATED APPLICATION

This application is a bypass continuation of PCT/US2022/077221, filed Sep. 29, 2022, which claims the benefit of priority of U.S. Provisional Application No. 63/250,563, filed Sep. 30, 2021, and U.S. Provisional Application No. 63/351,170, filed Jun. 10, 2022, the contents of which are each incorporated by reference herein in their entireties for any purpose.

### SEQUENCE LISTING

The instant application contains a Sequence Listing which has been submitted electronically in XML format and is hereby incorporated by reference in its entirety. Said XML copy, created on Nov. 22, 2022, is named “2022-11-22_01243-0028-00US_ST26” and is 1,424,744 bytes in size.

### DESCRIPTION

### Field

This disclosure relates to solid supports and methods for depleting library fragments prepared from unwanted RNA sequences and/or enriching library fragments prepared from desired RNA sequences. Libraries enriched or depleted with the present methods may be used to generate sequencing data. Also described are probes and methods for enzymatic depletion of ribosomal RNA from human microbiome samples.

### Background

Samples comprising RNA often have a high abundance of RNA that is not of interest to the user. For example, ribosomal RNA (rRNA) typically comprises most of the RNA molecules in total RNA (approximately 80%-95%). One challenge in RNA sequencing for gene expression analysis is that following RNA extraction most of the extracted material is dominated by a small number of highly abundant transcripts, such as the non-coding ribosomal ribonucleic acids (rRNAs). In a total RNA sample from human blood, globin messenger RNAs (mRNAs) can be present at a dominating level. Accordingly, sequencing RNA transcripts (RNA-Seq) is often inefficient and cost prohibitive for many users and applications. There is a need to deplete abundant transcripts, such as rRNAs and mRNAs, in a sample prior to RNA sequencing.

To circumvent the barrier of abundant unwanted RNA, several solutions have emerged including RNase H-mediated depletion. This method involves hybridizing DNA probes complementary to known rRNA sequences followed by DNA:RNA hybrid-specific cleavage by RNase H and subsequent removal via wash steps. This methodology is implemented as part of the current Illumina Total RNA Stranded Library Prep workflow and New England Biolabs NEBNext rRNA Depletion Kit and RNA depletion methods as described in U.S. Pat. Nos. 9,745,570 and 9,005,891. While these methods are effective, drawbacks include upfront depletion, increased costs, and increased hands-on time (HOT).

Improvements are needed for methods of RNA depletion from microbiome samples. The microbiome plays a critical role in human health and disease (Cho et al. Nat. Rev. Genet. 13:260-70 (2012)). Over the past decade, next-generation sequencing-based analyses have provided insights into the composition of the microbiome across body sites and life stages and have begun to uncover correlations between microbial taxa or microbial functions and disease states (see, for example, Gilbert, J. A. et al. Nat. Med. 24:392-400 (2018); Durack and Lynch J. Exp. Med. 216(1):20-40 (2019); Lloyd-Price et al. Genome Med. 8:51 (2016)). Beyond genomic analysis of microbiome composition, multi-omic data incorporate measurements of the microbiota-associated transcriptome, proteome, or metabolome providing further insights into microbiome activity and function. Although metagenomic and metatranscriptomic profiles tend to be generally consistent, microbial functional profiles derived from DNA sequencing are more conserved across donors than transcriptional profiles, which are highly donor specific (Franzosa, E. A. et al. Proc. Natl. Acad. Sci. U.S.A 111(22):E2329-38 (2014)). Importantly, many broadly encoded metagenomic pathways are expressed by a small number of organisms, highlighting the utility of metatranscriptomics to identify functional activities (Abu-Ali, G. S. et al. Nat. Microbiol. 3(3):356-366 (2018)). In particular, transcriptomic measurements of the human gut associated microbiome have been used to study microbial carbohydrate metabolism (Turnbaugh, P. J. et al. Proc. Natl. Acad. Sci. U.S.A 107:7503-7508 (2010)), have provided functional information about intestinal diseases, such as inflammatory bowel disease (IBD, Lloyd-Price, J. et al. Nature 569:655-662 (2019)), and mechanisms of drug metabolism (Haiser, H. J. et al. Science 341(6143):295-298 (2013)).

The microbiota that colonize the human gut and other tissues are dynamic, varying across individuals and over time, both in composition and functional state. In studying the function of the human microbiome and mechanisms of microbiota-mediated phenotypes, gene expression measurements provide additional insights to DNA-based measurements of microbiome composition. However, efficient, unbiased removal of microbial ribosomal RNA (rRNA) presents a barrier to acquiring metatranscriptomic data, as rRNA typically accounts >90% of total RNA in microbial cells.

In particular, acquiring metatranscriptomic data is hindered by the fact that the vast majority of microbial-derived RNA molecules correspond to ribosomal RNA (rRNA, as described in Giannoukos, G. et al. Genome Biol. 13(3):R23 (2012)). In eukaryotes, non-ribosomal RNA can be easily and efficiently enriched through selective reverse transcription or pull-down approaches that target the poly-A tail or using probes to specifically bind rRNA molecules prior to removal by capture or enzymatic digestion (Hrdlickova et al. Wiley Interdiscip. Rev. RNA 8(1):10.1002/wma.1364 (2017) and Zhao et al. Sci. Rep. 8(1):4781 (2018)). Although poly-A polymerase was first isolated from Escherichia coli (August et al. J. Biol. Chem. 237:3786-3793 (1962) and Modak and Srinivasan J. Biol. Chem. 248(19):6904-6910 (1973)), bacterial mRNA transcripts are not, as a rule, poly-adenylated, and when poly-adenylation does occur it is associated with RNA degradation (Mohanty and Kushner Mol. Microbiol. 34:1094-1108 (1999) and O'Hara et al. Proc. Natl. Acad. Sci. U S. A. 92:1807-1811 (1995)). Thus, for bacterial samples, selective enrichment of mRNA is not easily achievable and the depletion of rRNA must be accomplished by other means.

While a large number of studies have developed efficient methods to deplete rRNA in individual bacterial species using probe-based capture (Culviner et al. MBio 11(2): e00010-20 (2020), enzymatic depletion (Huang et al. Nucleic Acids Res. 48(4):E20 (2020)), or CRISPR-based methods (Prezza, G. et al. RNA 26:1069-1078 (2020) and Gu et al. Genome Biol. 17:1-13 (2016)), depleting rRNA in in complex human microbiome samples that can contain hundreds of species presents a significant technical challenge. In addition, the composition of the microbiota varies substantially across body sites and throughout different life stages, further expanding the taxonomic coverage required for robust depletion of rRNA across human microbiome samples. Probe-based sequence capture methods, such as were employed with Illumina's RiboZero Gold kit can provide strong rRNA depletion across a variety of sample types, including human gut microbiome samples (Reck, M. et al. BMC Genomics 16(1):494 (2015)). However, such probes are costly, difficult to manufacture, and tend to perform best with high quality RNA samples. Moreover, capture-based rRNA depletion methods can yield variable results based on operator skill. These factors led to the discontinuation of the capture based bacterial RiboZero Gold depletion kit.

Described herein is the development of a pan-human microbiome probe set for efficient and consistent enzymatic (RNase H) microbial rRNA depletion. Through an iterative design process, probes were designed that effectively deplete rRNA found in human oral, vaginal and adult and infant gut microbiome samples, substantially improving mapping rates to coding microbial gene databases. Using defined spike-ins, the rRNA depletion process was shown to not introduce substantial bias in the metatranscriptomic profiles. In addition, the resulting metatranscriptomics data allows the user to refine informatic pipelines for rRNA and host mapping and to examine gene expression and functional pathways across human microbiome sites. Thus, the method described here circumvents the limitations of sequence capture methods and represents a highly effective rRNA depletion option for metatranscriptomics studies of human-associated microbial communities.

For example, a main limitation of metatranscriptomic studies (i.e., sequencing of microbial communities in specific environmental samples without culturing of microbes) is overcoming the dominating abundance of ribosomal RNA (rRNA). Highly abundant rRNA are often of limited interest to the user (i.e., unwanted transcripts), but can dramatically reduce the sequencing coverage of mRNA (i.e., desired transcripts). In metatranscriptomic sequencing, rRNA depletion is often performed by using hybridization with 16S and 23S rRNA probes followed by separation or by using depletion of rRNAs using a method based on binding of probes following by exonuclease treatment. After rRNA depletion, library preparation can be performed.

Described herein is an iterative probe design strategy that was used to develop a probe set for efficient enzymatic rRNA removal of human-associated microbiota. This strategy resulted in custom probe sets that efficiently deplete rRNA from a range of human microbiome samples, including adult gut, infant gut, oral, and vaginal communities. Successful rRNA depletion allows for characterization of taxonomic and functional changes during the development of the gut microbiome. Further, the rRNA depletion process does not introduce substantial quantitative error in the resulting transcriptomic profiles. The pan-human microbiome enzymatic rRNA depletion probes described here provide a powerful tool for studying the transcriptional dynamics and function of the human microbiome.

In some assays, methods of “upfront depletion,” including RNase depletion, can be problematic for users with limited total RNA material for input into the assay. For example, if insufficient RNA remains after upfront depletion methods, downstream biochemical reactions can be inefficient resulting in poor assay performance and results. Further, upfront depletion with RNase H includes wash steps (potentially causing loss of desired RNA) and high temperature incubations (potentially causing degradation of desired RNA), which may be a concern with certain samples.

Described herein is a differentiated solution using a solid support (such as a flowcell-like device) with immobilized oligonucleotides that can bind to library fragments prepared from unwanted RNA. For example, library fragments prepared from rRNA sequences can be captured by flowcell-tethered oligonucleotides, while library fragments lacking these sequences can be siphoned for collection. After collection of the non-depleted library fragments, only a quick quality control step checking the concentration and size of the non-rRNA sequencing library may be performed prior to standard sequencing. This approach is advantageous as rRNA can act as a “carrier molecule” for low abundance RNA molecules throughout the library preparation process, making for a robust, sensitive assay. In addition to removal of unwanted library fragments (such as those prepared from rRNA), this method can be expanded to substitute traditional PCR amplification via thermal cycler in favor of a bridge amplification-like process to further reduce HOT and demonstrate additional library preparation functionality via sequencer fluidics chemistry. Similar methods can also be used for other unwanted RNA, such as for depleting host-derived RNA transcripts when a user wants to specifically evaluate microbiome RNA from a host.

In addition, disclosed herein are methods with designed to enrich for library fragments prepared from desired RNA. Both depletion and enrichment methods can generate libraries that have fewer unwanted library fragments, allowing for less expensive and/or deeper sequencing of desired library fragments.

### SUMMARY

In accordance with the description, described herein are methods of depleting library fragments prepared from unwanted RNA and methods of enriching library fragments prepared from desired RNA. These methods may be performed with standard lab equipment, such as flowcells comprised in sequencers. In some embodiments, standard sequencing consumables and platform (i.e., sequencer) can be used as a microfluidic device for enriching or depleting library fragments. In some embodiments, depletion or enrichment is performed after cDNA synthesis and amplification.

Also described are probes that may be used for enzymatic depletion of rRNA from human microbiome samples.

Embodiment 1. A method of depleting unwanted cDNA library fragments from a library of cDNA fragments prepared from RNA, wherein the unwanted library fragments comprise those prepared from unwanted RNA sequences, comprising (a) preparing a solid support comprising at least one immobilized oligonucleotide, wherein each immobilized oligonucleotide comprises a nucleic acid sequence corresponding to an unwanted RNA sequence or its complement, (b) adding the library of fragments to the solid support and hybridizing the library fragments to at least one immobilized oligonucleotide to allow binding of unwanted library fragments to at least one immobilized oligonucleotide, and (c) collecting library fragments not bound to at least one immobilized oligonucleotide.

Embodiment 2. The method of embodiment 1, wherein at least one unwanted RNA sequence has at least 90%, at least 95%, or at least 99% homology to a high-abundance RNA sequence in a sample used to prepare the library of fragments.

Embodiment 3. The method of embodiment 2, wherein all unwanted sequences have at least 90%, at least 95%, or at least 99% homology to a high-abundance RNA sequence in a sample used to prepare the library of fragments.

Embodiment 4. The method of embodiment 1, wherein the at least one unwanted RNA sequence is a high-abundance RNA sequence.

Embodiment 5. The method of any one of embodiments 2-4, wherein the high-abundance RNA sequence is a ribosomal RNA (rRNA) sequence.

Embodiment 6. The method of any one of embodiments 1-5, wherein the unwanted RNA sequence is comprised in a host transcriptome.

Embodiment 7. The method of any one of embodiments 1-6, wherein the unwanted RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

Embodiment 8. The method of any one of embodiments 1-7, wherein the unwanted RNA sequence is from human, rat, mouse, or bacteria.

Embodiment 9. The method of embodiment 8, wherein the unwanted RNA sequence is 28S, 18S, 5.8S, 5S, 16S, or 12S RNA from humans, or a fragment thereof.

Embodiment 10. The method of embodiment 8, wherein the unwanted RNA sequence is rat 16S, rat 28S, mouse 16S, or mouse 28S RNA.

Embodiment 11. The method of embodiment 8, wherein the bacteria are Archaea species, E. Coli, or B. subtilis.

Embodiment 12. The method of embodiment 8, wherein the unwanted RNA sequence is comprised in 23S, 16S, or 5S RNA from Gram-positive or Gram-negative bacteria.

Embodiment 13. The method of embodiment 8, wherein the unwanted RNA sequence is from an organism in the human microbiome.

Embodiment 14. The method of embodiment 13, wherein the at least one immobilized oligonucleotide comprises a sequence comprising any one or more of SEQ ID NOs: 1-1131 or its complement.

Embodiment 15. The method of any one of embodiments 1-14, wherein the at least one immobilized oligonucleotide comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131 or its complement.

Embodiment 16. The method of embodiment 15, wherein the at least one immobilized oligonucleotide comprises 500 or more, 600 or more, 700 or more, 800 or more, 900 or more, 1000 or more, 1100 or more, or 1131 sequences selected from SEQ ID NOs: 1-1131 or its complement.

Embodiment 17. The method of any one of embodiments 14-16, wherein the at least one immobilized oligonucleotide comprises at least one sequence comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

Embodiment 18. The method of embodiment 17, wherein the at least one immobilized oligonucleotide comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

Embodiment 19. The method of embodiment 18, wherein the at least one immobilized oligonucleotide comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

Embodiment 20. The method of any one of embodiments 17-19, wherein the at least one immobilized oligonucleotide further comprises at least one sequence comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

Embodiment 21. The method of embodiment 20, wherein the pool of oligonucleotides comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

Embodiment 22. The method of embodiment 21, wherein the pool of oligonucleotides comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

Embodiment 23. The method of any one of embodiments 14-16, wherein the pool of oligonucleotides comprises at least one sequence comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131 or its complement.

Embodiment 24. The method of embodiment 23, wherein the pool of oligonucleotides comprises 100 or more, 500 or more, or 1000 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131 or its complement.

Embodiment 25. The method of embodiment 24, wherein the at least one immobilized oligonucleotide comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131 or its complement.

Embodiment 26. The method of any one of embodiments 17-25, wherein the at least one immobilized oligonucleotide further comprises at least one sequence comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 or its complement.

Embodiment 27. The method of embodiment 26, wherein the at least one immobilized oligonucleotide comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 or its complement.

Embodiment 28. The method of embodiment 27, wherein the at least one immobilized oligonucleotide comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 or its complement.

Embodiment 29. The method of any one of embodiments 1-28, wherein the unwanted RNA sequences are selected by determining the most abundant sequences in a sample comprising RNA.

Embodiment 30. The method of embodiment 29, wherein the most abundant sequences comprise the 100 most abundant sequences, the 1,000 most abundant sequences, or the 10,000 most abundant sequences.

Embodiment 31. The method of any one of embodiments 1-30, wherein the unwanted RNA sequence comprises a sequence with homology of at least 90%, at least 95%, or at least 99% to a most abundant sequence in a sample comprising RNA.

Embodiment 32. The method of any one of embodiments 1-31, wherein the collected library fragments comprise a library depleted of unwanted library fragments.

Embodiment 33. The method of any one of embodiments 32, wherein unwanted library fragments serve as carrier molecules for other library fragments.

Embodiment 34. The method of any one of embodiments 1-33, wherein the library of fragments added to the solid support is prepared from RNA using a stranded method of cDNA preparation.

Embodiment 35. The method of any one of embodiments 1-34, wherein the library fragments comprise library adapters and the solid support further comprises immobilized oligonucleotides comprising solid support adapter sequences that can bind to library adapters.

Embodiment 36. The method of embodiment 35, wherein the solid support adapter sequences comprise a P5 sequence (SEQ ID NO: 1132), a P7 sequence (SEQ ID NO: 1133), and/or their complements.

Embodiment 37. The method of embodiment 35 or embodiment 36, wherein adapter complements that are all or partially complementary to the solid support adapter sequences are bound to the solid support adapter sequences.

Embodiment 38. The method of embodiment 37, wherein the binding of the adapter complements to the solid support adapter sequences is reversible.

Embodiment 39. The method of embodiment 37 or embodiment 38, wherein adapter complements bound to the solid support adapter sequences generate double-stranded immobilized oligonucleotides.

Embodiment 40. The method of embodiment 39, wherein solid support adapter sequences bound to adapter complements cannot bind to library adapters.

Embodiment 41. The method of embodiment 39 or embodiment 40, further comprising denaturing library fragments and/or adapter complements hybridized to the immobilized oligonucleotides.

Embodiment 42. The method of embodiment 41, wherein the denaturing is performed with a denaturing agent and/or heat.

Embodiment 43. The method of embodiment 42, wherein the denaturing agent is NaOH, optionally wherein the NaOH concentration is 0.2 N.

Embodiment 44. The method of embodiment 42, wherein the heat is 95° C.-98° C.

Embodiment 45. The method of any one of embodiments 41-44, wherein the denatured library fragments and/or adapter complements are siphoned to a waste compartment.

Embodiment 46. The method of any one of embodiments 41-45, wherein the steps of adding a sample, collecting, and denaturing are repeated, wherein the collected library fragments are added back to the solid support after the denaturing.

Embodiment 47. The method of any one of embodiments 1-46, wherein the collected library fragments are collected in a reservoir comprised in a sequencer comprising the flowcell.

Embodiment 48. The method of any one of embodiments 1-47, wherein the library fragments that hybridize to immobilized oligonucleotides comprise library fragments prepared from rRNA.

Embodiment 49. The method of any one of embodiments 1-48, wherein the library depleted of unwanted library fragments comprises fewer library fragments prepared from unwanted RNA sequences, as compared to the same library before it was added to the solid support.

Embodiment 50. The method of any one of embodiments 1-49, wherein the unwanted library fragments that hybridize to immobilized oligonucleotides comprise library fragments prepared from host RNA comprised in a sample comprising host RNA and non-host nucleic RNA.

Embodiment 51. The method of embodiment 50, wherein the non-host RNA is microbial.

Embodiment 52. The method of embodiment 51, wherein microbe is a bacterium, a virus, and/or a fungus.

Embodiment 53. The method of embodiment 52, wherein the microbe is a pathogen.

Embodiment 54. The method of embodiment 52, wherein the microbe is an organism in the host microbiome.

Embodiment 55. The method of any one of embodiments 50-54, wherein the host is human.

Embodiment 56. The method of any one of embodiments 29-55, further comprising adding the collected library fragments to the solid support after denaturing the hybridized library fragments and/or adapter complements.

Embodiment 57. The method of embodiment 1-56, wherein sequences comprised in library fragments specifically bind to solid support adapter sequences comprising P5 (SEQ ID NO: 1132), P7 (SEQ ID NO: 1133), and/or their complements.

Embodiment 58. The method of embodiment 1-57, wherein library adapter sequences are added to collected library fragments.

Embodiment 59. The method of embodiment 58, wherein the library adapter sequences are added by ligation.

Embodiment 60. The method of any one of embodiments 1-59, wherein the library of fragments added to the solid support is prepared by a method comprising incorporating one or more library adapters that specifically bind to solid support adapter sequences comprising P5 (SEQ ID NO: 1132), P7 (SEQ ID NO: 1133), and/or their complements.

Embodiment 61. The method of embodiment 60, wherein the method comprising incorporating one or more library adapters is tagmentation or fragmentation followed by adapter ligation.

Embodiment 62. The method of any one of embodiments 1-61, wherein the method does not require degradation of RNA.

Embodiment 63. The method of any one of embodiments 1-62, wherein the library depleted of unwanted library fragments is assessed for library size and/or concentration.

Embodiment 64. The method of any one of embodiments 1-63, wherein the library depleted of unwanted library fragments is sequenced.

Embodiment 65. The method of any one of embodiments 1-64, further comprising amplifying the library depleted of unwanted library fragments before sequencing.

Embodiment 66. The method of embodiment 65, wherein the amplifying is by PCR amplification.

Embodiment 67. The method of embodiment 65, wherein the amplifying is by bridge amplification.

Embodiment 68. The method of embodiment 67, wherein bridge amplification is performed after adding the collected library fragments to the solid support and allowing the library adapters comprised in the collected library fragments to bind to the solid support adapter sequences, wherein the adding is performed after denaturing the hybridized library fragments and/or adapter complements.

Embodiment 69. The method of embodiment 64, 65, 67, or 68, wherein the sequencing is performed without PCR amplification.

Embodiment 70. The method of any one of embodiments 64, 65, or 67-69, wherein the amplifying does not require a thermocycler.

Embodiment 71. The method of any one of embodiments 1-70, wherein the method is fully performed in a sequencer.

Embodiment 72. A method of enriching desired cDNA library fragments from a library of cDNA fragments prepared from RNA, wherein the desired library fragments comprise those prepared from desired RNA sequences, comprising (a) preparing a solid support comprising at least one immobilized oligonucleotide, wherein each immobilized oligonucleotide comprises a nucleic acid sequence corresponding to a desired RNA sequence or its complement, (b) adding the library of fragments to the solid support and hybridizing the library fragments to the at least one immobilized oligonucleotide to allow binding of desired library fragments to the at least one immobilized oligonucleotide, and (c) collecting library fragments bound to the at least one immobilized oligonucleotide.

Embodiment 73. The method of embodiment 72, wherein the library of fragments has been subjected to a method of depleting unwanted cDNA library fragments of any one of embodiments 1-71 before the adding.

Embodiment 74. The method of embodiment 72 or 73, wherein at least one desired RNA sequence has at least 90%, at least 95%, or at least 99% homology to an RNA sequence of interest in a sample used to prepare the library of fragments.

Embodiment 75. The method of embodiment 74, wherein all desired RNA sequences have at least 90%, at least 95%, or at least 99% homology to an RNA sequence of interest in a sample used to prepare the library of fragments.

Embodiment 76. The method of any one of embodiments 72-75, wherein the at least one desired RNA sequence is an RNA sequence of interest.

Embodiment 77. The method of any one of embodiments 72-76, wherein the desired RNA sequence is an exome sequence.

Embodiment 78. The method of any one of embodiments 72-77, wherein the desired RNA sequence is from human, rat, mouse, and/or bacteria.

Embodiment 79. The method of embodiment 78, wherein the desired RNA sequence is from an organism in the human microbiome.

Embodiment 80. The method of any one of embodiments 72-79, wherein the collected library fragments comprise a library enriched for desired library fragments.

Embodiment 81. The method of any one of embodiments 72-90, wherein the library of fragments added to the solid support is prepared from RNA using a stranded method of cDNA preparation.

Embodiment 82. The method of any one of embodiments 72-81, wherein the collecting comprises denaturing the library fragments hybridized to the at least one immobilized oligonucleotide and then collecting the library enriched for desired fragments in a reservoir comprised in a sequencer comprising the solid support.

Embodiment 83. The method of embodiment 82, wherein the denaturing is performed with a denaturing agent and/or heat.

Embodiment 84. The method of embodiment 83, wherein the heat is 95° C.-98° C.

Embodiment 85. The method of embodiment 83, wherein the denaturing agent is NaOH, optionally wherein the NaOH concentration is 0.2 N.

Embodiment 86. The method of any one of embodiments 82-85, wherein the steps of adding the library, denaturing, and collecting are repeated, wherein the collected library fragments are added to the solid support after the denaturing.

Embodiment 87. The method of any one of embodiments 82-86, wherein the library enriched for desired library fragments comprises a greater percentage of library fragments prepared from desired RNA sequences, as compared to the library before adding to the solid support.

Embodiment 88. The method of any one of embodiments 82-87, wherein the library enriched for desired library fragments is assessed for library size and/or concentration.

Embodiment 89. The method of any one of embodiments 82-88, wherein the library enriched for desired library fragments is sequenced.

Embodiment 90. The method of any one of embodiments 82-89, further comprising amplifying the library enriched for desired library fragments before sequencing.

Embodiment 91. The method of any one of embodiments 1-90, wherein the at least one immobilized oligonucleotide is 20-100 bases in length, optionally wherein the at least one immobilized oligonucleotides is 45-55 bases in length.

Embodiment 92. The method of any one of embodiments 1-91, wherein the at least one immobilized oligonucleotide is single-stranded.

Embodiment 93. The method of any one of embodiments 1-92, wherein single-stranded library fragments are prepared before adding the library of fragments to the solid support.

Embodiment 94. The method of any one of embodiments 1-93, wherein the solid support is a flowcell.

Embodiment 95. A solid support having two pools of immobilized oligonucleotides on its surface, wherein the first pool comprises immobilized oligonucleotides each comprising a nucleic acid sequence corresponding to an unwanted RNA sequence or its complement and the second pool comprises immobilized oligonucleotides each comprising a solid support adapter sequence that can bind to a library adapter comprised in library fragments.

Embodiment 96. The solid support of embodiment 95, wherein at least one unwanted RNA sequence has at least 90%, at least 95%, or at least 99% homology to a high-abundance RNA sequence in a sample used to prepare the library of fragments.

Embodiment 97. The solid support of embodiment 96, wherein all unwanted sequences have at least 90%, at least 95%, or at least 99% homology to a high-abundance RNA sequence in a sample used to prepare the library of fragments.

Embodiment 98. The solid support of any one of embodiments 95-97, wherein the at least one unwanted RNA sequence is a high-abundance RNA sequence.

Embodiment 99. The solid support of any one of embodiments 96-98, wherein the high-abundance RNA sequence is a ribosomal RNA (rRNA) sequence.

Embodiment 100. The solid support of any one of embodiments 95-99, wherein the unwanted RNA sequence is comprised in a host transcriptome.

Embodiment 101. The solid support of any one of embodiments 95-100, wherein the unwanted RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

Embodiment 102. The solid support of any one of embodiments 95-101, wherein the unwanted RNA sequence is from human, rat, mouse, or bacteria.

Embodiment 103. The solid support of embodiment 102, wherein the unwanted RNA sequence is 28S, 18S, 5.8S, 5S, 16S, or 12S RNA from humans, or a fragment thereof.

Embodiment 104. The solid support of embodiment 102, wherein the unwanted RNA sequence is comprised in rat 16S, rat 28S, mouse 16S, or mouse 28S RNA.

Embodiment 105. The solid support of embodiment 102, wherein the bacteria are Archaea species, E. Coli, or B. subtilis.

Embodiment 106. The solid support of embodiment 102, wherein the unwanted RNA sequence is comprised in 23S, 16S, or 5S RNA from Gram-positive or Gram-negative bacteria.

Embodiment 107. The solid support of embodiment 102, wherein the unwanted RNA sequence is from an organism comprised in the human microbiome.

Embodiment 108. The solid support of any one of embodiments 95-107, wherein the unwanted RNA sequence comprises any one or more of SEQ ID NOs: 1-1131.

Embodiment 109. The solid support of any one of embodiments 95-108, wherein the unwanted RNA sequence comprises a sequence with homology of at least 90%, at least 95%, or at least 99% to a most abundant sequence in a sample comprising RNA, wherein the most abundant sequences comprise the 100 most abundant sequences, the 1,000 most abundant sequences, or the 10,000 most abundant sequences.

Embodiment 110. The solid support of any one of embodiments 95-109, wherein the solid support adapter sequences comprise a P5 sequence (SEQ ID NO: 1132), a P7 sequence (SEQ ID NO: 1133), and/or their complements.

Embodiment 111. The solid support of any one of embodiments 95-110, wherein adapter complements that are all or partially complementary to the solid support adapter sequences are bound to the solid support adapter sequences.

Embodiment 112. The solid support of embodiment 111, wherein the binding of the adapter complements to the solid support adapter sequences is reversible.

Embodiment 113. The solid support of embodiment 111 or embodiment 112, wherein the solid support adapter sequences and adapter complements generate double-stranded immobilized oligonucleotides.

Embodiment 114. The solid support of any one of embodiments 95-113, wherein the at least one immobilized oligonucleotide is 20-100 bases in length, optionally wherein the at least one immobilized oligonucleotide is 45-55 bases in length.

Embodiment 115. The solid support of any one of embodiments 95-114, wherein the solid support is a flowcell.

Embodiment 116. The solid support of any one of embodiments 95-115, wherein the at least one immobilized oligonucleotide is single-stranded.

Embodiment 117. A composition comprising a single-stranded library fragment comprising cDNA prepared from a sample comprising RNA that is hybridized to the solid support of any one of embodiments 95-116.

Embodiment 118. The composition of embodiment 117, wherein the cDNA is complementary to RNA comprised in the sample.

Embodiment 119. A method for depleting unwanted RNA molecules comprised in a patient microbiome sample, wherein the patient microbiome sample comprises at least one target RNA or DNA sequence and at least one unwanted RNA molecule, comprising (a) sequencing a plurality of probe-development microbiome samples to determine at least one unwanted RNA molecule comprising a bacterial ribosomal RNA (rRNA) sequence from sequencing data; (b) preparing a probe set comprising at least one DNA probe complementary to the at least one unwanted RNA molecule; (c) contacting the patient microbiome sample with the probe set to prepare DNA:RNA hybrids; and (d) contacting the DNA:RNA hybrids with a ribonuclease that degrades the RNA from the DNA:RNA hybrids, thereby degrading the unwanted RNA molecules in the patient microbiome sample to form a degraded mixture.

Embodiment 120. A method for depleting unwanted RNA molecules comprised in a patient microbiome sample, wherein the patient microbiome sample comprises at least one target RNA or DNA sequence and at least one unwanted RNA molecule, comprising (a) contacting the patient microbiome sample with a probe set comprising at least one sequence comprising at least one of SEQ ID NOs: 1-1131 to prepare DNA:RNA hybrids; and

(b) contacting the DNA:RNA hybrids with a ribonuclease that degrades the RNA from the DNA:RNA hybrids, thereby degrading the unwanted RNA molecules in the patient microbiome sample to form a degraded mixture.

Embodiment 121. The method of embodiment 119 or embodiment 120, further comprising (a) degrading any remaining DNA probes by contacting the degraded mixture with a DNA digesting enzyme, optionally wherein the DNA digesting enzyme is DNase I, to form a DNA degraded mixture; and (b) separating the degraded RNA from the degraded mixture or the DNA degraded mixture.

Embodiment 122. The method of any one of embodiments 119-121, wherein the contacting with the probe set comprises treating the nucleic acid sample with a destabilizer.

Embodiment 123. The method of embodiment 122, wherein the destabilizer is heat and/or a nucleic acid destabilizing chemical.

Embodiment 124. The method of embodiment 123, wherein the nucleic acid destabilizing chemical comprises betaine, DMSO, formamide, glycerol, or a derivative thereof, or a mixture thereof.

Embodiment 125. The method of embodiment 124, wherein the nucleic acid destabilizing chemical comprises formamide.

Embodiment 126. The method of embodiment 125, wherein the formamide is present during the contacting with the probe set at a concentration of from about 10 to 45% by volume.

Embodiment 127. The method of any one of embodiments 123-126, wherein treating the sample with heat comprises applying heat above the melting temperature of the at least one DNA:RNA hybrid.

Embodiment 128. The method of any one of embodiments 119-127, wherein the ribonuclease is RNase H or hybridase.

Embodiment 129. The method of any one of embodiments 119-128, wherein the patient is human.

Embodiment 130. The method of any one of embodiments 119-129, wherein the microbiome sample is oral, vaginal, or from the gut.

Embodiment 131. The method of embodiment 119-130, wherein the sample from the gut is a stool sample.

Embodiment 132. The method of embodiment 131, wherein the oral sample is a sample from the tongue.

Embodiment 133. The method of any one of embodiments 119-132, wherein the at least one DNA probe comprise 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131 or its complement.

Embodiment 134. The method of embodiment 133, wherein the at least one DNA probe comprises 500 or more, 600 or more, 700 or more, 800 or more, 900 or more, 1000 or more, 1100 or more, or 1131 sequences selected from SEQ ID NOs: 1-1131 or its complement.

Embodiment 135. The method of any one of embodiments 119-134, wherein the patient is at least 12 months of age, at least 15 months of age, at least 24 months of age, or at least 36 months of age.

Embodiment 136. The method of any one of embodiments 119-135, wherein the microbiome sample comprises at least one unwanted RNA molecule from Faecalibacterium, Lachnospiraceae, and/or Clostridium.

Embodiment 137. The method of any one of embodiments 119-136, wherein the microbiome sample is vaginal and comprises at least one unwanted RNA molecule from Gardnerella, Lactobacillus and/or Olsenella.

Embodiment 138. The method of any one of embodiments 119-136, wherein the microbiome sample is from tongue and comprises at least one unwanted RNA molecule from Veillonella, Rothia, Streptococcus, and/or Prevotella.

Embodiment 139. The method of any one of embodiments 120-138, wherein the at least one DNA probe comprises at least one sequence comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 140. The method of embodiment 139, wherein the at least one DNA probe comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 141. The method of embodiment 140, wherein the at least one DNA probe comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 142. The method of any one of embodiments 139-141, wherein the patient is 3 months of age or younger, 6 months of age of younger, 12 months of age or younger, 18 months of age or younger, 24 months of age or younger, or 36 months of age or younger.

Embodiment 143. The method of embodiment 142, wherein the microbiome sample comprises at least one unwanted RNA molecules from Bifidobacterium bifidum and/or Blautia.

Embodiment 144. The method of any one of embodiments 139-143, wherein the at least one DNA probe further comprises at least one sequence comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 145. The method of embodiment 144, wherein the at least one DNA probe comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 146. The method of embodiment 145, wherein the at least one DNA probe comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 147. The method of any one of embodiments 120-138, wherein the at least one immobilized oligonucleotide comprises at least one sequence comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 148. The method of embodiment 147, wherein the at least one immobilized oligonucleotide comprises 100 or more, 500 or more, or 1000 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 149. The method of embodiment 148, wherein the at least one immobilized oligonucleotide comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 150. The method of any one of embodiments 139-149, wherein the at least one DNA probe further comprises at least one sequence comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 151. The method of embodiment 150, wherein the at least one DNA probe comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 152. The method of embodiment 151, wherein the at least one DNA probe comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 153. The method of any one of embodiments 119-152, wherein the method depletes 70% or greater, 80% or greater, 90% or greater, or 95% or greater of bacterial rRNA comprised in the microbiome sample.

Embodiment 154. A composition comprising a probe set comprising (a) at least one DNA probe comprising at least one sequence comprising at least one of SEQ ID NOs: 1-1131; and (b) a ribonuclease capable of degrading RNA in an DNA:RNA hybrid.

Embodiment 155. The composition of embodiment 154, wherein the ribonuclease is RNase H.

Embodiment 156. A kit comprising a probe set comprising (a) at least one DNA probe comprising at least one sequence comprising at least one of SEQ ID NOs: 1-1131; and (b) a ribonuclease capable of degrading RNA in an DNA:RNA hybrid.

Embodiment 157. The kit of embodiment 156, comprising (a) a probe set comprising at least one DNA probe comprising at least one of SEQ ID NOs: 1-1131; (b) a ribonuclease; (c) a DNase; and (d) RNA purification beads.

Embodiment 158. The kit of embodiment 157, wherein the ribonuclease is RNase H.

Embodiment 159. The kit of embodiment 157 or 158, further comprising an RNA depletion buffer, a probe depletion buffer, and a probe removal buffer.

Embodiment 160. The kit of any one of embodiments 157-160, further comprising a nucleic acid destabilizing chemical.

Embodiment 161. The kit of embodiment 160, wherein the nucleic acid destabilizing chemical comprises betaine, DMSO, formamide, glycerol, or a derivative thereof, or a mixture thereof.

Embodiment 162. The kit of embodiment 161, wherein the nucleic acid destabilizing chemical comprises formamide.

Embodiment 163. The composition or kit of any one of embodiments 154-162, wherein the at least one DNA probe comprise 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131.

Embodiment 164. The composition or kit of embodiment 163, wherein the at least one DNA probe comprises 500 or more, 600 or more, 700 or more, 800 or more, 900 or more, 1000 or more, 1100 or more, or 1131 sequences selected from SEQ ID NOs: 1-1131.

Embodiment 165. The composition or kit of any one of embodiments 154-164, wherein the at least one DNA probe comprises at least one sequence comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 166. The composition or kit of embodiment 165, wherein the at least one DNA probe comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 167. The composition or kit of embodiment 166, wherein the at least one DNA probe comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 168. The composition or kit of any one of embodiments 165-167, wherein the at least one DNA probe further comprises at least one sequence comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 169. The composition or kit of embodiment 168, wherein the at least one DNA probe comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 170. The composition or kit of embodiment 169, wherein the at least one DNA probe comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 171. The composition or kit of any one of embodiments 154-164, wherein the at least one immobilized oligonucleotide comprises at least one sequence comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 172. The composition or kit of embodiment 171, wherein the at least one immobilized oligonucleotide comprises 100 or more, 500 or more, or 1000 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 173. The composition or kit of embodiment 172, wherein the at least one immobilized oligonucleotide comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 174. The composition or kit of any one of embodiments 165-173, wherein the at least one DNA probe further comprises at least one sequence comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 175. The composition or kit of embodiment 174, wherein the at least one DNA probe comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 176. The composition or kit of embodiment 175, wherein the at least one DNA probe comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 177. A method of selecting cDNA library fragments from a library of cDNA fragments prepared from RNA, comprising (a) preparing a solid support comprising a pool of immobilized oligonucleotides, wherein each immobilized oligonucleotide in the pool comprises a nucleic acid sequence corresponding to an RNA sequence or its complement, (b) adding the library of fragments to the solid support and hybridizing the library fragments to at least one immobilized oligonucleotide to allow binding of library fragments to at least one immobilized oligonucleotide, and (c) collecting library fragments either bound or not bound to at least one immobilized oligonucleotide.

Embodiment 178. The method of embodiment 177, wherein (a) the selecting is depleting unwanted cDNA library fragments, wherein the RNA sequence comprises an unwanted RNA sequence, the unwanted library fragments comprise those prepared from unwanted RNA sequences, and the collecting comprises collecting library fragment not bound to at least one immobilized oligonucleotide; or (b) the selecting is enriching desired cDNA library fragments, wherein the RNA sequence comprises a desired RNA sequence, the desired library fragments comprise those prepared from desired RNA sequences, and the collecting comprises collecting library fragment bound to at least one immobilized oligonucleotide.

Embodiment 179. The method of embodiment 178, wherein the library of fragments is subjected to depleting unwanted cDNA library fragments and the collected library fragments not bound to at least one immobilized oligonucleotides are then subjected to enriching desired cDNA library fragments.

Embodiment 180. A solid support having two pools of immobilized oligonucleotides on its surface, wherein the first pool of oligonucleotides comprises immobilized oligonucleotides each comprising a nucleic acid sequence corresponding to an unwanted RNA sequence or its complement and the second pool of oligonucleotides comprises immobilized oligonucleotides each comprising a solid support adapter sequence that can bind to a library adapter comprised in library fragments.

Embodiment 181. The method of any one of embodiments 177-179 or the solid support of embodiment 180, wherein at least one unwanted RNA sequence has at least 90%, at least 95%, or at least 99% homology to a high-abundance RNA sequence in a sample used to prepare the library of fragments.

Embodiment 182. The method or solid support of embodiment 181, wherein the high-abundance RNA sequence is a ribosomal RNA (rRNA) sequence.

Embodiment 183. The method or solid support of embodiment 182, wherein the unwanted RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

Embodiment 184. The method or solid support of any one of embodiments 177-183, wherein each pool of immobilized oligonucleotides comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, or 1100 or more oligonucleotides.

Embodiment 185. The solid support of any one of embodiments 180-184, wherein adapter complements that are all or partially complementary to the solid support adapter sequences are bound to the solid support adapter sequences of the second pool and wherein the binding of the adapter complements to the solid support adapter sequences is reversible.

Embodiment 186. A method of amplifying desired cDNA library fragments from a library of cDNA fragments prepared from RNA, comprising (a) providing the solid support of embodiment 185; (b) adding the library of fragments to the solid support and hybridizing the library fragments to at least one immobilized oligonucleotide to allow binding of unwanted library fragments to the first pool of oligonucleotides; (c) collecting library fragments not bound to the first pool of oligonucleotides to prepare collected library fragments; (d) denaturing and removing library fragments bound to the first pool of oligonucleotides and adapter complements bound to the adapter sequences of the second pool of oligonucleotides; (e) adding the collected library fragments to the solid support and hybridizing the library fragments to at least one immobilized oligonucleotide to allow binding of desired library fragments to the second pool of oligonucleotides; and (f) amplifying the bound desired library fragments by bridge amplification on the solid support.

Embodiment 187. A method for depleting unwanted RNA molecules comprised in a patient microbiome sample, wherein the patient microbiome sample comprises at least one target RNA or DNA sequence and at least one unwanted RNA molecule, comprising (a) sequencing a plurality of probe-development microbiome samples to determine at least one unwanted RNA molecule comprising a bacterial ribosomal RNA (rRNA) sequence from sequencing data; (b) preparing a probe set comprising at least one DNA probe complementary to the at least one unwanted RNA molecule; (c) contacting the patient microbiome sample with the probe set to prepare DNA:RNA hybrids; and (d) contacting the DNA:RNA hybrids with a ribonuclease that degrades the RNA from the DNA:RNA hybrids, thereby degrading the unwanted RNA molecules in the patient microbiome sample to form a degraded mixture.

Embodiment 188. A method for depleting unwanted RNA molecules comprised in a patient microbiome sample, wherein the patient microbiome sample comprises at least one target RNA or DNA sequence and at least one unwanted RNA molecule, comprising (a) contacting the patient microbiome sample with a probe set comprising at least one sequence comprising at least one of SEQ ID NOs: 1-1131 to prepare DNA:RNA hybrids; and (b) contacting the DNA:RNA hybrids with a ribonuclease that degrades the RNA from the DNA:RNA hybrids, thereby degrading the unwanted RNA molecules in the patient microbiome sample to form a degraded mixture.

Embodiment 189. The method of embodiment 187 or embodiment 188, further comprising (a) degrading any remaining DNA probes by contacting the degraded mixture with a DNA digesting enzyme, optionally wherein the DNA digesting enzyme is DNase I, to form a DNA degraded mixture; and (b) separating the degraded RNA from the degraded mixture or the DNA degraded mixture.

Embodiment 190. A composition comprising a probe set comprising (a) at least one DNA probe comprising at least one sequence comprising at least one of SEQ ID NOs: 1-1131; and (b) a ribonuclease capable of degrading RNA in an DNA:RNA hybrid.

Embodiment 191. A kit comprising a probe set comprising (a) at least one DNA probe comprising at least one sequence comprising at least one of SEQ ID NOs: 1-1131; and (b) a ribonuclease capable of degrading RNA in an DNA:RNA hybrid.

Embodiment 192. The kit of embodiment 191, comprising (a) a probe set comprising at least one DNA probe comprising at least one of SEQ ID NOs: 1-1131; (b) a ribonuclease; (c) a DNase; and (d) RNA purification beads.

Embodiment 193. The method of any one of embodiments 177-179, 181 or 186-189, the solid support of any one of embodiments 180-185, the composition of embodiment 190, or the kit of embodiment 191 or 192, wherein the pool of oligonucleotides or the probe set comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131.

Embodiment 194. The method of any one of embodiments 177-179, 181-184, or 186-189, the solid support of any one of embodiments 180-185, the composition of embodiment 190, or the kit of embodiment 191 or 192, wherein the pool of oligonucleotides or the probe set comprises at least one sequence comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 195. The method, solid support, composition, or kit of embodiment 194, wherein the pool of oligonucleotides or the probe set comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 196. The method, solid support, composition, or kit of embodiment 195, wherein the pool of oligonucleotides or the probe set comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

Embodiment 197. The method, solid support, composition, or kit of any one of embodiments 194-196, wherein the pool of oligonucleotides or the probe set further comprises at least one sequence comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 198. The method, solid support, composition, or kit of embodiment 197, wherein the pool of oligonucleotides or the probe set comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 199. The method, solid support, composition, or kit of embodiment 198, wherein the pool of oligonucleotides or the probe set comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

Embodiment 200. The method of any one of embodiments 177-179, 181-184, or 186-189, the solid support of any one of embodiments 180-185, the composition of embodiment 190, or the kit of embodiment 191 or 192, wherein pool of oligonucleotides or the probe set comprises at least one sequence comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 201. The method, solid support, composition, or kit of embodiment 200, wherein the pool of oligonucleotides or the probe set comprises 100 or more, 500 or more, or 1000 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 202. The method, solid support, composition, or kit of embodiment 201, wherein the pool of oligonucleotides or the probe set comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

Embodiment 203. The method, solid support, composition, or kit of any one of embodiments 194-202, wherein the pool of oligonucleotides or the probe set further comprises at least one sequence comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 204. The method, solid support, composition, or kit of embodiment 203, wherein the pool of oligonucleotides or the probe set comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Embodiment 205. The method, solid support, composition, or kit of embodiment 204, wherein the pool of oligonucleotides or the probe set comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides an overview of a method of depleting unwanted library fragments derived from rRNA transcripts. A solid support, such as a flowcell, comprises at least one immobilized oligonucleotide comprising a tether to attach the oligonucleotide to the solid support. The immobilized oligonucleotide could comprise the complement of a sequence that would be comprised in a library fragment comprising an insert of cDNA prepared from the rRNA (as labeled as “rRNA complement”).

The library fragments can be flowed over the solid support, with fragment prepared from rRNA (i.e., library fragments comprising “rRNA library” sequence) hybridizing to immobilized oligonucleotides each comprising an rRNA complement. Library fragments that do not bind to the immobilized oligonucleotides can be siphoned for collection, and hybridized library fragments (i.e., unwanted library fragments) can then be denatured and siphoned to a waste container. The library fragments siphoned for collection can then be flowed over the solid support again to allow for binding of any additional unwanted library fragments, and steps of

(1) hybridizing unwanted library fragments, (2) collecting unbound library fragments, and (3) denaturing hybridized library fragment can be repeated, until a final set of collected unbound library fragments are collected that represent a library depleted of unwanted library fragments prepared from rRNA. Similar methods can be used for enrichment, wherein desired library fragments are bound to immobilized oligonucleotides comprising complementary sequences to these desired library fragments, except in the similar method the bound library fragments are used for sequencing and the library fragments that do not bind are siphoned for waste.

FIG. 2 shows an overview of a method for depleting unwanted library fragments and performing bridge amplification on the same solid support. The solid support used for this method would comprise immobilized oligonucleotides each comprising an rRNA complement, as well as immobilized oligonucleotides comprising adapter sequences that can bind to adapters comprised in library fragments. Such adapters comprised in immobilized oligonucleotides may be termed “solid support adapter sequences” and library fragments may comprise “library adapter sequences” that are all or partially complementary to the solid support adapter sequences. Solid support adapters may comprise as a P5 adapter sequence (SEQ ID NO: 1132) or a P7 adapter sequence (SEQ ID NO: 1133), and/or their complements.

Immobilized oligonucleotides comprising solid support adapter sequences may be bound to adapter complements that are all or partially complementary to the solid support adapter sequences, wherein the adapter complements hybridize to form double-stranded nucleic acid with the solid support adapter sequences. This hybridization inhibits binding of immobilized oligonucleotides comprising adapter sequences to library fragments (i.e., inhibits binding of solid support adapter sequences to library adapter sequences).

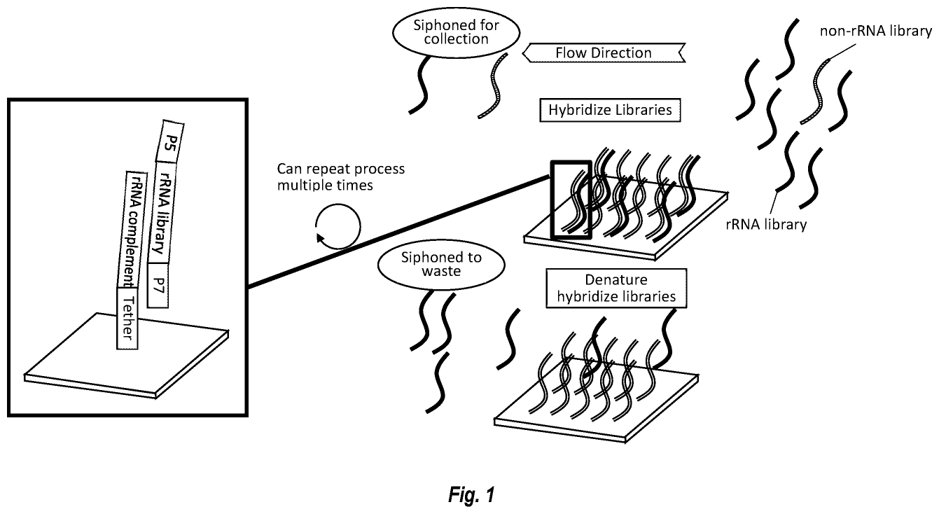

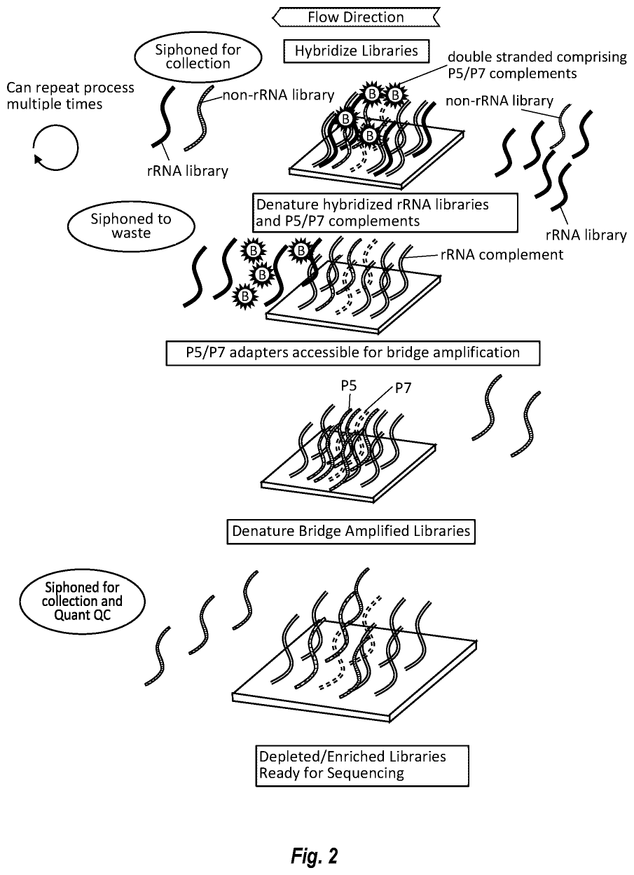

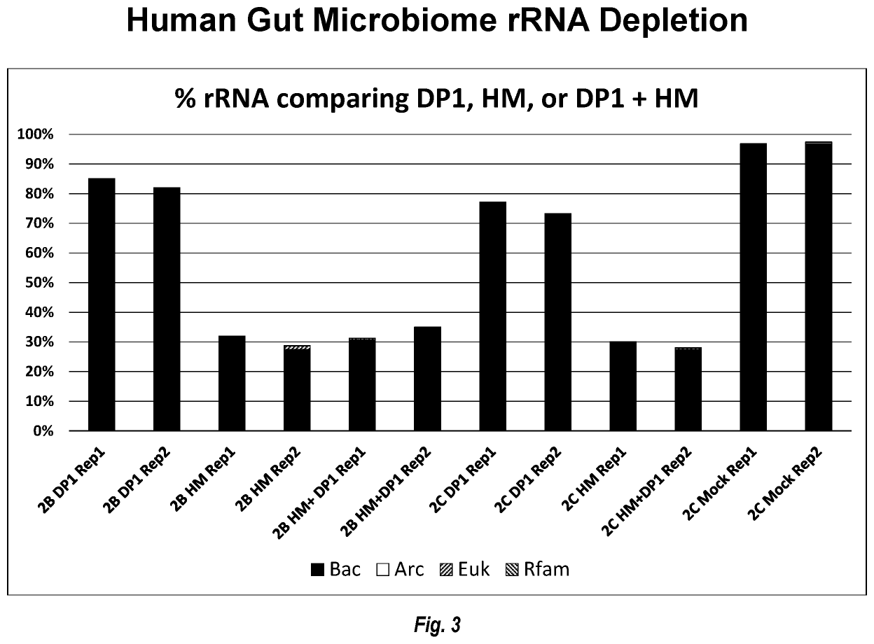

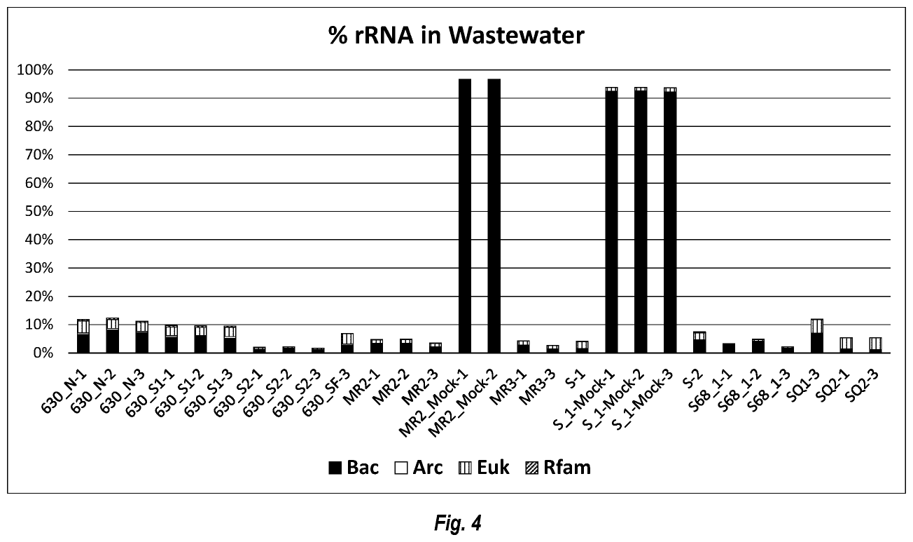

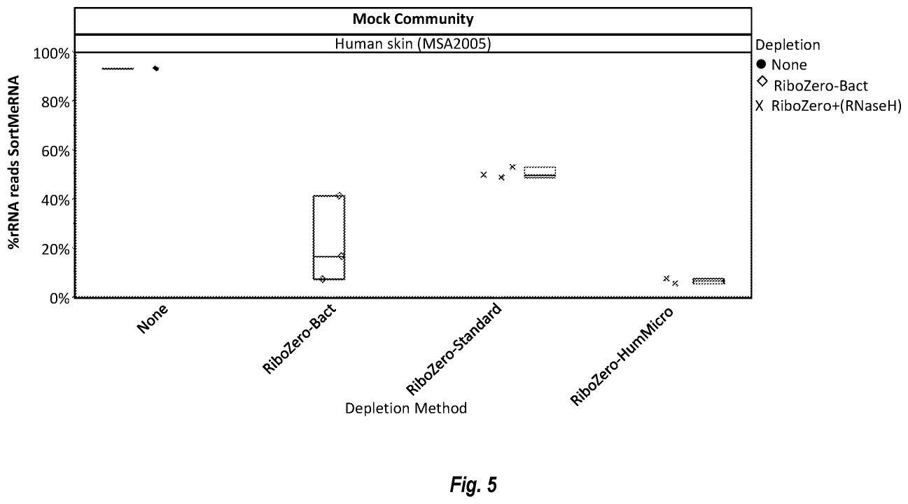

The fragments prepared from rRNA can bind to immobilized oligonucleotides each comprising an rRNA complement, as described in the legend for FIG. 1. After collecting desired library fragments (unbound to immobilized oligonucleotides each comprising an rRNA complement), unwanted library fragments and adapter complements can be denatured and siphoned to waste. The collected library fragments (comprising desired library fragments) can then be flowed over the flowcell and bound to the immobilized oligonucleotides comprising solid support adapter sequences by hybridization of library adapter sequences to solid support adapter sequences. Bridge amplification of bound library fragments can then be performed. The resulting amplified, depleted library could be sequenced, optionally after quantification and quality control.

FIG. 3 shows results on human gut microbiome rRNA depletion using the RiboZero method with RNase and standard probes (DP1) or human microbiome probes as described herein (HM, comprising HMv1 and HMv2 probes). Significantly more rRNA depletion was seen with the HM probes.

FIG. 4 shows results on rRNA depletion from wastewater samples for RiboZero depletion with HM probes or “Mock” depletion that did not include probes. Significantly more rRNA depletion was seen with the HM probes. Bac=bacterial rRNA; Arc=archaea rRNA; Euk=eukaryotic rRNA; Rfam=non-coding RNA as defined by Rfam database.

FIG. 5 shows results with a skin microbiome whole cell mix (ATCC MSA-2005™). The experiment compared results with the RiboZero RNase protocol (either with standard DP1 probes or with human microbiome HM probes) to those with the RiboZero-Bact that uses a probe-based hybridization approach to capture and deplete bacterial rRNAs from E. coli and B. subtilis.

### DESCRIPTION OF THE SEQUENCES

Table 1 provides a listing of certain sequences referenced herein.

TABLE 1
Description of the Sequences

| | | SEQ |
|---|---|---|
| Description | Sequences | ID NO |
| Representative | As shown in Table 2 | 1- |
| sequences comprised | | 1131 |
| in immobilized | | |
| oligonucleotides | | |
| P5 | AATGATACGGCGACCACCGAGA | 1132 |
| | UCTACAC | |
| P7 | CAAGCAGAAGACGGCATACGAG | 1133 |
| | AT | |
| A14 | TCGTCGGCAGCGTC | 1134 |
| B15 | GTCTCGTGGGCTCGG | 1135 |

### DESCRIPTION OF THE EMBODIMENTS

### I. Solid Supports for Enriching or Depleting

In some embodiments, solid supports can be prepared for enriching desired library fragments or depleting unwanted library fragments, wherein at least oligonucleotide is immobilized to the solid support. In some embodiments, the solid support is a flowcell.

In some embodiments, the at least one immobilized oligonucleotide comprises a sequence comprising any one or more of SEQ ID NOs: 1-1131.

In some embodiments, the at least one immobilized oligonucleotide comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131 or its complement. In some embodiments, the at least one immobilized oligonucleotide comprises 500 or more, 600 or more, 700 or more, 800 or more, 900 or more, 1000 or more, 1100 or more, or 1131 sequences selected from SEQ ID NOs: 1-1131 or its complement.

In some embodiments, the at least one immobilized oligonucleotide comprises at least one sequence from a bacterial ribosomal RNA (rRNA) or its complement. In some embodiments, the at least one immobilized oligonucleotide comprises at least one sequence comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

In some embodiments, the at least one immobilized oligonucleotide comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement. In some embodiments, the at least one immobilized oligonucleotide comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

In some embodiments, the at least one immobilized oligonucleotide comprises at least one sequences of B. Bifidum rRNA or its complement. In some embodiments, the at least one immobilized oligonucleotide further comprises at least one sequence comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement. In some embodiments, the at least one immobilized oligonucleotide comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement. In some embodiments, the at least one immobilized oligonucleotide comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

In some embodiments, the at least one immobilized oligonucleotide comprises 100 or more, 500 or more, or 1000 or more sequences comprising at least one of SEQ ID NOs: or its complement. In some embodiments, the at least one immobilized oligonucleotide comprises a sequence comprising each of SEQ ID NOs: or its complement.

In some embodiments, the at least one immobilized oligonucleotide further comprises at least one sequence comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127. In some embodiments, the at least one immobilized oligonucleotide comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 In some embodiments, the at least one immobilized oligonucleotide comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

Also disclosed herein are compositions comprising a library fragment bound to an immobilized oligonucleotide on a solid support. In some embodiments, a single-stranded library fragment comprising cDNA prepared from a sample comprising RNA is hybridized to a solid support comprising immobilized oligonucleotides. In some embodiments, the cDNA comprised in the composition is complementary to RNA comprised in the sample.

Disclosed herein are also kits for depleting or enriching libraries. In some embodiments, the kit comprises a solid support disclosed herein and instructions for using the solid support. Such a kit may further comprise reagents for preparing a cDNA library from RNA, such as reagents for a stranded method of cDNA preparation from a sample comprising RNA, as described below.

A. Types of Solid Supports

A wide variety of solid supports may be used to immobilize oligonucleotides for depleting or enriching as described herein, including those described in WO 2014/108810, which is incorporated in its entirety herein.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, a flowcell is comprised within an apparatus or device for sequencing nucleic acids, which may be referred to as a sequencer. In some embodiments, a sequence may also comprise reservoirs for collection of samples or tubing (such as for collecting samples in a reservoir of for exiting of waste). In some embodiments, one or more reservoirs are separate from the flowcell and are comprised in the sequencer. In some embodiments, modifications are made to standard sequencers to improve fluidics system recipes and/or hardware for use of reservoirs in the present methods.

As used herein, a “flowcell” may comprise a flowcell-like device that is not intended to be imaged. While standard flowcells used for imaging may be employed in the present methods, flowcells can also be engineered differently than flowcells intended for imaging. In some embodiments, a flowcell may have a high density of immobilized oligonucleotides, wherein imaging infrastructure would have difficulty separating out into different bridge-amplified clusters associated with different immobilized oligonucleotides. In some embodiments, a high density of immobilized oligonucleotides improves hybridization efficiency. In some embodiments, standard clear glass may be used in a flowcell. In other embodiments, hard plastic may be used in the flowcell. Use of glass in a flowcell may allow use of a standard flowcell without further optimization, whereas use of hard plastic may reduce the cost of manufacturing the flowcell and/or improve stability of a flowcell. Depending on the advantages desired, different materials may be used. In some embodiments, immobilized oligonucleotides are embedded in a substrate other than that of a standard flowcell (i.e., embedded in a substrate other than PAZAM) to improve immobilization of oligonucleotides of longer length.

B. Unwanted RNA

As used herein, “unwanted RNA” or “an unwanted RNA sequence” refers to any RNA that a user does not wish to analyze. As used herein, an unwanted RNA includes the complement of an unwanted RNA sequence. When RNA is converted into cDNA and this cDNA is prepared into a library, a user would sequence library fragments that were prepared from all RNA transcripts in the absence of enrichment or depletion. Methods described herein for depleting library fragments prepared from unwanted RNA can thus save the user time and consumables related to sequencing and analyzing sequencing data prepared from unwanted RNA.

As used herein, “unwanted RNA” or “unwanted RNA sequence” also includes fragments of such RNA. For example, an unwanted RNA may comprise part of the sequence of an unwanted RNA. In some embodiments, unwanted RNA sequence is from human, rat, mouse, or bacteria. In some embodiments, the bacteria are Archaea species, E. Coli, or B. subtilis.

As used herein, “unwanted library fragments” refers to library fragments prepared from cDNA prepared from unwanted RNA.

In some embodiments, the unwanted RNA sequence comprises any one or more of SEQ ID NOs: 1-1131.

In some embodiments, unwanted RNA sequences (or their complements) are immobilized to a solid support. A range of different types of RNA may be unwanted.

1. High-Abundance RNA

In some embodiments, the unwanted RNA is high-abundance RNA. High-abundance RNA is RNA that is very abundant in many samples and which users do not wish to sequence, but it may or may not be present in a given sample. In some embodiments, the high-abundance RNA sequence is a ribosomal RNA (rRNA) sequence. Exemplary high-abundance RNA are disclosed in WO2021/127191 and WO 2020/132304, each of which is incorporated by reference herein in its entirety.

In some embodiments, the high-abundance RNA sequences are the most abundant RNA sequences determined to be in a sample. In some embodiments, the high-abundance RNA sequences are the most abundant RNA sequences across a plurality of samples even though they may not be the most abundant in a given sample. In some embodiments, a user utilizes a method of determining the most abundant RNA sequences in a sample, as described herein.

In a given sample, the most abundant sequences are the 100 most abundant sequences. In some embodiments, the in addition to depleting the 100 most abundant sequences, the method also is capable of depleting the 1,000 most abundant sequences, or the 10,000 most abundant sequences in a sample. In some embodiments, the unwanted RNA sequence comprises a sequence with homology of at least 90%, at least 95%, or at least 99% to a most abundant sequence in a sample comprising RNA. In some embodiments, the unwanted RNA sequence comprises a sequence with homology of at least 90%, at least 95%, or at least 99% to a most abundant sequence in a sample comprising RNA, wherein the most abundant sequences comprise the 100 most abundant sequences. In some embodiments, homology is measured against the 1,000 most abundant sequences, or the 10,000 most abundant sequences.

In some embodiments, the high-abundance RNA sequences are comprised in RNA known to be highly abundant in a range of samples.

In some embodiments, the unwanted RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

In some embodiments, the unwanted RNA sequence is 28S, 18S, 5.8S, 5S, 16S, or 12S RNA from humans, or a fragment thereof. In some embodiments, the unwanted RNA sequence is rat 16S, rat 28S, mouse 16S, or mouse 28S RNA.

In some embodiments, the unwanted RNA sequence is comprised in mRNA related to one or more “housekeeping” genes. For example, a housekeeping gene may be one that is commonly expressed in a sample from a tumor or other oncology-related sample, but that is not implicated in tumor genesis or progression

In some embodiments, the unwanted RNA sequence is comprised in 23S, 16S, or 5S RNA from Gram-positive or Gram-negative bacteria. In some embodiments, the unwanted RNA sequence is from an organism in the human microbiome.

2. Host RNA

In some embodiments, the unwanted RNA sequence is comprised in a host transcriptome. For example, a user may wish to study library fragments prepared from RNA from organisms comprised in the human microbiome, without analyzing library fragments prepared from human RNA.

C. Desired RNA

As used herein, “desired RNA” or “a desired RNA sequence” refers to any RNA that a user wants to analyze. As used herein, a desired RNA includes the complement of a desired RNA sequence. Desired RNA may be RNA from which a user would like to collect sequencing data, after cDNA and library preparation. In some instances, the desired RNA is mRNA (or messenger RNA). In some instances, the desired RNA is a portion of the mRNA in a sample. For example, a user may want to analyze RNA transcribed from cancer-related genes, and thus this is the desired RNA. In another example, a user may wish to analyze RNA from organisms comprised in a human microbiome, and thus RNA from organisms comprised in the human microbiome is the desired RNA and human RNA is the unwanted RNA.

As used herein, “desired library fragments” refers to library fragments prepared from cDNA prepared from desired RNA.

In some embodiments, the desired RNA sequence is an exome sequence. In some embodiments, the present methods are for exome enrichment.

In some embodiments, the desired RNA sequence is from human, rat, mouse, and/or bacteria. In some embodiments, the desired RNA sequence is from an organism in the human microbiome.

D. Immobilized Oligonucleotides for Enriching or Depleting

In some embodiments, oligonucleotides for enriching or depleting are immobilized to a solid support. Such immobilized oligonucleotides may be referred to as tethered to the solid support. In some embodiments, the oligonucleotide may be immobilized to the solid support via a linker molecule. When referring to immobilization of oligonucleotides to a solid support, the terms “immobilized” and “attached” are used interchangeably herein and both terms are intended to encompass direct or indirect, covalent or non-covalent attachment, unless indicated otherwise, either explicitly or by context. In certain embodiments of the invention covalent attachment may be preferred, but generally all that is required is that the at least one immobilized oligonucleotide remains immobilized or attached to the support under the conditions in which it is intended to use the support, for example for enriching or depleting.

As used herein, a “tether” refers to any means of immobilizing an oligonucleotide to a solid support. In some embodiments, a solid support, such as a flowcell, is coated with a covalently attached polymer. In some embodiments, a flowcell contains a polymer coating. In some embodiments, the covalently attached polymer is PAZAM. In some embodiments, the polymer coating comprises reactive sites for reacting with oligonucleotides, such as oligonucleotides described herein. Such covalently attached polymers are described in WO 2013/184796, which is incorporated by reference in its entirety herein. In some embodiments, a polymer such as PAZAM is crosslinked using ultraviolet light.

In some embodiments, immobilized oligonucleotides may be designed to comprise a cleavage site. In some embodiments, a method may comprise a step to cleave immobilized oligonucleotides to remove them from the solid support. In some embodiments, after cleavage of the immobilized oligonucleotides, the resulting fragments from the immobilized oligonucleotides are collected in a waste container comprised in a sequencer. In some embodiments, a tether may comprise a cleavage site. In this way, some or all of the immobilized oligonucleotides on a solid surface can be removed at the user's discretion, potentially avoiding a requirement to transfer a sample to a different solid support.

In some embodiments, immobilized oligonucleotides described herein are single-stranded. In this way, the immobilized oligonucleotides are available to hybridize to single-stranded library fragments that are all of partially complementary to a sequence comprised in the immobilized oligonucleotides. One skilled in the art could design the length of immobilized oligonucleotides to allow for their preferred level of affinity for the interaction between immobilized oligonucleotides and library fragments that are all or partially complementary (i.e., longer immobilized oligonucleotides would be expected to exhibit higher affinity binding to single-stranded library fragments that are all or partially complementary).

In some embodiments, a sequence comprised in an immobilized oligonucleotide can be partly or completely complementary to the sequence of a library fragment prepared from an unwanted RNA for depletion. In some embodiments, a sequence comprised in an immobilized oligonucleotide can be partly or completely complementary to the sequence of a library fragment prepared from a desired RNA for enrichment.

In some embodiments, each immobilized oligonucleotide is from 10 to 100 nucleotides long, from 20 to 100 nucleotides long, from 20 to 80 nucleotides long, from 40 to 60 nucleotides long, from 45 to 55 nucleotides long, or 50 nucleotides long. In some embodiments, the at least one immobilized oligonucleotide is 45-55 bases in length, optionally wherein the at least one immobilized oligonucleotide is 50 bases in length. In some embodiments, an immobilized oligonucleotide has a molecular weight (M.W.) of 15,000 to 15,500 Daltons.

In some embodiments, multiple different oligonucleotides comprising a sequence all or partially complementary to an unwanted or desired RNA may be immobilized on a solid support. In some embodiments, these multiple different oligonucleotides are all or partially complementary to different sequences comprised in an unwanted or desired RNA. For example, if a user wants to deplete a given rRNA, the user may prepare multiple oligonucleotides with overlapping or non-overlapping sequences corresponding to this rRNA. In some embodiments, having multiple immobilized oligonucleotides corresponding to different sequences from a given unwanted RNA can improve efficiency of depleting of library fragments prepared from this RNA. In some embodiments, having multiple immobilized oligonucleotides corresponding to different sequences from a given desired RNA can improve efficiency of enrichment of library fragments prepared from this RNA. In part, this improved efficiency may be because library fragments may be generated randomly from cDNA prepared from a given RNA, and a user cannot predict the specific insert sequence of cDNA comprised in a given fragment.

In some embodiments, a sequence comprised in an immobilized oligonucleotide can be completely or partially complementary to a particular location on the RNA to be depleted or enriched (i.e., a target location), for example the sequence comprised in an immobilized oligonucleotide can be at least 80%, 85%, 90%, 95%, or 100% complementary, or any range in between, to a target location on an RNA transcript to be depleted or enriched.

In some embodiments, immobilized oligonucleotides may bind to a set of different sequences comprised in an RNA to be depleted. In some embodiments, multiple immobilized oligonucleotides may be designed that tile an RNA sequence intended for depletion, such as the tiling described in WO 2020132304, which is incorporated herein in its entirety. In some embodiments, multiple immobilized oligonucleotides designed against a target sequence can increase the likelihood of binding of a fragment generated from the target sequence to at least one immobilized oligonucleotide. For example, library inserts comprised in library fragments may comprise approximately 150 bp, and the immobilized oligonucleotides described herein may comprise 50-80 nucleotides. In such a scenario, if a fragmentation event occurs within the target sequence and disrupts binding of a given immobilized oligonucleotide to the fragment (such as if the fragmentation occurs within a sequence that can bind to a given immobilized oligonucleotide), an immobilized oligonucleotide designed to bind an adjacent target sequence may likely be able to hybridize to the fragment. In this way, tiling of sequences can increase the likelihood of successful depletion or enrichment of fragments prepared from an RNA sequence.

In some embodiments, the present oligonucleotides comprise modified or unmodified nucleic acid.

As used herein, a “modified nucleic acid” refers to any substitution from a naturally occurring nucleic acid. For example, a modified nucleic acid may comprise one or more modifications to the sugar-phosphate backbone or the pendant base groups. Such modifications can improve stability of immobilized oligonucleotides.

In some embodiments, one, at least one, or each of the one or more immobilized nucleic acids comprises RNA, deoxyribonucleic acid (DNA), xeno nucleic acid (XNA), or a combination thereof. The XNA can comprise 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), Fluoro Arabino nucleic acid (FANA), or a combination thereof.

In some embodiments, an immobilized nucleic acid consists of modified nucleic acids. In some embodiments, a certain percentage of the nucleic acids comprised in an immobilized nucleic acid are modified nucleic acids, for example every third nucleotide may be a modified nucleic acid.

In some embodiments, the at least one immobilized oligonucleotide comprises the sequence or complementary sequence of an unwanted RNA. Solid supports with such immobilized oligonucleotides comprising the sequence or complementary sequence of unwanted RNA may be used for depleting library fragments prepared from unwanted RNA using methods described herein.

In some embodiments, the at least one immobilized oligonucleotide comprises the sequence or complementary sequence of a desired RNA. Solid supports with such immobilized oligonucleotides comprising the sequence or complementary sequence of desired RNA may be used for enriching library fragments prepared from desired RNA using methods described herein.

1. Immobilized Oligonucleotides for Depleting

In some embodiments, oligonucleotides for depleting comprise one or more unwanted RNA sequence.

In some embodiments, immobilized oligonucleotides are designed to deplete unwanted library fragments from a library. In some embodiments, the unwanted library fragments comprise library fragments prepared from unwanted RNA. A representative example of a solid support with immobilized oligonucleotides for depleting unwanted library fragments is shown in FIG. 1.

In some embodiments, immobilized oligonucleotides are designed to deplete each of most abundant species that are determined from a sample.

Various unwanted types of unwanted RNA (such as rRNA) are well-known in the literature. The RiboZero+probes and nuclease-based depletion of abundant transcripts using the RiboZero+probes have been described in WO 2020/132304A1, the content of which is incorporated by reference in its entirety.

In some embodiments, immobilized oligonucleotides are designed for depleting abundant transcripts described in WO 2020/132304A1.

In some embodiments, unwanted RNA sequences are determined by assessing sequencing results to determine abundant sequences in a sample comprising RNA. In some embodiments, the unwanted RNA sequences are selected by determining the most abundant sequences in a sample comprising RNA. In some embodiments, the most abundant sequences comprise the 100 most abundant sequences, the 1,000 most abundant sequences, or the 10,000 most abundant sequences. In some embodiments, the unwanted RNA sequence comprises a sequence with homology of at least 90%, at least 95%, or at least 99% to a most abundant sequence in a sample comprising RNA. In some embodiments, the unwanted RNA sequence comprises a sequence with homology of at least 90%, at least 95%, or at least 99% to a most abundant sequence in a sample comprising RNA, wherein the most abundant sequences comprise the 100 most abundant sequences, the 1,000 most abundant sequences, or the 10,000 most abundant sequences.

WO 2021/127191, which is incorporated herein in its entirety, describes methods of selecting abundant regions from a sample comprising RNA. Immobilized oligonucleotides can be designed using methods from WO 2021/127191 of identifying abundant regions using standard publicly available software. In some embodiments, methods of identifying abundant regions can avoid bias towards known samples within an environmental sample.

An exemplary type of immobilized oligonucleotides for use in depleting library fragments prepared from unwanted RNA (i.e., unwanted library fragments), is shown in FIG. 1. In some embodiments, the unwanted library fragments are prepared from rRNA and may be termed an “rRNA library.” In some embodiments, the rRNA library comprises library fragments prepared from a first strand of cDNA prepared from RNA. When the unwanted library fragments are an rRNA library, an immobilized oligonucleotides may be an “rRNA complement” that can bind to the rRNA library. Immobilized oligonucleotides comprising an rRNA complement are one representative type of immobilized oligonucleotide for use in depleting, and one skilled in the art could design such oligonucleotides for depleting library fragments prepared from any type of unwanted RNA. In some embodiments, unwanted RNA may be comprised in some immobilized oligonucleotides, and the complement of unwanted RNA may be comprised in other immobilized oligonucleotides.

2. Representative Sequences Comprising in Immobilized Oligonucleotides for Depleting

Table 1 describes a set of sequences that may comprised in immobilized oligonucleotides. Immobilized oligonucleotides or their complements listed in Table 2 may have particular use in studies of microbiome samples.

The immobilized oligonucleotides listed in Table 2 were designed by sequencing total RNA derived from human fecal matter to identify abundant rRNA sequences that were detected using the publicly available rRNA classifier SortMeRNA (as described in Kopylova et al., Bioinformatics 28(24):3211-3217 (2012)). The most abundant transcripts were identified, and DNA probes were designed against these transcripts. The depletion has been tested with fecal, skin, oral and vaginal samples using the Total RNA stranded kit as well as with samples derived from various soil types with much better results in comparison to a standard depletion probe panel (data not shown). The oligonucleotides listed in Table 2 are designed to remove rRNA sequences from metatranscriptomics samples, such as stool, and are antisense to the rRNA sequence that they target. In some embodiments, the at least one immobilized nucleotide comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131 or its complement. In some embodiments, the at least one immobilized nucleotide comprises 500 or more, 600 or more, 700 or more, 800 or more, 900 or more, 1000 or more, 1100 or more, or 1131 sequences selected from SEQ ID NOs: 1-1131 or its complement.

In some embodiments, the at least one immobilized oligonucleotide comprises at least one sequence comprised in the HMv1 sequences and comprising SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

In some embodiments, the at least one immobilized oligonucleotide comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

In some embodiments, the at least one immobilized oligonucleotide comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130 or its complement.

In some embodiments, the at least one immobilized oligonucleotide further comprises at least one sequence comprised in the HMv2 sequences and comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

In some embodiments, the at least one immobilized oligonucleotide comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

In some embodiments, the at least one immobilized oligonucleotide comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131 or its complement.

In some embodiments, the at least one immobilized oligonucleotide further comprises at least one sequence comprised in the HM sequences (comprising both HMv1 and HMv2 probes) and comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131 or its complement.

In some embodiments, the at least one immobilized oligonucleotide comprises 10 or more, 20 or more, or 30 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131 or its complement.

In some embodiments, the at least one immobilized oligonucleotide comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131 or its complement.

In some embodiments, the at least one immobilized oligonucleotide further comprises at least one sequence comprised in the DP1 sequences and comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 or its complement.

In some embodiments, the at least one immobilized oligonucleotide comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 or its complement.

In some embodiments, the at least one immobilized oligonucleotide comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127 or its complement.

TABLE 2
Representative immobilized oligonucleotides

| | | Calc. | | |
|---|---|---|---|---|
| SEQ | | Molecular | M.W. | M.W. |
| ID NO | Sequence | Weight | Lower Limit | Upper Limit |
| 1 | CTCATCCCCACCCTTTTCAACGGATGTGGGTTCGGTCCTCCACTGCCTCT | 15127 | 15111.873 | 15142.127 |
| 2 | AGCCGGGGCTTCTTAGTCAGGTACCGTCATTTTTTCTTCCCTGCTGATAG | 15316 | 15300.684 | 15331.316 |
| 3 | TAGATGATCAACCTACCGGGTTAGAGTAGCCATCACACAAGGGTAGTATC | 15427 | 15411.573 | 15442.427 |
| 4 | CAGATGGCGGCATTGTCACTGCTCCGTCTCCACGTCACTCCTGAAGGTAG | 15314 | 15298.686 | 15329.314 |
| 5 | GGGAAGCAGGGTGGACCACCACCCAAGGCTAAATACTACCTGATGACCGA | 15432 | 15416.568 | 15447.432 |
| 6 | ACTAAACTTCACTCCGCATCACGTCTTCCCATTGCCGCACGGTTTTTCCA | 15103 | 15087.897 | 15118.103 |
| 7 | GTTCCTCCGCTTGTGCGGGCCCCCGTCAATTCCTTTGAGTTTCACCGTTG | 15229 | 15213.771 | 15244.229 |
| 8 | GCCCCAGACAACCATCGCTGGGGTTGAGCTACCTCACTGCGTCCCTCCGC | 15205 | 15189.795 | 15220.205 |
| 9 | CTTTCGTGCGGGTCGGAACTTACCCGACAAGGAATTTCGCTACCTTAGGA | 15377 | 15361.623 | 15392.377 |
| 10 | CAGGCGTCAGCTCGTATACGTCATCTTTCGATTTAGCACAAACCTGTGTT | 15302 | 15286.698 | 15317.302 |
| 11 | GGCTTCATGCTTAGATGCTTTCAGCACTTATCCCGTCCGCACATAGCTAC | 15223 | 15207.777 | 15238.223 |
| 12 | ATTACCGCGGCTGCTGGCACGTAGTTAGCCGGGGCTTCTTAGTCAGGTAC | 15425 | 15409.575 | 15440.425 |
| 13 | TTCACGCAAGATTTCTCGTGTCCCGCGCTACTCAGGATACCACTACGCTT | 15208 | 15192.792 | 15223.208 |
| 14 | ATCTAAAGTCTTCTCGTTTAAAATACTGGGCTGTTACCATCTGTGGCGGA | 15381 | 15365.619 | 15396.381 |
| 15 | GGGCTCTGACTTCTTGTAGGCATACGGTTTCAGGTTCTCTTTCACTCCGC | 15292 | 15276.708 | 15307.292 |
| 16 | GCTATGGATCGTCGGTTTGGTGGGCCGTTACCCCGCCAACTGCCTAATCC | 15321 | 15305.679 | 15336.321 |
| 17 | ATGACTTCAGCATGGGCGGTCATAACGCGGTACCAGAATATCAACTGGTT | 15434 | 15418.566 | 15449.434 |
| 18 | TTTCAGTTCAGGCGGTTCCCCTCATATACCTATGTATTCAGTATATGATG | 15307 | 15291.693 | 15322.307 |
| 19 | CGAAAGGGGAGACGGCACGGGCCCGGAGGTTAGCGCCCCAGGCCTCGGTT | 15529 | 15513.471 | 15544.529 |
| 20 | TTTCGTCCCTGCTCGACTTGTAGGTCTCGCAGTCAAGCTCCCTTGTGCCT | 15213 | 15197.787 | 15228.213 |
| 21 | CTCTTATCGATGACATCTCCTCTTAACCTTCCAGCACCGGGCAGGTGTCA | 15208 | 15192.792 | 15223.208 |
| 22 | TCGTCCCTGACAACAGAGCTTTACGATCCGAAAACCTTCTTCACTCACGC | 15170 | 15154.83 | 15185.17 |
| 23 | ACCCAACATCTCACGACACGAGCTGACGACAACCATGCACCACCTGTCAC | 15152 | 15136.848 | 15167.152 |
| 24 | GTCCTCTCGTACTAAGGACAGAGCTCCTCAAATATCCTGCGCCCACGACA | 15220 | 15204.78 | 15235.22 |
| 25 | TTATAGTTACGGCCGCCGTTTACCGGGGCTTCAATTCAGAGCTCTCACTC | 15279 | 15263.721 | 15294.279 |
| 26 | CGTTTCTACGAGTTAGAACTCAAATAATCAAAGGGCCGTATTTCAACAGC | 15361 | 15345.639 | 15376.361 |
| 27 | CACCAGTGTCGGTTTAGGGTACGGGCGGACCCGCCACCTCGCTCACGAAG | 15374 | 15358.626 | 15389.374 |
| 28 | CGTCCATCCCGGTCCTCTCGTACTAGGGACAGCTCCTCTCAAATATCCTG | 15169 | 15153.831 | 15184.169 |
| 29 | AGCTGACGCTCATGTTTCCAAGTCTCCCGCCTATCCTGTACATAGATTTC | 15198 | 15182.802 | 15213.198 |
| 30 | CTCTTTTAATGAGTGGCTGCTTCTAAGCCAACATCCTGGTTGTCTAAGCA | 15317 | 15301.683 | 15332.317 |
| 31 | ACAGCTTTTCTCGCCATCTTCCATCCCAGACTTCGGTACTAACTTCCCTC | 15054 | 15038.946 | 15069.054 |
| 32 | CATAGACCTGTGTTTTTGCTAAACAGTTGCTTGAGCCTATTCTCTGCGGC | 15324 | 15308.676 | 15339.324 |
| 33 | TCACGGTACTGGTTCACTATCGCTCACTCGTTTATATTTAGCCTTGGCGG | 15300 | 15284.7 | 15315.3 |
| 34 | ACTCACCCTGCCCCGATTAACGTTGGACAGGAACCCTTGGTCTTCCGGCG | 15259 | 15243.741 | 15274.259 |
| 35 | GGCTACAGTAAAGCTCCATGGGGTCTTTCCGTCTTGTCGCGGGTAACCGG | 15425 | 15409.575 | 15440.425 |
| 36 | GTACGATTTGATGTTACCTGATGCTTAGAGGCTTTTCCTGGAAGCAGGGC | 15478 | 15462.522 | 15493.478 |
| 37 | AAGTCATTGGCATTCGGAGTTTGACTGAATTCGGTAACCCGGTAGGGGCC | 15497 | 15481.503 | 15512.497 |
| 38 | GGTTACCTTGTTACGACTTCACCCCAGTCATGAATCACAAAGTGGTAAGT | 15344 | 15328.656 | 15359.344 |
| 39 | CCCTTCTCCCGTTGGCCTTAGAATCTTCTTCCTACCTACCTGTGTCGGTT | 15123 | 15107.877 | 15138.123 |
| 40 | TACCTTCACTAAGGTTCTTTCCGACGCTAGCCCTAAAGCTATTTCGGGGA | 15287 | 15271.713 | 15302.287 |
| 41 | CCCCCCTGCTTCCCACAGGGTTTCACGTGTCCCGTGGTACTCTGGATCAC | 15177 | 15161.823 | 15192.177 |
| 42 | GACCGGCCTTCCCATGCCGTTCGGTTAACAGATTAAGTCTTAAAAGCAGT | 15345 | 15329.655 | 15360.345 |
| 43 | TTCCTTTGACCCCCCCCCCCCCCCTCCCTATCCCCCCCCGCCCCCCCCCA | 14669 | 14654.331 | 14683.669 |
| 44 | CCCCCTCAGTTCTCCAGCGCCCACGGCAGATAGGGACCGAACTGTCTCAC | 15198 | 15182.802 | 15213.198 |
| 45 | CTTTGGGAGGCAACCGCCCCAGTTAAACTACCCGCCAGGCACTCTCCCCG | 15198 | 15182.802 | 15213.198 |
| 46 | ACATGATCGGTTCACACACTCACCACCACACAAGACCTCAAAGAGACCCC | 15160 | 15144.84 | 15175.16 |
| 47 | CCAGCACCGGGCAGGTGTCACCCCCTATACTTCGTCTTGCGACTTCGCAG | 15235 | 15219.765 | 15250.235 |
| 48 | GTACCGCTTTAATGGGCGAACAGCCCAACCCTTGGGACTGACTACAGCCC | 15301 | 15285.699 | 15316.301 |
| 49 | CCATTGCGGAAGATTCCCTACTGCTGCCTCCCGTAGGAGTCTGGGCCGTG | 15346 | 15330.654 | 15361.346 |
| 50 | TTCTCTGCGGCTCATGTTTCCATGAGCACCCCTTATCCCTAAGTTACGGG | 15230 | 15214.77 | 15245.23 |
| 51 | TTTGACTCATATCACACCTCACTGCTTAGACGTGCACTTCCAATCGCACG | 15176 | 15160.824 | 15191.176 |
| 52 | CCGGTTTGCCCTCTTCCGCGTTCGCTCGCCACTACTTACGGAATCTCGTT | 15158 | 15142.842 | 15173.158 |
| 53 | TACCTGATCGACTTGTCAGTCTCCCAGTCAAGCGCCCTTATGCCATTACA | 15192 | 15176.808 | 15207.192 |
| 54 | TCCCAAGCTTCGGTGTATGATTTAGCCCCGTTAAATTTTCGGCGCAGGGT | 15374 | 15358.626 | 15389.374 |
| 55 | CCTAGTCTTTTCAGTGCTCTACAAGCCGTGGTCATGGTTCGAGGCTGTAC | 15350 | 15334.65 | 15365.35 |
| 56 | TCGGGGTGCTTTTCACCTTTCCTTCACAGTACTCGTACGCTATCGGTCTC | 15212 | 15196.788 | 15227.212 |
| 57 | GGTCTGGGCTCTTTCCCTTTCGACTGCCCAACTTATCTCGTGCAGTCTGA | 15237 | 15221.763 | 15252.237 |
| 58 | GCACTCCACAGCTCCTTCCGGTACTGCTTCTTCGCGTTAAGAATGCTCCT | 15175 | 15159.825 | 15190.175 |
| 59 | GACTGCGAACCGTGAGCATTCGGAGTTCGTCAGGACTCGATAGGCGGTGA | 15532 | 15516.468 | 15547.532 |
| 60 | GTAAACAGTCGCTTGGGTCTATTCTCTGCGGCCCATTCCTGGGCACTCCT | 15271 | 15255.729 | 15286.271 |
| 61 | CCCACTTTCGTGCCTGCTCGACGTGTCTGTCTCGCAGTCAAGCCACCTTG | 15192 | 15176.808 | 15207.192 |
| 62 | TTTCCCTGCGGCTCCGGGACTTTATCCCTTAACCTTGCCAGTATGCACAA | 15199 | 15183.801 | 15214.199 |
| 63 | GGGCGCCTTCGCTTCGTAGCAGCTTTTCTCGCCAGCGTGAATTCAGCAGC | 15321 | 15305.679 | 15336.321 |
| 64 | TTCCGCCTGACCTTAGCTCCCGACTAACCCTGAGCGGACGAACCTTCCTC | 15139 | 15123.861 | 15154.139 |
| 65 | CTCTCAGGTCGGCTACTGATCGTCGGCTTGGTAGGCCGTTACCCCACCAA | 15290 | 15274.71 | 15305.29 |
| 66 | CTTCCTCCGGCTACTTAGATGTTTCAGTTCACCGGGTTCCCCTCCATACG | 15166 | 15150.834 | 15181.166 |
| 67 | TACCTGATCGACTTGTTAGTCTCCCAGTCAAGCGCCCTTATGCCATTACA | 15207 | 15191.793 | 15222.207 |
| 68 | GCAACCGCCCCAGTTAAACTACCCGCCAGGCACTGTCCCTGAACAGGATG | 15255 | 15239.745 | 15270.255 |
| 69 | TTCCTCGTGTCTCGCCGTACTCAGGATCCCATTAGGCTTCGATCGGATTT | 15261 | 15245.739 | 15276.261 |
| 70 | ACGGATCGTCGCCTTGGTAGGCCTTTACCCCACCAACTAGCTAATGCACC | 15227 | 15211.773 | 15242.227 |
| 71 | TGTCGGTTTGGGGTACGGGCGGCAACGCGCCTGACGCCGGGGCTTTTCTC | 15474 | 15458.526 | 15489.474 |
| 72 | CGGTTTCCGTTCGCGCTGAGGGAACCTTTGGGCGCCTCCGTTACATTTTG | 15358 | 15342.642 | 15373.358 |
| 73 | TTATAGTTACGGCCGCCGTTTACTGGGGCTTCAATTCAATGCTCTCACAT | 15293 | 15277.707 | 15308.293 |
| 74 | TGTAGCATGCGTGAAGCCCTGGACGTAAGGGGCATGATGATCTGACGTCA | 15531.1 | 15515.5689 | 15546.6311 |
| 75 | AGCACCGGGCAGGTGTCAGCACCTATACGTCAGCTCTCGCTTTCGCAGAT | 15323 | 15307.677 | 15338.323 |
| 76 | GCTGATAGGACGCGACCCCATCCCACGCCGATAGAATCTTTCCCACAATC | 15204.9 | 15189.6951 | 15220.1049 |
| 77 | GTTTCAGGTTCTATTTCACTCCCCTCCCGGGGTGCTTTTCACCTTTCCCT | 15114 | 15098.886 | 15129.114 |
| 78 | CGGCTCCCATTCCGTGTCACCCCTGCGCTCACCTACCACGGCTACGCTCC | 15052 | 15036.948 | 15067.052 |
| 79 | TAGAGGCTTTTCTTGGCAGTGTGGAATCAGGAACTTCGCTACTATATTTC | 15412 | 15396.588 | 15427.412 |
| 80 | GGGGAATCTCGGTTGATTTCTTTTCCTCGGGGTACTTAGATGTTTCAGTT | 15441 | 15425.559 | 15456.441 |
| 81 | CATACCAGAGGTTCGTCCACCCAGGTCCTCTCGTACTATGGGCAGGCCTC | 15259 | 15243.741 | 15274.259 |
| 82 | CGCGGGTCCATCTTATACCACCGGAGTTTTTCACACTGAGCCATGCAGCT | 15273 | 15257.727 | 15288.273 |
| 83 | CTCCCGCAACCCCGGCCACGCAACCCCCGACGGGTATCGCGCGCGGCCGG | 15211 | 15195.789 | 15226.211 |
| 84 | TTCTCTGCGGCTCCATCTCTGGAGCACCCCTTCTCCCGAAGTTACGGGGT | 15232 | 15216.768 | 15247.232 |
| 85 | GAACATCCGGCATTACCACCCGTTTCCAGGAGCTATTCCGGAGCATGGGG | 15372 | 15356.628 | 15387.372 |
| 86 | AGGTCCCGGGGTCTTTTCGTCCTTCTGCGCTTAACGAGCATCTTTACTCG | 15277 | 15261.723 | 15292.277 |
| 87 | GCTTCGGTGGCATGTTTTAGCCCCGGACATTTTCGGCGCAGGACCTCTCG | 15352 | 15336.648 | 15367.352 |
| 88 | GCTTCAAAGCCTCCGACCTATCCTACACATCACGTGCCCAGATTCAATGA | 15179 | 15163.821 | 15194.179 |
| 89 | TACTTTATTTCGCTCCACATCACGGCTTCGTCTCATGCACAGCGGATTTG | 15229 | 15213.771 | 15244.229 |
| 90 | CATGGGGTCTTTCCGTCCTGTCGCGGGTAACCTGCATCTTCACAGGTACT | 15311 | 15295.689 | 15326.311 |
| 91 | GACCTTCCTCTCAGAACCCCTACTGATCGTTGCCTTGGTGGGCCGTTACC | 15216 | 15200.784 | 15231.216 |
| 92 | ATGTTTCAGTTCCCCGGGTTCCCCTCCATACGTTATGGATTGGCGTATGG | 15341 | 15325.659 | 15356.341 |
| 93 | TTAACGCTTTCGCTTGGCCGCTTACTGTATATCGCAAACAGCGAGTATTC | 15302 | 15286.698 | 15317.302 |
| 94 | CCACGGAAAACCACCTCCGCGGCCGGCTCCCATTCCGTGTCACCCCTGCG | 15135 | 15119.865 | 15150.135 |
| 95 | TCGTAACTCGCCGGTTCATTCTACAAAAGGCACGCTCTCACCCATTAACG | 15210 | 15194.79 | 15225.21 |
| 96 | AGGATGCGACGAGCCGACATCGAGGTGCCAAACCTCCCCGCCGATATGGA | 15400 | 15384.6 | 15415.4 |
| 97 | TCCCCGGAGTACCTTTTATCCTTTGAGCGATGTCCCTTCCATACGGAAAC | 15223 | 15207.777 | 15238.223 |
| 98 | CGGCTTCCCTACTTTAATTTCGGTCCCTTACGCCCGGGTCAACCAACGCC | 15145 | 15129.855 | 15160.145 |
| 99 | CTGCTTCCAAGCCAACATCCTAGCTGTCTTAGCAGTCAGACTTCGTTAGT | 15247 | 15231.753 | 15262.247 |
| 100 | GCTACTCATACCGGCATTCTCACTTCTATGCGTTCCAGCGCTCCTCACGG | 15160 | 15144.84 | 15175.16 |
| 101 | GCCTTCGGTGTCTGCCTTATACCCGATTATTATCCATGCCCGGACCCTCG | 15191 | 15175.809 | 15206.191 |
| 102 | CCGGCTTTCCCAAAACCGTTCCACTAACATTGCAGAATCTTAAATGCAGT | 15233 | 15217.767 | 15248.233 |
| 103 | TACCTGTGTCGGTTTGCGGTACGGGCACCTTAGTATACACATAAGCTTTT | 15373 | 15357.627 | 15388.373 |
| 104 | TGTTACGCACTCTTTCAAGGGTGGCTGCTTCTGAGCCAACCTCCTGGCTG | 15310.9 | 15295.5891 | 15326.2109 |
| 105 | CTGGAGACCTTGGATATTCGGCCACAAGGATTCTCACCTTGTTCTCGCTA | 15303 | 15287.697 | 15318.303 |
| 106 | CAGTAACCCGCAAGGCTGCACCTAAATGCATTTCGGGGAGTACGAGCTAT | 15404 | 15388.596 | 15419.404 |
| 107 | AAACCTTGGATATTCGGCCTAGAGGATTCTCACCTCTATCTCGCTACTCA | 15231 | 15215.769 | 15246.231 |
| 108 | CGCTTGTGCGGGCCCCCGTCAATTTCTTTGAGTTTTAGCCTTGCGACCGT | 15292.9 | 15277.6071 | 15308.1929 |
| 109 | ACCGGGACACGTGATCCCACAACACCGGCAACGCAACCCCCGACGGGTAT | 15274 | 15258.726 | 15289.274 |
| 110 | GCTTTTCTCGCCTTCAGCCAAGTGTGCTTCCCTACTCTAATTTCGGTCCC | 15132 | 15116.868 | 15147.132 |
| 111 | CACTACTCACGGAGTATCCCTTCCTGCAGGTACTGAGATGTTTCACTTCC | 15223 | 15207.777 | 15238.223 |
| 112 | GATTGGAATTTCTCCGCTACCCACAGTTCATCCGCTACCATTTCAACGGG | 15232 | 15216.768 | 15247.232 |
| 113 | TTCCACGAGTCCCGCGCTACTCGGGAGACACCATCCATGGTGCACGCGCA | 15278 | 15262.722 | 15293.278 |
| 114 | GTCTTTTCGTCCCATCGCGGGTAATCGGCATCTTCACCGATACTACAATT | 15238 | 15222.762 | 15253.238 |
| 115 | CCGTACATCATCTCGATGGCATTCGGAGTTTGATATTCTTTGGTAAGCTT | 15363 | 15347.637 | 15378.363 |
| 116 | GGGCTTGGCTACCCGGCTATAGACTTGGCAGTCTAACCGGTGCACCAGCG | 15404 | 15388.596 | 15419.404 |
| 117 | ACTTTCGTTACTGCTCGACCCGTCAGTCTCGCAGTTAGGCTCGCTTCTGC | 15222 | 15206.778 | 15237.222 |
| 118 | CTACTGTTTCTCCGCGTATACAACGCTCCCCTACCCAATCCATTACTGGA | 15112 | 15096.888 | 15127.112 |
| 119 | ACTTATAGTCAGCGCCCCTTCTCCCGAAGTTACGGGGCCATTTTGCCGAG | 15289 | 15273.711 | 15304.289 |
| 120 | CTTCCAAGCCAACATCCTAGCTGTCTTAGCAATCTGACTTCGTTAGTTCA | 15206 | 15190.794 | 15221.206 |
| 121 | CCTCGGCAACTGGCGTTACCGATTCTCAGCCTCCCACCTATCCTGTACAT | 15129 | 15113.871 | 15144.129 |
| 122 | CCATAACGGCTCCCATCATCACACCTCGCCATGCATGCCATGCGGATTTG | 15187 | 15171.813 | 15202.187 |
| 123 | CGTGCAGGTCGGAACTTACCCGACAAGGAATTTCGCTACCTTAGGACCGT | 15371 | 15355.629 | 15386.371 |
| 124 | CATCCAAACACTTTTCAACGTGTCCTGGTTCGGTCCTCCAGTGCGTTTTA | 15229 | 15213.771 | 15244.229 |
| 125 | GCCCTAAAGCTATTTCGGGGAGAACCAGCTATATCCGGGTTCGATTGGAA | 15450 | 15434.55 | 15465.45 |
| 126 | CAGTAAAGCTCTACGGGGTCTCTCCGTCCAGTCGCGGGTAATGGGCATCT | 15394 | 15378.606 | 15409.394 |
| 127 | GGAACCTTTGGGCGCCTCCGTTACGCTTTAGGAGGCGACCGCCCCAGTCA | 15340 | 15324.66 | 15355.34 |
| 128 | CCCGCCGTGTGTCTCCCGTGATAACATTCTCCGGTATTCGCAGTTTGCAT | 15246 | 15230.754 | 15261.246 |
| 129 | CAGGTGTCAGCCCCTATACTTCATCTTTCGATTTGGCAGAGACCTGTGTT | 15309 | 15293.691 | 15324.309 |
| 130 | GACTCTTCCCAGAGTCTTCTTCTATTCCCTTGGCTGCTTTATCGCAGTCC | 15147 | 15131.853 | 15162.147 |
| 131 | GGCAACCCAACAACCCACACACCATCATCTTCAGCTACAGGACTATCACC | 15111 | 15095.889 | 15126.111 |
| 132 | AGCACCGGGCAGGTGTCAGGCTATATACCTCATGTTTCCATTTCGCATAG | 15352 | 15336.648 | 15367.352 |
| 133 | TTGCATACTATTAAGTTCAGCTCGGAAGGTGGATTTGCCTGCCTTCCTCA | 15333 | 15317.667 | 15348.333 |
| 134 | CCGGCGGATTTGCCAACCGGACACCCTACACCCTTGGACCAGGTCAATTC | 15237 | 15221.763 | 15252.237 |
| 135 | GCCGGTTATAACGGTTCATATCACCTTACCGACGCTTATCGCAGATTAGC | 15296 | 15280.704 | 15311.296 |
| 136 | CTGATACAACCAGTATCGCTCCGTCCATTTGCGCAGCACCAGTAATCATG | 15250 | 15234.75 | 15265.25 |
| 137 | TCTTTGAATGTATGGCTGCTTCTGAGCCAACATCCTAGTTGTCTTCGAGA | 15348 | 15332.652 | 15363.348 |
| 138 | TGGATTCTCGCCCTCTTGTACTCATTTCGACTACGGGACTGTTACCCTCT | 15196 | 15180.804 | 15211.196 |
| 139 | CAGTATCAACTGCAATTTTACGGTTGAGCCGCAAACTTTCACAACTGACT | 15288 | 15272.712 | 15303.288 |
| 140 | TTCTCTGCGGCTTACCTTCGTAAGCACCCCTTCTCCCGAAGTTACGGGGT | 15231 | 15215.769 | 15246.231 |
| 141 | ATTACTAGCGATTCCAGCTTCACGCAGTCGAGTTGCAGACTGCGATCCGA | 15346 | 15330.654 | 15361.346 |
| 142 | CATAGACCTGTGTTTTTGCTAAACAGTTGCTTGAGCCTATTCTCTGCGGC | 15324 | 15308.676 | 15339.324 |
| 143 | TATAAGTCGAGGCTGCACCTAAATGCATTTCGGGGAGTACGAGCTATCTC | 15425 | 15409.575 | 15440.425 |
| 144 | TCAACCTGTTGTCCATCGCCTACGCCTTTCGGCCTCGGCTTAGGTCCCGA | 15192 | 15176.808 | 15207.192 |
| 145 | GGGGTAGCTTTTATCCGTTGAGCGATGGCCCTTCCATGCGGAACCACCGG | 15410 | 15394.59 | 15425.41 |
| 146 | ATTAACCTATGGATTCAGTTAATGATAGTGTGTCGAAACACACTGGGTTT | 15453 | 15437.547 | 15468.453 |
| 147 | CCTCTTAACCTTCCAGCACCGGGCAGGCGTCAGCCCCTATACTTCGCCTT | 15130 | 15114.87 | 15145.13 |
| 148 | AAAAAGCAAGCTCTCTCAAGTTCCGTTCGACTTGCATGTGTTAGGCGCGC | 15361 | 15345.639 | 15376.361 |
| 149 | GGGCCCGTGTCTCAGTGCCCATGTGGGGGACCCTCCTCAGGCCGGCTATC | 15348 | 15332.652 | 15363.348 |
| 150 | GACTTAACAAACCGCCTGCGTGCGCTTTACGCCCAGTAATTCCGATTAAC | 15250 | 15234.75 | 15265.25 |
| 151 | CAACCTGTTGTCCATCGGCTACGCTTTTCAGCCTCACCTTAGGTCCCGAC | 15160 | 15144.84 | 15175.16 |
| 152 | CACACACCACCACCACCCGAAAGCGGAGGCGGGGCGCGGGCAGATTGGTT | 15426 | 15410.574 | 15441.426 |
| 153 | CCGTTCGACTTGCATGTGTTAAGCACGCCGCCAGCGTTCATCCTGAGCCA | 15274 | 15258.726 | 15289.274 |
| 154 | GGCACCCTCTACGGCCAGGCCTTCAAGCCTGTTCCCCTGGCAAGCCGTTT | 15211 | 15195.789 | 15226.211 |
| 155 | GCCCTTCAAAAGCGTCCCTGTGTTTAAATCTTCGGAGGTTACGGAATTTC | 15342 | 15326.658 | 15357.342 |
| 156 | TCGTGGTGTGACGGGCGGTGTGTACAAGGCCCGGGAACGTATTCACCGCG | 15540 | 15524.46 | 15555.54 |
| 157 | TCCCGGGGTTCTTTTCACCGTTCCTTCACAGTACTATGCGCTATCGGTCA | 15221 | 15205.779 | 15236.221 |
| 158 | GACTGTTCGAGGTTAGACATCAAACGAGAACAGAGCGGTATTTCACCTTG | 15458 | 15442.542 | 15473.458 |
| 159 | CACCTTAGAGTGCCCAACTGAATGCTGGCAACTAAGATCAAGGGTTGCGC | 15404 | 15388.596 | 15419.404 |
| 160 | TATGGCACTTAAGCCGACACCTCACGGCACGAGCTGACGACAACCATGCA | 15303 | 15287.697 | 15318.303 |
| 161 | TCTCGTCCATTGACCAATATTCCTCACTGCTGCCTCCCGTAGGAGTTTGG | 15230 | 15214.77 | 15245.23 |
| 162 | TTTTCACCTTTCCCTCACGGTACTGGTTCGCTATCGGTCTCTCGGGAGTA | 15252 | 15236.748 | 15267.252 |
| 163 | TTCCCCATTCAGAGATCTCCGGATCAATGGATATTTGCTCCTCCCCGAAG | 15232 | 15216.768 | 15247.232 |
| 164 | TGAGCCAACATCCTGGTTGTCTGCGTATCTTCACATCGTTTTCCACTTAA | 15228 | 15212.772 | 15243.228 |
| 165 | TCGGAGTTTGATATTCTTCGGTAGGCTTTGACGCCCCCTAGGAAATTCAG | 15398 | 15382.602 | 15413.398 |
| 166 | CCTTCGGCTCCCCTATTCGGTTAACCTTGCTACAGAATATAAGTCGCTGA | 15247 | 15231.753 | 15262.247 |
| 167 | GTCTGGACCGTGTCTCAGTTCCAGTGTGGCTGGTCATCCTCTCAGACCAG | 15336 | 15320.664 | 15351.336 |
| 168 | TTATCCGTTCCGTACATAGCTGCCCAGCCGTGCCATTGGCATGACCACTG | 15273 | 15257.727 | 15288.273 |
| 169 | TTCACAGTACTATGCGCTATCGGTCACTAAGGAGTATTTAGCCTTGCGGG | 15407 | 15391.593 | 15422.407 |
| 170 | GACTCACCCGGGGACGACGAACGTGGCCCCGGAACCCTTGGTCATCCAGC | 15328 | 15312.672 | 15343.328 |
| 171 | GGCAACTTCAACCTGCACATGGATAGATCACCCGGTTTCGGGTCTACGTA | 15346 | 15330.654 | 15361.346 |
| 172 | ACCACGAATTCCGCCTGCCTCAACTGCACTCAAGATATCCAGTATCAACT | 15163 | 15147.837 | 15178.163 |
| 173 | ACCACGCATTGCTGCATCCCAAGCTTCGGTTACATGCTTAGCCCCGTTAC | 15193 | 15177.807 | 15208.193 |
| 174 | CCAGAGCTTTTCTCGCCTCCGTCCAAGCATGCTTCCCTACTAAATTTCAG | 15143 | 15127.857 | 15158.143 |
| 175 | GCTGCACCTAAATGCATTTCGGAGAGAACCAGCTATCACGGAATTTGATT | 15939.1 | 15923.1609 | 15955.0391 |
| 176 | CCTGGTTCGGGCCTCCAGTGAGTTTTACCTCACCTTCACCCTGCTCATGG | 15207 | 15191.793 | 15222.207 |
| 177 | ACTCACCCGGGGACGACGAACGTGGCCCCGGAACCCTTGGTCATCCAGCG | 15328 | 15312.672 | 15343.328 |
| 178 | AACATCCTGGTTGTCTGTGCAATTCCACATCCTTCTCCACTTAACGTGAA | 15206 | 15190.794 | 15221.206 |
| 179 | CTACGACTTCTCCCCATACAGAACGCTCTCCTACCATACATTAGATGTAT | 15144 | 15128.856 | 15159.144 |
| 180 | CACACTTAGCCCCGGACAACCATCACCGGGGATGAGCTACCTCACTGCGT | 15246 | 15230.754 | 15261.246 |
| 181 | GGGCGACCCTCCAACAGCGGCGGAACACATTTCGACTACGGGACTCTCAC | 15311 | 15295.689 | 15326.311 |
| 182 | CTCCGGTGCTTAACCTTGCCAGTGAGCGCAACTCGCCGGACCGTTCTACA | 15259 | 15243.741 | 15274.259 |
| 183 | TTCGCAGGCTTACAGAACGCTCCCCTACCCAACAACGCATAAGCGTCGCT | 15205 | 15189.795 | 15220.205 |
| 184 | CCGTCAAGCCATGGGAGCCGGGTGTACCTAAAGTCGGTAACCGCAAGGAG | 15495 | 15479.505 | 15510.495 |
| 185 | TTACCTACACCATCACCTACACGCTTACACCAACAATCCACTAAGCGGCA | 15101 | 15085.899 | 15116.101 |
| 186 | GCGTACACCTGCAGCCTATCTACCTCGTAGTCTTCAAGGGGTCTTACCTG | 15264 | 15248.736 | 15279.264 |
| 187 | GCCGTCGCCCGTTAGTACCGGTCGGCTCCACCCCTCGCGGGGCTTCCACC | 15189 | 15173.811 | 15204.189 |
| 188 | CACAGTGCTGTGTTTTTAATAAACAGTTGCAGCCAGCTGGTATCTTCGAC | 15366 | 15350.634 | 15381.366 |
| 189 | CTGTTATCCCCAGGGTAGCTTTTATCCGTTGAGCGACGGCATTTCCACTC | 15270 | 15254.73 | 15285.27 |
| 190 | ACTTAGATGCTTTCAGCACTTATCCAATCCCGACTTAGATACCCGGCAAT | 15224 | 15208.776 | 15239.224 |
| 191 | GCTTGCGCTAACCTCTCCTCTTAACCTTCCAGCACCGGGCAGGCGTCAGC | 15195 | 15179.805 | 15210.195 |
| 192 | ACCTATCCTGTACATGTGGTACAGATACTCAATATCAAACTGCAGTAAAG | 15345 | 15329.655 | 15360.345 |
| 193 | CTCCACCAGACTAAAACGAGGCTAGCCCTAAAGCTATTTCGAGGAGAACC | 15326 | 15310.674 | 15341.326 |
| 194 | CCCGGCTTACCTTGGGCGGACGAACCTTCCCCAAGAAACCTTAGATTTTC | 15242 | 15226.758 | 15257.242 |
| 195 | GCAGAACAACTGGTACACCAGCGGTGCGTCCATCCCGGTCCTCTCGTACT | 15268 | 15252.732 | 15283.268 |
| 196 | GACCAGGTCGATTCCATTGCCTGGCCCGGCTACCTTCCTGCGTCACACCT | 15186 | 15170.814 | 15201.186 |
| 197 | CTCTGAGACTTCAAATGTGTCCCTGTGCTTAACTCTTTTGGTGGTGACGG | 15380 | 15364.62 | 15395.38 |
| 198 | ACCTCGCGGTACGCCTTCGACGCTGACTGGAATGCTCCCCTACCGATCAT | 15219 | 15203.781 | 15234.219 |
| 199 | CGTCCATCCTGAGGGAACCTTTGGGCGCCTCCGATACCCTTTCGGAGGCG | 15331 | 15315.669 | 15346.331 |
| 200 | CACCTATCGGTCTCTCCTTAGGTCCCGACTAACCCAGGGCGGACGAGCCT | 15244 | 15228.756 | 15259.244 |
| 201 | CGCTCGCCGCTACTAAGGAAATCGATGTTTCTTTCTCTTCCTCCGGCTAC | 15190 | 15174.81 | 15205.19 |
| 202 | CGCGAGTCCATCTTCAAGCGATAAAATCTTTGATATCAAAACCATGTGGT | 15352 | 15336.648 | 15367.352 |
| 203 | TGACTGGAGTTTGTCCAGCCGGGTTTCCCCATTCAGAGATCTGCGGATCA | 15384 | 15368.616 | 15399.384 |
| 204 | CCTACTTAGCTACCCGGCTATGCCCCTGGCGGAACAACCGGTGCACCAGC | 15238 | 15222.762 | 15253.238 |
| 205 | ACGCTTAAACCGGGACAACCGTCGCCCGGCCAACATAGCCTTCTCCGTCC | 15182 | 15166.818 | 15197.182 |
| 206 | GATTTGCCTGGGATAATCAACATCTACACCCTTTAACGGACTATTCCGTC | 15255 | 15239.745 | 15270.255 |
| 207 | CTAATGCGCCGCGGGTCCATCTGTAAGTGGTAGCCGAAGCCACCTTTTAT | 15353 | 15337.647 | 15368.353 |
| 208 | GGATCTTAGCACTCGCAGTCTGACTGCCGACCATAAATCAATGGCATTCG | 15330 | 15314.67 | 15345.33 |
| 209 | ACCTATCCTGTACATGTGGTACAGGTACTCAATATCAAACTGCAGTAAAG | 15361 | 15345.639 | 15376.361 |
| 210 | TCACCGGGGATGAGCTACCTCACTGCGTCCCTCCGCAGCTTGCCTACTAC | 15195 | 15179.805 | 15210.195 |
| 211 | GCCATGCAGATTCTCACTGCATTCGCGCTACTCATTCCGGCATTCTCACT | 15159 | 15143.841 | 15174.159 |
| 212 | CTTCACCTCACATACGACGCTCCCCTACCCCTGACAATTACTTGTCAAGC | 15066 | 15050.934 | 15081.066 |
| 213 | CCCTACTGATCGTCGCCTTGGTGGGCCGTTACCCCGCCAACAAGCTAATC | 15219 | 15203.781 | 15234.219 |
| 214 | ACGCATTCGGAGTTTGTCAAGACTTGATAGGCGGTGAAGCCCTCGCATCT | 15417 | 15401.583 | 15432.417 |
| 215 | ACATTTTAGGAGGCGACCGCCCCAGTCAAACTGCCCGTCAGACACTGTCT | 15276 | 15260.724 | 15291.276 |
| 216 | GGTGGGTTTCCCCATTCGGAAATCTCCGGATCAAAGCTTGCTTACAGCTC | 15328 | 15312.672 | 15343.328 |
| 217 | CTCATCCCCACCCTTTTCAACGGATGTGGGTTCGGTCCTCCATTGCCTTT | 15157 | 15141.843 | 15172.157 |
| 218 | AGGTCACTTGGTTTCGGGTCTACATCTACGTACTTAACCGCCCTTTTCAG | 15269 | 15253.731 | 15284.269 |
| 219 | ACACACTCACCACACCACCACAACATCAAAGACATCACAATGGCAGGCTC | 15153 | 15137.847 | 15168.153 |
| 220 | TGACAACTGGTGCACCAGAGGTGCGTCCATCCCGGTCCTCTCGTACTAGG | 15339 | 15323.661 | 15354.339 |
| 221 | TCTGCCTCTGCACATTGCTCCTCTACCGCGCATCTTCTTCAGACGCACCC | 15056 | 15040.944 | 15071.056 |
| 222 | CTTTTCTCGACAGTACGGGATCACCAACTTCACCAATTAAGGCTACGCAT | 15249 | 15233.751 | 15264.249 |
| 223 | CCCTCATGTCACTATTTATTCATGACATGATGACACGCTGTTAACGTGCC | 15246 | 15230.754 | 15261.246 |
| 224 | GTACGCAGTCACACGCCTAAGCGTGCTCCCACTGCTTGTACGTACACGGT | 15283 | 15267.717 | 15298.283 |
| 225 | GGCGACCACCCCAGTCAAACTACCCACCAAGCAATGTCCGCGCATAGCGC | 15209 | 15193.791 | 15224.209 |
| 226 | GACTTAGTCCCAATCACGAGCCTCACCTTAGACGGCTCCATCCCACAAGG | 15204.9 | 15189.6951 | 15220.1049 |
| 227 | GCGCTTATGCGGTATTAGCAGTCATTTCTAACTGTTATCCCCCTGTATAA | 15292 | 15276.708 | 15307.292 |
| 228 | CGCTTTCACTGCGGCTACGTGTCTCGTGACACTCAACCTCGCCAGTGACG | 15250 | 15234.75 | 15265.25 |
| 229 | ATGCTTTTCGCTTACAGGACTATAACCTTCTTTGGTGTGCCTTCCCATAC | 15219 | 15203.781 | 15234.219 |
| 230 | CGACTAACCCAGGGCGGACGAGCCTTCCCCTGGAAACCTTAGTCTTACGG | 15317 | 15301.683 | 15332.317 |
| 231 | TAGGACCCGACTAACCCTGATCCGATTAGCGTTGATCAGGAAACCTTAGT | 15354 | 15338.646 | 15369.354 |
| 232 | ACAGCTTTTCTCGTCTCTTTCCAAACTGACTTCCGCTTACGCGTCCCTTA | 15100 | 15084.9 | 15115.1 |
| 233 | TAAGACTTGCTCTCGCTGCGGCTTCAGACCTTAAGTCCTTAACCTTGCCA | 15223 | 15207.777 | 15238.223 |
| 234 | CTCTCAAACCAGCTATGGATCGTCGGCTTGGTAGGCCATTACCCCACCAA | 15251 | 15235.749 | 15266.251 |
| 235 | GGAATTTCTCCCCTATCCACACGTCATCTCCACCCTTTTCAACGGATGTG | 15143 | 15127.857 | 15158.143 |
| 236 | CCGGTCCATGGTCGGTACGGGAATATCCACCCGTTCATCCATTCGACTAC | 15258 | 15242 .742 | 15273.258 |
| 237 | CCCCCGACCGGTTTCACGGCCGCAGGTTAGAATTCCAGAAACCTAAGGGC | 15326 | 15310.674 | 15341.326 |
| 238 | AAGTTTCGGTGGCTACGGAATTTCAACCGTATGTGCATCGACTACGCCTC | 15352 | 15336.648 | 15367.352 |
| 239 | TGCGCTCCCTTTACACCCAGTAAATCCGGATAACGCTTGCCCCCTACGTA | 15162 | 15146.838 | 15177.162 |
| 240 | ATTTCGCCTACGGGACTGTCACCCTCTATGGTCCACCTTTCCAGGTGAGT | 15255 | 15239.745 | 15270.255 |
| 241 | GCTTCGGTGGCATGTTTTAGCCCCGGACATTTTCGGCGCAGGACCTCTCG | 15352 | 15336.648 | 15367.352 |
| 242 | GACATGTCTCCACATCATTCAGTTGCAATTCAAGCCCGGGTAAGGTTCCT | 15296 | 15280.704 | 15311.296 |
| 243 | CGATAACTGGCACACCAGAGGTGCGTCCTTCCCGGTCCTCTCGTACTAGG | 15299 | 15283.701 | 15314.299 |
| 244 | AACGCTTATCGGTGCGGACCTCCATCCCGTGTTACCGGGACTTCATCCTG | 15265 | 15249.735 | 15280.265 |
| 245 | CCACTCCGTCGATGTGAACTCTTGGGAGTGATAAGCCTGTTATCCCCAGG | 15353 | 15337.647 | 15368.353 |
| 246 | GCCGCCTTTTCAACGGAGGTCGGTTCGGCCCTCCATGGAGTTTTACCTCC | 15272 | 15256.728 | 15287.272 |
| 247 | ACCGTTATAGTTACGGCCGCCGTTTACTGGGGCTTCAATTCGCACCTTCG | 15295 | 15279.705 | 15310.295 |
| 248 | AGGTGTTCTCATGTGGGTTTCCCCATTCAGAGATCTGCGGGTCAATGGAT | 15454 | 15438.546 | 15469.454 |
| 249 | AGCCTGTTCCCCTGGCAAGCCGTTTTATGACTCCCGCCCGGTCCGTCGGA | 15242 | 15226.758 | 15257.242 |
| 250 | GCTGACCTACTACGAGGGGGGATCCCAACGCGCCCGCGCCGCGACCCCCC | 15250 | 15234.75 | 15265.25 |
| 251 | GTTATCCCCCTGTATGAGGCAGGTTACCCACGCGTTACTCACCCGTCCGC | 15210 | 15194.79 | 15225.21 |
| 252 | CGGACATCTTCGGCGCACAATCACTCGACCAGTGAGCTATTACGCACTCT | 15251 | 15235.749 | 15266.251 |
| 253 | TGCTTGATGCCCGATTATTATCCACGCCAAACTCCTCGACTAGTGAGCTG | 15272 | 15256.728 | 15287.272 |
| 254 | CTCCATTCGGAAATCTGCGGATCAAAGCCTACTTACGGCTCCCCGCAGCT | 15227 | 15211.773 | 15242.227 |
| 255 | GCTGTTGGTCCGGATTGTTCTCCTTTAGGACATGGACCTTAGCACCCATG | 15350 | 15334.65 | 15365.35 |
| 256 | TGCTGGCACGGAGTTAGCCGTCACTTCCTTGTTGAGTACCGTCATTATCT | 15325 | 15309.675 | 15340.325 |
| 257 | GCTATCGGTCAGACAGGTATGCTTAGACTTACCCAACGGTCTGGGCTGAT | 15417 | 15401.583 | 15432.417 |
| 258 | TATTCCTCACTGCTGCCTCCCGTAGGAGTTTGGACCGTGTCTCAGTTCCA | 15246 | 15230.754 | 15261.246 |
| 259 | TCCCGCTGGCCTTAGAATTCTCTTCCTGTCCACCTGTGTCGGTTTGCGGT | 15244 | 15228.756 | 15259.244 |
| 260 | CGACTATTGTCCTCGGCTTAGGTCCCGACTTACCCTGAGAGGACGAGCCT | 15314 | 15298.686 | 15329.314 |
| 261 | GGTCCTTTTCACCTTTCCTTCACAGTACTATGCGCTATCGGTCACTAAGT | 15204 | 15188.796 | 15219.204 |
| 262 | TCGGCTACTGATCGTCGCCTTGGTAGGCCGTTGCCCTGCCAACTAGCTAA | 15305 | 15289.695 | 15320.305 |
| 263 | CTTGGGAGTATGTTTACACGCACTATTACCGTTTTCCGAGGAAATTGGTA | 15421 | 15405.579 | 15436.421 |
| 264 | CACACAACCCCTACCAGGTATCACATGCACACGGTTTAGCCTCATCCACG | 15149 | 15133.851 | 15164.149 |
| 265 | CCACGGCTTCGGTGTTGTGTTTTAGCCCCGGACATTTTCGGCGCAGGGCC | 15368 | 15352.632 | 15383.368 |
| 266 | CCACCTTCCTCCAGTTTATCACTGGCAGTCTCCTTTGAGTTCCCGGCCGG | 15167 | 15151.833 | 15182.167 |
| 267 | AGCTTTCGGGGAGAACCAGCTATCTCCCGGTTTGATTGGCCTTTCACCCC | 15280 | 15264.72 | 15295.28 |
| 268 | CGAGCCTTCCTCAGGAAACCTTAGGCATTCGGTGGAGGGGATTCTCACCC | 15363 | 15347.637 | 15378.363 |
| 269 | CCCAGGGCTAGATCATCCCGCTTCGGGTCCAGGACAAGCGACTGAAAACG | 15375 | 15359.625 | 15390.375 |
| 270 | AAAATCATGGGAAATCTCATCTTGAGGGGGGCTTCGCACTTAGATGCTTT | 15455 | 15439.545 | 15470.455 |
| 271 | ATCCTGTACAAGCTGTACCAACATTCAATATCAGGCTGCAGTAAAGCTCC | 15282 | 15266.718 | 15297.282 |
| 272 | TTAGCAGGTGGTCCGGATTCTTCTCCTCTCGGGCACGGACCTTAGCACCC | 15281 | 15265.719 | 15296.281 |
| 273 | GTCCGTTTACGGTACGGGTACCTCAAGGATAAGTTTAGCGGGTTTTCTAG | 15478 | 15462.522 | 15493.478 |
| 274 | CACTGGCGTGCTGCCTTCTCTGCCTCCCACCTATCCTGTACATGAAATAC | 15144 | 15128.856 | 15159.144 |
| 275 | TGCGGTATTAGCAGTCATTTCTAACTGTTATCCCCCTGTATAAGGCAGGT | 15357 | 15341.643 | 15372.357 |
| 276 | GCTATCGGTCAGACAGGTATGCTTAGACTTACACCACGGTCGGTGCGGAT | 15442 | 15426.558 | 15457.442 |
| 277 | TTTACTCCTTTCGGATGGGATATCTCATCTTGAGGGGGGCTTCACGCTTA | 15380 | 15364.62 | 15395.38 |
| 278 | TGGCCGGTCGCCCTCTCAGGCCGGCTACCCGTCGAAGCCTTGGTGAGCCG | 15332.9 | 15317.5671 | 15348.2329 |
| 279 | AAGCCTGTTCCCCTGGCAAGCCGTTTTATGACTCCCGCCCGGCCCGTCGG | 15227 | 15211.773 | 15242.227 |
| 280 | AAGGTTAAGCCTCACGGTTCATTAGTACCGGTTAGCTCAACGCATCGCTG | 15361 | 15345.639 | 15376.361 |
| 281 | GACATCATACTAACGCGCCCTATTAAGACTCGGTTTCCCTACGGCTCCGT | 15217 | 15201.783 | 15232.217 |
| 282 | TGTGTTTTTGTTAAACAGTTGCCTGGACCGATTCTCTGCGCCTCAAGTCG | 15340 | 15324.66 | 15355.34 |
| 283 | GCCCCAGTCAAACTACCCACCAGACACTGTCCGCAACCCGGATTACGGGT | 15215 | 15199.785 | 15230.215 |
| 284 | GCGTCACACCTGTTAATGCGCTTGCCTTACCGGTTCAGGTCCCGCGCTCC | 15217 | 15201.783 | 15232.217 |
| 285 | GCGATGGCCCTTCCATGCGGAACCACCGGATCACTAAGCCCGACTTTCGT | 15268 | 15252.732 | 15283.268 |
| 286 | AAGCTCCATGGGGTCTTTCCGTCTAGTCGCGGGTAACCGGCATCTTCACC | 15305 | 15289.695 | 15320.305 |
| 287 | CGCTAGCCCTAAAGCTATTTCGGAGAGAACCAGCTATCTCCAAGTTCGTT | 15305 | 15289.695 | 15320.305 |
| 288 | TCCCATCCGCACTTCGCTTCCCTGCTATGCCGTTGGCACGACAACAGTTG | 15185 | 15169.815 | 15200.185 |
| 289 | TTTCACTCCCCTCCCGGGGTCCTTTTCACCTTTCCTTCACAGTACTCTGC | 15028 | 15012.972 | 15043.028 |
| 290 | CGTCCTCGGCTTAGGCCCCGACTTACCCTGGGCGGATGAACCTTCCCCAG | 15236 | 15220.764 | 15251.236 |
| 291 | CGACATCGAGGTGCCAAACCTCCCCGTCGATGTGGACTCTTGGGGGAGAT | 15428 | 15412.572 | 15443.428 |
| 292 | TACCTGATCGACTTGTCAGTCTCCCAGTCAAGCGCCCTTATGCCATTACA | 15192 | 15176.808 | 15207.192 |
| 293 | CTTCCAAGCCAACATCCTAGCTGTCTTAGCAATCTGACTTCGTTAGTTCA | 15206 | 15190.794 | 15221.206 |
| 294 | ACGCCTTAACCATGTGAAGGGTAGATTTTCTGACCCCTTCGGCCTGAACG | 15337 | 15321.663 | 15352.337 |
| 295 | CTCAAGGATTAAGTTTAGCGGATTTTCTCGGGAGTATGTTTACACGCACT | 15421 | 15405.579 | 15436.421 |
| 296 | CCCCATCCATCACCGATAAATCTTTAATCTCTTTCAGATGTCTTCTAGAG | 15165 | 15149.835 | 15180.165 |
| 297 | ATACTTTGGGACCTTAGCTGTGGGTCTGGGCTGTTTCCCTTTTGACAATG | 15411 | 15395.589 | 15426.411 |
| 298 | CGCCCATAGGCGGTGCCGGCCCATGACGGCCGGCGGGTTCCCCCATTCGG | 15343 | 15327.657 | 15358.343 |
| 299 | AAAATCATGGGAAATCTCATCTTGAGGTGGGCTTCGCACTTAGATGCTTT | 15430 | 15414.57 | 15445.43 |
| 300 | ACAACTTGATACCCGATTATTATCCACGCCCGACTCCTCGACTAGTGAGC | 15210 | 15194.79 | 15225.21 |
| 301 | CTGAGTTTGATAAGCTTCGCTAACCTCTCGGCCGCTAGGCTATTCAGTGC | 15319 | 15303.681 | 15334.319 |
| 302 | GCCCAGATCGTTGCGCCTTTCGTGCGGGTCGGAACTTACCCGACAAGGAA | 15388 | 15372.612 | 15403.388 |
| 303 | TTATAGTTACGGCCGCCGTTCACTGGGGCTTCGGATCACTGCTTCAGATC | 15335 | 15319.665 | 15350.335 |
| 304 | GGCATTGTCCCACCGCCGGGTCACGGCGGCTGGTTAGAAACCCAATACTG | 15373 | 15357.627 | 15388.373 |
| 305 | GTCCACACATTTAGCCCCAGACAACCATCGCTGGGGTTGAGCTACCTCAC | 15236 | 15220.764 | 15251.236 |
| 306 | TCTCACGACGTTCTGAACCCAGCTCGCGTGCCGCTTTAATGGGCGAACAG | 15323 | 15307.677 | 15338.323 |
| 307 | ATGCGACGAGCCGACATCGAGGTGCCAAACCTCCCCGTCGATGTGAACTC | 15326 | 15310.674 | 15341.326 |
| 308 | CCTGTGTCGGTTTAGGGTACGGGCAGTTTGAACCTCGCGCCGATGCTTTT | 15422 | 15406.578 | 15437.422 |
| 309 | CGATATTGCAAGGGTGGTATCCCAACAGCGCCTCCTCAGAGACTGGCGTC | 15372 | 15356.628 | 15387.372 |
| 310 | CCCCCGACCGGATTCACGGCCGCAGGTTAGAATTTCAGCACCTCAAGAGT | 15301 | 15285.699 | 15316.301 |
| 311 | TCAGATGGCGGCATTGTCACTACTGCGTCTCCACATCACTCCTGGAGGTA | 15313 | 15297.687 | 15328.313 |
| 312 | CTTTTCGTCCCATCGCGGGTAATCGGCATCTTCACCGATACTACAATTTC | 15198 | 15182.802 | 15213.198 |
| 313 | ACAACGAATTCCGCCAACTTCCCGCGCACTCAAGCCCTCCAGTTCGCGCT | 15117 | 15101.883 | 15132.117 |
| 314 | CCCGAAGTTACGGGGCCAATTTGCCGAGTTCCTTAACAACCCTTCTCCCG | 15218 | 15202.782 | 15233.218 |
| 315 | TCAAGGGGGTTTACTTCTTTCGAATGGGATATCTCATCTTAAGGGGGGCT | 15493 | 15477.507 | 15508.493 |
| 316 | CTTCACAGTACTATACGCTATCGGTCACTGGGTAGTATTTAGGGTTGGAG | 15462 | 15446.538 | 15477.462 |
| 317 | ATTCCGTCAGACGGCCGGACTGTCACTTCTCCGTCACCACATCGCTCTCT | 15145 | 15129.855 | 15160.145 |
| 318 | CGGTACTGGTTCACTATCGGTCACTAGGGAGTATTTAGGGTTGGGAGATG | 15583 | 15567.417 | 15598.583 |
| 319 | AGCTGATGGTCCGGATTCTTCTCCTTTAGGACATGGACCTTAGCACCCAT | 15303 | 15287.697 | 15318.303 |
| 320 | CGTATTACCGCGGCTGCTGGCACGGAATTAGCCGGTCCTTATTCATAAGG | 15393 | 15377.607 | 15408.393 |
| 321 | ACGGGTTAGCCTCGCCACGCACCACTGACTCGCAGACTCATTTTTCGATA | 15242 | 15226.758 | 15257.242 |
| 322 | ACGGCGTGGACTACCAGGGTATCTAATCCTGTTCGCTCCCCACGCTTTCG | 15264.9 | 15249.6351 | 15280.1649 |
| 323 | TGCGCATTCGGAGTTTATCAAGACTTGATAGGCGGTGAAGCCCTCGCATC | 15417 | 15401.583 | 15432.417 |
| 324 | CTGTTGTCCATCGGCTACGACTCTCGTCCTCACCTTAGGCCCCGACTTAC | 15136 | 15120.864 | 15151.136 |
| 325 | GGCTCACGCCTCACCTTCGACGCGGAGTGGAATGCTCCCCTACCGATGTT | 15275 | 15259.725 | 15290.275 |
| 326 | GATGTTTCAGTTCAGGCGGTTCCCTCGATATACCTATTTTTAAGTTCAGT | 15338 | 15322.662 | 15353.338 |
| 327 | CATTGTCTAAGATTCCCCACTGCTGCCTCCCGTAGGAGTCTGGGCCGTGT | 15296 | 15280.704 | 15311.296 |
| 328 | TCACAGTACTATGCGCTATCGGTCACTAAGTGGTATTTAGCCTTAGGGGG | 15447 | 15431.553 | 15462.447 |
| 329 | GTAGTATTTAGGCTTGGAGGATGGTCCCTCCTGCTTCCCACAGGGTTTCA | 15390 | 15374.61 | 15405.39 |
| 330 | TTGGGACCTTAGCTGCGGGTCTGGGCTCTTTCCCTTTTGACTATCCAACT | 15292 | 15276.708 | 15307.292 |
| 331 | CAGCTTGGTGGCGCAGAACTAAGCATTTGACTCAGTCCTCACCTCACTGC | 15282 | 15266.718 | 15297.282 |
| 332 | ACCAAGTACAGGAATATTAACCTGTTTCCCATCGACTACGCCTTTCGGCC | 15225 | 15209.775 | 15240.225 |
| 333 | AAGCCCGCTTGTGCGATTACACTCGACACCCGATTGCCAACCGGGCCGAG | 15302 | 15286.698 | 15317.302 |
| 334 | CCTTAAATACGCACAACCATCGGCGCACTGCAGCTACCTGTCTGCGTCAC | 15196 | 15180.804 | 15211.196 |
| 335 | CTACCCAGCGATGCCTTTGGCAAGACAACTGGTACACCAGCGGTAAGTCC | 15325 | 15309.675 | 15340.325 |
| 336 | CCTGTGTCGGTTTACGGTACGGGCGCATGGCAAACAATAGCGGCTTTTCT | 15424 | 15408.576 | 15439.424 |
| 337 | CCGCGCTTACCCTATCCTCCTGCGTCCCCCCATTGCTCAAATGGTGAGGA | 15170 | 15154.83 | 15185.17 |
| 338 | GGCTCTCTGTACTGTCAGGTTTCAGCAAGGACTAACTCTTAATCTGCCCC | 15263 | 15247.737 | 15278.263 |
| 339 | GGATCACCGGATTCGGGCCGTAAGGCCCCCATCATCGCGCCTCGCCCCGA | 15254.8 | 15239.5452 | 15270.0548 |
| 340 | TGGTCTCCGCTCGTTCAGACAAGGTTTCACGTGTCTCGTCCTACTCTGGA | 15286 | 15270.714 | 15301.286 |
| 341 | CAATCCCACTTTATGCCACCGGATCACTAAGTCCTACTTTCGTACCTGCT | 15127 | 15111.873 | 15142.127 |
| 342 | GTCACCAAGTAGTATTTAGCCTTGGGGGGTGGGCCCCCCGTCTTCCCACC | 15306 | 15290.694 | 15321.306 |
| 343 | ATCCCCGGAGTACCTTTTATCCGTTGAGCGATGGCCCTTCCATTCAGAAC | 15248 | 15232.752 | 15263.248 |
| 344 | TACCTCTCACGGTGACCATCCGACGCGGCACCTAAATGCCTTTCGGGGAG | 15308 | 15292.692 | 15323.308 |
| 345 | CCGTACTCCCCAGGCGGAGTGCTTAATGCGTTAGCTGCAGCACTAAGGGG | 15428 | 15412.572 | 15443.428 |
| 346 | ATCACCAGTTTTACCCTAGGGCGCTCCTTGCGGTTACGCACTTCAGGTAC | 15264 | 15248.736 | 15279.264 |
| 347 | GGAGGGCACCTTTAGAAGCCTCCGTTACGCTTTTGGAGGCGACCACCCCA | 15348 | 15332.652 | 15363.348 |
| 348 | CTGGAGACCTTGGATATTCGGCCACAAGGATTCTCACCTTGTTCTCGCTA | 15303 | 15287.697 | 15318.303 |
| 349 | GGGCTTTCACCCTCTTTGGCTGGCTTTCCCAAAACCATTCTGCTAGGATC | 15230 | 15214.77 | 15245.23 |
| 350 | GTGGGATTGGCTTAACCTCGCGGTTTCGCTGCCCTTTGTTCTGTCCATTG | 15339 | 15323.661 | 15354.339 |
| 351 | ATGCTACGCAGAGAAGTCCGGATATCAATGCCAGACTAGAGTAAAGCTCC | 15421 | 15405.579 | 15436.421 |
| 352 | TCCGTATACTCTCAGGTTCGACTCTCCCCGCGGATTTGCCTACGGGAATC | 15240 | 15224.76 | 15255.24 |
| 353 | CTGGACCTATTCTCTGCGCCTCACATTGCTGTGAGGACCCTTTATCCCGA | 15215 | 15199.785 | 15230.215 |
| 354 | TTAGCAGGTGGTCCGGATTCTTCTCCTCTCGGGCACGGACCTTAGCACCC | 15281 | 15265.719 | 15296.281 |
| 355 | GCCTGTACACCTGCATCCTATCAACGTCATAGTCTTTGACGACCCTGAGA | 15241 | 15225.759 | 15256.241 |
| 356 | AGACTCCAATCCGGACTACGACGCACTTTATGAGGTCCGCTTGCTCTCGC | 15258 | 15242 .742 | 15273.258 |
| 357 | GGTTTGCCCTCCTGCCTCTTCGCTCGCCGCTACTGAGGCAATCGCTCTTG | 15199 | 15183.801 | 15214.199 |
| 358 | ACCTTTCCCTCACGGTACTGGTACGCTATCGGTCAGACAGGTATGCTTAG | 15328 | 15312.672 | 15343.328 |
| 359 | CCGGTCCTCTCGTACTAGGGACAGCTCCCATCAAATATCCTGCGCCCACG | 15188 | 15172.812 | 15203.188 |
| 360 | CCATTGGCATGACAACCCGAACACCAGTGATGCGTCCACTCCGGTCCTCT | 15212 | 15196.788 | 15227.212 |
| 361 | ATGTGCTTGTAAGCACAGAGTTTCAGGTTCTTTTCACTCCCCTCCCGGGG | 15310 | 15294.69 | 15325.31 |
| 362 | CCCTTCTCCCGAAGTTACGGGGTAATTTTGCCGAGTTCCTTAACAACCCT | 15223 | 15207.777 | 15238.223 |
| 363 | CCTGAGTCGGTTTAGGGTACGGGCGCGTTATGCCCTCACGTCGAGGCTTT | 15432 | 15416.568 | 15447.432 |
| 364 | ATCTGGGCTGTTTCCCTTTCGACAATGAAACTTATCTCACACTGTCTGAC | 15237 | 15221.763 | 15252.237 |
| 365 | CGTATTTCAAGGATGGCTCCACAAACACTGGCGTGCCTGCTTCAAAGCCT | 15306 | 15290.694 | 15321.306 |
| 366 | GGTCATTGCCTGCTTGCGGCTGACCATGGCTTATCGCAGCTGACCACGTC | 15321 | 15305.679 | 15336.321 |
| 367 | CCTGGCGCGGGTAACCAGCATCTTCACTGGTACTTCAATTTCACCGGGTG | 15329 | 15313.671 | 15344.329 |
| 368 | GTAACTCACAAGGCTGCACCTAAATGCATTTCGGGGAGTACGAGCTATCT | 15394 | 15378.606 | 15409.394 |
| 369 | GTCGGTTTGGGGTACGGGCGGCCATAGCCCTCACGCCGAGGCTTTTCTCG | 15418 | 15402.582 | 15433.418 |
| 370 | CACCGTCTATGGTCCCATTTTCCAAAGGGTTCTACTCATGAAATGTCTTG | 15277 | 15261.723 | 15292.277 |
| 371 | CCGGCAACGCAACCCCCGACGGGTATCACGCGCAACCGGTTTGGTCTGAT | 15318 | 15302.682 | 15333.318 |
| 372 | TTATCCTTCTGTGTCACTGCTTCATTCCATCGGTAGTGCAGGAATCTACA | 15268 | 15252.732 | 15283.268 |
| 373 | CAGAGCACCCCTTCTCCCGAAGTTACGGGGTCATTTTGCCGAGTTCCTTA | 15264 | 15248.736 | 15279.264 |
| 374 | ATACTATCAGGTTCGATTCTCATGGTGGATTTGCCTGCCAAGATCAACAT | 15350 | 15334.65 | 15365.35 |
| 375 | CTTACGGGGCTTTCACCCTCTCTGGCCGGCTTTCCCAAAACCGTTCTGCT | 15167 | 15151.833 | 15182.167 |
| 376 | GACCGGCCTTCCCATGCCGTTCGGTTAACAACTTAAGTCCTAAATGCGGT | 15297 | 15281.703 | 15312.297 |
| 377 | CGTTTATCCGATCCGTACGTAGTTGCCCAGCTATGCTCCTGGCGGAACAA | 15313 | 15297.687 | 15328.313 |
| 378 | GTATCTAATCCTGTTTGATACCCACACTTTCGAGCATCAGCGTCAGTTAC | 15246 | 15230.754 | 15261.246 |
| 379 | GGTGCTTGTAAACACAAGGTTTCAGGTTCTTTTTCACTCCCCGTCAGGGG | 15374 | 15358.626 | 15389.374 |
| 380 | GTAGGCGCACGGTTTCAGGAACTCTTTCACTCCCCTCCCGGGGTGCTTTT | 15287 | 15271.713 | 15302.287 |
| 381 | ACTTCTGAGTTCGGCATGGGGTCAGGTGGGACCACCGCGCTACGGCCGCC | 15421 | 15405.579 | 15436.421 |
| 382 | TTCCGTGTTCGGTATGGGAACGGGTGTGACCTCTTCGCTATCGCCACCAA | 15360 | 15344.64 | 15375.36 |
| 383 | TCGCCTTAGGACCCGACTCACCCGGGGACGTTAACCGTGGCCCCGGAACC | 15279 | 15263.721 | 15294.279 |
| 384 | CACTCACCCACAACCATGGGCTCCCCATCATGCCTCAACCTTCACGCCCA | 15006 | 14990.994 | 15021.006 |
| 385 | CTCCGAGACTTCATATGTGTCCCTGTGTTTAACTCTTTTGGTGGTGACGG | 15371 | 15355.629 | 15386.371 |
| 386 | AAAATTCCCTACTGCTGCCTCCCGTAGGAGTTTGGGCCGTGTCTCAGTCC | 15280 | 15264.72 | 15295.28 |
| 387 | GACCAGGTAAGGTTCTTCGCGTTGCATCGAATTAAACCACATGCTCCACC | 15290 | 15274.71 | 15305.29 |
| 388 | CGAAGTTTGATAGGGTTCGGTAAGCTTTGTGGCCCCCTAGCCCATTCAGT | 15399 | 15383.601 | 15414.399 |
| 389 | AGGCTTGCGCCGCCGCTTCGCCCCGATGGGGACGCTCTCCTACCCAGCGT | 15252.8 | 15237.5472 | 15268.0528 |
| 390 | CGAACAGAGCGGTATTTCACCTTACGGCTCCGCGCGATCTGGCGACCGCG | 15349 | 15333.651 | 15364.349 |
| 391 | ACCGTTCTACAAAAAGTACGCGGTTGTACTCGTATGGTACTTCCACAGTT | 15335 | 15319.665 | 15350.335 |
| 392 | CGTTTCGCTCGCCGCTACTCAGGGAATCGCATTTGCTTTCTCTTCCTCCG | 15173 | 15157.827 | 15188.173 |
| 393 | GCTACTTGGGACAACACGATCGGAAGACGGCTCACGTCCAGGTACGGGGC | 15471 | 15455.529 | 15486.471 |
| 394 | AAGGTCCCCCTCTTTGGTCTTGCGACGTTATGCGGTATTAGCTACCGTTT | 15316 | 15300.684 | 15331.316 |
| 395 | GTTCTGAACCCAGCTCGCGTACCACTTTAATCGGCGAACAGCCGAACCCT | 15236 | 15220.764 | 15251.236 |
| 396 | TGATTCAAAGCCTCCGGCCTATCCTACACATCAATCACCCAAATTCAATG | 15162 | 15146.838 | 15177.162 |
| 397 | GTCTTTTCGTCCCATCGCGGGTAATCGGCATCTTCACCGATACTACAATT | 15238 | 15222.762 | 15253.238 |
| 398 | CCCCCCCCCCCCTTCCCCCCTCTCCTCCCCCTTCCCCCTTTCGCGCCCCC | 14627 | 14612.373 | 14641.627 |
| 399 | CAGGTGTCACCCCATATACGTCATCTTTCGATTTAGCATAGAGCTGTGTT | 15317 | 15301.683 | 15332.317 |
| 400 | CTCCACCAGACTAAAACGAGGCTAGCCCTAAAGCTATTTCGAGGAGAACC | 15326 | 15310.674 | 15341.326 |
| 401 | TTCCGTCAGCCGGCAGGACTGTCACTTCTCCGTCTCCACGTCACTCCATG | 15161 | 15145.839 | 15176.161 |
| 402 | CGCTAATTTTTCAACATTAGTCGGTTCGGTCCTCCAGTTAGTGTTACCCA | 15268 | 15252.732 | 15283.268 |
| 403 | CTTGGCAGTGTGACATCACTAACTTCGCTACTAAACTTCGCTCCCCATCA | 15176 | 15160.824 | 15191.176 |
| 404 | CCCGTTAAATTTTCGGCGCAGAGTCACTCGACCAGTGAGCTATTACGCAC | 15306 | 15290.694 | 15321.306 |
| 405 | CCCGGAGTACCTTTTATCCTTTGAGCGATGTCCCTTCCATGCGGAAACAC | 15248 | 15232.752 | 15263.248 |
| 406 | TTCTCTGCGGCTCCATCGCTGCAGCACCCCTTCTCCCGAAGTTACGGGGT | 15217 | 15201.783 | 15232.217 |
| 407 | AAGCTACCTACTTCTTTTGCAACCCACTCCCATGGTGTGACGGGCGGTGT | 15304 | 15288.696 | 15319.304 |
| 408 | GCACAGCCATGTGTTTTTGTTAAACAGTTGCCTGGACCTATTCTCTGCGC | 15309 | 15293.691 | 15324.309 |
| 409 | GCCAACATCCTGGTTGTCTGTGCAATTCCACATCCTTTTCCACTTAACTA | 15157 | 15141.843 | 15172.157 |
| 410 | GGTCACCCGGTTTCGGGCCCATTATATGCAACTTAACGCCCTTTTCAAAC | 15232 | 15216.768 | 15247.232 |
| 411 | TTATAGTTACGGCCGCCGTTCACTGGGGCTTCGATTCAATGCTTGCACAT | 15334 | 15318.666 | 15349.334 |
| 412 | GTTTATCTGAGATTGGTAATCCGGGATGGACCCCTCAATCAAACAGTGCT | 15400 | 15384.6 | 15415.4 |
| 413 | CGAAGTTACGGGGTCATTTTGCCGAGTTCCTTGACAATGCTTCTTCCGCC | 15310 | 15294.69 | 15325.31 |
| 414 | GTCCACACACGCGTGTGTCCCTCATCAGTTCTCACCCTCCATGCCCCCCG | 15051 | 15035.949 | 15066.051 |
| 415 | CCGGCCCGTCGGGGCCGGGACACACGCTCCCGCAACCCCGGCCACGCAAC | 15220 | 15204.78 | 15235.22 |
| 416 | CCGGTACATTTTCGGCGCAGGGTCACTCGACTAGTGAGCTATTACGCACT | 15353 | 15337.647 | 15368.353 |
| 417 | CTCGAACTTCTTGTAAGCACACGGTTTCAGGTTCTCTTTCACTCCCCTTC | 15140 | 15124.86 | 15155.14 |
| 418 | TTTCAGTTCAGGCGGTTCCCCCCGTATCCCTATGGATTCAGAATACGGTG | 15319 | 15303.681 | 15334.319 |
| 419 | TCCGTTACATTTTGGGAGGCGACCGCCCCAGTCAAACTGCCTACCTGACA | 15267 | 15251.733 | 15282.267 |
| 420 | CCGCTCCTTCCATCAAGGTTCCACGTGTCTCGATGTACTCTGGATCCTGC | 15191 | 15175.809 | 15206.191 |
| 421 | CCACGTGTTACTCACCCGTCCGCCGCTAACATCAGGGAGCAAGCTCCCAT | 15197 | 15181.803 | 15212.197 |
| 422 | GACTCCGTACTGTCAGGTTCGGCTCAACGGGTGGATTTGCCTGCCCATCT | 15336 | 15320.664 | 15351.336 |
| 423 | ACGTGTCCGGCGGTACTCTGGATACAGATGGCTGTTCAGGCTTTTCGTGT | 15446 | 15430.554 | 15461.446 |
| 424 | TGGGCTGTTTCCCTTTGGACAATGAAACTTATCTCCCACTGTCTGACTCC | 15229 | 15213.771 | 15244.229 |
| 425 | ACATAGCTACCCAGCCATGCCCTTGGCAGAACAACTGGTACACCAGCGGT | 15294 | 15278.706 | 15309.294 |
| 426 | CAGAGGTCAGTCCAACACGGTCCTCTCGTACTAGTGTCAGAGCCACGCAA | 15325 | 15309.675 | 15340.325 |
| 427 | GTTTGATAGGGTTCAGTAACTTCTCAGCCCCTAGCCCATTCAGTGCTTTA | 15293 | 15277.707 | 15308.293 |
| 428 | CGGCACCGGGCAGGCGTCACACCCTATACGTCCACTGTTCGTGTTGGCAG | 15340 | 15324.66 | 15355.34 |
| 429 | AACCCAATAAATCCGGATAACGCTTGCCCCCTACGTATTACCGCGGCTGC | 15220 | 15204.78 | 15235.22 |
| 430 | CCATACATCAATTATCTGGCATTCTGAGTTTGATAGGGTTCAGTAACCTC | 15325 | 15309.675 | 15340.325 |
| 431 | CCTCCGTTACACTTTGGGAGGCGACCGCCCCAGTCAAACTGCCCGCCAAG | 15238 | 15222.762 | 15253.238 |
| 432 | CTGTTATCCCCGAGGTAGCTTTTATCCGTTAAGCGACGGCTTTTCCACTC | 15245 | 15229.755 | 15260.245 |
| 433 | TAGCCCATTCAGTGCTTTACCTCCGGTAATCTAAATCAACGCTAGCCCTA | 15200 | 15184.8 | 15215.2 |
| 434 | TCCACAGCTCCTTACGGTACTGCTTCGTCCCGCATGCAATGCTCCTCTAC | 15120 | 15104.88 | 15135.12 |
| 435 | CCATCGCGGGTAATCGGCATCTTCACCGATACTACAATTTCACCGAGCTC | 15226 | 15210.774 | 15241.226 |
| 436 | CTGGACCTATTCTCTGCGCCCAACTCTCGTTGGGACCCTTTATCCCGAAG | 15200 | 15184.8 | 15215.2 |
| 437 | CTTTTACCTTTACACTCTACGATTGATTTCCAACCAATCTGAGCCAACCT | 15125 | 15109.875 | 15140.125 |
| 438 | TTATAGTTACGGCCGCCGTTTACCGGGGCTTCAATTCAAAGCTTCATATT | 15317 | 15301.683 | 15332.317 |
| 439 | GCCATTAAGATTCTCACTTAATTCTCGCTACTTATTCCGGCATTCTCACT | 15147 | 15131.853 | 15162.147 |
| 440 | GGCCGATCACCCTCTCAGGTCGGCTACGCATCGTCGCCTTGGTGAGCCGT | 15307 | 15291.693 | 15322.307 |
| 441 | CTTCTCCCGCTGGCCTTAGAATCTTCTTCCTATCTACCTGTGTCGGTTTG | 15178 | 15162.822 | 15193.178 |
| 442 | TTCCTTCACCCGAGTTCTCTCAAGCGCCTTGGTATTCTCTACCTGACCAC | 15110 | 15094.89 | 15125.11 |
| 443 | GCTAGTCCTAAAACTATTTCGGGGAGAACCAGCTATCTCCGGGTTCGATT | 15376 | 15360.624 | 15391.376 |
| 444 | CCTCCGGCCGGTTTCACGGCCGCAAGTTAGAATTCCAGCACTACAAGAGT | 15316 | 15300.684 | 15331.316 |
| 445 | TGTTCGTCCCGTCCTTCATCGGCTCCTAGTGCCAAGGCATCCACCGTGCG | 15217 | 15201.783 | 15232.217 |
| 446 | GCCAGGCCTTCAAGCCTGTTCCCCTGGCTAGCCGCTTTATGACTCCCGCC | 15162 | 15146.838 | 15177.162 |
| 447 | CTTTCTTTTCCTCCGGCTACTTAGATGTTTCAGTTCACCGGGTTCCCTTC | 15153 | 15137.847 | 15168.153 |
| 448 | ATGATTCTCACATAATTCTCGCTACTCATTCCGGCATTCTCACTCGTATG | 15172 | 15156.828 | 15187.172 |
| 449 | CGGGCACGGACCTTAGCACCCATGCCCTTACTGCCGGACTGCAGACCGTG | 15294 | 15278.706 | 15309.294 |
| 450 | GTGAGTTTCCTCATTCAGAGATCTCCGGATCAATGCTTATTTGCAGCTCC | 15293 | 15277.707 | 15308.293 |
| 451 | TAAATGCAGTCCGAACCCCGGAGTGCACGCACTCCGGTTTGGGCTCTTTC | 15314 | 15298.686 | 15329.314 |
| 452 | GCCCAAGGGTAGATCACTTGGTTTCGCGTCTACTCCTTCCGACTATACGC | 15264 | 15248.736 | 15279.264 |
| 453 | AGCTTAGCGGATTTTCTCGGGAGTCTGATTACCGGCGCTATTGGATTCCA | 15414 | 15398.586 | 15429.414 |
| 454 | CTCGCAGTCAAGCTCCCTTCTGCCTTTGCACTCTCCGAATGATTTCCAAC | 15119 | 15103.881 | 15134.119 |
| 455 | GTCTAGTCCCACGTACTTGTGCGCCCTGTTCAGACTCGCTTTCGCTCCGC | 15183 | 15167.817 | 15198.183 |
| 456 | TTCTCCGCTATCCACACCTCATCGCCACCCTTTTCAACGGATGTGCGTTC | 15095 | 15079.905 | 15110.095 |
| 457 | GCCGGCTCCCATTCCGTGTCACCCCTGCGCTCACCTACCACGGCTACGCT | 15092 | 15076.908 | 15107.092 |
| 458 | TCCCGGGGTCCTTTTCACCTTTCCTTCACAGTACTATGCGCTATCGGTCA | 15181 | 15165.819 | 15196.181 |
| 459 | CCAACATCCTGGTTGTCTGTGCAATTCCACATCCTTTTCCACTTAAATCC | 15117 | 15101.883 | 15132.117 |
| 460 | GCTGGCGCCGCGGCTTCGAAGCCTCCCGCCTATGCTACACAATCCGCACC | 15190 | 15174.81 | 15205.19 |
| 461 | ACGCCCAATAATTCCGGACAACGCTTGCCACCTACGTATTACCGCGGCTG | 15236 | 15220.764 | 15251.236 |
| 462 | CCCTACCAGGTATCACATGCACACGGTTTAGCCTCATCCACGTTCGTTCG | 15193 | 15177.807 | 15208.193 |
| 463 | AGCACCGGGCAGGTGTCAGGCTGTATACGTGATCTTTCAATTTGGCACAG | 15457 | 15441.543 | 15472.457 |
| 464 | CTCCCCATCATGCCTCAACCTTCACGCCCAGCGGATTTACCTACCAGACA | 15076 | 15060.924 | 15091.076 |
| 465 | CTTCAACTTAACCTCGCACGTAAACGTAACTCGCCGGTTCATTCTACAAA | 15193 | 15177.807 | 15208.193 |
| 466 | AGAGTAGCCATAACACAAGGGTAGTATCCCAACAACGCCTCAGTCGAAAC | 15359 | 15343.641 | 15374.359 |
| 467 | GCTCGCGTACCACTTTAAATGGCGAACAGCCATACCCTTGGGACCTACTT | 15266 | 15250.734 | 15281.266 |
| 468 | CATAGACCTGTGTTTTTGCTAAACAGTTGCTTGAGCCTATTCTCTGCGGC | 15324 | 15308.676 | 15339.324 |
| 469 | ACACACAACCCCTACCAAGTATCACATGCACACGGTTTAGCCTCATCCAC | 15117 | 15101.883 | 15132.117 |
| 470 | TCTACGACCACGTACTCATGCGCCCTATTCAGACTCGCTTTCGCTGCGGC | 15185 | 15169.815 | 15200.185 |
| 471 | CATTCGGATATCTCTGGATCAAGGCTTACTTACAGCTCCCCAAAGCATGT | 15280 | 15264.72 | 15295.28 |
| 472 | GCTCTCCTACCACTGTTCGAAGAACAGTCCGCAGCTTCGGTGATACGTTT | 15288 | 15272.712 | 15303.288 |
| 473 | TCTTTTCGTCCCATCGCGGGTAATCGGCATCTTCACCGATACTACAATTT | 15213 | 15197.787 | 15228.213 |
| 474 | TGTACCCCCCATTGTAACACGTGTGTAGCCCCGGACGTAAGGGCCGTGCT | 15339 | 15323.661 | 15354.339 |
| 475 | TCCCCGGAGTACCTTTTATCCTTTGAGCGATGTCCCTTCCATACGGAAAC | 15223 | 15207.777 | 15238.223 |
| 476 | CGTTGAGCGATGGCCCTTCCTTTCGGTACCACCGGATCACTAAGCCCGAC | 15259 | 15243.741 | 15274.259 |
| 477 | TTCAAGGGGTCTTACTCGTTATACGATGGGATATCTAATCTTGGAGTCGG | 15477 | 15461.523 | 15492.477 |
| 478 | CCTCCTGATGTCCGACCAGGATTAGCCAACCTTCGTGCTCCTCCGTTACT | 15160 | 15144.84 | 15175.16 |
| 479 | ACCTTGGTCTTACGGCGGGAGGGAATCTCACCCTCCTTATCGTTACTTAT | 15294 | 15278.706 | 15309.294 |
| 480 | CGTGCCCCGCCCTACTCAGGATACTGCTAGCCACGATCAACTTTTAGGTA | 15242 | 15226.758 | 15257.242 |
| 481 | CACCCTCAGTTCATCCGGAAGCTTTTCAACGCTTATCGGTTCGGTCCTCC | 15175 | 15159.825 | 15190.175 |
| 482 | TCTACCTCCATGAGACTAATACGAGGCTAGCCCTAAAGCTATTTCGAGGA | 15338 | 15322.662 | 15353.338 |
| 483 | TACCTGTGTCGGTTTGCGGTACGGGCACCTTAGCATACACTAGAACTTTT | 15358 | 15342.642 | 15373.358 |
| 484 | AGCGGTTCCACAGCTTGTAAACATATGGTTTCAGGTTCTCTTTCACTCCC | 15253 | 15237.747 | 15268.253 |
| 485 | TTATAGTTACGGCCGCCGTTCACTGGGGCTTCGGGTCAAAGCTTGCACTC | 15360 | 15344.64 | 15375.36 |
| 486 | TTATAGTTACGGCCGCCGTTTACTGGGGCTTCGGTTCGATGCTTCGATTG | 15412 | 15396.588 | 15427.412 |
| 487 | GCCTTACGGGGTGGTCCCCGCTCATTCCCACAAGGTTTCTCGTGTCTCGT | 15263 | 15247.737 | 15278.263 |
| 488 | CCGGAGTTTTTCACACTGAGCCATGCAGCTCTGTGCGCTTATGCGGTATT | 15350 | 15334.65 | 15365.35 |
| 489 | CTTCTCCCGTTGGCCTTAGAATCTTCTTCCTACCTACCTGTGTCGGTTTG | 15178 | 15162.822 | 15193.178 |
| 490 | TGCCGCTTTAATGGGCGAACAGCCCAACCCTTGGGACCGACTACAGCCCC | 15262 | 15246.738 | 15277.262 |
| 491 | GGAGTTCTTCGTGATATCTAAGCATTTCACCGCTACACCACGAATTCCGC | 15256 | 15240.744 | 15271.256 |
| 492 | AGTGATGGGCAGGTTGGATACGCGTTACTCACCCGTGCGCCGGTCGACGC | 15460 | 15444.54 | 15475.46 |
| 493 | TCACGGTACTCGTACGCTATCGGTCAGACAGGTATACTCAGGCTTACCCG | 15322 | 15306.678 | 15337.322 |
| 494 | ACGCATTCGGAGTTTGTCAAGACTTGATAGGCGGTGAAGCCCTCGCATCT | 15417 | 15401.583 | 15432.417 |
| 495 | CATCATCTGTATGGCATTCGGAGTTTGATATCCCTTAGTAAGCTTTGACG | 15372 | 15356.628 | 15387.372 |
| 496 | TTCTCCGCTATCCACACCTCATCGCCACCCTTTTCAACGGATGTGCGTTC | 15095 | 15079.905 | 15110.095 |
| 497 | AAGCACTTTGGTTTGGGCTGTTCCCCGTTCGCTCGCCGCTACTTAGGGAA | 15351 | 15335.649 | 15366.351 |
| 498 | CACTTATGCCCGATTATTATCCACGCCAAACTCCTCGACTAGTGAGCTGT | 15216 | 15200.784 | 15231.216 |
| 499 | CTTAGGACCCGACTCACCCAGGGCAGACAAACTTGACCCTGGAACCCTTG | 15270 | 15254.73 | 15285.27 |
| 500 | CTCATCAGTTCTCACCCCCAATGTCCCCCGGATTTACCTGAGGGACGGGC | 15219 | 15203.781 | 15234.219 |
| 501 | CCCATGGTGCACGCACCATGGTTTGGGCTCTTCCGCGTTCGCTCGCCGCT | 15249 | 15233.751 | 15264.249 |
| 502 | GCTAGTCCTAAAACTATTTCGGGGAGAACCAGCTATCTCCGGGTTCGATT | 15376 | 15360.624 | 15391.376 |
| 503 | ACCCCATCAATTAACCTTCCGGCACCGGGCAGGCGTCACACCGTATACGT | 15221 | 15205.779 | 15236.221 |
| 504 | CATTCCGGCATTCTCACTCGAATACAATCCACCGCTGCTTCCGCTACGAC | 15122 | 15106.878 | 15137.122 |
| 505 | GTTTCAGTTCGCCGGGTACCTCTCTTGCAGGCCATGTATTCACCTGCAGA | 15295 | 15279.705 | 15310.295 |
| 506 | ACCTGAGGCTACTCGCCTCGACTACCTGTGTCGGTTTGCGGTACGGGTAG | 15401 | 15385.599 | 15416.401 |
| 507 | AAGGCTAGCCCTAAAGCTATTTCGAGGAGAACCAGCTATCTCCGGGTTCG | 15395 | 15379.605 | 15410.395 |
| 508 | ATTATTATTTTCTCCTCCTACGGGTACTGAGATGTTTCACTTCCCCGCGT | 15210 | 15194.79 | 15225.21 |
| 509 | GCTTGCGCTAACCTCTCCTCTTAACCTTCCAGCACCGGGCAGGCGTCAGC | 15195 | 15179.805 | 15210.195 |
| 510 | CAGAGGTCTGTCCAACACGGTCCTCTCGTACTAGTGTCAGAGCCACGCAA | 15316 | 15300.684 | 15331.316 |
| 511 | ATCCTCTCAGACCAGTTACGGATCGTCGCCTTGGTAGGCCTTTACCCCAC | 15209 | 15193.791 | 15224.209 |
| 512 | TCACGCAGAATTCCTCGTGCTCCGCGCTACTCAGGATACCACTAGGCTTC | 15218 | 15202.782 | 15233.218 |
| 513 | CGCGTCTTCGGTGGCGTGCTTGAGCCCCGCTACATTGTCGGCGCGGAACC | 15362.9 | 15347.5371 | 15378.2629 |
| 514 | TACTTATGCCCGATTATTATCCACGCCAAACTCCTCGACTAGTGAGCTGT | 15231 | 15215.769 | 15246.231 |
| 515 | ACCGTAGTGCCTCGTCATCACGCCTCAGCCTTGATTTTCCGGATTTGCCT | 15206 | 15190.794 | 15221.206 |
| 516 | AGCTGACGCCTGTATTTCCCAGTCTCCCACCTATCCTGTACATGAAATAC | 15176 | 15160.824 | 15191.176 |
| 517 | GGCGTTGCTGATCCGCGATTACTAGCGACTCCGCCTTCACGGAGCCGGGT | 15371 | 15355.629 | 15386.371 |
| 518 | GGGTGCCGCATGGGTTAAGCTTAGCGGATTTTCTCGGGAGTATGGTTACC | 15535 | 15519.465 | 15550.535 |
| 519 | TCTTCAGCCCCAGGATGCGATGAGCCGACATCGAGGTGCCAAACTTCCTC | 15292 | 15276.708 | 15307.292 |
| 520 | CGCCGGCACCGGATCACTATCTCCGACTTTCGTCCCTGCTCGATCCGTCG | 15161.8 | 15146.6382 | 15176.9618 |
| 521 | CACACTATCCGTCTCCGTCACTCCTTCGCTCCATATACGGGTGCAGGAAT | 15193 | 15177.807 | 15208.193 |
| 522 | ACTGTCAGGTTCGACTCTTCCTGCGGATTTGCCTGCAGGAATCAACATCT | 15303 | 15287.697 | 15318.303 |
| 523 | TCTTTCGGCGAGGGGGTTTCCCACCCCCTTTATCGTTACTTATACCTACA | 15205 | 15189.795 | 15220.205 |
| 524 | CTTTTCAGTGCTCTACAGGACACATCCATCACCTGAGGCTGTACCTCAAT | 15216 | 15200.784 | 15231.216 |
| 525 | ATGACCCTCCCCGGTTGAGCCGGGGGCTTTCACATCAGACTTAAGAAACC | 15316 | 15300.684 | 15331.316 |
| 526 | TTTCACAACTGACTTAAATATCCATCTACGCTCCCTTTAAACCCAATAAA | 15135 | 15119.865 | 15150.135 |
| 527 | CTACTTATTTTCGGTCCCTTACGCCCGGGTCAACCAACGCCCGGGTCCAG | 15210 | 15194.79 | 15225.21 |
| 528 | GTATTTAGGCTTACCGGGTGGTCCCGGCAGATTCACAGCAGATTCCACGA | 15402 | 15386.598 | 15417.402 |
| 529 | CTTCAACCTGGACATGGATAGGTCACCCGGTTTCGGGTCTGCACACACTG | 15338 | 15322.662 | 15353.338 |
| 530 | TCCGGAAGCCACGCCTCAAGGGCACAACCTCCAAGTCGACATCGTTTACG | 15270 | 15254.73 | 15285.27 |
| 531 | GGTCACCCGGTTTCGGGCCCATTGTATGCAACTTAACGCCCTTTTCAAAC | 15248 | 15232.752 | 15263.248 |
| 532 | GGCTACACATTTTAAAATGCTTAACCTTGCCGGAAAAAGTAACTCGTAGG | 15401 | 15385.599 | 15416.401 |
| 533 | CAAATTTCCTGCGCCCGCGACGGATAGGGACCGAACTGTCTCACGACGTT | 15332 | 15316.668 | 15347.332 |
| 534 | GCCAGGGTAGTATCCCACCGATGCCTCCACCGAAGCTGGCGCTCCGGTTT | 15300 | 15284.7 | 15315.3 |
| 535 | TTCACTGAAGGGTAACACCCCATAACAGGTGCCAGGTTTCCCCATTCGGA | 15315 | 15299.685 | 15330.315 |
| 536 | TCCAGCTAATCAGACGCGGGTCCATCTTATACCACCGGAGTTTTTCACAC | 15241 | 15225.759 | 15256.241 |
| 537 | CTTTATGAATATGCTTAGCGGATTTTCTTGGGAGCCTGATTACGTCCATT | 15378 | 15362.622 | 15393.378 |
| 538 | CATCAGGTAGTATTCAGGCTTACCAGGTGGTCCTGGCAGATTCACACGAA | 15410 | 15394.59 | 15425.41 |
| 539 | CATGCACCACGGATTTGCCTATGATGCGCGCTGCGTGCTTGACCACGGAA | 15363 | 15347.637 | 15378.363 |
| 540 | GACAGCCTGGCCATCATTACGCCATTCGTGCAGGTCGGAACTTACCCGAC | 15292 | 15276.708 | 15307.292 |
| 541 | TCACTGCTTTAAGCAGCTCCGACCGCTTGTAGGCGCACGGTTTCAGGAAC | 15338 | 15322.662 | 15353.338 |
| 542 | GCTCCCAACACCACGCGGCGATACCAACCCGAAGGAAGGAACCACCACGA | 15276 | 15260.724 | 15291.276 |
| 543 | GACTTCCCATTCCATTCCACTAAACCTTTACAATACCGTTTTCTGTCCGA | 15101 | 15085.899 | 15116.101 |
| 544 | ACTTAACGACCCGTCTGCGCTCCCTTTAAACCCAATAAATCCGGATAACG | 15203 | 15187.797 | 15218.203 |
| 545 | GGGGTGGGTTTCATACTTAGATGCTTTCAGCAGTTATCCGCTCCGCACTT | 15365 | 15349.635 | 15380.365 |
| 546 | GAAATCCTCGGATCAAAGCCCTGCTGGCGGCTCCCCGAGGCATATCGCAG | 15342 | 15326.658 | 15357.342 |
| 547 | CTTTCATGGCCCCTACTGATCATCGCCTTGGTAGGCCATTACCCTACCAA | 15168 | 15152.832 | 15183.168 |
| 548 | CTGTTATCCCCAGGGTAACTTTTATCCGTTGAGCGATGGCATTTCCACTC | 15269 | 15253.731 | 15284.269 |
| 549 | CCTACCCTCAGCTCATCCAGAAGCTTTTCAACGCTTATTGGTGCGGTCCT | 15199 | 15183.801 | 15214.199 |
| 550 | ACCAAGAAGGTGCTCCGACCGCTTGTAGGCACATGGTTTCAGGAACTATT | 15410 | 15394.59 | 15425.41 |
| 551 | CTTCTCCCGTTGGCCTTAGAATCTTCTTCCTACCTACCTGTGTCGGTTTG | 15178 | 15162.822 | 15193.178 |
| 552 | CCTGGCCAAGGGTAGATCACTTGGTTTCGCGTCTGCCACTGCCGACTATA | 15329 | 15313.671 | 15344.329 |
| 553 | GGGGGTCTCCCTTATGCCGAAGGCACGGGAGCAATTTGCCGAGTTCCTTG | 15450 | 15434.55 | 15465.45 |
| 554 | CATGGTTTAGCCCCGTTACATCTTCCGCGCAGGCCGACTCGACCAGTGAG | 15299 | 15283.701 | 15314.299 |
| 555 | ATCCGCCGCCTTTTCAACGGAGGTCGGTTCGGTCCTCCATGGAATTTTAC | 15295 | 15279.705 | 15310.295 |
| 556 | CCAAAGTCAATGCTAAGCTGTAGTAAAGGTTCACGGGGTCTTTTCGTCCC | 15376 | 15360.624 | 15391.376 |
| 557 | AAAGTTCGGTGGTTACGGAATTTCTACCGTATGTGCATCGACTACGCCGT | 15407 | 15391.593 | 15422.407 |
| 558 | CAGGTGTCAGCCCCTATACTTCATCTTTCGATTTAGCAGAGACCTGTGTT | 15293 | 15277.707 | 15308.293 |
| 559 | ACTTAAAGCCAGCGCCCCTTCTCCCGAAGTTACGGGGCCATTTTGCCGAG | 15283 | 15267.717 | 15298.283 |
| 560 | ACTTAGATGCTTTCAGCACTTATCCGATCCAGACTTAGATACCCGGCAAT | 15264 | 15248.736 | 15279.264 |
| 561 | CTACAGGATTTAGTTTAGCGGATTTTCTTGGCAGCATGATTACATGCACT | 15396 | 15380.604 | 15411.396 |
| 562 | CCTTAACCTTCCGGCACTGGGCAGGTGTCAGCCCGTATACGTCGTATCTC | 15265 | 15249.735 | 15280.265 |
| 563 | TGAGCCAACATCCTAGTTGTCTTCGAAATCCCACATCCTTTTCCACTTAA | 15150 | 15134.85 | 15165.15 |
| 564 | CAGGATGTGACGAGCCGACATCGAGGTGCCAAACCCCTCCGTCGATATGA | 15390 | 15374.61 | 15405.39 |
| 565 | GGTTTTGCCGGTCCATGGTCGGTACGGGAATATCCACCCGTTCATCCATT | 15335 | 15319.665 | 15350.335 |
| 566 | CTTTACGCTATCGGTCATTGGGTAGTATTTAGGCTTGGAGGGTGGTCCCC | 15461 | 15445.539 | 15476.461 |
| 567 | GCATGGATTAAGTTTAGCGGATTTTCTAGGAAGTATGATTACCTACGCTA | 15469 | 15453.531 | 15484.469 |
| 568 | ACTGTCCATCCTCTGGTTTCACAGAGCTATGTTAGAATTTCAGTAACCGA | 15310 | 15294.69 | 15325.31 |
| 569 | ACCTCGCGGTACGCCTTCGACGCCGACTGGAATGCTCCCCTACCGATCAT | 15204 | 15188.796 | 15219.204 |
| 570 | CTCTTGCGATGAGCTCTCCTCTTAACCTTCCAGCACCGGGCAGGTGTCAG | 15265 | 15249.735 | 15280.265 |
| 571 | AGCTGACGCCTTGGCTTCCCAGTCTCCCACCTATCCTGTACATGTAATAC | 15168 | 15152.832 | 15183.168 |
| 572 | GAATGAATGGCTGCTTCCAAGCCAACATCCTAGCTGTCACTGGGACCAGA | 15364 | 15348.636 | 15379.364 |
| 573 | TGAGCCAACATCCTGGTTGTCTACGTATCTTCACATCGTTTTCCACTTAA | 15212 | 15196.788 | 15227.212 |
| 574 | TGAGGGCACCTTTAGAAGCCTCCGTTACGCTTTTGGAGGCGACCACCCCA | 15323 | 15307.677 | 15338.323 |
| 575 | TTAAATCGACCGAAGTTTCAATAAAGTAATTCCCGTTCGACTTGCATGTG | 15358 | 15342.642 | 15373.358 |
| 576 | AGTCGGGTTGCAGACTCCAATCCGAACTGAGAGAGGCTTTAGGGATTAGC | 15515 | 15499.485 | 15530.515 |
| 577 | CCTGTGTCGGTTTACGGTACGGGTATGGTATGAACAATAGCGGCTTTTCT | 15469 | 15453.531 | 15484.469 |
| 578 | CTCCCGGATTCCGACGGAATTTCACGTGTTCCGCCGTACTCAGGATCCAC | 15234 | 15218.766 | 15249.234 |
| 579 | AAACATTAAAGGGTGGTATTTCAAGGTCGGCTCCATGCAGACTGGCGTCC | 15450 | 15434.55 | 15465.45 |
| 580 | CCTGAGTATATTCAACCCGACTACGTGTGTCCGTTTACGGTACGGGTACC | 15328 | 15312.672 | 15343.328 |
| 581 | ACCACGAATTCCGCCTGCCTCAACTGCACTCAAGATATCCAGTATCAACT | 15163 | 15147.837 | 15178.163 |
| 582 | AGTGAGCTATTACGCACTCTTTTAATGAGTGGCTGCTTCTAAGCCAACAT | 15350 | 15334.65 | 15365.35 |
| 583 | GGCTCACGCCCCGCCTTCAACGCCGAGTGGAATGCTCCCCTACCGATGAT | 15229 | 15213.771 | 15244.229 |
| 584 | AGGGCACCTTTAGAAGCCTCCGTTACACTTTTGGAGGCGACCACCCCAGT | 15307 | 15291.693 | 15322.307 |
| 585 | CTCTGCCATCGCCATCGCCGTTCGGCTTAGACTTAGGACCCGACTGACCC | 15195 | 15179.805 | 15210.195 |
| 586 | GCCGAGTTCCTTAACAAGGGTTCTCCCGCTCGTCTTAGGATTCTCTCCTC | 15206 | 15190.794 | 15221.206 |
| 587 | CTCCCCCCCCCCCCTTCCCCTCCGCGGCCACCTTTCCCCCCCCCTCCCCA | 14685 | 14670.315 | 14699.685 |
| 588 | CCCATATACACGGGTTAGAATCCAAACAAATGAAGGGTCGTATTTCAACA | 15379 | 15363.621 | 15394.379 |
| 589 | CCCGCATCAGCGGGTTAGAACTCAAATAATCAAAGGGCCGTATTTCAACA | 15356 | 15340.644 | 15371.356 |
| 590 | CTTCACAGTACTATACGCTATCGGTCACTGGGTAGTATTTAGGGTTGGAG | 15462 | 15446.538 | 15477.462 |
| 591 | CATTCCCACTTAATACCACCGGATCACTAAGCCCTACTTTCGTACCTGCT | 15096 | 15080.904 | 15111.096 |
| 592 | CTTCCGTCGCCCCGCGGTGGTTTCACTGCTCCGTCTCCACGTCGCCCCAT | 15105 | 15089.895 | 15120.105 |
| 593 | GCGGGTAACCTGCATCTTCACAGGTACTAAAATTTCACCGAGTCTCTCGT | 15296 | 15280.704 | 15311.296 |
| 594 | AAAAGTACGCGGTTGAGCTAATAATGCTCTTCCACAGCTTGTAAACACAG | 15386 | 15370.614 | 15401.386 |
| 595 | CGGTACGGGAATATCAACCCGTTCATCCATTCGACTACGCCTGTCGGCCT | 15258 | 15242 .742 | 15273.258 |
| 596 | CCTCATCTACCTGTGTCGGTTTGCGGTACGGGCGCCTTAGTATACCTCAT | 15286 | 15270.714 | 15301.286 |
| 597 | GTAGTATTTAGCCTTGGAGGGTGGTCCCTCCTGCTTCCCACAGGGTTTCA | 15366 | 15350.634 | 15381.366 |
| 598 | TTCCGTCAGGTGGCGGCACTTACGTTCCTTCGTCTCTCCATCGAGGTATA | 15286 | 15270.714 | 15301.286 |
| 599 | CTTCAAAGTCTCCGGCCTATCCTACACATCAATTACCCAAATTCAATGTT | 15143 | 15127.857 | 15158.143 |
| 600 | CTCTCAGGGCTCTTACTAACTGAACGTTATGGGAAATCTCATCTTGAGGG | 15391 | 15375.609 | 15406.391 |
| 601 | AAGTCCTCGAGCGATTAGTATTGGTCCGCTTCACGTCTCACAACGCTTCC | 15248 | 15232.752 | 15263.248 |
| 602 | ACGCCTTTCGTGCAGGTCGGAACTTACCCGACAAGGAATTTCGCTACCTT | 15297 | 15281.703 | 15312.297 |
| 603 | CCTGATCGACTTGTATGTCTCCCAGTCAAGCGCCCTTATGCCATTACACT | 15183 | 15167.817 | 15198.183 |
| 604 | CGTTTTCCACTTAGCATGTATTAGGGACCTTAGCTGTGGGTCTGGGCTGT | 15436 | 15420.564 | 15451.436 |
| 605 | TAGTCAAGTATCGTCTCTCTTCTTCCTTGCTGATAGACCTTTACATACCG | 15203 | 15187.797 | 15218.203 |
| 606 | GACACATGGTTTTCTGCAACTGCCGGCCGGCCCGTCGGAGCCGGCGCACG | 15366 | 15350.634 | 15381.366 |
| 607 | TTTCTCGTGTCTCGTGGTACTCTGGATCCCGCCTTGCCGCTCCCGGTTTC | 15196 | 15180.804 | 15211.196 |
| 608 | CTAATGAGATGTTTCAGTTCACAGCGTTTACCTCCAACTAGACTATGAAT | 15318 | 15302.682 | 15333.318 |
| 609 | ATCCTTTCCCACTTAGCACGCGCTTGGGGACCTTAGACGACGATCTGGGC | 15313.9 | 15298.5861 | 15329.2139 |
| 610 | GTTTCACGTGTCTGGCCGTACTCTGGAACTCGCTCAGCTCTTGTCGTTTT | 15283 | 15267.717 | 15298.283 |
| 611 | ATGGTTATAGTTACCACCGCCGTTTACCGGGGCTTGAATTCACCGCTTCG | 15319 | 15303.681 | 15334.319 |
| 612 | CCGCACGGAATGGCCGTCTCGTCTCGGGGCGGGCTTCCCGCTTAGATGCT | 15363 | 15347.637 | 15378.363 |
| 613 | TGCTCGACTTGTCTGTCTCGCAGTCAAGCTCCCTTATACCTTTACACTCT | 15140 | 15124.86 | 15155.14 |
| 614 | ATGCATTGCCAGAAGCTTTTCCTGGAAGCCGTCATCATGTGCTTCGCTAC | 15303 | 15287.697 | 15318.303 |
| 615 | TCTTGCGGCGAGCAGGTTTCTCACCTGCTTTATCGTTACTTATACCTACA | 15244 | 15228.756 | 15259.244 |
| 616 | CGCGCACGCAACCCCCGACGGGTATCACGCGCACGCGGTTTGGTCTGATC | 15310 | 15294.69 | 15325.31 |
| 617 | CGCTTTATCGTTACTTATGTCAGCATTCGCACTTCTGATACCTCCAGCAT | 15188 | 15172.812 | 15203.188 |
| 618 | GACAGTGCCCAAATCATTACGCCTTTCGTGCGGGTCGGAACTTACCCGAC | 15307 | 15291.693 | 15322.307 |
| 619 | TCCCATCTATCCTGTGCATGCAACACCGAAACCCAATATTAGGCTACAGT | 15218 | 15202.782 | 15233.218 |
| 620 | CCCGGGTCATGCCCTTTCAGAGTGTCCCTCTGCTTAAAACTTTCGGTGGT | 15286 | 15270.714 | 15301.286 |
| 621 | GGGATCCCATTCCCGGCTTCCGCTCTCTGCACGTGTCCCCACAGTTCTGT | 15168 | 15152.832 | 15183.168 |
| 622 | CACCTCGCCATACACGCCGCACGGATTTGCCTATGCGACTGGCTGCGTGC | 15260 | 15244.74 | 15275.26 |
| 623 | TCGCTCCTCAGCGTCAGTTACAGACCAGAGAGTCGCCTTCGCCACTGGTG | 15299 | 15283.701 | 15314.299 |
| 624 | TATCGAACCATAACGGCTCCCATCATCACACCTCGCCATGCATGCCATGC | 15140 | 15124.86 | 15155.14 |
| 625 | TTCACCGGGGCTTCAATTCGGAGCTTGCACCCCTCCTCTTGACCTTCCGG | 15192 | 15176.808 | 15207.192 |
| 626 | CTGCAGGATTAAGTTTAGCGGATTTTCTCGGCAGCATGCTTACGCGCACT | 15383 | 15367.617 | 15398.383 |
| 627 | TCTCCTACCATACCTATAAAGGTATCCACAGCTTCGGTAATATGTTTTAG | 15269 | 15253.731 | 15284.269 |
| 628 | GGGCGCGTCATGCCCTCACGTCGAGGCTTTTCTCGGCAGCATAGGATCAC | 15355 | 15339.645 | 15370.355 |
| 629 | CTCCGACGGATTGTAGGCGCACGGTTTCAGGAACTCTTTCACTCCCCTCC | 15225 | 15209.775 | 15240.225 |
| 630 | CACTCGACTAGTGAGCTATTACGCACTCTTTGAATGAATAGCTGCTTCTA | 15310 | 15294.69 | 15325.31 |
| 631 | ACTCCCCTCGCCGGGGTTCTTTTCGCCTTTCCCTCACGGTACTGGTTCAC | 15134 | 15118.866 | 15149.134 |
| 632 | CCCTCCCGGGGTTCTTTTCACCTTTCCCTCACGGTACTATGCGCTATCGG | 15158 | 15142.842 | 15173.158 |
| 633 | CTGGTCCTCTCGTACTAGGAGCAGATCCTCTCAAATTTCCTTCGCCCGCG | 15200 | 15184.8 | 15215.2 |
| 634 | ACTTTCGTTACTGCTCGGGCCGTCACCCTCGCAGTTAGGCTAGCTTTTGC | 15262 | 15246.738 | 15277.262 |
| 635 | TGTAATAGCCACGTAATTTAAAACTGAAATTGAGAGAGACTTACCCAGAG | 15458 | 15442.542 | 15473.458 |
| 636 | GGTGGTCTACCGGGAGACTTACCCTCATGTGAGGTGGGAATACTCATCTT | 15448 | 15432.552 | 15463.448 |
| 637 | TGGCGGTCTGGGCTGTTTCCCTTTCGACTACGGATCTTATCACTCGCAGT | 15317 | 15301.683 | 15332.317 |
| 638 | TCTCCACATCACTCTTATAGGTAGTACAGGAATATTAACCTGTTCTGCCA | 15254 | 15238.746 | 15269.254 |
| 639 | CCATTCTGAGGGTACCTTTGGGCGCCTCCGTTACTCTTTCGGAGGCGACC | 15312 | 15296.688 | 15327.312 |
| 640 | GATGGCAGGACTGTCACTTCTCCGTCTCCACATCGCTCCATAAAGTAGTA | 15281 | 15265.719 | 15296.281 |
| 641 | TCGGCGCAGAGTCACTCGACCAGTGAGCTATTACGCACTCTTTAAATGGT | 15361 | 15345.639 | 15376.361 |
| 642 | CGCGGCATGGCTGCATCAGGCTTGCGCCCATTGTGCAGTATTCCCCACTG | 15306 | 15290.694 | 15321.306 |
| 643 | CGGACATCCTTAATGACATTCGCAGTTTGATTGTATTCAGTACCCCGGGA | 15351 | 15335.649 | 15366.351 |
| 644 | TACCGGCATTCTCACTTCTAAGCGCTCCACCAGTCCTTCCGGTCTGGCTT | 15151 | 15135.849 | 15166.151 |
| 645 | TTCGGGCCTCCATTCAGTGTTACCTGAACTTCACCCTGGACATGGGTAGA | 15328 | 15312.672 | 15343.328 |
| 646 | CGGAGGCGACCGCCCCAGTCAAACTCCCCGCCTGGCATTGTCCCACCGCC | 15160 | 15144.84 | 15175.16 |
| 647 | ACCTTTTAGGAGGCGACCGCCCCAGTCAAACTGCCCGTCAGACACTGTCT | 15252 | 15236.748 | 15267.252 |
| 648 | ACAGCCCAGCCTTCCGTTGTGCGTACTTCACTACACAACAGCCTCACTGC | 15147 | 15131.853 | 15162.147 |
| 649 | TCATACCACCGGAGTTTTTACCCCTGCACCATGCGGTGCTGTGGTCTTAT | 15270 | 15254.73 | 15285.27 |
| 650 | CACTCACCCGAAGGCTTGCTCCCAAACAAAAGAGGTTTACAACCCGAAGG | 15311 | 15295.689 | 15326.311 |
| 651 | CGTCAATTCATTTGAGTTTTAACCTTGCGGCCGTACTCCCCAGGCGGTCG | 15295 | 15279.705 | 15310.295 |
| 652 | ACTTTCGTTCCTGCTCGACTTGTCAGTCTCGCAGTCAGGCTGGCTTGTGC | 15293 | 15277.707 | 15308.293 |
| 653 | CCACCAGGGAGGCTCCGACGGTTTGTGGGCGCACGGTTTCAGGAACTGTT | 15475 | 15459.525 | 15490.475 |
| 654 | ACTGGCGTGCACGTCTCTTTGTCTCCCACCTATCCTGTACATGTATGACC | 15190 | 15174.81 | 15205.19 |
| 655 | TGATAGCGTGAGGTCCGAAGATCCCCCACTTTCTCCCTCAGGACGTATGC | 15298 | 15282.702 | 15313.298 |
| 656 | AAATCTTTAATCTCTTTCAGATGTCTTCTAGAGACGTCATTGGGTATTAG | 15370 | 15354.63 | 15385.37 |
| 657 | CACCGGGGCCCCAAGACCCACACACACCAACAAACCCGAAGGCTTAGTGG | 15267 | 15251.733 | 15282.267 |
| 658 | TACTTTTCCAATTTTTTTTTTTTTTTTTTTTTTTTTTTTCTTCCAATAAA | 15130 | 15114.87 | 15145.13 |
| 659 | CTCTGCCTATCCTTCTGTGTCACTGCATCCGGTTGCTCGGCGGTATCGGA | 15278 | 15262.722 | 15293.278 |
| 660 | ATGCCTGGCAGTTCCCTACTCTCGCATGGGGAGACCCCACACTACCATCG | 15228 | 15212.772 | 15243.228 |
| 661 | AACATCCTGGTTGTCTAAGCAACTCCACATCCTTTTCCACTTAACGTATA | 15174 | 15158.826 | 15189.174 |
| 662 | CTCCGGCCGGGCCCGCCAGGACCCGGACACACGCTCCCTCAACACCACGC | 15138.7 | 15123.5613 | 15153.8387 |
| 663 | TTCTCTGCGGCTCTTTCGAGCACTCCTTATTCCGAAGTTACGGAGTCAAT | 15269 | 15253.731 | 15284.269 |
| 664 | GGCACAGCCCTGTGTTTTTGTTAAACAGTTGCCTGGACCGATTCTCTGCG | 15350 | 15334.65 | 15365.35 |
| 665 | TGCTCCCCACGCTTTCGAGCCTCAACGTCAGTTACTGTCCAGTAAGCCGC | 15194 | 15178.806 | 15209.194 |
| 666 | ATGCGTCCCACGGATTTGCCTATGGGACGGGCTGCGTGCTTGACCACGGA | 15435 | 15419.565 | 15450.435 |
| 667 | CCCAGACAACCATCGCTGGGGTTGAGCTACCTCCCTGCGTCCCTCCGCAG | 15205 | 15189.795 | 15220.205 |
| 668 | ACGCCGTTAGGCCTCACCTTAGCTCCCGACTGACCTGGAGCGGACGAACC | 15278 | 15262.722 | 15293.278 |
| 669 | GCCTTTAGCCTTAACCTTGCCAGCCGGCGTAACTCGCCGGACCGTTCTAC | 15210 | 15194.79 | 15225.21 |
| 670 | TGGCCGTTCAACCTCTCAGTCCGGCTACTGATCGTCGCCATGGTGAGCCG | 15306 | 15290.694 | 15321.306 |
| 671 | CGCTTTCGCTCGCCACTACTCACGGAGTATCCCTTCCTGCAGGTACTGAG | 15225 | 15209.775 | 15240.225 |
| 672 | AGGACCCGACTCACCCGGGGACGACGAACGTGGCCCCGGAACCCTTGGTC | 15353 | 15337.647 | 15368.353 |
| 673 | CATTGCGGAAGATTCCCCACTGCTGCCTCCCGTAGGAGTCTGGACCGTGT | 15330 | 15314.67 | 15345.33 |
| 674 | GCATGTATTAGGCACGCCGCCAGCGTTCGTCCTGAGCCAGGATCAAACTC | 15332 | 15316.668 | 15347.332 |
| 675 | CCCGTTACCCATCATCGCCATGGTAGGCCTTTACCCTACCATCTAGCTAA | 15137 | 15121.863 | 15152.137 |
| 676 | GCCCTCACCCGATTAGTAACAGTCAGCTCCATGTGTTGCCACACTTCCAC | 15162 | 15146.838 | 15177.162 |
| 677 | ACCCCAAGTCATCCCCCGGTTTTCAACCCAGGTGGGTTCGGTCCTCCACG | 15194.8 | 15179.6052 | 15209.9948 |
| 678 | CGCCTTAGGACCCGACTAACCCAGGGCGGATAAACCTAGCCCTGGAACCC | 15280 | 15264.72 | 15295.28 |
| 679 | TTCCGTCTTGCCGCGGGTACACTGCATCTTCACAGCGAGTTCAATTTCAC | 15239 | 15223.761 | 15254.239 |
| 680 | GTACGGGTAACACAGAAATATGCTTAGCGGGTTTTCTTGGGAGCCGGTTT | 15527 | 15511.473 | 15542.527 |
| 681 | AAGCTCCATGGGGTCTTTCCGTCTTGTCGCGGGTAACCGGCATCTTCACC | 15296 | 15280.704 | 15311.296 |
| 682 | AACTTTATTCCCTTATAGAAGCAGTTTACAACCCATAGGGCCGTCTTCGT | 15270 | 15254.73 | 15285.27 |
| 683 | GGGCGGGATTCGCACCCGCCTCTCGCTACTCATGTCTGCATTCTCACTCC | 15176.8 | 15161.6232 | 15191.9768 |
| 684 | ATACTATCAGGTTCGGATCTCATGGTGGATTTGCCTGCCATGATCGACTC | 15358 | 15342.642 | 15373.358 |
| 685 | ACGCCGTCGGGCATATAAAGCCCTCCGACAGTTTGTAAACACAGGGTTTC | 15355 | 15339.645 | 15370.355 |
| 686 | GCCTATCGACCACGTGTTCTGCATGGGGTCTTCAGCGGCTCGGGGCCGCA | 15387 | 15371.613 | 15402.387 |
| 687 | GGATAAGGGTTGCGCTCGTTGCGGGACTTAACCCAACATTTCACAACACG | 15395 | 15379.605 | 15410.395 |
| 688 | GCCCCCGAGCCTTGGCAGTGCTCTACACGGCGTGAGGTTCATCCGAGGCT | 15356 | 15340.644 | 15371.356 |
| 689 | TTCCTTAACCAAGAATCTCTCAACGCCTTAGTATGTTCTACCCGACCACG | 15160 | 15144.84 | 15175.16 |
| 690 | TTTCCCTGCGGCTCCGGGACTTTATCCCTTAACCTTGCCAGTATGCACAA | 15199 | 15183.801 | 15214.199 |
| 691 | TACTGTCAGGTTCGACTCTTGCACCGGATTTGCCTGGCACAATCAACATC | 15272 | 15256.728 | 15287.272 |
| 692 | GCCTTCCCATGCCATTCTGCTAGATACCTTCCATACCGTGCGCTGTCCGA | 15160 | 15144.84 | 15175.16 |
| 693 | ATGAGCCGACATCGAGGTGCCAAACACCGCCGTCGATATGAACTCTTGGG | 15405 | 15389.595 | 15420.405 |
| 694 | TTCGGCTCAAAGTCCGGATTTGCCTGGACCTCTCATCACCTACACTCTTC | 15159 | 15143.841 | 15174.159 |
| 695 | ACGCATTTCACCGCTACACGTGGAATTCCACTCTCCTCTTCTGCACTCAA | 15112 | 15096.888 | 15127.112 |
| 696 | TTTCCGTTTCGCCTACGGGGCTCTCACCCTCTCTGGCCGGTCTTTCCAGA | 15174 | 15158.826 | 15189.174 |
| 697 | GCCCCGGACAACCATCGCCGGGGATGAGCTACCTCCCTGCGTCCCTCCGC | 15191 | 15175.809 | 15206.191 |
| 698 | TGTCGCGGGTAACCGGCATCTTCACCGGTACTACAATTTCGCCGGGCGGG | 15395 | 15379.605 | 15410.395 |
| 699 | AAGCCCTCGATCTATTAGTACACACTTGCTGAATGGATCGCTCCACTTAC | 15240 | 15224.76 | 15255.24 |
| 700 | CCTTGGCAACAGTTCTCTCGCTCACCTCGGGATACTCTCCCTGCCCACCT | 15081 | 15065.919 | 15096.081 |
| 701 | TCTCCGCCAAAGCCAAAGCCTTGGTTTCCCAGAGTCCCATCTATCCTGTG | 15193 | 15177.807 | 15208.193 |
| 702 | AGGAGTATTCAGGCTTACCAGGTGGTCCTGGCAGATTCACACGAGATTTC | 15441 | 15425.559 | 15456.441 |
| 703 | CAGGATGTGACGAGCCGACATCGAGGTGCCAAACCACTCCGTCGATATGA | 15414 | 15398.586 | 15429.414 |
| 704 | CAACCTGTTGTCCATCGGCTACGCTTTTCAGCCTCACCTTAGGTCCCGAC | 15160 | 15144.84 | 15175.16 |
| 705 | TCAGATGGCGGCACTGCCACGACTCCGTCTCCACGTCACTCCCCAAGGTA | 15213 | 15197.787 | 15228.213 |
| 706 | CTACGGGGCCATCACCCTCTGCGGCCCGGCATTCAATCCGGTTCGCCTCA | 15195.8 | 15180.6042 | 15210.9958 |
| 707 | CCAGGTCATAAGGGGCATGATGATTTGACGTCATCCCCACCTTCCTCCGG | 15298 | 15282.702 | 15313.298 |
| 708 | CCTTTAATCATGTGAACATGCGGACTCATGATGCCATCTTGTATTAATCT | 15300 | 15284.7 | 15315.3 |
| 709 | TTTTCACACCTGACTTAAGATCCCGCCTTAAGCTTCCCTTTACACCCAGT | 15102 | 15086.898 | 15117.102 |
| 710 | CCTACCCTCAGCTCATCCAGAAGCTTTTCAACGCTTATTGGTGCGGTCCT | 15199 | 15183.801 | 15214.199 |
| 711 | GTCACACTGAGTATTTAGGCTTACCGGGTGGTCCCGGCAGATTCACAGCA | 15402 | 15386.598 | 15417.402 |
| 712 | CCAGGATAACTTACGTACACCATTCGACGCCGTGAGTATGCTCCCCTACC | 15211 | 15195.789 | 15226.211 |
| 713 | AGAGAACCAGCTATCTCCAAGTTCGTTTGGAATTTCTCCGCTACCCACAA | 15249 | 15233.751 | 15264.249 |
| 714 | CCCGAAGTTACGGGGTAATTTTGCCGAGTTCCTTAACAACCCTTCTCCCG | 15248 | 15232.752 | 15263.248 |
| 715 | GGCTCACGCCCCACCTTCGACGCGGAGTGGAATGCTCCCCTACCGATGTT | 15260 | 15244.74 | 15275.26 |
| 716 | GTATCTAATCCTGTTTGCTCCCCACGCTTTCGCACTGAGCGTCAGTCTTC | 15181 | 15165.819 | 15196.181 |
| 717 | CGCGAGTCCATCCTGAAGCGAATAAATCCTTTTCCCTCAGCACCATGCGG | 15251 | 15235.749 | 15266.251 |
| 718 | TTATCGCAGCTTATCACGTCTTTCTTCGGCTCTTAGTGCCAAGGCATCCA | 15229 | 15213.771 | 15244.229 |
| 719 | CGGCAAAGATTCTCACTTTGCTCTCGCTACTCATGCCGGCATTCTCTCTC | 15150 | 15134.85 | 15165.15 |
| 720 | CCGGCAGACCGATCAAGAAAAAACCCACAACCCCGCACGCGCAACCCCTG | 15196 | 15180.804 | 15211.196 |
| 721 | GGGCTGTTTCCCTTTTGACTATGAGACTTATCTCACATAGTCTGACTGCT | 15299 | 15283.701 | 15314.299 |
| 722 | CCCCACTGCTGCCTCCCGTAGGAGTCTGGACCGTGTCTCAGTTCCAGTGT | 15257 | 15241.743 | 15272.257 |
| 723 | TTGTGACTATTCTCTGCGGCCTGCTCTCGCAGGCACCCCTTATCCCGAAG | 15216 | 15200.784 | 15231.216 |
| 724 | TTACCTCCACTTCAACCTGGACATGGGTAGGTCACCCGGTTTCGGGTCGA | 15329 | 15313.671 | 15344.329 |
| 725 | TCGCAAGGTTATCCCCAAGTGAAGGGCAGGTTGGATACGCGTTACTCACC | 15411 | 15395.589 | 15426.411 |
| 726 | CGCGATCGGCAGACCATGCGCGTTCAGGTACGGGGCCCTCACCCTCTGCG | 15326 | 15310.674 | 15341.326 |
| 727 | GCCTTTCACTCCTACACTCGGCTCATCCAGAAGCTTTTCAACGCTTATTG | 15158 | 15142.842 | 15173.158 |
| 728 | AGTTTGATAAGGTTCAGTAACCTCTCGGCCCCTAGCCAATTCAGTGCTTT | 15302 | 15286.698 | 15317.302 |
| 729 | GGCTGCAACACGGTGACGTGAAGCGAATCCCAAAAACCATCTCTCAGTTC | 15333 | 15317.667 | 15348.333 |
| 730 | CCGGTCTCTCGACTAGTGAGCTGTTACGCACTCTTTGAATGAATGGCTGC | 15359 | 15343.641 | 15374.359 |
| 731 | GGATCACTAACTCCAACTTTCGTTACTGCTCGAACTGTCGCTCTCGCAGT | 15223 | 15207.777 | 15238.223 |
| 732 | CTCGCGTACCGCTTTAATGGGCGAACAGCCCAACCCTTGGGACCGACTAC | 15277 | 15261.723 | 15292.277 |
| 733 | CGGCTACGCCTTTCGGCCTCACCTTAGCTCCCGACTAACTTGGAGCGGAC | 15235 | 15219.765 | 15250.235 |
| 734 | ACCTTTCCCTCACGGTACTGGTTCACTATCGGTCACTAGGGAGTATTTAG | 15318 | 15302.682 | 15333.318 |
| 735 | ATACTGTCAGGTTCGACTCTTGCACCGGATTTGCCTGGCGCAATCAGCAT | 15328 | 15312.672 | 15343.328 |
| 736 | TGTCATGCTCTATGGTCTTTCTTTCCAGAAAGTTCTTCTCCGATGTCTTC | 15216 | 15200.784 | 15231.216 |
| 737 | ATCACCTTAGGATTCTCTCCTCGCCTACCTGTGTCGGTTTGCGGTACGGG | 15302 | 15286.698 | 15317.302 |
| 738 | ACGTATTCACCGTGGCATTCTGATCCACGATTACTAGCGATTCCGACTTC | 15247 | 15231.753 | 15262.247 |
| 739 | TAGAGCATTTTCTTGGAAGCAGGATTACCCACACTATTGGTTTACTCCGA | 15350 | 15334.65 | 15365.35 |
| 740 | CATTGACCAATATTCCTCACTGCTGCCTCCCGTAGGAGTTTGGGCCGTGT | 15295 | 15279.705 | 15310.295 |
| 741 | ATCCGCCGCCTTTTCAACGGAGGTCGGTTCGGTCCTCCATGGAATTTTAC | 15295 | 15279.705 | 15310.295 |
| 742 | CCTGTGTCGGTTTACGGTACGGGCGCATGGCAAACGATAGCGGCTTTTCT | 15440 | 15424.56 | 15455.44 |
| 743 | GCCCAAGGGTAGATCACTTGGTTTCGCGTCTACTCCTTCCGACTATACGC | 15264 | 15248.736 | 15279.264 |
| 744 | GGCGGATTTTCCCAAATCCTTCGACTATCAAGTTCTTTGGTAACTCAAAT | 15285 | 15269.715 | 15300.285 |
| 745 | CTTTCGGGGAGTACGAGCTATCTCCGAGTTTGATTGGCCTTTCACTCCTA | 15325 | 15309.675 | 15340.325 |
| 746 | CTCTAGTTAGCCTGCTGCGTCCCTCCTTCACTCAATACTCTAGTACAGGA | 15183 | 15167.817 | 15198.183 |
| 747 | CGCCGTCGATGTGAACTCTTGGGCGAGATCAGCCTGTTATCCCCAGGGTA | 15394 | 15378.606 | 15409.394 |
| 748 | AGTCGTTTCCAACTGTTGTCCCCCACTCCAGGGCAGGTTACTCACGCGTT | 15240 | 15224.76 | 15255.24 |
| 749 | GCATGCTTAAAGTTCGGCGGCTACGGAATTTCAACCGTATGTGCATCGAC | 15401 | 15385.599 | 15416.401 |
| 750 | ATTACCGCGGCTGCTGGCACGGAATTAGCCGGTCCTTATTCTTATGGTAC | 15359 | 15343.641 | 15374.359 |
| 751 | CGCACAGCCCTGTGTTTTTGTTAAACAGTTGCCTGGACCTATTCTCTGCG | 15285 | 15269.715 | 15300.285 |
| 752 | CATAATTTTATTTTCTTCTCCTACGGGTACTGAGATGTTTCACTTCCCCG | 15209 | 15193.791 | 15224.209 |
| 753 | ACCTTGGGCGGACGAACCTTCCCCAAGAAACCTTAGATTTTCGGCCATTA | 15290 | 15274.71 | 15305.29 |
| 754 | TACTATCAGGTTCGGCTCTCAAGGTGGATTTGCCTGCCTCGATCTGCGCC | 15311 | 15295.689 | 15326.311 |
| 755 | CTGTACATGCAATACCAAGCTCCAGTACCAAACTGGAGTAAAGCTCCATG | 15316 | 15300.684 | 15331.316 |
| 756 | TGCTTGACCACGGAAAACCACCTCCGCGGCCGGCTCCCATTCCGTGTCAC | 15189 | 15173.811 | 15204.189 |
| 757 | CAGTAACCCGCAAGGCTGCACCTAAATGCATTTCGGGGAGTACGAGCTAT | 15404 | 15388.596 | 15419.404 |
| 758 | AAGCCAACATCCTGGTTGTCTACGCAATTGCACATCCTTTTCCACTTAAC | 15175 | 15159.825 | 15190.175 |
| 759 | CACATCTTACGACGGCAGTCTCGACAGAGTCCCCAGCATCACCTGATGGT | 15276 | 15260.724 | 15291.276 |
| 760 | TTATAGTTACGGCCGCCGTTCACTGGGGCTTCGATTCAATGCTTGCACAT | 15334 | 15318.666 | 15349.334 |
| 761 | CATCTTTACTCGTACTGCAATTTCGCCGAGCTCCTGGTCGAGACAGTGGG | 15344 | 15328.656 | 15359.344 |
| 762 | ACACCGAGCCATGCAGCTCTGTGCGCTTATGCGGTATTAGCAGTCATTTC | 15328 | 15312.672 | 15343.328 |
| 763 | AGGTCCCGCGCTCCCCACCACCGTCCCCGTCAAAGACGGGGTTCGGGATG | 15295 | 15279.705 | 15310.295 |
| 764 | ATCGAGCTCACAGCATGTGCATTTTTGTGTACGGGGCTGTCACCCTGTAT | 15374 | 15358.626 | 15389.374 |
| 765 | GGAATTTCTCCCCTAGCCACAAGTCATCCGCTAACTTTTCAACGGTAGTC | 15216 | 15200.784 | 15231.216 |
| 766 | GCTCTACCTCCAAGACTCTTACCTTGAGGCTAGCCCTAAAGCTATTTCGG | 15232 | 15216.768 | 15247.232 |
| 767 | TTATAGTTACGGCCGCCGTTTACTGGGGCTTCAATTCAATGCTTCTCTTG | 15315 | 15299.685 | 15330.315 |
| 768 | CTTCAACCTGGACATGGATAGGTCACCCGGTTTCGGGTCTGCACDCACTG | 15338 | 15322.662 | 15353.338 |
| 769 | GAGGCTAGCCCTAAAGCTATTTCGAGGAGAACCAGCTATCTCCGGGTTCG | 15411 | 15395.589 | 15426.411 |
| 770 | TGGGCTGTTTCCCTTTCGACTACGGATCTTAGCACTCGCAGTCTGACTGC | 15286 | 15270.714 | 15301.286 |
| 771 | CTCCGGCCTATCCTACACATCGATTGCCCAAATTCAATGTAAAGCTATAG | 15233 | 15217.767 | 15248.233 |
| 772 | CCACTTCACCTAACAACAATGCAAAAAGGGCGTGCCACTGGTAGATGACA | 15350 | 15334.65 | 15365.35 |
| 773 | ACCCTCAGGTCATCCAGAAGCTTTTCAACGCTTATTGGTTCGGTCCTCCA | 15223 | 15207.777 | 15238.223 |
| 774 | AGTATCCCTTCCTGCAGGTACTGAGATGTTTCACTTCCCTGCGTACCCCC | 15175 | 15159.825 | 15190.175 |
| 775 | ACTTGGTATCCCTTCGGCTCCGCACCTTAAGTGCTTAACCTCGCCAGTAT | 15199 | 15183.801 | 15214.199 |
| 776 | TCGGATACGTGTGTCGTCACACTTAACCTTGCCGGCAAAGGCAACTCGTA | 15346 | 15330.654 | 15361.346 |
| 777 | GGATCACTAACTCCAACTTTCGTTACTGCTCGAACTGTCGCTCTCGCAGT | 15223 | 15207.777 | 15238.223 |
| 778 | CGAACGCCTTAGTATTTTCAACCTGACTACCTGTGTCGGTTTGGGGTACG | 15374 | 15358.626 | 15389.374 |
| 779 | TTCTGCTTCTGCCCGTACACGTTGCTCCCCTACCCAGAAGTTTCCTTCTG | 15117 | 15101.883 | 15132.117 |
| 780 | TCACGGTACTAGTTCGCTATCGGTCAGACAGGTATATCTAGGCTTACCCC | 15312 | 15296.688 | 15327.312 |
| 781 | ACTTCTTACAAAGCTCCGACCGCTTGTAGGCGCATGGTTTCAGGGACTAT | 15352 | 15336.648 | 15367.352 |
| 782 | TCTTTAAAGGATGGCTGCTTCTGAGCCAACCTCCTAGTTGTCTGGGCATC | 15334 | 15318.666 | 15349.334 |
| 783 | CCCCATTGGGGCCCACAACACCGCACACACAACCCCTACCAAGTATCACA | 15097 | 15081.903 | 15112.097 |
| 784 | CTCAACTTCAACCTGCTCATGGCTAGATCACCCGGTTTCGGGTCTGCAAC | 15233 | 15217.767 | 15248.233 |
| 785 | GCATACGCCACACGGCTTATGCTCGCCACCCGCCACTGACTCGCAGACTC | 15158 | 15142.842 | 15173.158 |
| 786 | GTTCGTCTATATGCCCGCACCTCACTGCGCCATGCCGGCAGACATGACCA | 15228 | 15212.772 | 15243.228 |
| 787 | ATCTGGGCTGTTTCCCTTTTGACAATGACATTTATCTGACACTGTCTGAC | 15283 | 15267.717 | 15298.283 |
| 788 | CTATTAGTAGCAGTCAGCTCCATGTGTTACCACACTTCCACCCCTGCCCT | 15128 | 15112.872 | 15143.128 |
| 789 | TTTCACAACTGACTTAAACATCCATCTACGCTCCCTTTAAACCCAATAAA | 15120 | 15104.88 | 15135.12 |
| 790 | CCGTTGAATTTTCGGCGCAGAGTCACTCGACCAGTGAGCTATTACGCACT | 15337 | 15321.663 | 15352.337 |
| 791 | TCCTTAACGAGAGTTCGCTCGCTCACCTGAGGCTACTCGCCTCGACTACC | 15194 | 15178.806 | 15209.194 |
| 792 | CCACTCCGTCGATGTGAACTCTTGGGAGTGATAAGCCTGTTATCCCCAGG | 15353 | 15337.647 | 15368.353 |
| 793 | CAACAGGATGAAGTTTAGCGGATTTTCTCGGGAGTATGATTACATGCGCT | 15495 | 15479.505 | 15510.495 |
| 794 | GACGGGCTGCGTGCTTGACCACGGAAAACCACCTCCGCGGCCGGCTACCC | 15304 | 15288.696 | 15319.304 |
| 795 | CGGATTTGCCTATGATGCGCGCTGCGTGCTTGACCACGGAAAACCACCTC | 15323 | 15307.677 | 15338.323 |
| 796 | CTGAGTTTGATAAGCTTCGCTAACCTCTCGGCCGCTAGGCTATTCAGTGC | 15319 | 15303.681 | 15334.319 |
| 797 | TGCAGCACCTGTCTCACGGTTCCCGAAGGCACATTCTCATCTCTGAAAAC | 15226 | 15210.774 | 15241.226 |
| 798 | AGGCTAGCCCTAAAGCTATTTCGGGGAGAACCAGCTATCTCCGAGTTCGA | 15395 | 15379.605 | 15410.395 |
| 799 | GACGTCCTATCTCTAGGATTGTCAGAGGATGTCAAGACCTGGTAAGGTTC | 15456 | 15440.544 | 15471.456 |
| 800 | GTTTTGACTACAGGGCTGTTACCTCCTATGGCGGGCCTTTCCAGACCTCT | 15286 | 15270.714 | 15301.286 |
| 801 | CTGGGGCTTCAATTCAGATCTTCGCTAACGCTAAACCCTCCTCTTAACCT | 15167 | 15151.833 | 15182.167 |
| 802 | CCTTAGTATATTCAACCCGACTACGTGTGTCCGTTTACGGTACGGGTACC | 15303 | 15287.697 | 15318.303 |
| 803 | CTATACATCATCTTACGATTTAGCAGAGAGCTGTGTTTTTGATAAACAGT | 15388 | 15372.612 | 15403.388 |
| 804 | CTAACAATGTCCCCCGACTCGATTCAGAGCCGCAGGTTAGAATTCCAATA | 15283 | 15267.717 | 15298.283 |
| 805 | TTTGGCCTCTTCCGCGTTCGCTCGCCACTACTTACGGAATCTCAGTTGAT | 15221 | 15205.779 | 15236.221 |
| 806 | CCCGCCAACTGGCTAATCAGACGCGGGTCCATCTTATACCACCGGAGTTT | 15267 | 15251.733 | 15282.267 |
| 807 | GCTACTTGGGACACGCGATCGGAAGACGGCAAGCGTCCAGGTACGGGGCT | 15527 | 15511.473 | 15542.527 |
| 808 | CATCACCGGGGATGAGCTACCTCACTGCGTCCCTCCGCAGCTTGCCTACT | 15195 | 15179.805 | 15210.195 |
| 809 | ACAACTTAATACCCGATTATTATCCACGCCAGACTCCTCGACTAGTGAGC | 15218 | 15202.782 | 15233.218 |
| 810 | CTCTCAGACCAGTTACGGATCGTCGCCTTGGTAGGCCTTTACCCCACCAA | 15218 | 15202.782 | 15233.218 |
| 811 | TCACGTAGTCTGACTGCTGATCATCAATTAGCCGGCATTCAGAGTTTGAT | 15366 | 15350.634 | 15381.366 |
| 812 | TAGGTCACCCGGTTTCGGGTGTACTGCATGCAACTTTACGCCCTTTTCAG | 15310 | 15294.69 | 15325.31 |
| 813 | TACTTTAGTTCGCTCCACATCACGGCTTCGTCTCATGCACAGCGGATTTG | 15254 | 15238.746 | 15269.254 |
| 814 | CTTACGGGGCTTTCACCCTCTCTGGCAGGCTTTCCCAAAAACCTTTCTGC | 15175 | 15159.825 | 15190.175 |
| 815 | GGCCGGGCTTTCGATCCCGTTCTTCTATCCTCTCTCTTGCCATATCATGG | 15188 | 15172.812 | 15203.188 |
| 816 | ACGGCTTCTACTCGTATACAACGCTCCCCTACCACTATAGTTTCCTACAA | 15120 | 15104.88 | 15135.12 |
| 817 | ATCGAGTTTTCTTTCTCTTCCTCCGGCTACTTAGATGTTTCAGTTCACCG | 15201 | 15185.799 | 15216.201 |
| 818 | GCTTTACATACCGAAATACTTCTTCACTCACGCGGCGTCGCTGCATCAGG | 15257 | 15241.743 | 15272.257 |
| 819 | TCCCTTCTGCCTTTGCACTCTTCTAATGGTTTCCGACCATTATGAGGGAA | 15244 | 15228.756 | 15259.244 |
| 820 | CTCCATCAGGCAGTTTCCCAGACATTACTCACCCGTCCGCCACTCGTCAG | 15123 | 15107.877 | 15138.123 |
| 821 | TGCCAAACCTCCCCGTCGATGTGAACTCTTGGGGGAGATAAGCCTGTTAT | 15377 | 15361.623 | 15392.377 |
| 822 | GCCTGGACCTATTCTCTGCGCCTCACATTACTGTGAGGACCCTTTATCCC | 15175 | 15159.825 | 15190.175 |
| 823 | ACCTTTACACCTGCATCCTATCAACGTCGTAGTCTACAACGACCCTCAGA | 15154 | 15138.846 | 15169.154 |
| 824 | GTATTCATTAACGCTAGAAGCTTTTCTTGGCAGAGTGACATCACTAGCTT | 15365 | 15349.635 | 15380.365 |
| 825 | GCTGTTGGTCCGGATTGTTCTCCTTTAGGACATGGACCTTAGCACCCATG | 15350 | 15334.65 | 15365.35 |
| 826 | AAAAACCCTCCCCCCCCCCCCTTCCCCTCCGCGGCCACCTTTCCCCCCCC | 14767 | 14752.233 | 14781.767 |
| 827 | CTGTCGGTACCCGATACGGGCCCTCAAGCATCCAGTAGCTCTACCCCCCG | 15188.8 | 15173.6112 | 15203.9888 |
| 828 | ATCTACGCATTTCACCGCTACACTAGGAATTCCGCTTACCTCTGTTGCAC | 15167 | 15151.833 | 15182.167 |
| 829 | TCTGTCCCACCTTCGGCGGCTGGCTCCTAAAAGGTTACCTCACCGACTTC | 15185 | 15169.815 | 15200.185 |
| 830 | TGACCAAGGGTAGATCACTTGGTTTCGCGTCTACTCCTTCCGACTAATCG | 15303 | 15287.697 | 15318.303 |
| 831 | TGTGCACTTGCACTCGCCACCCGATTGCCAACCGGGCTGAGCGGACCTTT | 15275 | 15259.725 | 15290.275 |
| 832 | CAGCCTCACTCCCAGGCTGTAAAATATGCCCCTTCGGAGTTTGATAAGGT | 15321 | 15305.679 | 15336.321 |
| 833 | ACGCTTCCACTAACACACACACTGATTCAGGCTCTGGGCTGCTCCCCGTT | 15178 | 15162.822 | 15193.178 |
| 834 | CTGTCAAGGTCGACTCTCCCTGCGGATTTGCCTACAGGAATCTACATCTA | 15272 | 15256.728 | 15287.272 |
| 835 | CCTGTGTTTTTGGTAAACAGTCGCTACCCCCTGGCCTGTGCCACCCCCCG | 15176.8 | 15161.6232 | 15191.9768 |
| 836 | ATCTGATAGCGTGAGGTCCGAAGATCCCCCACTTTCTCCCTCAGGACGTA | 15282 | 15266.718 | 15297.282 |
| 837 | ACACTTTGGGACCTTAGCCGGTGGTCTGGGCTCTTTCCCTTTTGACTACC | 15277 | 15261.723 | 15292.277 |
| 838 | CTACAAGGGATCTTACCTGATTGAATCAGTGGGATATCTTATCTTTGGGT | 15436 | 15420.564 | 15451.436 |
| 839 | CTGAAGGGTAACCCCACATAACCAGGGCCAGGTTTCCCCATTCGGACATC | 15285 | 15269.715 | 15300.285 |
| 840 | TCAGTCCGCGGCGCTGTCACGCCTCCGTCTCCACGTCACTCCTTAAGGTA | 15186 | 15170.814 | 15201.186 |
| 841 | TTAACAAGGGTTCTCCCGTTCGTCTCAGGATTCTCTCCTCGCCCACCTGC | 15151 | 15135.849 | 15166.151 |
| 842 | CTAACATCCTAGTTGTCTGTGCAACCCCACATCCTTTTCCACTTAACAAT | 15110 | 15094.89 | 15125.11 |
| 843 | GATAAATCTTTCCCCCGTAGGGCACATTCGGTATTACTCCCAGTTTCCCG | 15223 | 15207.777 | 15238.223 |
| 844 | GTTTACAATCCGAAGACCTTCTTCCCACACGCGGCGTTGCTGCATCAGGG | 15298 | 15282.702 | 15313.298 |
| 845 | CGGCGCACTGCAGCTACCTGTCTGCGTCACCCCTGTTAACACGCTTGCCT | 15186 | 15170.814 | 15201.186 |
| 846 | ATGAAGCTGGAATCGCTAGTAATCGTATATCAGCAATGATACGGTGAATA | 15505 | 15489.495 | 15520.505 |
| 847 | CGGATTTGCCTATGGGACGGGCTGCGTGCTTGACCACGGAAAACCACCTC | 15388 | 15372.612 | 15403.388 |
| 848 | GGATGACCCCCTTGCCGAAACAGTGCTCTACCCCCGGAGATGAATTCACG | 15301 | 15285.699 | 15316.301 |
| 849 | GGTACGGGTAACATATACTATAACTTAGAAGATTTTCTCGGAAGTCGACT | 15447 | 15431.553 | 15462.447 |
| 850 | CTTTGTAACTCCGTACAGAGTGTCCTACAACCCCAAGAGGCAAGCCTCTT | 15250 | 15234.75 | 15265.25 |
| 851 | TCTTACTTCTTGCGAATGGGAGATCTCATCTTGGAGTAGGCTTCGTGCTT | 15395 | 15379.605 | 15410.395 |
| 852 | GTCAAGCTCCCTTATACCTTTACACTCTGCGATTGATTTCCAACCAATCT | 15141 | 15125.859 | 15156.141 |
| 853 | CCACCTATCCTACACATCAAGGCTCAATGTTCAGTGTCAAGCTATAGTAA | 15257 | 15241.743 | 15272.257 |
| 854 | AAAAGCAGTTTACAACCCATAGGGCCGTCATCCTGCACGCTACTTGGCTG | 15315 | 15299.685 | 15330.315 |
| 855 | TGAGGGCACCTTTAGAAGCCTCCGTTACACTTTTGGAGGCGACCACCCCA | 15307 | 15291.693 | 15322.307 |
| 856 | ACGCTCTAACCTTATGGTAACCGGATTTGCCTGGTAACCAGCCGCTTCGC | 15273 | 15257.727 | 15288.273 |
| 857 | GCTTCCAAGCCAACATCCTAGCTGTCTTAGCAATCTGACTTCGTTAGTTC | 15222 | 15206.778 | 15237.222 |
| 858 | TGGCCGTTCACCCTCTCAGGCCGGCTATGGATCGTCGCCTTGGTAGGCCG | 15338 | 15322.662 | 15353.338 |
| 859 | TGAGCCAACATCCTGGTTGTCTTCGAAATCCCACATCCTTTTCCACTTAA | 15166 | 15150.834 | 15181.166 |
| 860 | CTAGAGAGTATTTAGGGTTAGGAGATGGTCCTCCCAGATTCCGACGAGAT | 15505 | 15489.495 | 15520.505 |
| 861 | GCCTTTCGGCCTCGCGTTAGGTCCCGACTTACCCAGGGCGGACGAACCTT | 15291 | 15275.709 | 15306.291 |
| 862 | GTCAAACTGCCCACCTGACACTGTCTCCCCGCCCGATAAGGGCGGCGGGT | 15294 | 15278.706 | 15309.294 |
| 863 | TGGAGTAAAGCTCCATGGGGTCTTTCCGTCCTGGCGCAGGTAACCAGCAT | 15418 | 15402.582 | 15433.418 |
| 864 | TTTCTTCTCCTACGGGTACTGAGATGTTTCACTTCCCCGCGTAACCCCCA | 15150 | 15134.85 | 15165.15 |
| 865 | ACCAGCTATGGATCGTCGGCTTGGTAGGCCATTACCCCACCAACTACCTA | 15251 | 15235.749 | 15266.251 |
| 866 | GGGGCAAGTTTCGTGCTTAGATGCTTTCAGCACTTATCTCTTCCGCATTT | 15315 | 15299.685 | 15330.315 |
| 867 | CACCAGTGTCGGTTTGGGGTACGGGCGGCCATAGCCCTCACGCCGAGGCT | 15421 | 15405.579 | 15436.421 |
| 868 | GACGTTCTGAACCCAGCTCGCGTGCCGCTTTAATGGGCGAACAGCCCAAC | 15317 | 15301.683 | 15332.317 |
| 869 | GGTTAGAATTCCAATATCGCAAGGATGGTATCCCAACGGCCTCTCCGCCA | 15315 | 15299.685 | 15330.315 |
| 870 | AGGTTACCCACGCGTTACTCACCCGTCCGCCACTAGAAACAATCTAAATC | 15188 | 15172.812 | 15203.188 |
| 871 | CAGGTGTCACCCCATATACGTCATCTTTCGATTTAGCATAGAGCTGTGTT | 15317 | 15301.683 | 15332.317 |
| 872 | TCTTTCGGCGAGGGGGTTTCCCACCCCCTTTATCGTTACTTATACCTACA | 15205 | 15189.795 | 15220.205 |
| 873 | CTTAGGACCGTTATAGTTACGGCCGCCGTTTACCGGGGCTTCGATCAAGA | 15393 | 15377.607 | 15408.393 |
| 874 | CCACTTAGTGATGATTTGGGGACCTTAGCTGGCGGTCTGGGTTGTTTCCC | 15437 | 15421.563 | 15452.437 |
| 875 | TCCCCCATTCGGACACCTCCGCTTCTTCGCTTCCTTACAGCTTCACGGAG | 15096 | 15080.904 | 15111.096 |
| 876 | ATAGATCACCCGGTTTCGGGTCTGCCCCCACTGACTCTGGCCCTCTTAAG | 15225 | 15209.775 | 15240.225 |
| 877 | GCCTATCAAACACGTGTTCCACATGCGGGCTTCAGGACCCCGAAGGGCCC | 15302 | 15286.698 | 15317.302 |
| 878 | CCATTTCTGACTGTTATCCCCCTGTATAAGGCAGGTTGCCCACGCGTTAC | 15239 | 15223.761 | 15254.239 |
| 879 | CATCATCTGTATGGCATTCGGAGTTTGATATCCCTTAGTAAGCTTTGACG | 15372 | 15356.628 | 15387.372 |
| 880 | GTTTGGGGTACGGGCGGCTAAAACCTCGCGCCGATGCTTTTCTAGGCAGC | 15450 | 15434.55 | 15465.45 |
| 881 | GCGATGGCCCTTCCATACGGTACCACCGGATCACTAAGCCCGACTTTCGT | 15243 | 15227.757 | 15258.243 |
| 882 | GAGTTAACCCCGGCGGTCCCCCGTGAGTTCCCACCATAACGTGCTGGCAA | 15292.9 | 15277.6071 | 15308.1929 |
| 883 | GGATAATCGGCGGACGGGATTCCCACCCGTCACACGCTACTCATGCCTGC | 15293 | 15277.707 | 15308.293 |
| 884 | TACCTCTTCGTTATGATATGTCCGCAACCCCAATAAAGAAAACTTTATTG | 15262 | 15246.738 | 15277.262 |
| 885 | ACGTGTCCGGCGGTACTCTGGATTCAGCTGGCGGATCTTCTCTTTCGCAT | 15342 | 15326.658 | 15357.342 |
| 886 | TCGAGACCAGACTTCGTTAGACTAACTCAGACAGGATTCCGGGACCTTAG | 15379 | 15363.621 | 15394.379 |
| 887 | TGGCCGTTCAACCTCTCAGTCCGGCTACCAATCGTCGCCTTGGTGGGCCG | 15282 | 15266.718 | 15297.282 |
| 888 | TATAAGTCAAGGCTGCACCTAAATGCATTTCGGGGAGTACGAGCTATCTC | 15409 | 15393.591 | 15424.409 |
| 889 | CTACTGTTTCACCGCGTATACAACGCTCCCCTACCCAGCATGTAAACATG | 15170 | 15154.83 | 15185.17 |
| 890 | TTATAGTTACGGCCGCCGTTTACTGGGGCTTCAATTCACACCTTCGACAA | 15287 | 15271.713 | 15302.287 |
| 891 | GGATGGACCCCTCACCCAAACAGTGCTCTACCTCCATGATTCTTAATGTC | 15201 | 15185.799 | 15216.201 |
| 892 | TTGGGACCTTAGCTGCGGGTCTGGGCTCTTTCCCTTTTGACTATCCAACT | 15292 | 15276.708 | 15307.292 |
| 893 | GGCTCTGACTACTTGTAGGCACACGGTTTCAGGATCTCTTTCACTCCCCT | 15230 | 15214.77 | 15245.23 |
| 894 | TCGCTACTCATTCCGGCATTCTCACTCGTGTACAGTCCACCGCTGCTTTC | 15126 | 15110.874 | 15141.126 |
| 895 | CCTCCCCCCCCCCCCCCCCCCCCCCCCCTTCCCCCCTCTCCTCCCCCTTC | 14517 | 14502.483 | 14531.517 |
| 896 | TAACACCCCATAACAGGTGCCAGGTTTCCCCATTCGGACATCCTCGGATC | 15211 | 15195.789 | 15226.211 |
| 897 | ACCTCGACACGGACGGTGACAAGCCGGTACCAGAATATCAACTGGTTACC | 15358 | 15342.642 | 15373.358 |
| 898 | ATAGATCACCCGGTTTCGGGTCTACTCCGGCTGACTCGCTCGCCCTATTC | 15216 | 15200.784 | 15231.216 |
| 899 | TAAATGATGGCTGCTTCTAAGCCAACATCCTGGCTGTCTGGGCCTTCCCA | 15288 | 15272.712 | 15303.288 |
| 900 | CAGCTTATAGGGTTGCGTACTTCACTACAACCCAACCTTGATGCTTGCAC | 15256 | 15240.744 | 15271.256 |
| 901 | GCTTGGGCCTTTTCACTGCGGCTGACTTATCGCCAGCGCCCCTTCTCCCG | 15184 | 15168.816 | 15199.184 |
| 902 | TGAGGTCGGCTTCACGCTTAGATGCTTTCAGCGTTTATCCGTTCCGCACT | 15301 | 15285.699 | 15316.301 |
| 903 | CTCCGGGTACTGTCAGGTTCGACTCTCAGGGCGGATTTGCCTACCCCGAT | 15321 | 15305.679 | 15336.321 |
| 904 | GCTTGGGCCTCTTCACTGCGGCTTAATTGCTTAAGCACTCCTTCTCGCTA | 15221 | 15205.779 | 15236.221 |
| 905 | TTTATCCCGAAGTTACAGGGTCAGTTTGCCTAGTTCCTTAACCGTGAATC | 15317 | 15301.683 | 15332.317 |
| 906 | GTAGTTAGCCGGAGCTTCCTCCTAAAGTACCGTCATTATCGTCCTTTAAG | 15302 | 15286.698 | 15317.302 |
| 907 | TCTTTCGGCGAGGGGGTTTCCCGCCCCCTTTATCGTTACTTATACCTACA | 15221 | 15205.779 | 15236.221 |
| 908 | GGATGTACTAGCAGCTTTTCTCGCCAGCGTGAACTCACTCGCTTCCCTAC | 15224 | 15208.776 | 15239.224 |
| 909 | TTAGTATCAGTGCTTTATCAGGGGCGCATATACTCGGGTACCAGAATATC | 15415 | 15399.585 | 15430.415 |
| 910 | GCTTGGCGGCGTCCTACTCTCACAGGGGGAAACCCCCGACTACCATCGGC | 15294 | 15278.706 | 15309.294 |
| 911 | AGATTCACGCAGAATTCCTCGTGCTCCGCGCTACTCAGGATACTACTATG | 15281 | 15265.719 | 15296.281 |
| 912 | TATCAACCTGATCATCTTTCAGGGATCTTACTTCCTTGCGGAATGGGAAA | 15350 | 15334.65 | 15365.35 |
| 913 | TCAATAGGCACGCCACCACACTCTTATGGAGCGGTGACTGCTTGTAAGTC | 15346 | 15330.654 | 15361.346 |
| 914 | CTACTATATTTCGGTCCCTTACGCCCGGGGCAACCATCGCCCGGGATAAC | 15243 | 15227.757 | 15258.243 |
| 915 | TGCCATGACTGCTTGTAAGTCCACGGTTTCAGGTTCTCTTTCACTCCCCT | 15196 | 15180.804 | 15211.196 |
| 916 | TCCATTTGCGCAGCACCAGTAATCATGTTCTTAACATAGTCAGCATGTCC | 15255 | 15239.745 | 15270.255 |
| 917 | TCTCAGTCCCAATGTGGCCGGTCACCCTCTCAGGTCGGCTACTGATCGTC | 15241 | 15225.759 | 15256.241 |
| 918 | TGGCCGTTCAACCTCTCAGTCCGGCTACTGATCGTCGCCTTGGTGGGCCT | 15288 | 15272.712 | 15303.288 |
| 919 | TTATAGTTACGGCCGCCGTTTACCGGGGCTTCAATTCGGAGCTCTCACTC | 15295 | 15279.705 | 15310.295 |
| 920 | TAGTGAAAGGTAGATTTTCTGACCCTTTCGACCTGAACGTACCAACCAGC | 15329 | 15313.671 | 15344.329 |
| 921 | TCTTGGCAGTGTGACATCACTAACTTCGCTACTAAACTTCGCTCCCCATC | 15167 | 15151.833 | 15182.167 |
| 922 | ACCTGCTTTCGCACCTGCTCGCGCCGTCACGCTCGCAGTCAAGCTGGCTT | 15202 | 15186.798 | 15217.202 |
| 923 | TCGGAGTTTGATATTCTTCGGTAAGCTTTGACGCCCCCTAGGAAATTCAG | 15382 | 15366.618 | 15397.382 |
| 924 | ACCCACCGAGTGGGCGCCCATCAGGTCTCAAGCACATAGCCGGCGGATTT | 15342 | 15326.658 | 15357.342 |
| 925 | TACGGGTGCCGCATGGATAAGTTTAGCGGATTTTCTCGGGAGCATGGTTA | 15543 | 15527.457 | 15558.543 |
| 926 | TTCAAACAACCATCCGGTATTAGCCCCGGTTTCCCGGAGTTATCCCAGTC | 15217 | 15201.783 | 15232.217 |
| 927 | TCCTTAACCACGCTGCATACCATAACTCGCCGGACCATTCTACAAAAGGT | 15203 | 15187.797 | 15218.203 |
| 928 | CCGGCACCGGGCAGGTGTCAGGCTGTATACGTCATCTTTCGAGTTTGCAC | 15385 | 15369.615 | 15400.385 |
| 929 | CAGGAATATTCAGGCTTACCCAACGGTCTGGGCGGATTCGCACGGGGTTC | 15443 | 15427.557 | 15458.443 |
| 930 | TTTATCCCGAAGTTACAGGGTCAGTTTGCCTAGTTCCTTAACCGTGAATC | 15317 | 15301.683 | 15332.317 |
| 931 | CTTCTGCAATTGCACTCGTCGATTGGTTTCCATCCAATCTGAGCGTACCT | 15229 | 15213.771 | 15244.229 |
| 932 | TCGGTTTGCCCTCTTCCGCGTTCGCTCGCCACTACTTACGGAATCTCGTT | 15173 | 15157.827 | 15188.173 |
| 933 | AAGCTCCATGGGGTCTTTCCGTCTTGTCGCGGGTAACCGGCATCTTCACC | 15296 | 15280.704 | 15311.296 |
| 934 | CATCGGCCTCACCGTTCGGCTGAGCCTTAGGACCCGACTAACCCTGATCC | 15204 | 15188.796 | 15219.204 |
| 935 | CCTCGCCATACACGCCGCACGGATTTGCCTATGCGACTGGCTGCGTGCTT | 15266 | 15250.734 | 15281.266 |
| 936 | CCTGTCGCGGGTAACCTGCATCTTCACAGGTACTATAATTTCACCGAGTC | 15272 | 15256.728 | 15287.272 |
| 937 | TCAGCCTTATGGGAAACGGATTTGCCTATTTCCCAGCCTAACTGCTTGGA | 15327 | 15311.673 | 15342.327 |
| 938 | TTTCACAACACGCTTAAAAGGCGGCCTACGCTCCCTTTAAACCCAATAAA | 15211 | 15195.789 | 15226.211 |
| 939 | CCCCGCGGTACTCTGGATCCTGCTAGCTCTCGCTCCTTTTCGTCTACGTG | 15174 | 15158.826 | 15189.174 |
| 940 | ATCGGTTCACACACTCACCCACCCCAGAAGCATCAAAAACACTCCCAAGA | 15144 | 15128.856 | 15159.144 |
| 941 | TAGAAAGGAGGTGATCCAGCCGCACCTTCCGATACGGCTACCTTGTTACG | 15371 | 15355.629 | 15386.371 |
| 942 | GCCCATTGTCCAATATTCCCCACTGCTGCCTCCCGTAGGAGTCTGGACCG | 15210 | 15194.79 | 15225.21 |
| 943 | TCACCTTTCCCTCACGGTACTGGTTCGCTATCGGTCTCTCGGGAGTATTT | 15252 | 15236.748 | 15267.252 |
| 944 | CGAAGTTACGGGGTCATTTTGCCGAGTTCCTTGACAATGCTTCTCCCGCC | 15295 | 15279.705 | 15310.295 |
| 945 | AGATCCTCTCAAATTTCCTACGCCCGCGACGGATAGGGACCGAACTGTCT | 15291 | 15275.709 | 15306.291 |
| 946 | TCTCAGTCCCAATGTGGCCGGTCACCCTCTCAGGTCGGCTACTGATCGTC | 15241 | 15225.759 | 15256.241 |
| 947 | GGCAACCCAACAACCCACACATCATCATCTTCAGCTACAGGACTCTCACC | 15102 | 15086.898 | 15117.102 |
| 948 | GCACTATTGCCTTGTCCCGGAGGACGCGGCATACTGTCAGGTTCGAATCA | 15378 | 15362.622 | 15393.378 |
| 949 | CCGTGGCTTTCTGGTTAGGTACCGTCAAGGTACCGCCCTATTCGAACGGT | 15360 | 15344.64 | 15375.36 |
| 950 | ATACTATCAGGTTCGACTCTTATCCCGGATTTGCCTGGGATAATCAACAT | 15310 | 15294.69 | 15325.31 |
| 951 | TAAGTCCTTAACCTTGCTGCATACAATCGCTCGCCGGACCGTTCTACAAA | 15225 | 15209.775 | 15240.225 |
| 952 | ATCTGGGCTGTTTCCCTTTTGACAATGACATTTATCTGACACTGTCTGAC | 15283 | 15267.717 | 15298.283 |
| 953 | AGAGTAACCATAACACAAGGGTAGTATCCCAACAACGCCTCCTCCGAAAC | 15279 | 15263.721 | 15294.279 |
| 954 | TGGACAGGATTCTCACCTGTCTTACGCTACTCATACCGGCATTCTCACTT | 15198 | 15182.802 | 15213.198 |
| 955 | GCCCGGCTACCTTCCTGCGTCACACCTGTTAATACGCTTGGCTCCCCAGT | 15161 | 15145.839 | 15176.161 |
| 956 | GTCAAGCTCCCTTATACCTTTACACTCTGCGAATGATTTCCAACCATTCT | 15141 | 15125.859 | 15156.141 |
| 957 | CCCAACCCTTGGAACATACTACAGCCCCAGGTGGCGAAGAGCCGACATCG | 15304 | 15288.696 | 15319.304 |
| 958 | TCTTTCGGCGAGGGGGTTTCCCACCCCCTTTATCGTTACTTATACCTACA | 15205 | 15189.795 | 15220.205 |
| 959 | GGGTGTTCCCCTTTTGCCCGCGGAACTTATCTCTCGCGGACTGACTCCCA | 15232 | 15216.768 | 15247.232 |
| 960 | ACCCGGTTTCGGGTCTATGGCATACAACTTCTCGCCCTTGTCAGACTCGC | 15240 | 15224.76 | 15255.24 |
| 961 | CTGCCTGGCTTACGCCTACGGGGCTTTCACCCTCTCCGGCGCCGGCATTC | 15194 | 15178.806 | 15209.194 |
| 962 | GCTGCGGGGCTGAGCCCCTTAACCTCGCCGGAAAAAGTAACTCGTAGGTT | 15412 | 15396.588 | 15427.412 |
| 963 | AAGGATGGCTCTCTTCAAATCTCCTGCGCCCGCGACGGATAGGGACCGAA | 15381 | 15365.619 | 15396.381 |
| 964 | CAGGCCCCACAACACCGCACACACAACCCCCGCCGGGTATCACATGCACA | 15123 | 15107.877 | 15138.123 |
| 965 | CCCCTACGGATCCATGCCTTGGTGGGCCATTACCCCACCAACTAGCTAAT | 15187 | 15171.813 | 15202.187 |
| 966 | ACTTAGCACTCATCGTTTACGGCGTGGACTACCAGGGTATCTAATCCTGT | 15327 | 15311.673 | 15342.327 |
| 967 | TATCCATCGAAGACTAGGTGGGCCGTTACCCCGCCTACTATCTAATGGAA | 15330 | 15314.67 | 15345.33 |
| 968 | CAGGCGTCAGCTCGTATACGTCATCTTTCGATTTAGCACAAACCTGTGTT | 15302 | 15286.698 | 15317.302 |
| 969 | TGGCCGTTCAACCTCTCAGTCCGGCTACCGATCGCGGTCTTGGTGAGCCG | 15322 | 15306.678 | 15337.322 |
| 970 | CCTGTGTTTTTGCTAAACAGTCGCCTGGGCCTATTCACTGCGGCTCTCTC | 15237 | 15221.763 | 15252.237 |
| 971 | ACGCCTTTCGGCCTGACCTTAGCTCCCGACTTACTTGGAGCGGACGAACC | 15259 | 15243.741 | 15274.259 |
| 972 | GGTCTGGGCTCTTTCCCTTTTGACTGCCCAACTTATCTCGTGCAGTCTGA | 15252 | 15236.748 | 15267.252 |
| 973 | GAATGAATGGCTGCTTCTGAGCCAACATCCTAGTTGTCTTAGAGATCCCA | 15360 | 15344.64 | 15375.36 |
| 974 | CCCCATCATGCCTCAACCTTCACGCCCAGCGGATTTACCTACCAGACAGT | 15116 | 15100.884 | 15131.116 |
| 975 | AAAAGTACGCGGTTCATCATATAAAGATGTTCCACAGCTTGTAAACACAG | 15394 | 15378.606 | 15409.394 |
| 976 | ATCTGAAGTCTTCTCGTTTAACATACAGGACTATTACCTTCTGTGGTGAG | 15356 | 15340.644 | 15371.356 |
| 977 | GGTCACACCCTTTTGAAGTGTCCCTTTGCTTAAATTACAGATGGTTACGG | 15357 | 15341.643 | 15372.357 |
| 978 | CAGCTTATCACGTCTTTCATCGGCTCTTAGTGCCAAGGCATCCACCCTGC | 15184 | 15168.816 | 15199.184 |
| 979 | TTCCATTCGGCACCGCCGGATCACTATTCCCGACTTTCGTCCCTGTTCGA | 15151 | 15135.849 | 15166.151 |
| 980 | TCCAGGTTCGATTGGCATTTCACCCCTACCCACACCTCATCCCCGCACTT | 15049 | 15033.951 | 15064.049 |
| 981 | TACACCTTCTGCGTACATAGAACGCTCTCCTACCATCCCCTAAGGGATCC | 15146 | 15130.854 | 15161.146 |
| 982 | GCTTGCGCTAACCTCTCCTCTTAACCTTCCAGCACCGGGCAGGCGTCAGC | 15195 | 15179.805 | 15210.195 |
| 983 | CGCCCGTTAGTACCGGTCGGCTCCACCCCTCGCGGGGCTTCCACCTCCGG | 15189 | 15173.811 | 15204.189 |
| 984 | CTCCGGGACCTTAGACGGCGGTCTGGATTCTTCTCCTCTCGGGGACGGAC | 15362 | 15346.638 | 15377.362 |
| 985 | TGGTTAAGTCCTCGATCGATTAGTATCTGTCAGCTCCATGTGTCGCCACA | 15318 | 15302.682 | 15333.318 |
| 986 | TAAGTCCTTAACCTTGCTGCATACAATCGCTCGCCGGACCGTTCTACAAA | 15225 | 15209.775 | 15240.225 |
| 987 | ACCGGACTTTCCATTTCCGGCCCATGTTTCCCTCCCGTGTCCCCACAGTT | 15087 | 15071.913 | 15102.087 |
| 988 | CGGCTCCCACCTATGCTACGCAGAAGAATCCGGATATCAATGCCAGACTA | 15293 | 15277.707 | 15308.293 |
| 989 | ACCCCACATCCTTTTCCACTTAACATATATTTGGGGACCTTAGCTGGTGG | 15262 | 15246.738 | 15277.262 |
| 990 | CCACACCACTTCACCTAACAACAACACACAAGCACGATGATGGTAGTCAC | 15199 | 15183.801 | 15214.199 |
| 991 | TCATCCCCGCACTTTTCACGTACGTGTGGTTCGGACCTCCACGACGTCTT | 15191 | 15175.809 | 15206.191 |
| 992 | CCCTTCAAAGCCTCCGACCTATCCTACACATCACGTGCCCAGATTCAATG | 15115 | 15099.885 | 15130.115 |
| 993 | CTTCACCTAACAACAATGCGCAAGCAGGACGTCAGTAGCCATCCTCATCA | 15237 | 15221.763 | 15252.237 |
| 994 | GGGGTACGGGCGGCAACGCGCCTGACGCCGAAGCTTTTCTCGGCACCACG | 15415 | 15399.585 | 15430.415 |
| 995 | ATGGCTAGATCACCGGGTTTCGGGTCTATACCCTGCAACTTAACGCCCAG | 15322 | 15306.678 | 15337.322 |
| 996 | ATTAAACCACATGCTCCACCGCTTGTGCGGGCCCCCGTCAATTCCTTTGA | 15193 | 15177.807 | 15208.193 |
| 997 | GCCGGCTTTCCCAAAGCCGTTCTGCTACCTCTCGCGGATCAATTATGCGG | 15265 | 15249.735 | 15280.265 |
| 998 | ACGCCTTCCGGCCTCACCTTAGCTCCCGACTAACTTGGAGCGGACGAACC | 15213 | 15197.787 | 15228.213 |
| 999 | ACACCACGCGGCGATACCAACCCGAAGGAAGGAACCACCACGAGGCGGAG | 15421 | 15405.579 | 15436.421 |
| 1000 | CCGAACCCCGAGATGCACGCATCTCGGTTTGGCCTCTTTCGCGTTCGCTC | 15217 | 15201.783 | 15232.217 |
| 1001 | GGGACTTCATCCTGGCCAAGTGTAGATCACTTGGTTTCGCGTCTACCCCC | 15280 | 15264.72 | 15295.28 |
| 1002 | AGCCCTCGACCTATTAGTACTGCCAAGCTGAATGCCTCACGGCACTTACA | 15235 | 15219.765 | 15250.235 |
| 1003 | GGGAGCGGGATTACCTTCACTATCAATCCACCCGAAGGTTTCATGTACTA | 15345 | 15329.655 | 15360.345 |
| 1004 | CACGCGGGATTCCACGAGGCCCGCGCTACTTGGGACAACACGATCGGAAG | 15416 | 15400.584 | 15431.416 |
| 1005 | CCTACACCCTTCAACCATCTATTCCGTCAGATGGCGGCACTGTCACTACT | 15137 | 15121.863 | 15152.137 |
| 1006 | CCCCGTACCTGTTCTCGATACCAGGTTAGAACCCCGGTCACACAAGAGTG | 15276 | 15260.724 | 15291.276 |
| 1007 | GTTTCACGTGTCTGGCCGTACTCTGGATCCTGCGCAGCTCTCTCCGTTTT | 15244 | 15228.756 | 15259.244 |
| 1008 | TTCCCGCTTAGATGCTTTCAGCGGTTATCCCTCCCGAACGTAGCCAACCG | 15209 | 15193.791 | 15224.209 |
| 1009 | GCACTCCCACAGCTTGTAGACACAGGGTTTCAGGTTCTCTTTCACTCCCC | 15184 | 15168.816 | 15199.184 |
| 1010 | CCTGGCCAAGGGTAGATCACTTGGTTTCGCGTCTGCCACTGCCGACTATA | 15329 | 15313.671 | 15344.329 |
| 1011 | CCGCGAGGGACCTCACCTACATATCAGCGTGCCTTCTCCCGAAGTTACGG | 15268 | 15252.732 | 15283.268 |
| 1012 | AAGCTCCATGGGGTCTTTCCGTCTTGCCGCAGGTAACCGGCATCTTCACC | 15265 | 15249.735 | 15280.265 |
| 1013 | CGTCGGCTTGGTGGGCCGTTACCTCACCAACTACCTAATCCAACGCGGGT | 15299 | 15283.701 | 15314.299 |
| 1014 | GCTCCCACCTATCCTGTACATGCAATACCAAGCTCCAGTACCAAACTGGA | 15188 | 15172.812 | 15203.188 |
| 1015 | ACCGGACTTTCCATTTCCGGCCCATGTTTCCCTCCCGTGTCCCCACAGTT | 15087 | 15071.913 | 15102.087 |
| 1016 | CAGTTCCCCGGGTCTGCCTTCTCATATCCTATGAATTCAGATATGGATAC | 15262 | 15246.738 | 15277.262 |
| 1017 | GGTCCCGGCAGATTCGCGCAGGATTCCTCGTGTCCCGCGTTACTCAGGAT | 15346 | 15330.654 | 15361.346 |
| 1018 | GTATTAACTTTACTCCCTTCCTCCCCGCTGAAAGTACTTTACAACCCGAA | 15135 | 15119.865 | 15150.135 |
| 1019 | GGGGGCGGGGAGCGGGGCGTGGGCGGGAGGAGGGGAGGAGGCGTGGGGGG | 16068 | 16051.932 | 16084.068 |
| 1020 | CACGAGGCCCGCGCTACTTGGGACACGCGATCGGGAGACGGCAAGCGTCC | 15433 | 15417.567 | 15448.433 |
| 1021 | CGTTTATCCCCTCCCTACTTAGCTACCCAGCGATGCTCTTGGCAGAACAA | 15177 | 15161.823 | 15192.177 |
| 1022 | CCTCTTAACCTTCCGGCACCGGGCAGGCGTCAGAGCGTATACAGCGGCTT | 15323.9 | 15308.5761 | 15339.2239 |
| 1023 | ACCTTGGGCGGACGAACCTTCCCCAAGAAACCTTAGATTTTCGGCCATTA | 15290 | 15274.71 | 15305.29 |
| 1024 | TTCGTTCGCCACTACTAGCAGAATCATAATTTTATTTTCTTCTCCTACGG | 15202 | 15186.798 | 15217.202 |
| 1025 | GTTTCTCGCATGCCTCTCGCTACTCATACCGGCATTCTCTCTTGTGCAGT | 15172 | 15156.828 | 15187.172 |
| 1026 | CCTATCAACGTCGTCGTCTTCAACGTTCCTTCAGGACCCTTAAAGGGTCA | 15232 | 15216.768 | 15247.232 |
| 1027 | CTGTTATCCCCAGGGTAGCTTTTATCCGTTGAGCGACGGCATTTCCATTC | 15285 | 15269.715 | 15300.285 |
| 1028 | CAACAATATATGGAACACCTACCTGGCGAGACAATAGAATGTGTTCCCTC | 15331 | 15315.669 | 15346.331 |
| 1029 | TTATAGTTACGGCCGCCGTTTACTGGGGCTTCAATTCAATGCTTCTCTTG | 15315 | 15299.685 | 15330.315 |
| 1030 | ACAACAGAGCTTTACGATCCGAAAACCTTCATCACTCACGCGGCGTTGCT | 15259 | 15243.741 | 15274.259 |
| 1031 | CCCGTTCCACGGGTTAGAATCCAAACAAATAAAGGGTCGTATTTCAACAG | 15371 | 15355.629 | 15386.371 |
| 1032 | CCCCCTTCCCCCCTCTCCTCCCCCTTCCCCCTTTCGCGCCCCCTTTTCCC | 14687 | 14672.313 | 14701.687 |
| 1033 | TGGTGTTCCAACCAATTCGGCTTGGGGGGATGGATCTTAAAAACTGGTCC | 15472 | 15456.528 | 15487.472 |
| 1034 | CTCGTGTCCCGCCGTACTCAGGATCCTGCTTGGCATCAAGTGAATTTCAA | 15288 | 15272.712 | 15303.288 |
| 1035 | AGCTTCTACACCCTTCAACCATCTATTCCGTCAGATGGCGGCACTGTCAC | 15177 | 15161.823 | 15192.177 |
| 1036 | CCGATTAGTACCAGTCAACTCCGTACATCACTGCACTTCCATCCCTGGCC | 15122 | 15106.878 | 15137.122 |
| 1037 | CGCTTGAACCACACATCAGGCCCCACGGCTTGCCACCATGTTAACCCGAA | 15190 | 15174.81 | 15205.19 |
| 1038 | TGGCGAGACAATAGAATGTGTTCCCTCGTTTGTGGCATAGGACCATCAGC | 15441 | 15425.559 | 15456.441 |
| 1039 | CGTCCATCCCGGTCCTCTCGTACTAGGGACAGCTCCTCTCAAATATCCTG | 15169 | 15153.831 | 15184.169 |
| 1040 | TCGAGGTGCCAAACCTCCCCGTCGATGTGAACTCTTGGGGGAGATAAGCC | 15412 | 15396.588 | 15427.412 |
| 1041 | CTTAACAACTTAACCTCGCTGCACACAGTAACTCGCCGGCCCGTTCTACA | 15155 | 15139.845 | 15170.155 |
| 1042 | GTCAACAGGTAGTATTCAGGCTTACCAGGTGGTCCTGGCAGATTCACACG | 15426 | 15410.574 | 15441.426 |
| 1043 | AGGCACGCCGTCACACATTGCTGTGCTCCGACCGCTTGTAGGCGTATGGT | 15370 | 15354.63 | 15385.37 |
| 1044 | TCCCTTTCCCCCTTCCCCCCCCCCCCCCCCCCCCCCCCCTTTCCCCCCCC | 14532 | 14517.468 | 14546.532 |
| 1045 | AACCATGACTTTGGGACCTTAGCTGGCGGTCTGGGTTGTTTCCCTCTTCA | 15341 | 15325.659 | 15356.341 |
| 1046 | TGCCATTACACTCTATGAGACCGGTTACCAATCGGTCCGAAGGGCACCTT | 15306 | 15290.694 | 15321.306 |
| 1047 | GATTGGAATTTCTCCGCTACCCACACCTCATCCGCTACCATTTCAACGGG | 15177 | 15161.823 | 15192.177 |
| 1048 | TTCTCGTGTCCCGCGGTACTCTGGATCCTGCTCAGTCTGCTCTGTTTTCG | 15235 | 15219.765 | 15250.235 |
| 1049 | GTAAACCCCCACAACAGCTATGAATTCACTGAAGGGTAACACCCCATAAC | 15254 | 15238.746 | 15269.254 |
| 1050 | TCCCGAAGTTACAGGGTCAATTTGCCTAGTTCCTTAACCGTGAATCACTC | 15271 | 15255.729 | 15286.271 |
| 1051 | CCCCCGACGGGTATCACACGCGCAAGGTTTGGCCATCATCCGCTTTCGCT | 15235 | 15219.765 | 15250.235 |
| 1052 | CCCTTGTCTCAGTGCCCATCTCCGGGCTCCTCCTTCCAGAGCCCGTACCC | 15058 | 15042.942 | 15073.058 |
| 1053 | TCAGACTTGCTCTCGCTGCGGCTTCACACCTTAAGTGCTTAACCTCGCCG | 15200 | 15184.8 | 15215.2 |
| 1054 | CTCCATTCGGAAATCCACGGATCAATGCCTACTTACGGCTCCCCGTGGCT | 15218 | 15202.782 | 15233.218 |
| 1055 | TTTTACGGTTGAGCCGCAAACTTTCACAACTGACTTAACAACCCGCCTAC | 15209 | 15193.791 | 15224.209 |
| 1056 | CGGTTTAGGCTCTTCCGCGTTCGCTCGCCGCTACTTACGGAATCGAGTTT | 15302 | 15286.698 | 15317.302 |
| 1057 | CTTCACTATATACTCTAGTACAGGAATATCAACCTGTTGGCCATCGGATA | 15303 | 15287.697 | 15318.303 |
| 1058 | TGTTTCAGTTCACTGCGTCTTCCTTCTCATAACCTTAACAGTTATGGATA | 15242 | 15226.758 | 15257.242 |
| 1059 | GACGGAGCTTATCCCCCGCCGACTCACTGCCGGGATACGCGTCACGGGTA | 15333.9 | 15318.5661 | 15349.2339 |
| 1060 | CCGAACTGTCTCACGACGTTCTGAACCCAGCTCGCGTACCGCTTTAATGG | 15258 | 15242 .742 | 15273.258 |
| 1061 | GACGGTGACAAGCCGGTACCAGAATATCAACTGGTTACCCATCGACTACG | 15373 | 15357.627 | 15388.373 |
| 1062 | GATGCGCATTCGGAGTTTGTCAAGACTTGATAGGCGGTGAAGCCCTCGCA | 15482 | 15466.518 | 15497.482 |
| 1063 | TAGGTGAGCCGTTACCCCACCTACTAGCTAATCCCATCTGGGCACATCCG | 15227 | 15211.773 | 15242.227 |
| 1064 | TGGTCCCCGCTCATTCCATCAAGGTTTCTCGTGTCTCGATGTACTCTGGA | 15261 | 15245.739 | 15276.261 |
| 1065 | ATGCTCCCCTACCGATACTTTTTAATGCTATCCCGCGCCTTCGGTACCTG | 15150 | 15134.85 | 15165.15 |
| 1066 | TTACCTTTACTTCAACCTGACCATGGGTAGGTCACCCGGTTTCGGGTCGA | 15319 | 15303.681 | 15334.319 |
| 1067 | GTAGTATTTAGCCTTGGAGGATGGTCCCTCCTGCTTCCCACAGGGTTTCA | 15350 | 15334.65 | 15365.35 |
| 1068 | GATTTCCAACCATTCTGAGGGAACCTTTGGGCGCCTCCGTTACCTTTTAG | 15294 | 15278.706 | 15309.294 |
| 1069 | ATCCCTTCCGGGCTTGGCTACTCGGCCGTAGACTTGGCAGTCTAACCGAT | 15305 | 15289.695 | 15320.305 |
| 1070 | GATGCGCATTCGGAGTTTGTCAAGACTTGATAGGCGGTGAAGCCCTCGCA | 15482 | 15466.518 | 15497.482 |
| 1071 | GTAATCGCCTTGGTGGGCCATTACCCCACCAACAAGCTGATAGGCCGCAG | 15341 | 15325.659 | 15356.341 |
| 1072 | ACCCTCAGGTCATCCAGAAGCTTTTCAACGCTTATTGGTTCGGTCCTCCA | 15223 | 15207.777 | 15238.223 |
| 1073 | AGCTCCATGGGGTCTTTCCGTCTAGTTGCGGGTAACCTGCATTTTCACAG | 15350 | 15334.65 | 15365.35 |
| 1074 | CGTGGGGATTAAGTTTAGCGGATTTTCTCGGGAGTATGATTACGTGCGCT | 15549 | 15533.451 | 15564.549 |
| 1075 | TATTTTGGGACCTTAACTGGCGGTCTGGGCTGTTTCCCTCTTGACCATGG | 15372 | 15356.628 | 15387.372 |
| 1076 | TAACCTTGCACGGGATCGTAACTCGCCGGTTCATTCTACAAAAGGCACGC | 15315 | 15299.685 | 15330.315 |
| 1077 | GACGGCCCAGAGACCTGCCTTCGCCATCGGTGTTCTTCCCGATATCTACA | 15233.9 | 15218.6661 | 15249.1339 |
| 1078 | TCACACGGGATTCCACGAGTCCCGCGCTACTTGGGAGACACGATCCGGAG | 15382 | 15366.618 | 15397.382 |
| 1079 | AGTATTTAGCCTTGGAGGATGGTCCCCCCATATTCAGACAGGATACCACG | 15370 | 15354.63 | 15385.37 |
| 1080 | TTTGGCCTCTTCCGCGTTCGCTCGCCACTACTAGCGGAATCTCGGTTGAT | 15262 | 15246.738 | 15277.262 |
| 1081 | CTGCTTCCAAGCCAACATCCTAGCTGTCTTAGCAGTCAGACTTCGTTAGT | 15247 | 15231.753 | 15262.247 |
| 1082 | CTGGGGCTTCAATTCACACCTTCGCTTACGCTAAGCGCTCCTCTTAACCT | 15159 | 15143.841 | 15174.159 |
| 1083 | GTTTGGGCTTCTCCCCTTTCGCTCGCCGCTACTCAGGGAATCACTGTTGT | 15253 | 15237.747 | 15268.253 |
| 1084 | ACAATCCACACCGAATGCCAATACCAAGGTATAGTAAAGGTCCCGGGGTC | 15366 | 15350.634 | 15381.366 |
| 1085 | CAGGGTAGCTTTTATCCGTTGAGCGATGGCCCTTCCATACGGTACCACCG | 15329 | 15313.671 | 15344.329 |
| 1086 | ATAGGCGGTGAAGCCCTCTTGACCTATCGGTCGCTCTACCTCTCACGGTG | 15305 | 15289.695 | 15320.305 |
| 1087 | GCCATGCAGATTCTCACTGCATTCGCGCTACTCATTCCGGCATTCTCACT | 15159 | 15143.841 | 15174.159 |
| 1088 | CGGTACGCCGCCGGTACGGGAATATCCACCCGTTCATCCATTCGACTACG | 15268 | 15252.732 | 15283.268 |
| 1089 | GCACTCCACAGCTCCTTCCGGTACTGCTTCTTCGCGTTAAGAATGCTCCT | 15175 | 15159.825 | 15190.175 |
| 1090 | CGTTCACTCTTCCTTGGCTCCTACCTATCCTGTACATGTGTAACAGATAC | 15173 | 15157.827 | 15188.173 |
| 1091 | CCCCTGACCTGATTCAAGGCCACAGGTTAGAATTTCAGCACTTCAAGAGT | 15314 | 15298.686 | 15329.314 |
| 1092 | CTACCCAGCAATGCCTTTGGCAAGACAACTGGTACACCAGCGGTAAGTCC | 15309 | 15293.691 | 15324.309 |
| 1093 | CCAGCACCGGGCAGGCGTCACCCCCTATACTTCATCTTACGATTTCGCAG | 15203 | 15187.797 | 15218.203 |
| 1094 | ATTCCTCACTGCTGCCTCCCGTAGGAGTTTGGACCGTGTCTCAGTCCCAA | 15240 | 15224.76 | 15255.24 |
| 1095 | CTACGAGACTCAAGCTTGCCAGTATCAGATGCAGTTCCCAGGTTGAGCCC | 15331 | 15315.669 | 15346.331 |
| 1096 | CTCTCAACGATGACGTCTCCTCTTAACCTTCCAGCACCGGGCAGGTGTCA | 15218 | 15202.782 | 15233.218 |
| 1097 | ATTACCGCGGCTGCTGGCACGGAGTTAGCCGGTGCTTCTTCTGCGGGTAA | 15441 | 15425.559 | 15456.441 |
| 1098 | GCGATGGACTTTCACACCGGACGCGACGAGCCGCCTACGAGCCCTTTACG | 15317.9 | 15302.5821 | 15333.2179 |
| 1099 | CCCACACCGGATATGGACCGAACTGTCTCACGACGTTCTGAACCCAGCTC | 15221 | 15205.779 | 15236.221 |
| 1100 | GAATGAATGGCTGCTTCTGAGCCAACATCCTAGTTGTCTTAGAGATCCCA | 15360 | 15344.64 | 15375.36 |
| 1101 | TCCCCGGAGTACCTTTTATCCTTTGAGCGATGTCCCTTCCATACGGAAAC | 15223 | 15207.777 | 15238.223 |
| 1102 | GTAAAGCCACCTTATACCCTTGCATTCTACAGGAGATTTCTGACCTCCTT | 15206 | 15190.794 | 15221.206 |
| 1103 | TCCGCCTGCGCACCCTTTAAACCCAATAAATCCGGATAACGCTCGTATCC | 15155 | 15139.845 | 15170.155 |
| 1104 | AGGAAGTATTCAGGCTTACCAGGTGGTCCTGGCAGATTCACACACGATTC | 15410 | 15394.59 | 15425.41 |
| 1105 | GTGTAGGATTCTCACCTACATCTCGCTACTCACACCGGCATTCTCACTTC | 15143 | 15127.857 | 15158.143 |
| 1106 | GAACTGAGACCGGTTTTCAGGGATCCGCTCCATGTCGCCATGTCGCATCC | 15313.9 | 15298.5861 | 15329.2139 |
| 1107 | TTCCTGAAGTTGATTCTTCGGGTTAGACAGCCAAACTTCTCAGGGTGGTA | 15422 | 15406.578 | 15437.422 |
| 1108 | CGGTACTGGTACGCTATCGGTCAGACAGGTATGCTTAGACTTACGCCACG | 15402 | 15386.598 | 15417.402 |
| 1109 | GTTTCCCCTCGACTTGCATGTGTTAAGCCTGTAGCTAGCGTTCATCCTGA | 15285 | 15269.715 | 15300.285 |
| 1110 | CGAAGTTACGGGGTCATTTTGCCGAGTTCCTTGACAATGCTTCTCCCGCC | 15295 | 15279.705 | 15310.295 |
| 1111 | CTTGGGAATGATCAGCCTGTTATCCCCGGGGTACCTTTTATCCGTTGAGC | 15350 | 15334.65 | 15365.35 |
| 1112 | GTCTATAAGTACTTCGATTTTTGCAAGTCCGAACCCCGAACGTCCGTAGA | 15320 | 15304.68 | 15335.32 |
| 1113 | CACCTTTCCTTCACAGTACTGGTTCACTATCGGTCTCTCGGGAGTATTTA | 15244 | 15228.756 | 15259.244 |
| 1114 | CCGGGAATTCCAGTCTCCCCTACCGCACTCCAGCCCGCCCGTACCCGGCG | 15110.7 | 15095.5893 | 15125.8107 |
| 1115 | ACAGCTTTTCTCGCCATCTTCCATCTCGGACTTCGGTACTAATTTCCCTC | 15100 | 15084.9 | 15115.1 |
| 1116 | TCTTTCGGCGAGGGGGGTTCCCGCCCCCTTTATCGTTACTTATACCTACA | 15246 | 15230.754 | 15261.246 |
| 1117 | TGTATGCGCCATTGTAGCACGTGTGTAGCCCTGGTCGTAAGGGCCATGAT | 15464 | 15448.536 | 15479.464 |
| 1118 | CTTTCGTCTCTGATCGAGTTGTCACTCTCGCAGTCAGGCACCCTTCTGCC | 15182 | 15166.818 | 15197.182 |
| 1119 | GATACTACAATTTCACTGAGCTCTTGGTTGAGACAGCGTCCGGATCATTA | 15375 | 15359.625 | 15390.375 |
| 1120 | GATGTTTCAGTTCAGGCGGTTCCCTCAATACACCTATTTTAAATTTCAGT | 15291 | 15275.709 | 15306.291 |
| 1121 | AAAAAAAAACAAAAAAAAAAACCCTCCCCCCCCCCCCTTCCCCTCCGCGG | 15058 | 15042.942 | 15073.058 |
| 1122 | GCCCTGTTAAGACTTGGTATCCCTTCGGCTCCGCACCTTAAGTGCTTAAC | 15239 | 15223.761 | 15254.239 |
| 1123 | ACCACGAATTCCGCCTGCCTCAACTGCACTCAAGATATCCAGTATCAACT | 15163 | 15147.837 | 15178.163 |
| 1124 | GAGTTTTTCACACTGTGCCATGCAGCACTGTGCGCTTATGCGGTATTAGC | 15374 | 15358.626 | 15389.374 |
| 1125 | TGCCTAGTTCCTTAACCATGAATCTCTCAACGCCTCAGTATGTTCTACCC | 15142 | 15126.858 | 15157.142 |
| 1126 | GGTGTGTACAAGGCCCGGGAACGTATTCACCGCGCCGTGGCTGATGCGCG | 15485 | 15469.515 | 15500.485 |
| 1127 | TTCGCCACCGGTATTCCTCCAGATCTCTACGCATTTCACCGCTACACCTG | 15104 | 15088.896 | 15119.104 |
| 1128 | CGCTTAACGCGTTAGCTCCGACACGGAACACGTGGAACGTGCCCCACATC | 15285.9 | 15270.6141 | 15301.1859 |
| 1129 | ACACGAGCCGAAACCCGTGTCTCTCAGACTCCCACCTATCCTGTGCATCA | 15156 | 15140.844 | 15171.156 |
| 1130 | ACTCGATTTCTCTTCGGCTCCACACCTTAAGTGCTTAACCTTGCCGGCAC | 15159 | 15143.841 | 15174.159 |
| 1131 | TGAACCCGCCCCGAAGGGAAACGCCATCTCTGGCGTCGTCGGGAACATGT | 15382 | 15366.618 | 15397.382 |

3. Immobilized Oligonucleotides for Enriching

In some embodiments, immobilized oligonucleotides are designed for enriching desired library fragments. In some embodiments, oligonucleotides for enriching comprise one or more desired RNA sequence. A user can design oligonucleotides for enriching using similar means of selecting probes as described above for depleting. For example, a user could prepare immobilized oligonucleotides of desired RNA sequences comprised in organisms of interest in the human microbiome, for use in enriching library fragments prepared from these desired RNA sequences. Likewise, a user could prepare immobilized oligonucleotides of desired mRNA sequences from an organism of interest.

In some embodiments, desired RNA may be comprised in some immobilized oligonucleotides, and the complement of desired RNA may be comprised in other immobilized oligonucleotides.

E. Immobilized Oligonucleotides Comprising Adapter Sequences and Library Fragments Comprising Adapter Sequences

In some embodiments, solid supports comprise immobilized oligonucleotides comprising adapter sequences. In some embodiments, the adapter sequences comprised in immobilized oligonucleotides are solid support adapter sequences. As used herein, “solid support adapter sequences” refer to adapter sequences that are comprised in oligonucleotides immobilized to the solid support. In some embodiments, solid support adapter sequences bind to library adapter sequences. As used herein, a “library adapter sequence” refers to an adapter sequence incorporated into library fragments, wherein the library adapter sequence can bind to the solid support adapter sequence. In some embodiments, solid support adapter sequences can serve to immobilize library fragments to a solid support, wherein this immobilizing is not due to the cDNA sequence comprised in the library fragment, but due to binding to a library adapter comprised in library fragments. In some embodiments, binding of a library adapter sequence comprised in a library fragment to a solid support adapter sequence comprised in an immobilized oligonucleotide serves to immobilize the library fragment to the solid support.

In some embodiments, library adapter sequences are incorporated into library fragments during library preparation. In some embodiments, the library of fragments added to the solid support is prepared by a method comprising incorporating one or more library adapters that specifically bind to solid support adapter sequences comprising P5 (SEQ ID NO: 1132), P7 (SEQ ID NO: 1133), and/or their complements. Such methods for incorporating one or more library adapters may be tagmentation or fragmentation followed by adapter ligation.

In some embodiments, library adapter sequences are incorporated into library fragments after performing enriching or depleting as described herein. In other words, enriching or depleting may be performed, and then library adapters may be added to the enriched or depleted library. In some embodiments, library adapter sequences are added to collected library fragments. In some embodiments, the library adapter sequences are added to collected library fragments by ligation.

In some embodiments, library fragments comprise library adapters and the solid support comprises immobilized oligonucleotides comprising solid support adapter sequences that can bind to library adapters.

In some embodiments, the solid support adapter sequences comprise a P5 sequence (SEQ ID NO: 1132), a P7 sequence (SEQ ID NO: 1133), and/or their complements. In some embodiments, library adapter sequences comprise a sequence complementary to P5 sequence or P7 sequence. In some embodiments, library adapter sequences comprise a P5 sequence or P7 sequence.

In some embodiments, a solid support comprises immobilized oligonucleotides comprising P5 and/or its complement. In some embodiments, a solid support comprises immobilized oligonucleotides comprising P7 and/or its complement. In some embodiments, a solid support comprises more than one pool of immobilized oligonucleotides, wherein one or more pool may comprise immobilized oligonucleotides comprising a P5 sequence, a P7 sequence, and/or their complements.

In some embodiments, library adapter sequences comprised in library fragments specifically bind to solid support adapter sequences comprising P5 (SEQ ID NO: 1132), P7 (SEQ ID NO: 1133), and/or their complements.

F. Adapter Complements for Binding Solid Support Adapter Sequences

In some embodiments, adapter complements can bind to solid support adapter sequences. As used herein, an “adapter complement” is an oligonucleotide that can bind to a solid support adapter sequence. In some embodiments, the solid support adapter sequence is single-stranded and the adapter complement is single-stranded. In some embodiments, adapter complements that are all or partially complementary to the solid support adapter sequences are bound to the solid support adapter sequences. In some embodiments, the binding of adapter complements to solid support adapter sequences serves to prevent binding of library adapter sequences comprised in library fragments to solid support adapter sequences. In this way, a user can control when library fragments can bind to solid support adapter sequences comprised in immobilized oligonucleotides. For example, a user can block binding of library adapter sequences (using adapter complements) to solid support adapter sequences during enriching or depleting steps.

In some embodiments, adapter complements bound to the solid support adapter sequences generate double-stranded immobilized oligonucleotides. In some embodiments, solid support adapter sequences bound to adapter complements cannot bind to library adapters. In some embodiments, double-stranded immobilized oligonucleotides comprising a solid support adapter sequence and an adapter complement cannot bind to library adapter sequences.

In some embodiments, the binding of the adapter complements to the solid support adapter sequences is reversible. In some embodiments, an increase in temperature or a denaturing agent can be used to denature library adapter sequences from the solid support adapter sequences. After the denaturing of adapter complements, solid support adapter sequences comprised in immobilized oligonucleotides can be available for binding to library adapter sequences.

G. Solid Support Comprising More than One Pool of Immobilized Oligonucleotides

In some embodiments, a solid support comprises more than one pool of immobilized oligonucleotides on its surface.

For example, a solid support may comprise a first pool of immobilized oligonucleotides for depleting and a second pool of immobilized oligonucleotides for enriching. In some embodiments, one pool of immobilized oligonucleotides may be blocked (such as with complementary nucleic acid sequences) to avoid binding to complementary library fragments during certain steps of methods using the solid support. For example, blocking may be used to inhibit binding of P5/P7 sequences until a user wishes to perform bridge amplification after depletion/enrichment (as shown in FIG. 2).

In some embodiments, a solid support has two pools of immobilized oligonucleotides on its surface, wherein the first pool comprises immobilized oligonucleotides each comprising an unwanted RNA sequence and the second pool comprises immobilized oligonucleotides each comprising a solid support adapter sequence that can bind to a library adapter comprised in library fragments (as shown in FIG. 2). In some embodiments, solid support adapter sequences are bound by adapter complements, wherein the adapter complements can be denatured during a method to allow binding of solid support adapter sequences to library adapters in library fragments. Such a solid support can be used for methods of preparing a depleted library and amplifying the depleted library on the same solid support (such as described in Example 2).

In some embodiments, at least one unwanted RNA sequence has at least 90%, at least 95%, or at least 99% homology to a high-abundance RNA sequence in a sample used to prepare the library of fragments. In some embodiments, all unwanted sequences have at least 90%, at least 95%, or at least 99% homology to a high-abundance RNA sequence in a sample used to prepare the library of fragments.

### II. Methods of Enriching or Depleting of Library Fragments Using Immobilized Oligonucleotides

In some embodiments, a method selects cDNA library fragments from a library of cDNA fragments prepared from RNA. This selecting may be depleting unwanted library fragments by removing them, or this selecting may be enriching desired library fragments and collecting them. In some embodiments, selecting includes both depleting unwanted library fragments and enriching desired library fragments.

In some embodiments, a method of selecting cDNA library fragments from a library of cDNA fragments prepared from RNA comprises (a) preparing a solid support comprising a pool of immobilized oligonucleotides, wherein each immobilized oligonucleotide in the pool comprises a nucleic acid sequence corresponding to an RNA sequence or its complement, (b) adding the library of fragments to the solid support and hybridizing the library fragments to at least one immobilized oligonucleotide to allow binding of library fragments to at least one immobilized oligonucleotide, and (c) collecting library fragments either bound or not bound to at least one immobilized oligonucleotide.

In some embodiments, the selecting is depleting unwanted cDNA library fragments, wherein the RNA sequence comprises an unwanted RNA sequence, the unwanted library fragments comprise those prepared from unwanted RNA sequences, and the collecting comprises collecting library fragment not bound to at least one immobilized oligonucleotide.

In some embodiments, the selecting is enriching desired cDNA library fragments, wherein the RNA sequence comprises a desired RNA sequence, the desired library fragments comprise those prepared from desired RNA sequences, and the collecting comprises collecting library fragment bound to at least one immobilized oligonucleotide.

In some embodiments, the library of fragments is subjected to depleting unwanted cDNA library fragments and the collected library fragments not bound to at least one immobilized oligonucleotides are then subjected to enriching desired cDNA library fragments.

In some embodiments, the library fragments are prepared from a sample comprising RNA. In some embodiments, library fragments are prepared from cDNA prepared from RNA in a sample. Such a sample may be any type comprising RNA and any method of cDNA and library preparation may be combined with the present method.

In some embodiments, the present methods using solid supports decrease library preparation costs and hands-on-time, as compared to prior art methods of depleting unwanted RNA, followed by library preparation. In some embodiments, the present methods reduce degradation and/or loss of rare RNA transcripts that may be seen with RNase-H-mediated depletion methods that are performed before library preparation. Methods described herein can be used for depletion of unwanted rRNA transcripts, as well as unwanted non-rRNA transcripts (such as for depleting host transcriptome when evaluating microbiome samples).

In some embodiments, methods of depleting or enriching library fragments as described herein improves yield of the resulting library after the enriching or depleting in comparison to methods wherein RNA is depleted or enriched prior to library preparation. Such an improvement in yield may be due to the fact that library preparation itself can be limited when a starting RNA sample has very low concentrations of RNA. The present methods of enriching or depleting after library preparation can avoid or reduce the impact of low RNA concentration in the starting sample on library yield.

The present methods of depleting and enriching are flexible for use with any upstream methods of cDNA and library preparation that a user prefers. In other words, a user can choose the best method of cDNA preparation and the best method of library preparation for their particular sample, and then the user can deplete or enrich the resulting library fragments using methods described herein.

In some embodiments, cDNA is prepared using a stranded method. In some embodiments, library preparation comprises incorporating one or more adapter sequence into library fragments. Alternatively, one or more adapter sequence may be incorporated into fragments after the present methods of enriching or depleting.

In some embodiments, single-stranded library fragments are preparing before adding a library of fragments to a solid support. In this way, single-stranded library fragments can bind to single-stranded immobilized oligonucleotides on the surface of a solid support.

In some embodiments, the method is performed after library preparation from cDNA prepared from RNA. In some embodiments, the method does not require degradation of RNA.

In some embodiments, the library depleted of unwanted library fragments or enriched for desired library fragments is assessed for library size and/or concentration. The library depleted of unwanted library fragments or enriched for desired library fragments may also be amplified and/or sequenced.

In some embodiments a method comprises steps of both depleting unwanted library fragments and enriching desired library fragments. For example, a depletion flowcell may be used to deplete unwanted library fragments, and the depleted library can then be enriched for desired library fragments using an enrichment flowcell. Such a workflow comprising both depletion and enrichment may have particular use for generating data from desired library fragments that are relatively rare in a sample. For example, data from library fragments generated from a particular microorganism comprised in a metatranscriptomics sample may be improved by a method of depletion followed by enrichment.

In some embodiments, a method comprises amplifying and/or sequencing on the same flowcell used for depleting and/or enriching. Such a method comprising amplifying and/or sequencing on the same flowcell used for depleting and/or enriching may be termed a “one-pot” or “single flowcell” method.

In some embodiments, amplifying and sequencing are not performed on the flowcell used for depleting and/or enriching. For example, a collected library may be amplified in a thermocycler and then the amplified library fragments are sequenced on a flowcell that is different from the flowcell used for the depleting and/or enriching.

In some embodiments, amplifying is performed on the flowcell used for depleting and/or enriching, and the amplified library fragments are then sequenced on a flowcell that is different from the flowcell used for the depleting and/or enriching. Such a method may comprise bridge amplification on the flowcell used for depleting and/or enriching (as described below), and amplified library fragments are then sequenced on a flowcell that is different from the flowcell used for the depleting and/or enriching.

A. Methods of Depleting

In some embodiments, library fragments prepared from one or more abundant RNA transcripts, sequences thereof, or subsequences thereof, have been depleted from the sample using a plurality of immobilized oligonucleotides after RNA transcripts are reverse transcribed to generate complementary DNAs (cDNAs) and library fragments are prepared from the cDNA. In some embodiments, the library fragments are sequenced after the depleting to generate a plurality of sequence reads. In some embodiments, the one or more abundant RNA transcripts can be ribosomal RNA transcripts and/or globin mRNA transcripts.

In some embodiments, a method of depleting unwanted cDNA library fragments from a library of cDNA fragments prepared from RNA comprises (a) preparing a solid support comprising at least one immobilized oligonucleotide, wherein each immobilized oligonucleotide comprises a nucleic acid sequence corresponding to an unwanted RNA sequence or its complement, (b) adding the library of fragments to the solid support and hybridizing the library fragments to the at least one immobilized oligonucleotide to allow binding of unwanted library fragments to the at least one immobilized oligonucleotide, and (c) collecting library fragments not bound to the at least one immobilized oligonucleotide. In some embodiments, the solid support for depleting comprises a pool of oligonucleotides. In some embodiments, the pool of oligonucleotides comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, or 1100 or more oligonucleotides.

In some embodiments, unwanted library fragments comprise those prepared from unwanted RNA sequences. In some embodiments, library fragments that hybridize to immobilized oligonucleotides comprise library fragments prepared from unwanted RNA sequences. In some embodiments, the unwanted RNA sequences comprise rRNA.

In some embodiments, the collected library fragments comprise a library depleted of unwanted library fragments. In some embodiments, the collected library fragments are collected in a reservoir comprised in a sequencer comprising the flowcell. Collected library fragments can then be removed from the reservoir, and the user can perform any additional steps of interest, such as quantification, amplification, quality control, or sequencing.

In some embodiments, all unwanted sequences have at least 90%, at least 95%, or at least 99% homology to a high-abundance RNA sequence in a sample used to prepare the library of fragments. In some embodiments, all unwanted sequences have at least 90%, at least 95%, or at least 99% homology to a high-abundance RNA sequence in a sample used to prepare the library of fragments.

In some embodiments, the library depleted of unwanted library fragments comprises fewer library fragments prepared from unwanted RNA sequences, as compared to the same library before it was added to the solid support. In other words, the present method of depleting may decrease the number of library fragments prepared from unwanted RNA sequences that are comprised in the collected library.

1. Denaturing in Methods of Depleting

In some embodiments, a method of depleting further comprises a step of denaturing one or more nucleic acid bound to an immobilized oligonucleotide.

In some embodiments, a method further comprises denaturing library fragments hybridized to immobilized oligonucleotides. In some embodiments, the denatured library fragments are unwanted library fragments. In some embodiments, unwanted library fragments are denatured from immobilized oligonucleotides, and unwanted library fragments are siphoned to a waste container.

In some embodiments, a method further comprises denaturing adapter complements hybridized to immobilized oligonucleotides. In some embodiments, adapter complements are denatured from immobilized oligonucleotides, and adapter complements are siphoned to a waste container.

In some embodiments, a single step denatures both adapter complements and unwanted library fragments. In some embodiments, both adapter complements and unwanted library fragments are siphoned to a waste container.

In some embodiments, the denaturing is performed with a denaturing agent or heat. In some embodiments, the denaturing agent is NaOH.

In some embodiments, a method comprises repeating steps. In some embodiments, the steps of adding a sample, collecting, and denaturing are repeated, wherein the collected library fragments are added back to the solid support after the denaturing. In this way, multiple rounds of depleting of unwanted library fragments (by binding of unwanted library fragments to immobilized oligonucleotides) can be performed. Multiple rounds of depleting may increase the percentage of unwanted fragments that are depleted from a library.

In some embodiments, a method further comprises adding the collected library fragments to the solid support after denaturing the hybridized library fragments and/or adapter complements.

2. Depleting of Host RNA

In some embodiments, a method of depleting is for depleting library fragments prepared from host RNA. In some embodiments, host RNA are unwanted RNA sequences, while non-host RNA are desired RNA sequences.

In some embodiments, the unwanted library fragments that hybridize to immobilized oligonucleotides comprise library fragments prepared from host RNA comprised in a sample comprising host RNA and non-host nucleic RNA. In other words, the depleting method may be for depleting library fragments prepared from host RNA from a sample that comprises both library fragments prepared from host RNA and library fragments prepared from non-host RNA. Representative samples that could comprise host RNA and non-host RNA (and be used for library preparation) include samples for assessing a patient's microbiome or assessing fluids from a patient for an infectious organism (such as a virus, fungus, or bacterium).

In some embodiments, the non-host RNA is microbial. In some embodiments, the microbe is a bacterium, a virus, and/or a fungus. In some embodiments, the microbe is a pathogen. In some embodiments, the microbe is an organism in the host microbiome. In some embodiments, the host is human.

B. Methods of Enriching

In some embodiments, a method of enriching desired cDNA library fragments from a library of cDNA fragments prepared from RNA comprises (a) preparing a solid support comprising at least one immobilized oligonucleotide, wherein each immobilized oligonucleotide comprises a nucleic acid sequence corresponding to a desired RNA sequence or its complement, (b) adding the library of fragments to the solid support and hybridizing the library fragments to the at least one immobilized oligonucleotide to allow binding of desired library fragments to the at least one immobilized oligonucleotide, and (c) collecting library fragments bound to the at least one immobilized oligonucleotide. In some embodiments, the desired library fragments comprise those prepared from desired RNA sequences.

In some embodiments, the solid support for enriching comprises a pool of oligonucleotides. In some embodiments, the pool of oligonucleotides comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, or 1100 or more oligonucleotides.

In some embodiments, the desired RNA sequence has homology to an RNA sequence that a user wishes to study, i.e., an RNA sequence of interest. In some embodiments, at least one desired RNA sequence has at least 90%, at least 95%, or at least 99% homology to an RNA sequence of interest in a sample used to prepare the library of fragments. In some embodiments, all desired RNA sequences have at least 90%, at least 95%, or at least 99% homology to an RNA sequence of interest in a sample used to prepare the library of fragments. In some embodiments, at least one desired RNA sequence is an RNA sequence of interest.

In some embodiments, the collected library fragments comprise a library enriched for desired library fragments. In some embodiments, the library of fragments added to the solid support is prepared from RNA using a stranded method of cDNA preparation.

In some embodiments, the collecting comprises denaturing the library fragments hybridized to the immobilized oligonucleotides and then collecting the library enriched for desired fragments in a reservoir comprised in a sequencer comprising the solid support. In other words, the library fragments bound to immobilized oligonucleotides may comprise desired library fragments, and these desired library fragments may be denatured and then collected.

In some embodiments, the denaturing is performed with a denaturing agent or heat. In some embodiments, the denaturing agent is NaOH.

In some embodiments, the steps of adding the library, denaturing, and collecting library fragments not bound to the solid support are repeated, wherein the collected library fragments not bound to the solid support are then added back to the solid support after the denaturing. Multiple rounds of these steps may lead to greater enrichment of desired library fragments, as more unwanted library fragments may be removed.

In some embodiments, the library enriched for desired library fragments comprises a greater percentage of library fragments prepared from desired RNA sequences, as compared to the library before adding to the solid support. This enrichment can be due to the removal of unwanted library fragments that do not bind to immobilized oligonucleotides comprising desired RNA sequences.

Additional steps may be performed once an enriched library is prepared (i.e., bound desired library fragments are denatured and collected). In some embodiments, the library enriched for desired library fragments is assessed for library size and/or concentration. In some embodiments, the library enriched for desired library fragments is sequenced. In some embodiments, the method further comprises amplifying the library enriched for desired library fragments before sequencing.

C. Samples

The present methods are not limited to a specific type of sample comprising RNA, and these methods can be used with libraries prepared from any sample comprising RNA. Described below are a few exemplary types of samples comprising RNA, wherein sequencing of library fragments prepared from this RNA can be improved by enriching or depleting.

In some embodiments, the sample comprises a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof. In some embodiments, the sample comprises an organism of a species that is not predetermined, an unknown species, or a combination thereof. As used herein, “a species not predetermined” means that a user has not already characterized a given species to be present in the sample. For example, the spectrum of bacterial species present in a sample from, for example, soil or gut microbiome may not be predetermined, although the bacterial species later determined to be in the sample may be generally known in the art. As used herein, “unknown species” refers to a species that has not been previously characterized.

In some embodiments, the sample comprises organisms of at least two species.

1. Metatranscriptomic and Microbiome Samples

In some embodiments, methods are used to assess RNA from metatranscriptomic samples. As used herein, “metatranscriptomic samples” refer to samples for generating culturable and non-culturable microbial transcriptome information by large-scale, high-throughput sequencing of transcripts from all microbial communities in specific environmental samples. Metatranscriptomic sequencing allows a user to randomly sequence RNA for understanding complex microbial communities. Methods that can avoid culturing of microbes can allow for data that avoids bias introduced by methods related to individual bacterial isolation and culture.

In some embodiments, the metatranscriptomic sample is a “microbiome sample” from a patient. As used herein, a microbiome sample refers to microorganisms that are present in one or more part of the patient's body.

In some embodiments, the patient is human. In some embodiments, the microbiome sample is oral, vaginal, or from the gut. In some embodiments, the sample from the gut is a stool sample. In some embodiments, the oral sample is a sample from the tongue.

In some embodiments, the patient is at least 12 months of age, at least 15 months of age, at least 24 months of age, or at least 36 months of age. In some embodiments, the microbiome sample comprises at least one unwanted RNA molecule from Faecalibacterium, Lachnospiraceae, and/or Clostridium. In some embodiments, the microbiome sample is vaginal and comprises at least one unwanted RNA molecule from Gardnerella, Lactobacillus, and/or Olsenella. In some embodiments, the microbiome sample is from tongue and comprises at least one unwanted RNA molecule from Veillonella, Rothia, Streptococcus and/or Prevotella.

The spectrum of bacterial species present in a sample from, for example, soil or gut microbiome may not be predetermined. Further, bacteria species present in a sample can involve hundreds or perhaps thousands of different species. Consequently, depletion protocols designed against only two representative bacterial species can be insufficient for the needs of the metatranscriptome field. Methods described herein can be used with designing of immobilized oligonucleotides for depleting abundant sequences (e.g., abundant transcripts, such as rRNAs and globin mRNAs) from a sample, such as a complex sample including a metatranscriptomic biosample.

Metatranscriptomic analysis has a number of applications. In some embodiments, a user wants to evaluate the microbial population in a patient, as specific bacteria comprised in the patient's microbiome are linked to either positive or negative effects on the patient. For example, a user might want to evaluate the microbiome of a patient exhibiting symptoms of an overactive immune response. In some embodiments, a user may wish to evaluate the impact of a treatment on a patient's microbiome using metatranscriptomic analysis.

Metatranscriptomic samples may comprise a broad spectrum of organisms. In some embodiments, immobilized oligonucleotides for use in the present methods are designed in an unbiased fashion. In other words, the present methods can be used to prepare enriched libraries from a broad spectrum of organisms, including those which may not be identified, without biasing the library towards known organisms.

In some embodiments, the present methods may be used to deplete known sequences from a metatranscriptomic sample (in which case known sequences would be the unwanted RNA sequences) to prepare a library with a greater percentage of library fragments from unknown sequences. When a greater percentage of library fragments are from unknown sequences, the user could sequence these library fragments at greater depth.

In some embodiments, the sample comprises an organism of a species that is not predetermined, an unknown or unidentified species, or a combination thereof. In some embodiments, the sample comprises organisms of, of about, of at least, or of at most, 1, 2, 3, 4, 5, 6, 7, 8, 9, 10, 20, 30, 40, 50, 60, 70, 80, 90, 100, or a number or a range between any two of these values, species. The one or more abundant RNA transcripts can comprise RNA transcripts from organisms of, of about, of at least, or of at most, 1, 2, 3, 4, 5, 6, 7, 8, 9, 10, 20, 30, 40, 50, 60, 70, 80, 90, 100, or a number or a range between any two of these values, species. The sample can comprise, comprise about, comprise at least, or comprise at most, 1 ng, 2 ng, 3 ng, 4 ng, 5 ng, 6 ng, 7 ng, 8 ng, 9 ng, 10 ng, 20 ng, 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, or 1000 ng of RNA transcripts.

2. Oncology Samples

In some embodiments, samples may be from a cancer patient (i.e., an oncology sample). For example, oncology samples may be used to evaluate changes in RNA expression in tumor cells, and to potentially monitor these changes over time or over the course of a therapeutic treatment. In such cases, RNA related to tumor markers may be desired RNA. In the present method, RNA from known tumor markers may be used as desired RNA to design oligonucleotides for immobilizing to a solid support for enriching library fragments related to cancer markers. Alternatively or together with an enrichment method described herein, oncology samples may be depleted of rRNA and/or mRNA related to other “housekeeping” genes that are not implicated in tumor genesis or progression.

D. Unwanted Library Fragments Functioning as Carrier Molecules

In some embodiments, unwanted RNA can function as carrier nucleic acid. In some embodiments, unwanted RNA serves as carrier molecules for other library fragments. In some embodiments, unwanted RNA serves as carrier molecules for desired library fragments.

It is well known that samples with low nucleic acid concentration perform poorly in a variety of biochemical reactions, such as having limited percentage yield in purification methods (See, for example, Higgins et al., Forensic Sci Med Pathol 10:56-61 (2014)). Low input concentrations can be associated with low library complexity and can result in difficulties with cDNA conversion or other aspects of library construction. Accordingly, “upfront depletion” methods (such as depletion methods using RNase) may results in RNA samples that produce low library yield that reduces downstream data quality (such as poor sequencing results). In some embodiments, depletion methods described herein have an advantage of unwanted RNA functioning as carrier nucleic acid for desired RNA during cDNA and library preparation. In some embodiments, the present methods of depletion of library fragments improve the yield of desired library fragments in comparison to prior art method of depletion of RNA followed by library preparation.

In some embodiments, the yield of library fragments after depletion of unwanted library fragments via the present method is greater than the yield of library fragments after depletion of unwanted RNA followed by library preparation in prior art methods.

In some embodiments, sequencing results after library preparation and depletion with the present method (with downstream depletion of unwanted library fragments after library preparation) may be improved as compared to sequencing results with prior art methods (with upstream depletion of unwanted RNA before library preparation), when aliquots of the same sample comprising RNA is used for the present and prior art methods.

Performance of prior art depletion methods that rely on depletion of unwanted RNA samples before library preparation may have performance issues with low input (for example, less than 100 ng of starting RNA). As used herein, “starting RNA” refers to the RNA present in a biological sample, before methods of depletion and library preparation. In some embodiments, the present methods yield sequencable libraries after depletion when the starting sample comprises less than 100 ng of RNA. In some embodiments, starting samples comprise less than 100 ng of RNA, less than 50 ng of RNA, less than 20 ng of RNA, less than 10 ng of RNA, or less than 1 ng of RNA.

E. Stranded cDNA Preparation

A variety of methods are known in the art that allow sequencing data to identify the mRNA strand that was the origin of a library fragment. Use of such “stranded” methods can allow the user to determine the sequence of the original mRNA strand using the sequence of the first strand of cDNA (without confounding data from a second strand of cDNA).

In some embodiments, a library of fragments added to the solid support is prepared from RNA using a stranded method of cDNA preparation.

In the present methods, use of a stranded method of cDNA preparation means that most library fragments after an amplification step will correspond to the complementary sequence of an undesired RNA. In this way, unwanted fragments after amplification can generally be depleted by immobilized oligonucleotides corresponding to the undesired RNA.

In some embodiments, a user may prefer to use a non-stranded method of cDNA preparation. When cDNA is prepared by a non-stranded method and a user wants to deplete unwanted RNA, the user may prefer to immobilize oligonucleotides corresponding to both the unwanted RNA sequence and its complement to increase efficiency of the depleting. When cDNA is prepared by a non-stranded method and a user wants to enrich desired RNA, the user may prefer to immobilize oligonucleotides corresponding to both the desired RNA sequence and its complement to increase efficiency of the enriching.

An exemplary method of stranded cDNA preparation is outlined in “TruSeq Stranded Total RNA Reference Guide,” Illumina, 2017. The mRNA is copied into a first strand of cDNA using reverse transcriptase in a First Strand Synthesis Actinomycin Mix, which allows RNA-dependent synthesis and prevents undesired DNA-dependent synthesis. The First Strand Synthesis Actinomycin Mix can improve strand specificity when generating a first strand of cDNA. Second strand cDNA synthesis is performed using DNA polymerase I and RNase H in a Second Strand Marking Mix, wherein dTTP has been replaced by dUTP. Incorporation of dUTP in the second strand of cDNA can quench amplification of this strand when a uracil-intolerant DNA polymerase is used.

In some embodiments, the nucleoside trisphosphates comprised in a composition for first strand cDNA synthesis comprises dCTP, dATP, dGTP, and dTTP.

In some embodiments, dTTP is replaced with dUTP in a second strand cDNA synthesis reaction for strand specificity. In some embodiments, a composition for second strand cDNA synthesis comprises dCTP, dATP, dGTP, and dUTP. In some embodiments, incorporation of dUTP in the second strand of cDNA suppresses amplification of the second strand of cDNA in the index PCR reaction during library preparation. In some embodiments, suppression of amplification of the second strand of cDNA allows for strand-specific methods.

In some embodiments, a uracil-intolerant DNA polymerase may be used in stranded methods of cDNA preparation comprising amplification. In some embodiments, the presence of uracil in a second strand of cDNA prepared from RNA in a sample can quench amplification of this second strand when a uracil-intolerant DNA polymerase is used. In this way, the amplified cDNA is limited to that generated from the first strand of cDNA from an RNA that was comprised in the sample.

In some embodiments, cDNA preparation is by a non-stranded method that does retain strand information from the mRNA.

F. Library Preparation

Libraries prepared by any method can be used together with the present methods of enriching or depleting. In some embodiments, a method of library preparation prepares double-stranded library fragments, and the double-stranded library fragments are denatured before being added to a solid support. In this way, a library fragment may be single stranded when they are available to hybridize to an immobilized oligonucleotide comprising a sequence all or partially complementary to the library fragment. Similarly, in some embodiments, immobilized oligonucleotides are single-stranded to allow for hybridizing and capturing of single-stranded library fragments that are complementary. In some embodiments, specific binding of a single-stranded library fragment to an immobilized oligonucleotide generates a double-stranded oligonucleotide. The immobilized oligonucleotide specifically bound to the library fragment may be bound with a high-enough affinity to avoid denaturing of this double-stranded oligonucleotide in standard washing steps. In this way, library fragments with specific binding to an immobilized oligonucleotide may remain bound during washing steps and removal of unbound library fragments.

G. Library Adapter Sequences

In some embodiments, one or more adapter sequence are incorporated into library fragments. Such adapter sequences comprised in library fragments may be termed “library adapters.” In some embodiments, a given library adapter sequence may universal, meaning that all or most library fragments comprise this library adapter sequence.

In some embodiments, library adapter sequences are incorporated into library fragments during library preparation. In some embodiments, library adapter sequences are incorporated into library fragments after methods of depleting or enriching as described herein.

Adapter sequences can be any known in the art, and one skilled in the art can choose adapter sequences based on any downstream method (such as sequencing) and what platform will be used for the downstream method (such as a particular sequencer). Further, a library adapter sequence can be designed to bind to a solid support adapter sequence comprised in an immobilized oligonucleotide on a solid support.

In some embodiments, a library fragment comprises one or more adapter sequence in addition to the library adapter sequence for binding to the solid support adapter. In some embodiments, an adapter sequence comprises a primer sequence, an index tag sequence, a capture sequence, a barcode sequence, a cleavage sequence, or a sequencing-related sequence, or a combination thereof. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adapter to nucleic acid fragments. In some embodiments, the adapter sequence comprises a P5 (SEQ ID NO: 1132) or P7 sequence (SEQ ID NO: 1133), and/or their complement, to facilitate binding to a flowcell in certain sequencing methods. This disclosure is not limited to the type of adapter sequences which could be used and a skilled artisan will recognize additional sequences which may be of use for library preparation and next generation sequencing.

In some embodiments, an adapter comprises a region for cluster amplification. In some embodiments, an adapter comprises a region for priming a sequencing reaction.

In some embodiments an adapter comprises an A14 primer binding sequence (SEQ ID NO: 1134). In some embodiments, an adapter comprises a B15 primer binding sequence (SEQ ID NO: 1135).

H. Amplifying

In some embodiments, methods described herein comprise one or more amplification step. In some embodiments, library fragments are amplified before being added to a solid support. In some embodiments library fragments are amplified after a method of enriching or depleting described herein. In some embodiments, amplifying is by PCR amplification.

1. Amplification with a Uracil-Intolerant Polymerase

In some embodiments, library fragments are amplified before being added to a solid support. In some embodiments, amplifying of library fragments is comprised in a method of library preparation. For example, in a stranded method of cDNA preparation, amplification with a uracil-intolerant DNA polymerase is used to selectively amplify cDNA strands prepared as a first strand from RNA (without amplifying second strands of DNA that comprise uracil). Accordingly, the library fragments added to the solid support may comprise mostly fragments comprising a sequence complementary to a desired RNA or unwanted RNA. In other words, the library fragments may comprise mostly fragments prepared from a first strand of cDNA. In some embodiments, more than 70%, more than 80%, more than 90%, or more than 95% of library fragments comprise cDNA from a first strand of cDNA.

2. Amplification after Depleting or Enriching

In some embodiments, collected library fragments are amplified after a method of depleting or enriching. In some embodiments, a depleted library is amplified. In some embodiments, an enriched library is amplified.

In some embodiments, the amplifying is performed with a thermocycler. In some embodiments, the amplifying is by PCR amplification.

In some embodiments, the amplifying is performed without PCR amplification. In some embodiments, the amplifying does not require a thermocycler. In some embodiments, enriching/depleting and amplifying after the enriching/depleting is performed in a sequencer.

In some embodiments, the amplifying is performed without a thermocycler. In some embodiments, the amplifying is performed by bridge or cluster amplification. As shown in FIG. 2, library fragments comprising library adapter sequences can bind to immobilized oligonucleotides comprising solid support adapter sequences. This binding can allow for standard bridge amplification. In some embodiments, bridge amplification is performed on the same solid support used for enriching or depleting.

In some embodiments, bridge amplification is performed after adding the collected library fragments to the solid support and allowing the library adapters comprised in the collected library fragments to bind to the solid support adapter sequences, wherein the adding is performed after denaturing the hybridized library fragments and/or adapter complements. Such a method is described in FIG. 2 and Example 2 herein.

In some embodiments, a method of amplifying desired cDNA library fragments from a library of cDNA fragments prepared from RNA, comprises:
a. providing a solid support having two pools of immobilized oligonucleotides on its surface, wherein the first pool comprises immobilized oligonucleotides each comprising an unwanted RNA sequence and the second pool comprises immobilized oligonucleotides each comprising a solid support adapter sequence that can bind to a library adapter comprised in library fragments, wherein adapter complements are reversibly bound to the solid support adapter sequences,
b. adding the library of fragments to the solid support and hybridizing the library fragments to at least one immobilized oligonucleotide to allow binding of unwanted library fragments to the first pool of oligonucleotides,
c. collecting library fragments not bound to the first pool of oligonucleotides to prepare collected library fragments;
d. denaturing and removing library fragments bound to the first pool of oligonucleotides and adapter complements bound to the adapter sequences of the second pool of oligonucleotides;
e. adding the collected library fragments to the solid support and hybridizing the library fragments to at least one immobilized oligonucleotide to allow binding of desired library fragments to the second pool of oligonucleotides; and
f. amplifying the bound desired library fragments by bridge amplification on the solid support.

For example, in some embodiments, the immobilized DNA fragments can be amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays.” The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized library fragment is used to produce immobilized amplicons.

I. Sequencing of Depleted or Enriched Libraries

In some embodiments, a library depleted of unwanted library fragments is sequenced. In some embodiments, a library enriched for desired library fragments is sequenced.

After methods of depleting or enriching described herein, the collected library may comprise less than 15%, 13%, 11%, 9%, 7%, 5%, 3%, 2% or 1% or any range in between of unwanted RNA species. In some embodiments, the collected library after enriching or depleting comprises at least 99%, 98%, 97%, 95%, 93%, 91%, 89% or 87% or any range in between of desired RNA. In other words, the library for sequencing after the enriching or depleting mainly comprises library fragments that were prepared from RNA of interest.

In some embodiments, sequencing data generated after depleting of unwanted library fragments has fewer sequences corresponding to unwanted RNA as compared to the same library sequenced without the depleting.

In some embodiments, sequencing data generated after enriching of desired library fragments has a higher percentage of sequences corresponding to desired RNA as compared to the same library sequenced without the enriching.

Depleted or enriched libraries prepared by the present method can be used with any type of RNA sequencing, such as RNA-seq, small RNA sequencing, long non-coding RNA (lncRNA) sequencing, circular RNA (circRNA) sequencing, targeted RNA sequencing, exosomal RNA sequencing, and degradome sequencing.

For example, for circRNA sequencing, a user may prepare by depleted of linear RNA with digestion of linear RNA, followed by library preparation and depleting of rRNA by a method described herein. As such, the present methods can easily be combined with other steps in known protocols related to RNA sequencing.

Depleted or enriched libraries can be sequenced according to any suitable sequencing methodology, such as direct sequencing, including sequencing by synthesis, sequencing by ligation, sequencing by hybridization, nanopore sequencing and the like. In some embodiments, the depleted or enriched libraries are sequenced on a solid support. In some embodiments, the solid support for sequencing is the same solid support on which the enriching or depleting is performed. In some embodiments, the solid support for sequencing is the same solid support upon which amplification occurs after the enriching or depleting.

Flowcells provide a convenient solid support for performing sequencing. One or more library fragments (or amplicons produced from library fragments) in such a format can be subjected to an SBS or other detection technique that involves repeated delivery of reagents in cycles. For example, to initiate a first SBS cycle, one or more labeled nucleotides, DNA polymerase, etc., can be flowed into/through a flowcell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flowcell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

Performing sequencing, and optionally performing amplifying, on the same solid support used for the depleting and/or enriching can reduce the number of hands-on steps for the user and sample loss that would be associated with transferring sample from one solid support to another.

III. Methods of Depleting rRNA from a Microbiome Sample from a Patient Using DNA Probes and RNase

Creating nucleic acid libraries from RNA for sequencing is often times difficult due to an abundance of unwanted transcripts such as ribosomal RNA (rRNA) that can dominate a sample and swamp out the RNA sequences of interest. If the unwanted transcripts are not removed, analysis of the transcriptome could be compromised. Therefore, depleting unwanted RNA from a microbiome sample comprising nucleic acid prior to analysis such as sequencing or other downstream applications can increase the specificity and accuracy of the desired analysis. Exemplary methods of depleting rRNA are described in WO 2020132304 A1, which is incorporated herein in its entirety.

The present disclosure describes methods and materials useful in depleting rRNA species from a nucleic acid sample such that the RNA of importance can be studied and is not lost in the sea of undesired RNA transcripts. The nucleic acid sample may be any described herein, such as a metatranscriptomic sample.

A microbiome sample may contain RNA or DNA or both, including both undesired (off-target or unwanted) and desired (target) nucleic acids. The DNA or RNA in the sample can be either unmodified or modified and includes, but is not limited to, single or double stranded DNA or RNA or derivatives thereof (e.g., some regions of the DNA or RNA are double stranded whereas concurrently other regions of the DNA or RNA are single stranded) and the like. However, a microbiome sample may also contain cells from the host. For example, a gut microbiome patient from a human patient (i.e., the “host”) may comprise microorganisms present in the gut as well as host cells, such that the sample comprises nucleic acids from both the host and microorganisms.

A microbiome sample may include any chemically, enzymatically, and/or metabolically modified forms of nucleic acids as well as any unmodified forms of nucleic acids, or combinations thereof. A microbiome sample can contain both wanted and unwanted nucleic acids. Unwanted nucleic acids include those nucleic acids from the host as well as rRNA from microorganisms. Wanted or desired nucleic acids are those nucleic acids that are the basis or focus of study, the target nucleic acids. For example, a researcher may desire to study mRNA expression analysis from microorganisms comprised in a microbiome, wherein rRNA from microorganisms would be considered unwanted nucleic acids and other RNA from microorganisms is the target nucleic acid. In some embodiments, unwanted RNA is rRNA.

For example, a microbiome sample could contain the desired RNA (such as mRNA) from microorganisms while also including undesired rRNA. General methods for RNA extraction from a gross sample, like blood, tissue, cells, fixed tissues, etc., are well known in the art, as found in Current Protocols for Molecular Biology (John Wiley & Sons) and multitude molecular biology methods manuals. RNA isolation can be performed by commercially available purification kits, for example Qiagen RNeasy mini-columns, MasterPure Complete DNA and RNA Purification Kits (Epicentre), Parrafin Block RNA Isolation Kit (Ambion), RNA-Stat-60 (Tel-Test) or cesium chloride density gradient centrifugation. The current methods are not limited by how the RNA is isolated from a sample prior to RNA depletion.

In some embodiments, methods include use of probes to host unwanted RNA and/or microbial unwanted RNA. For example, methods described herein may include the use of probes directed to non-microbial RNA (such as the DP1 probe set described herein) as well as probes directed to microbial rRNA (such as HMv1 and/or HMv2 probe sets described herein), as described in Example 5.

In some embodiments, a method for depleting unwanted RNA molecules comprised in a patient microbiome sample, wherein the patient microbiome sample comprises at least one target RNA or DNA sequence and at least one unwanted RNA molecule, comprises

(a) sequencing a plurality of probe-development microbiome samples to determine at least one unwanted RNA molecule comprising a bacterial ribosomal RNA (rRNA) sequence from sequencing data; (b) preparing a probe set comprising at least one DNA probe complementary to the at least one unwanted RNA molecule; (c) contacting the patient microbiome sample with the probe set to prepare DNA:RNA hybrids; and (d) contacting the DNA:RNA hybrids with a ribonuclease that degrades the RNA from the DNA:RNA hybrids, thereby degrading the unwanted RNA molecules in the patient microbiome sample to form a degraded mixture.

In some embodiments, a method for depleting unwanted RNA molecules comprised in a patient microbiome sample, wherein the patient microbiome sample comprises at least one target RNA or DNA sequence and at least one unwanted RNA molecule, comprises

(a) contacting the patient microbiome sample with a probe set comprising at least one sequence comprising at least one of SEQ ID NOs: 1-1131 to prepare DNA:RNA hybrids; and (b) contacting the DNA:RNA hybrids with a ribonuclease that degrades the RNA from the DNA:RNA hybrids, thereby degrading the unwanted RNA molecules in the patient microbiome sample to form a degraded mixture.

In some embodiments, a method further comprises (a) degrading any remaining DNA probes by contacting the degraded mixture with a DNA digesting enzyme, optionally wherein the DNA digesting enzyme is DNase I, to form a DNA degraded mixture; and

(b) separating the degraded RNA from the degraded mixture or the DNA degraded mixture.

In some embodiments, the addition of a destabilizer such as formamide helps remove some unwanted RNA that was shown to be more problematic to deplete if formamide was not present. In some embodiments, formamide may serve to relax structural barriers in the unwanted RNA (such as rRNA) so that the DNA probes can bind more efficiently. Further, the addition of formamide has demonstrated the added benefit of improving the detection of some non-targeted transcripts possibly by denaturing/relaxing regions of the RNAs, for example, which have very stable secondary or tertiary structures and are not normally well represented well in other library preparation methods.

In some embodiments, the contacting with the probe set comprises treating the nucleic acid sample with a destabilizer. In some embodiments, the destabilizer is heat and/or a nucleic acid destabilizing chemical. In some embodiments, the nucleic acid destabilizing chemical comprises betaine, DMSO, formamide, glycerol, or a derivative thereof, or a mixture thereof. In some embodiments, the nucleic acid destabilizing chemical comprises formamide. In some embodiments, the formamide is present during the contacting with the probe set at a concentration of from about 10 to 45% by volume. In some embodiments, treating the sample with heat comprises applying heat above the melting temperature of the at least one DNA:RNA hybrid. In some embodiments, the ribonuclease is RNase H or hybridase.

In some embodiments, the unwanted RNA is converted to a DNA:RNA hybrid by hybridizing partially or completely complementary DNA probes to the unwanted RNA molecules. Methods for hybridizing nucleic acid probes to nucleic acids are well-established in the sciences and whether a probe is partially or completely complementary with the partner sequence, the fact that a DNA probe hybridizes to the unwanted RNA species following washes and other manipulations of the sample demonstrates a DNA probe that can be used in methods of the present disclosure. DNA can also be considered an unwanted nucleic acid if the target for study is an RNA, at which point DNA can also be removed by depletion.

In some embodiments, an RNA sample is denatured in the presence of DNA probes. In some embodiments, the DNA probes are added to the denatured RNA sample (denatured at 95° C. for 2 min.) whereupon cooling the reaction to 37° C. for 15-30 minutes results in hybridization of the DNA probes to their respective target RNA sequences thereby creating DNA:RNA hybrid molecules.

In some embodiments, contacting with the probe set comprises treating the nucleic acid sample with a destabilizer. In some embodiments, a destabilizer is heat or a nucleic acid destabilizing chemical. In some embodiments, a nucleic acid destabilizing chemical is betaine, DMSO, formamide, glycerol, or a derivative thereof, or a mixture thereof. In some embodiments, a nucleic acid destabilizing chemical is formamide or a derivative thereof, optionally wherein the formamide or derivative thereof is present at a concentration of from about 10 to 45% of the total hybridization reaction volume. In some embodiments, treating the sample with heat comprises applying heat above the melting temperature of the at least one DNA:RNA hybrid.

In some embodiments, formamide is added to the hybridization reaction regardless of RNA sample source (e.g., human, mouse, rat, etc.). For example, in some embodiments, hybridizing to the DNA probes is performed in the presence of at least 3%, 5%, 10%, 20%, 25%, 30%, 35%, 40%, or 45% by volume of formamide. In one embodiment, a hybridization reaction for RNA depletion includes approximately 25% to 45% by volume of formamide.

Following the hybridization reaction, a ribonuclease that degrades RNA from a DNA:RNA hybrid may be added to the reaction. In some embodiments, a ribonuclease is RNase H or Hybridase. RNase H (NEB) or Hybridase (Lucigen) are examples of enzymes that will degrade RNA from a DNA:RNA hybrid. Degradation by a ribonuclease such as RNase H or Hybridase degrades the RNA into small molecules that can then be removed. For example, RNase H is reported to digest RNA from a DNA:RNA hybrid approximately every 7-21 bases (Schultz et al., J. Biol. Chem. 2006, 281:1943-1955; Champoux and Schultz, FEBS J. 2009, 276:1506-1516). In some embodiments, the digestion of the RNA of the DNA:RNA hybrid can occur at 37° C. for approximately 30 minutes.

In some embodiments, following DNA:RNA hybrid molecule digestion, the remaining DNA probes and any unwanted DNA in the nucleic acid sample are degraded. Thus, in some embodiments, the methods comprise contacting the ribonuclease-degraded mixture with a DNA digesting enzyme, thereby degrading DNA in the mixture. In some embodiments, the digested sample is exposed to a DNA digesting enzyme such as DNase I, which degrades the DNA probes. The DNase DNA digestion reaction is incubated, for example, at 37° C. for 30 minutes, after which point the DNase enzyme can be denatured at 75° C. for a period of time as necessary to denature the DNase, for example for up to 20 minutes.

In some embodiments, the depletion method comprises separating the degraded RNA from the degraded mixture. In some embodiments, separating comprises purifying the target RNA from the degraded RNA (and degraded DNA if present), for example, using a nucleic acid purification medium, such as RNA capture beads, such as RNAClean XP beads (Beckman Coulter). Thus, in some embodiments, following the enzymatic digestion(s), the target RNA can be enriched by removing the degraded products while leaving the desired and longer RNA targets behind. Suitable enrichment methods include treating the degraded mixture with magnetic beads which bind to the desired fragment size of the enriched RNA targets, spin columns, and the like. In some embodiments, magnetic beads such as AMPure XP beads, SPRISelect beads, RNAClean XP beads (Beckman Coulter) can be used, as long as the beads are free of RNases (e.g., Quality Controlled to be RNase free). These beads provide different size selection options for nucleic acid binding, for example RNAClean XP beads target 100 nucleotides or longer nucleic acid fragments and SPRISelect beads target 150 to 800 nucleotide nucleic acid fragments and do not target shorter nucleic acid sequences such as the degraded RNA and DNA that results from the enzymatic digestions of RNase H and DNase. If mRNA is the target RNA to be studied, then the mRNA can be further enriched by capture using, for example, beads that comprise oligodT sequences for capturing the mRNA adenylated tails. Methods of mRNA capture are well-known by skilled artisans.

Once the target RNA has been purified away from the reaction components including the undesired degraded nucleic acids, additional sample manipulation can occur. In some embodiments, the enriched target total RNA followed by an exemplary library preparation workflow that is typical for subsequent sequencing on, for example, an Illumina sequencer. However, it should be understood that these workflows are exemplary only and a skilled artisan will understand that the enriched RNA can be used in multitude additional applications such as PCR, qPCR, microarray analysis, and the like either directly or following additional manipulation such as converting the RNA to cDNA by using established and will understood protocols.

The methods described herein for RNA depletion may result in a sample enriched with the target RNA molecules. For example, the methods described herein result is a depleted RNA sample comprising less than 15%, 13%, 11%, 9%, 7%, 5%, 3%, 2% or 1% or any range in between of the unwanted RNA species. The enriched RNA sample then comprises at least 99%, 98%, 97%, 95%, 93%, 91%, 89% or 87% or any range in between of the target total RNA. Once the sample has been enriched it can be used for library preparation or other downstream manipulations.

In some embodiments, the DNA probes do not hybridize to the entire contiguous length of an RNA species to be deleted. In some embodiments, the full-length sequence of a RNA species targeted for depletion need not be targeted with a full-length DNA probe, or a probe set that tiles contiguously over the entire RNA sequence. In some embodiments, DNA probes described herein leave gaps such that the DNA:RNA hybrids formed are not contiguous. In some embodiments, gaps of at least 5 nucleotides, 10 nucleotides, 15 nucleotides or 20 nucleotides between DNA:RNA hybrids provided efficient RNA depletion. Further, probe sets that include gaps can hybridize more efficiently to the unwanted RNA, as the DNA probes do not hinder hybridization of adjacent probes as could potentially occur with probes that cover the whole RNA sequence targeted for depletion, or probes that overlap one another.

In some embodiments, the at least one DNA probe comprise 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131 or its complement.

In some embodiments, the at least one DNA probe comprises 500 or more, 600 or more, 700 or more, 800 or more, 900 or more, 1000 or more, 1100 or more, or 1131 sequences selected from SEQ ID NOs: 1-1131 or its complement.

In some embodiments, the at least one DNA probe comprises at least one HMv1 sequence and comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

In some embodiments, the at least one DNA probe comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

In some embodiments, the at least one DNA probe comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

In some embodiments, the at least one DNA probe further comprises at least one sequence of the HMv2 sequences and comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131. In some embodiments, the at least one DNA probe comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131. In some embodiments, the at least one DNA probe comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

In some embodiments, the at least one DNA probe comprises at least one HMv1 sequence or HMv2 sequence and comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

In some embodiments, the at least one DNA probe comprises 100 or more, 500 or more, or 1000 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

In some embodiments, the at least one DNA probe comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

In some embodiments, the at least one DNA probe further comprises at least one sequence of the DP1 sequences and comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127. In some embodiments, the at least one DNA probe comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127. In some embodiments, the at least one DNA probe comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

In some embodiments, the method depletes 70% or greater, 80% or greater, 90% or greater, or 95% or greater of bacterial rRNA comprised in the microbiome sample.

A. Kits and Compositions

In some embodiments, at least one probe is comprised in a kit or composition. The at least one probe may be any combination of probes disclosed herein.

In some embodiments, a composition comprising a probe set comprises at least one DNA probe comprising at least one sequence comprising at least one of SEQ ID NOs: 1-1131; and a ribonuclease capable of degrading RNA in an DNA:RNA hybrid. In some embodiments, the ribonuclease is RNase H.

In some embodiments, a kit comprising a probe set comprises at least one DNA probe comprising at least one sequence comprising at least one of SEQ ID NOs: 1-1131; and a ribonuclease capable of degrading RNA in an DNA:RNA hybrid. In some embodiments, kit comprises a probe set comprising at least one DNA probe comprising at least one of SEQ ID NOs: 1-1131; a ribonuclease; a DNase; and RNA purification beads. In some embodiments, the ribonuclease is RNase H.

In some embodiments, a kit further comprises an RNA depletion buffer, a probe depletion buffer, and a probe removal buffer. In some embodiments, a kit further comprises a nucleic acid destabilizing chemical. In some embodiments, the nucleic acid destabilizing chemical comprises betaine, DMSO, formamide, glycerol, or a derivative thereof, or a mixture thereof. In some embodiments, the nucleic acid destabilizing chemical comprises formamide.

### EXAMPLES

### Example 1. Method of rRNA Depletion Using a Flowcell

A method of rRNA depletion followed by amplification via thermal cycler can be performed. This method would utilize current flowcells used for sequencing, featuring inlet ports for the sequence fluidics system to pump buffers and reagents onto the flowcell and to siphon reagents to a waste container. Like current flowcells for sequencing, oligonucleotide sequences would be tethered (i.e., immobilized) to the surface of the flowcell, and rRNA sequences would be comprised in these immobilized oligonucleotides. The user would load RNA libraries (i.e. library fragments prepared from cDNA prepared from RNA) onto a sequencer stage or inside the sequencer chiller for the fluidics system to load the library onto the flowcell. A user may use a commercially available method of stranded cDNA preparation, such as that described in “TruSeq Stranded Total RNA Reference Guide,” Illumina, 2017.

FIG. 1 outlines a representative method for depletion. As library molecules flow in solution, library fragments generated from rRNA transcripts will hybridize to complementary sequences tethered to the flowcell while library fragments generated from non-rRNA transcripts will continue to flow unimpeded to a storage chamber for collection. After the hybridization step is complete, the user would discard the flowcell and collect the siphoned non-rRNA library fragments for PCR amplification, cleanup quantification, quality control, and sequencing.

This method would leverage the advantages of current flowcell/sequencer capabilities for a user-friendly method of depleting unwanted library fragments, such as those library fragments prepared from rRNA.

### Example 2. Depletion and Bridge Amplification on the Same Flowcell

Methods can also be designed to deplete library fragments prepared from rRNA and amplify library fragments prepared from non-rRNA on the same solid support. This flowcell-like solid support would comprise a pool of immobilized oligonucleotides comprising rRNA sequences. The solid support would also comprise another pool of immobilized oligonucleotides comprising double-stranded P5 and/or P7 oligonucleotides immobilized on the surface. The double-stranded P5 and/or P7 oligonucleotides would comprise an adapter complement that is an oligonucleotide reversibly bound to the P5 and/or P7 adapter sequence (i.e., a solid support adapter sequence).

A representative method is shown in FIG. 2. Library fragments could be prepared by standard methods after cDNA preparation from a sample comprising RNA. These library fragments can be prepared by incorporating library adapter sequences that can bind to P5 and/or P7. Library fragments generated from rRNA transcripts would bind to the surface of the flowcell based on hybridizing to immobilized oligonucleotides comprising rRNA sequences, while library fragments prepared from non-rRNA transcripts would flow unimpeded and be siphoned for temporary storage in a reservoir.

After this step, a denaturing reagent such as NaOH would be pumped across the flowcell device causing the hybridized library fragments prepared from rRNA and the untethered strand of the double-stranded P5 and/or P7 oligonucleotides to dissociate from the flowcell into a waste reservoir. Then the collected library fragments (comprising library fragments prepared from non-rRNA) would be reintroduced to the flowcell from the temporary storage chamber for binding to the single-stranded immobilized oligonucleotides comprising P5 and/or P7. Once bound, bridge amplification chemistry can amplify the library fragments. After bridge amplification has generated enough library fragments, a cleavage step can be done as in current sequencing chemistry to release both the forward and reverse strands for subsequent collection, quantification, and quality control prior to sequencing.

### Example 3. Enrichment of Desired cDNA Library Fragments

A solid support, such as a flowcell, can be prepared for enrichment. A user could prepare oligonucleotides corresponding to desired RNA and immobilize these oligonucleotides to a solid support. For example, a user may want to enrich for RNA sequences associated with cancer markers for evaluating treatment response, tumor progression, or other means of evaluation (i.e., desired RNA), and the user can immobilize oligonucleotides comprising sequences from such RNA to a solid support. A flowcell with such immobilized oligonucleotides may be termed an enrichment flowcell.

The user can then prepare a cDNA library as described above in Example 1 from a patient sample comprising RNA. Library fragments can then be added to the enrichment flowcell. Library fragments prepared from desired RNA would bind to the enrichment flowcell, and the user can siphon fluid that does not bind to the enrichment flowcell (comprising library fragments not prepared from desired RNA) to a waste container. The user can then denature the bound library fragments, collect them, and sequence them (with optional amplification before sequencing). In this way, the library that is sequenced will be enriched for library fragments prepared from desired RNA.

### Example 4. Preparation of Depletion Probes for Human Microbiome Samples

To improve enzymatic depletion using the Ribo-Zero Plus kit, an iterative design process was used to develop an additional probe set specifically targeting human gut microbiome samples. A goal was to develop probes for enzymatic rRNA depletion of human-associated microbiomes to enable metatranscriptomic analysis.

Some human-associated microbiome samples may have significant amounts of host (human) RNA in addition to bacterial RNA (such as rRNA). For example, skin, oral, and vaginal sample are expected to have a lot of human cells included, so probes against human sequences and bacterial sequences unwanted sequences together may provide the best results for depleting unwanted sequences from human microbiome samples.

Using sequencing data from stool samples depleted with Ribo-Zero Plus, the most abundant rRNA sequences that were not effectively depleted across 9 adult healthy stool RNA samples were identified. For these experiments, total RNA from gut microbiome samples of 9 donors (Petersen et al. Microbiome 5(1):98 (2017)) was processed in triplicate with the Ribo-Zero Plus rRNA Depletion Kit, converted into RNAseq libraries using the TruSeq Stranded Total RNAseq kit and sequenced on a NextSeq (PE 76), producing between 11 to 36 million reads per sample. The FASTQ files (as described in Cock et al. Nucleic Acids Res. 38(6):1767-71 (2010)) from each donor were then aligned to the SILVA (v119, see Quast et al. Nucleic Acids Res 41:D590-6 (2013)) using SortMeRNA (see Kopylova et al. Bioinformatics 28:3211-3217 (2012)) to identify the sequences of rRNA to target for depletion. Any sequence regions that align in close proximity (1-3 nucleotides) were merged and sorted by coverage depth and then filtered to remove any with less than 500× coverage. The top 50 most abundant regions were collected from each sample (donor) and combined to create a list of abundant regions. Any regions that overlapped were then merged and the list converted into a FASTA file. To identify and remove redundancies, a pairwise alignment of each region was performed and any regions that demonstrate equal to or greater than 80% identity were flagged and only one region was chosen for probe design. The existing RiboZero Plus probes (termed DP1) were then aligned to the selected, non-redundant regions and any regions where the probes were aligned at equal to or greater than 80% identity were eliminated. The remaining regions were collected, probe locations were determined, and antisense probe sequences were created for the HMv1 probe set. In addition, the HMv1 probe set also includes probes that were designed directly against the rRNA sequences from all 38 species present in the ATCC mock community samples (MSA-2002, -2005 & -2006) as well as E. coli and B. subtilis.

### Example 5. Preparation of Additional Probes to Improve rRNA Depletion of Infant Stool Microbiome Samples

Human gut microbiome profiles are known to change rapidly during the first few years of life (see, for example, Stewart et al. Nature 562:583-588 (2018)). In young infants, the gut microbiota is significantly different from adult samples and tends to be dominated by different taxa such as Bifidobacteria (see Turroni et al. PLoS One 7(5):e36957 (2012)). Experiments with the Ribo-Zero Plus HMv1 probe set showed that it can efficiently remove rRNA in most infant stool samples with <26% of reads mapping to bacterial rRNA reads on average (data not shown). Interestingly, rRNA depletion was less efficient for a subset of donors in the 9- to 15-months old group. Taxonomic analysis revealed that these samples had high levels of Bifidobacterium bifidum. Lack of depletion suggests that the HMv1 probe set relatively poorly targets rRNA from this particular species.

Additional probes targeting Bifidobacterium bifidum were designed using the present iterative process and added to the HMv1 probe pool to create a second human microbiome pool (HMv2). Further experiments were performed with the HM probes set comprising both HMv1 probes and HMv2 probes.

### Example 6. Evaluation of Depletion Probes for Human Microbiome Samples

A set of human microbiome samples were analyzed using either the standard RiboZero Plus probes (termed DP1), human microbiome probes (HM, comprising HMv1+HMv2 probes), or a combination of HM probes and DP1 probes (HM+DP1). Experiments were performed following standard RiboZero protocols. Results are shown in FIG. 3, with the HM probes alone or in combination with the DP1 probes showing much greater reduction in the percentage of reads that were rRNA as compared to the DP1 probes. Thus, use of the HM probes can significantly reduce the amount of sequencing of unwanted rRNA.

Experiments with wastewater also showed that a RiboZero protocol using the HM probes significantly reduced the amount of sequenced rRNA, in comparison to “Mock” samples that were not subjected to a RiboZero protocol (FIG. 4). While more than 90% of the sample comprised rRNA in Mock samples, this was reduced to less than 15% in the samples subjected to a RiboZero protocol with HM probes.

Experiments were also performed to evaluate rRNA depletion for an ATCC mock community sample of skin microbiome (skin microbiome whole cell mix, ATCC MSA-2005™). The experiment compared results with the RiboZero RNase protocol (either with standard DP1 probes or with human microbiome HM probes) to those with the RiboZero-Bact kit that uses a probe-based hybridization approach to capture and deplete bacterial rRNAs from E. coli and B. subtilis. The RiboZero-Bact probes are contained in the commercial Ribo-Zero Plus rRNA Depletion Kit (Illumina).

As shown in FIG. 5, more than 90% of reads from the skin microbiome sample represented rRNA without depletion. The RiboZero-Bact kit reduced levels of rRNA, but there was substantial variation between samples. The RiboZero standard (with the DP1 probes) only reduced rRNA reads by about 50%. In contrast, the RiboZero human microbiome (HM) treatment reduced rRNA reads to less than 10% of total reads. These results indicate that the RiboZero RNase method with the HM probes improves depletion of rRNA from human microbiome samples as compared to the RiboZero standard method (with DP1 probes) or the RiboZero-Bact kit using probe-based hybridization and probes designed for depleting rRNA from E. coli and B. subtilis. Thus, the HM probes are well-suited for depleting rRNA from human microbiome samples.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method of selecting cDNA library fragments from a library of cDNA fragments prepared from RNA, comprising:
a. preparing a solid support comprising a pool of immobilized oligonucleotides, wherein each immobilized oligonucleotide in the pool comprises a nucleic acid sequence corresponding to an RNA sequence or its complement,
b. adding the library of fragments to the solid support and hybridizing the library fragments to at least one immobilized oligonucleotide to allow binding of library fragments to at least one immobilized oligonucleotide, and
c. collecting library fragments either bound or not bound to at least one immobilized oligonucleotide.

2. The method of claim 1, wherein:
a. the selecting is depleting unwanted cDNA library fragments, wherein the RNA sequence comprises an unwanted RNA sequence, the unwanted library fragments comprise those prepared from unwanted RNA sequences, and the collecting comprises collecting library fragment not bound to at least one immobilized oligonucleotide; or
b. the selecting is enriching desired cDNA library fragments, wherein the RNA sequence comprises a desired RNA sequence, the desired library fragments comprise those prepared from desired RNA sequences, and the collecting comprises collecting library fragment bound to at least one immobilized oligonucleotide.

3. The method of claim 2, wherein the library of fragments is subjected to depleting unwanted cDNA library fragments and the collected library fragments not bound to at least one immobilized oligonucleotides are then subjected to enriching desired cDNA library fragments.

4. A solid support having two pools of immobilized oligonucleotides on its surface, wherein the first pool of oligonucleotides comprises immobilized oligonucleotides each comprising a nucleic acid sequence corresponding to an unwanted RNA sequence or its complement and the second pool of oligonucleotides comprises immobilized oligonucleotides each comprising a solid support adapter sequence that can bind to a library adapter comprised in library fragments.

5. The method of claim 1, wherein at least one unwanted RNA sequence has at least 90%, at least 95%, or at least 99% homology to a high-abundance RNA sequence in a sample used to prepare the library of fragments.

6. The method of claim 5, wherein the high-abundance RNA sequence is a ribosomal RNA (rRNA) sequence.

7. The method of claim 5, wherein the unwanted RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

8. The method of claim 1, wherein each pool of immobilized oligonucleotides comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, or 1100 or more oligonucleotides.

9. The solid support of claim 4, wherein adapter complements that are all or partially complementary to the solid support adapter sequences are bound to the solid support adapter sequences of the second pool and wherein the binding of the adapter complements to the solid support adapter sequences is reversible.

10. A method of amplifying desired cDNA library fragments from a library of cDNA fragments prepared from RNA, comprising:
a. providing the solid support of claim 9;
b. adding the library of fragments to the solid support and hybridizing the library fragments to at least one immobilized oligonucleotide to allow binding of unwanted library fragments to the first pool of oligonucleotides;
c. collecting library fragments not bound to the first pool of oligonucleotides to prepare collected library fragments;
d. denaturing and removing library fragments bound to the first pool of oligonucleotides and adapter complements bound to the adapter sequences of the second pool of oligonucleotides;
e. adding the collected library fragments to the solid support and hybridizing the library fragments to at least one immobilized oligonucleotide to allow binding of desired library fragments to the second pool of oligonucleotides; and
f. amplifying the bound desired library fragments by bridge amplification on the solid support.

11. A method for depleting unwanted RNA molecules comprised in a patient microbiome sample, wherein the patient microbiome sample comprises at least one target RNA or DNA sequence and at least one unwanted RNA molecule, comprising:
a. sequencing a plurality of probe-development microbiome samples to determine at least one unwanted RNA molecule comprising a bacterial ribosomal RNA (rRNA) sequence from sequencing data;
b. preparing a probe set comprising at least one DNA probe complementary to the at least one unwanted RNA molecule;
c. contacting the patient microbiome sample with the probe set to prepare DNA:RNA hybrids; and
d. contacting the DNA:RNA hybrids with a ribonuclease that degrades the RNA from the DNA:RNA hybrids, thereby degrading the unwanted RNA molecules in the patient microbiome sample to form a degraded mixture.

12. A method for depleting unwanted RNA molecules comprised in a patient microbiome sample, wherein the patient microbiome sample comprises at least one target RNA or DNA sequence and at least one unwanted RNA molecule, comprising:
a. contacting the patient microbiome sample with a probe set comprising at least one sequence comprising at least one of SEQ ID NOs: 1-1131 to prepare DNA:RNA hybrids; and
b. contacting the DNA:RNA hybrids with a ribonuclease that degrades the RNA from the DNA:RNA hybrids, thereby degrading the unwanted RNA molecules in the patient microbiome sample to form a degraded mixture.

13. The method of claim 11, further comprising:
a. degrading any remaining DNA probes by contacting the degraded mixture with a DNA digesting enzyme, optionally wherein the DNA digesting enzyme is DNase I, to form a DNA degraded mixture; and
b. separating the degraded RNA from the degraded mixture or the DNA degraded mixture.

14. A composition comprising a probe set comprising:
a. at least one DNA probe comprising at least one sequence comprising at least one of SEQ ID NOs: 1-1131; and
b. a ribonuclease capable of degrading RNA in an DNA:RNA hybrid.

15. A kit comprising a probe set comprising:
a. at least one DNA probe comprising at least one sequence comprising at least one of SEQ ID NOs: 1-1131; and
b. a ribonuclease capable of degrading RNA in an DNA:RNA hybrid.

16. The kit of claim 15, comprising:
a. a probe set comprising at least one DNA probe comprising at least one of SEQ ID NOs: 1-1131;
b. a ribonuclease;
c. a DNase; and
d. RNA purification beads.

17. The method of claim 1, wherein the pool of oligonucleotides or the probe set comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 1131 sequences selected from SEQ ID NOs: 1-1131.

18. The method of claim 1, wherein the pool of oligonucleotides or the probe set comprises at least one sequence comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

19. The method of claim 18, wherein the pool of oligonucleotides or the probe set comprises 100 or more, 500 or more, or 1000 or more sequences comprising at least one of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

20. The method of claim 19, wherein the pool of oligonucleotides or the probe set comprises a sequence comprising each of SEQ ID NOs: 1-10, 12-18, 21, 22, 24-33, 35, 39-43, 45-48, 50-73, 75, 77, 78, 81-84, 86-103, 105-107, 109-127, 129-134, 136-140, 142, 143, 148, 149, 151-155, 157, 160-165, 168-174, 176-182, 184-187, 189-194, 196-204, 206, 208-215, 217-223, 225, 227-246, 248-265, 269, 270, 272-277, 279, 281, 282, 284-290, 292-301, 303-321, 323-331, 333-336, 338, 340-342, 344, 346-349, 351-355, 357-359, 361-372, 374-380, 383-386, 388, 390, 391, 393, 395-401, 403-406, 408-420, 422-439, 441, 443, 444, 446-460, 462-466, 468, 469, 471, 473-477, 479-502, 504-512, 514, 516, 518, 519, 521-524, 526-529, 531, 532, 535-539, 541-545, 547-552, 555-577, 580-608, 610, 612-616, 618-622, 624-630, 632-636, 638-640, 643, 646-649, 652-659, 663-673, 675, 676, 678, 680-682, 684, 685, 688-692, 694, 696-705, 708-715, 717-731, 733, 735, 736, 739-763, 765, 767-779, 781-796, 798, 801-819, 821-826, 828, 830-832, 834, 836-847, 849, 851, 852, 854-861, 863-865, 867-872, 874-881, 883-892, 894-898, 900-909, 911, 913-921, 923-925, 927-935, 938-940, 942-948, 950-965, 967-969, 971-979, 981-984, 986-994, 997-1010, 1012-1015, 1017, 1019-1021, 1023-1025, 1027-1029, 1031-1044, 1046-1058, 1060-1062, 1064-1067, 1069-1075, 1080-1094, 1096, 1099-1105, 1107-1110, 1112, 1113, 1115, 1116, 1118-1126, 1129, and 1130.

21. The method claim 18, wherein the pool of oligonucleotides or the probe set further comprises at least one sequence comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

22. The method of claim 21, wherein the pool of oligonucleotides or the probe set comprises 10 or more, 20 or more, or 30 or more sequences comprising at least one of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

23. The method of claim 22, wherein the pool of oligonucleotides or the probe set comprises a sequence comprising each of SEQ ID NOs: 19, 74, 76, 85, 104, 108, 158, 175, 226, 278, 322, 339, 389, 513, 517, 520, 546, 553, 609, 611, 650, 662, 677, 683, 686, 706, 780, 827, 835, 882, 1022, 1059, 1077, 1078, 1098, 1106, 1111, 1114, 1128, and 1131.

24. The method of claim 1, wherein pool of oligonucleotides or the probe set comprises at least one sequence comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

25. The method of claim 24, wherein the pool of oligonucleotides or the probe set comprises 100 or more, 500 or more, or 1000 or more sequences comprising at least one of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

26. The method of claim 25, wherein the pool of oligonucleotides or the probe set comprises a sequence comprising each of SEQ ID NOs: 1-10, 12-19, 21, 22, 24-33, 35, 39-43, 45-48, 50-78, 81-127, 129-134, 136-140, 142, 143, 148, 149, 151-155, 157, 158, 160-165, 168-182, 184-187, 189-194, 196-204, 206, 208-215, 217-223, 225-246, 248-265, 269, 270, 272-279, 281, 282, 284-290, 292-301, 303-331, 333-336, 338-342, 344, 346-349, 351-355, 357-359, 361-372, 374-380, 383-386, 388-391, 393, 395-401, 403-406, 408-420, 422-439, 441, 443, 444, 446-460, 462-466, 468, 469, 471, 473-477, 479-502, 504-514, 516-524, 526-529, 531, 532, 535-539, 541-553, 555-577, 580-616, 618-622, 624-630, 632-636, 638-640, 643, 646-650, 652-659, 662-673, 675-678, 680-686, 688-692, 694, 696-706, 708-715, 717-731, 733, 735, 736, 739-763, 765, 767-796, 798, 801-819, 821-828, 830-832, 834-847, 849, 851, 852, 854-861, 863-865, 867-872, 874-892, 894-898, 900-909, 911, 913-921, 923-925, 927-935, 938-940, 942-948, 950-965, 967-969, 971-979, 981-984, 986-994, 997-1010, 1012-1015, 1017, 1019-1025, 1027-1029, 1031-1044, 1046-1062, 1064-1067, 1069-1075, 1077, 1078, 1080-1094, 1096, 1098-1116, 1118-1126, and 1128-1131.

27. The method of claim 24, wherein the pool of oligonucleotides or the probe set further comprises at least one sequence comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

28. The method of claim 27, wherein the pool of oligonucleotides or the probe set comprises 10 or more, 20 or more, or 30 or more sequences comprising at least one of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.

29. The method of claim 28, wherein the pool of oligonucleotides or the probe set comprises a sequence comprising each of SEQ ID NOs: 11, 20, 23, 34, 36-38, 44, 49, 79, 80, 128, 135, 141, 144-147, 150, 156, 159, 166, 167, 183, 188, 195, 205, 207, 216, 224, 247, 266-268, 271, 280, 283, 291, 302, 332, 337, 343, 345, 350, 356, 360, 373, 381, 382, 387, 392, 394, 402, 407, 421, 440, 442, 445, 461, 467, 470, 472, 478, 503, 515, 525, 530, 533, 534, 540, 554, 578, 579, 617, 623, 631, 637, 641, 642, 644, 645, 651, 660, 661, 674, 679, 687, 693, 695, 707, 716, 732, 734, 737, 738, 764, 766, 797, 799, 800, 820, 829, 833, 848, 850, 853, 862, 866, 873, 893, 899, 910, 912, 922, 926, 936, 937, 941, 949, 966, 970, 980, 985, 995, 996, 1011, 1016, 1018, 1026, 1030, 1045, 1063, 1068, 1076, 1079, 1095, 1097, 1117, and 1127.


SEQUENCE LISTING (1135 sequences)

SEQ ID NO: 1 - DNA, 50 bp, synthetic construct
  misc_feature 1..50: note = Synthetic
  source 1..50: mol_type = other DNA
ctcatccccacccttttcaacggatgtgggttcggtcctccactgcctct

SEQ ID NO: 2 - DNA, 50 bp, synthetic construct
  misc_feature 1..50: note = Synthetic
  source 1..50: mol_type = other DNA
agccggggcttcttagtcaggtaccgtcattttttcttccctgctgatag

SEQ ID NO: 3 - DNA, 50 bp, synthetic construct
  misc_feature 1..50: note = Synthetic
  source 1..50: mol_type = other DNA
tagatgatcaacctaccgggttagagtagccatcacacaagggtagtatc

SEQ ID NO: 4 - DNA, 50 bp, synthetic construct
  misc_feature 1..50: note = Synthetic
  source 1..50: mol_type = other DNA
cagatggcggcattgtcactgctccgtctccacgtcactcctgaaggtag

SEQ ID NO: 5 - DNA, 50 bp, synthetic construct
  misc_feature 1..50: note = Synthetic
  source 1..50: mol_type = other DNA
gggaagcagggtggaccaccacccaaggctaaatactacctgatgaccga

SEQ ID NO: 6 - DNA, 50 bp, synthetic construct
  misc_feature 1..50: note = Synthetic
  source 1..50: mol_type = other DNA
actaaacttcactccgcatcacgtcttcccattgccgcacggtttttcca

SEQ ID NO: 7 - DNA, 50 bp, synthetic construct
  misc_feature 1..50: note = Synthetic
  source 1..50: mol_type = other DNA
gttcctccgcttgtgcgggcccccgtcaattcctttgagtttcaccgttg

SEQ ID NO: 8 - DNA, 50 bp, synthetic construct
  misc_feature 1..50: note = Synthetic
  source 1..50: mol_type = other DNA
gccccagacaaccatcgctggggttgagctacctcactgcgtccctccgc

SEQ ID NO: 9 - DNA, 50 bp, synthetic construct
  misc_feature 1..50: note = Synthetic
  source 1..50: mol_type = other DNA
ctttcgtgcgggtcggaacttacccgacaaggaatttcgctaccttagga

SEQ ID NO: 10 - DNA, 50 bp, synthetic construct
  misc_feature 1..50: note = Synthetic
  source 1..50: mol_type = other DNA
caggcgtcagctcgtatacgtcatctttcgatttagcacaaacctgtgtt

SEQ ID NO: 11 - DNA, 50 bp, synthetic construct
  misc_feature 1..50: note = Synthetic
  source 1..50: mol_type = other DNA
ggcttcatgcttagatgctttcagcacttatcccgtccgcacatagctac

SEQ ID NO: 12 - DNA, 50 bp, synthetic construct
  misc_feature 1..50: note = Synthetic
  source 1..50: mol_type = other DNA
attaccgcggctgctggcacgtagttagccggggcttcttagtcaggtac

SEQ ID NO: 13 - DNA, 50 bp, synthetic construct
  misc_feature 1..50: note = Synthetic
  source 1..50: mol_type = other DNA
ttcacgcaagatttctcgtgtcccgcgctactcaggataccactacgctt

SEQ ID NO: 14 - DNA, 50 bp, synthetic construct
  misc_feature 1..50: note = Synthetic
  source 1..50: mol_type = other DNA
atctaaagtcttctcgtttaaaatactgggctgttaccatctgtggcgga

SEQ ID NO: 15 - DNA, 50 bp, synthetic construct
  misc_feature 1..50: note = Synthetic
  source 1..50: mol_type = other DNA
gggctctgacttcttgtaggcatacggtttcaggttctctttcactccgc

SEQ ID NO: 16 - DNA, 50 bp, synthetic construct
  misc_feature 1..50: note = Synthetic
  source 1..50: mol_type = other DNA
gctatggatcgtcggtttggtgggccgttaccccgccaactgcctaatcc

SEQ ID NO: 17 - DNA, 50 bp, synthetic construct
  misc_feature 1..50: note = Synthetic
  source 1..50: mol_type = other DNA
atgacttcagcatgggcggtcataacgcggtaccagaatatcaactggtt

SEQ ID NO: 18 - DNA, 50 bp, synthetic construct
  misc_feature 1..50: note = Synthetic
  source 1..50: mol_type = other DNA
tttcagttcaggcggttcccctcatatacctatgtattcagtatatgatg

SEQ ID NO: 19 - DNA, 50 bp, synthetic construct
  misc_feature 1..50: note = Synthetic
  source 1..50: mol_type = other DNA
cgaaaggggagacggcacgggcccggaggttagcgccccaggcctcggtt

SEQ ID NO: 20 - DNA, 50 bp, synthetic construct
  misc_feature 1..50: note = Synthetic
  source 1..50: mol_type = other DNA
tttcgtccctgctcgacttgtaggtctcgcagtcaagctcccttgtgcct

SEQ ID NO: 21 - DNA, 50 bp, synthetic construct
  misc_feature 1..50: note = Synthetic
  source 1..50: mol_type = other DNA
ctcttatcgatgacatctcctcttaaccttccagcaccgggcaggtgtca

SEQ ID NO: 22 - DNA, 50 bp, synthetic construct
  misc_feature 1..50: note = Synthetic
  source 1..50: mol_type = other DNA
tcgtccctgacaacagagctttacgatccgaaaaccttcttcactcacgc

SEQ ID NO: 23 - DNA, 50 bp, synthetic construct
  misc_feature 1..50: note = Synthetic
  source 1..50: mol_type = other DNA
acccaacatctcacgacacgagctgacgacaaccatgcaccacctgtcac

SEQ ID NO: 24 - DNA, 50 bp, synthetic construct
  misc_feature 1..50: note = Synthetic
  source 1..50: mol_type = other DNA
gtcctctcgtactaaggacagagctcctcaaatatcctgcgcccacgaca

SEQ ID NO: 25 - DNA, 50 bp, synthetic construct
  misc_feature 1..50: note = Synthetic
  source 1..50: mol_type = other DNA
ttatagttacggccgccgtttaccggggcttcaattcagagctctcactc

SEQ ID NO: 26 - DNA, 50 bp, synthetic construct
  misc_feature 1..50: note = Synthetic
  source 1..50: mol_type = other DNA
cgtttctacgagttagaactcaaataatcaaagggccgtatttcaacagc

SEQ ID NO: 27 - DNA, 50 bp, synthetic construct
  misc_feature 1..50: note = Synthetic
  source 1..50: mol_type = other DNA
caccagtgtcggtttagggtacgggcggacccgccacctcgctcacgaag

SEQ ID NO: 28 - DNA, 50 bp, synthetic construct
  misc_feature 1..50: note = Synthetic
  source 1..50: mol_type = other DNA
cgtccatcccggtcctctcgtactagggacagctcctctcaaatatcctg

SEQ ID NO: 29 - DNA, 50 bp, synthetic construct
  misc_feature 1..50: note = Synthetic
  source 1..50: mol_type = other DNA
agctgacgctcatgtttccaagtctcccgcctatcctgtacatagatttc

SEQ ID NO: 30 - DNA, 50 bp, synthetic construct
  misc_feature 1..50: note = Synthetic
  source 1..50: mol_type = other DNA
ctcttttaatgagtggctgcttctaagccaacatcctggttgtctaagca

SEQ ID NO: 31 - DNA, 50 bp, synthetic construct
  misc_feature 1..50: note = Synthetic
  source 1..50: mol_type = other DNA
acagcttttctcgccatcttccatcccagacttcggtactaacttccctc

SEQ ID NO: 32 - DNA, 50 bp, synthetic construct
  misc_feature 1..50: note = Synthetic
  source 1..50: mol_type = other DNA
catagacctgtgtttttgctaaacagttgcttgagcctattctctgcggc

SEQ ID NO: 33 - DNA, 50 bp, synthetic construct
  misc_feature 1..50: note = Synthetic
  source 1..50: mol_type = other DNA
tcacggtactggttcactatcgctcactcgtttatatttagccttggcgg

SEQ ID NO: 34 - DNA, 50 bp, synthetic construct
  misc_feature 1..50: note = Synthetic
  source 1..50: mol_type = other DNA
actcaccctgccccgattaacgttggacaggaacccttggtcttccggcg

SEQ ID NO: 35 - DNA, 50 bp, synthetic construct
  misc_feature 1..50: note = Synthetic
  source 1..50: mol_type = other DNA
ggctacagtaaagctccatggggtctttccgtcttgtcgcgggtaaccgg

SEQ ID NO: 36 - DNA, 50 bp, synthetic construct
  misc_feature 1..50: note = Synthetic
  source 1..50: mol_type = other DNA
gtacgatttgatgttacctgatgcttagaggcttttcctggaagcagggc

SEQ ID NO: 37 - DNA, 50 bp, synthetic construct
  misc_feature 1..50: note = Synthetic
  source 1..50: mol_type = other DNA
aagtcattggcattcggagtttgactgaattcggtaacccggtaggggcc

SEQ ID NO: 38 - DNA, 50 bp, synthetic construct
  misc_feature 1..50: note = Synthetic
  source 1..50: mol_type = other DNA
ggttaccttgttacgacttcaccccagtcatgaatcacaaagtggtaagt

SEQ ID NO: 39 - DNA, 50 bp, synthetic construct
  misc_feature 1..50: note = Synthetic
  source 1..50: mol_type = other DNA
cccttctcccgttggccttagaatcttcttcctacctacctgtgtcggtt

SEQ ID NO: 40 - DNA, 50 bp, synthetic construct
  misc_feature 1..50: note = Synthetic
  source 1..50: mol_type = other DNA
taccttcactaaggttctttccgacgctagccctaaagctatttcgggga

SEQ ID NO: 41 - DNA, 50 bp, synthetic construct
  misc_feature 1..50: note = Synthetic
  source 1..50: mol_type = other DNA
cccccctgcttcccacagggtttcacgtgtcccgtggtactctggatcac

SEQ ID NO: 42 - DNA, 50 bp, synthetic construct
  misc_feature 1..50: note = Synthetic
  source 1..50: mol_type = other DNA
gaccggccttcccatgccgttcggttaacagattaagtcttaaaagcagt

SEQ ID NO: 43 - DNA, 50 bp, synthetic construct
  misc_feature 1..50: note = Synthetic
  source 1..50: mol_type = other DNA
ttcctttgaccccccccccccccctccctatccccccccgccccccccca

SEQ ID NO: 44 - DNA, 50 bp, synthetic construct
  misc_feature 1..50: note = Synthetic
  source 1..50: mol_type = other DNA
ccccctcagttctccagcgcccacggcagatagggaccgaactgtctcac

SEQ ID NO: 45 - DNA, 50 bp, synthetic construct
  misc_feature 1..50: note = Synthetic
  source 1..50: mol_type = other DNA
ctttgggaggcaaccgccccagttaaactacccgccaggcactctccccg

SEQ ID NO: 46 - DNA, 50 bp, synthetic construct
  misc_feature 1..50: note = Synthetic
  source 1..50: mol_type = other DNA
acatgatcggttcacacactcaccaccacacaagacctcaaagagacccc

SEQ ID NO: 47 - DNA, 50 bp, synthetic construct
  misc_feature 1..50: note = Synthetic
  source 1..50: mol_type = other DNA
ccagcaccgggcaggtgtcaccccctatacttcgtcttgcgacttcgcag

SEQ ID NO: 48 - DNA, 50 bp, synthetic construct
  misc_feature 1..50: note = Synthetic
  source 1..50: mol_type = other DNA
gtaccgctttaatgggcgaacagcccaacccttgggactgactacagccc

SEQ ID NO: 49 - DNA, 50 bp, synthetic construct
  misc_feature 1..50: note = Synthetic
  source 1..50: mol_type = other DNA
ccattgcggaagattccctactgctgcctcccgtaggagtctgggccgtg

SEQ ID NO: 50 - DNA, 50 bp, synthetic construct
  misc_feature 1..50: note = Synthetic
  source 1..50: mol_type = other DNA
ttctctgcggctcatgtttccatgagcaccccttatccctaagttacggg

SEQ ID NO: 51 - DNA, 50 bp, synthetic construct
  misc_feature 1..50: note = Synthetic
  source 1..50: mol_type = other DNA
tttgactcatatcacacctcactgcttagacgtgcacttccaatcgcacg

SEQ ID NO: 52 - DNA, 50 bp, synthetic construct
  misc_feature 1..50: note = Synthetic
  source 1..50: mol_type = other DNA
ccggtttgccctcttccgcgttcgctcgccactacttacggaatctcgtt

SEQ ID NO: 53 - DNA, 50 bp, synthetic construct
  misc_feature 1..50: note = Synthetic
  source 1..50: mol_type = other DNA
tacctgatcgacttgtcagtctcccagtcaagcgcccttatgccattaca

SEQ ID NO: 54 - DNA, 50 bp, synthetic construct
  misc_feature 1..50: note = Synthetic
  source 1..50: mol_type = other DNA
tcccaagcttcggtgtatgatttagccccgttaaattttcggcgcagggt

SEQ ID NO: 55 - DNA, 50 bp, synthetic construct
  misc_feature 1..50: note = Synthetic
  source 1..50: mol_type = other DNA
cctagtcttttcagtgctctacaagccgtggtcatggttcgaggctgtac

SEQ ID NO: 56 - DNA, 50 bp, synthetic construct
  misc_feature 1..50: note = Synthetic
  source 1..50: mol_type = other DNA
tcggggtgcttttcacctttccttcacagtactcgtacgctatcggtctc

SEQ ID NO: 57 - DNA, 50 bp, synthetic construct
  misc_feature 1..50: note = Synthetic
  source 1..50: mol_type = other DNA
ggtctgggctctttccctttcgactgcccaacttatctcgtgcagtctga

SEQ ID NO: 58 - DNA, 50 bp, synthetic construct
  misc_feature 1..50: note = Synthetic
  source 1..50: mol_type = other DNA
gcactccacagctccttccggtactgcttcttcgcgttaagaatgctcct

SEQ ID NO: 59 - DNA, 50 bp, synthetic construct
  misc_feature 1..50: note = Synthetic
  source 1..50: mol_type = other DNA
gactgcgaaccgtgagcattcggagttcgtcaggactcgataggcggtga

SEQ ID NO: 60 - DNA, 50 bp, synthetic construct
  misc_feature 1..50: note = Synthetic
  source 1..50: mol_type = other DNA
gtaaacagtcgcttgggtctattctctgcggcccattcctgggcactcct

SEQ ID NO: 61 - DNA, 50 bp, synthetic construct
  misc_feature 1..50: note = Synthetic
  source 1..50: mol_type = other DNA
cccactttcgtgcctgctcgacgtgtctgtctcgcagtcaagccaccttg

SEQ ID NO: 62 - DNA, 50 bp, synthetic construct
  misc_feature 1..50: note = Synthetic
  source 1..50: mol_type = other DNA
tttccctgcggctccgggactttatcccttaaccttgccagtatgcacaa

SEQ ID NO: 63 - DNA, 50 bp, synthetic construct
  misc_feature 1..50: note = Synthetic
  source 1..50: mol_type = other DNA
gggcgccttcgcttcgtagcagcttttctcgccagcgtgaattcagcagc

SEQ ID NO: 64 - DNA, 50 bp, synthetic construct
  misc_feature 1..50: note = Synthetic
  source 1..50: mol_type = other DNA
ttccgcctgaccttagctcccgactaaccctgagcggacgaaccttcctc

SEQ ID NO: 65 - DNA, 50 bp, synthetic construct
  misc_feature 1..50: note = Synthetic
  source 1..50: mol_type = other DNA
ctctcaggtcggctactgatcgtcggcttggtaggccgttaccccaccaa

SEQ ID NO: 66 - DNA, 50 bp, synthetic construct
  misc_feature 1..50: note = Synthetic
  source 1..50: mol_type = other DNA
cttcctccggctacttagatgtttcagttcaccgggttcccctccatacg

SEQ ID NO: 67 - DNA, 50 bp, synthetic construct
  misc_feature 1..50: note = Synthetic
  source 1..50: mol_type = other DNA
tacctgatcgacttgttagtctcccagtcaagcgcccttatgccattaca

SEQ ID NO: 68 - DNA, 50 bp, synthetic construct
  misc_feature 1..50: note = Synthetic
  source 1..50: mol_type = other DNA
gcaaccgccccagttaaactacccgccaggcactgtccctgaacaggatg

SEQ ID NO: 69 - DNA, 50 bp, synthetic construct
  misc_feature 1..50: note = Synthetic
  source 1..50: mol_type = other DNA
ttcctcgtgtctcgccgtactcaggatcccattaggcttcgatcggattt

SEQ ID NO: 70 - DNA, 50 bp, synthetic construct
  misc_feature 1..50: note = Synthetic
  source 1..50: mol_type = other DNA
acggatcgtcgccttggtaggcctttaccccaccaactagctaatgcacc

SEQ ID NO: 71 - DNA, 50 bp, synthetic construct
  misc_feature 1..50: note = Synthetic
  source 1..50: mol_type = other DNA
tgtcggtttggggtacgggcggcaacgcgcctgacgccggggcttttctc

SEQ ID NO: 72 - DNA, 50 bp, synthetic construct
  misc_feature 1..50: note = Synthetic
  source 1..50: mol_type = other DNA
cggtttccgttcgcgctgagggaacctttgggcgcctccgttacattttg

SEQ ID NO: 73 - DNA, 50 bp, synthetic construct
  misc_feature 1..50: note = Synthetic
  source 1..50: mol_type = other DNA
ttatagttacggccgccgtttactggggcttcaattcaatgctctcacat

SEQ ID NO: 74 - DNA, 50 bp, synthetic construct
  misc_feature 1..50: note = Synthetic
  source 1..50: mol_type = other DNA
tgtagcatgcgtgaagccctggacgtaaggggcatgatgatctgacgtca

SEQ ID NO: 75 - DNA, 50 bp, synthetic construct
  misc_feature 1..50: note = Synthetic
  source 1..50: mol_type = other DNA
agcaccgggcaggtgtcagcacctatacgtcagctctcgctttcgcagat

SEQ ID NO: 76 - DNA, 50 bp, synthetic construct
  misc_feature 1..50: note = Synthetic
  source 1..50: mol_type = other DNA
gctgataggacgcgaccccatcccacgccgatagaatctttcccacaatc

SEQ ID NO: 77 - DNA, 50 bp, synthetic construct
  misc_feature 1..50: note = Synthetic
  source 1..50: mol_type = other DNA
gtttcaggttctatttcactcccctcccggggtgcttttcacctttccct

SEQ ID NO: 78 - DNA, 50 bp, synthetic construct
  misc_feature 1..50: note = Synthetic
  source 1..50: mol_type = other DNA
cggctcccattccgtgtcacccctgcgctcacctaccacggctacgctcc

SEQ ID NO: 79 - DNA, 50 bp, synthetic construct
  misc_feature 1..50: note = Synthetic
  source 1..50: mol_type = other DNA
tagaggcttttcttggcagtgtggaatcaggaacttcgctactatatttc

SEQ ID NO: 80 - DNA, 50 bp, synthetic construct
  misc_feature 1..50: note = Synthetic
  source 1..50: mol_type = other DNA
ggggaatctcggttgatttcttttcctcggggtacttagatgtttcagtt

SEQ ID NO: 81 - DNA, 50 bp, synthetic construct
  misc_feature 1..50: note = Synthetic
  source 1..50: mol_type = other DNA
cataccagaggttcgtccacccaggtcctctcgtactatgggcaggcctc

SEQ ID NO: 82 - DNA, 50 bp, synthetic construct
  misc_feature 1..50: note = Synthetic
  source 1..50: mol_type = other DNA
cgcgggtccatcttataccaccggagtttttcacactgagccatgcagct

SEQ ID NO: 83 - DNA, 50 bp, synthetic construct
  misc_feature 1..50: note = Synthetic
  source 1..50: mol_type = other DNA
ctcccgcaaccccggccacgcaacccccgacgggtatcgcgcgcggccgg

SEQ ID NO: 84 - DNA, 50 bp, synthetic construct
  misc_feature 1..50: note = Synthetic
  source 1..50: mol_type = other DNA
ttctctgcggctccatctctggagcaccccttctcccgaagttacggggt

SEQ ID NO: 85 - DNA, 50 bp, synthetic construct
  misc_feature 1..50: note = Synthetic
  source 1..50: mol_type = other DNA
gaacatccggcattaccacccgtttccaggagctattccggagcatgggg

SEQ ID NO: 86 - DNA, 50 bp, synthetic construct
  misc_feature 1..50: note = Synthetic
  source 1..50: mol_type = other DNA
aggtcccggggtcttttcgtccttctgcgcttaacgagcatctttactcg

SEQ ID NO: 87 - DNA, 50 bp, synthetic construct
  misc_feature 1..50: note = Synthetic
  source 1..50: mol_type = other DNA
gcttcggtggcatgttttagccccggacattttcggcgcaggacctctcg

SEQ ID NO: 88 - DNA, 50 bp, synthetic construct
  misc_feature 1..50: note = Synthetic
  source 1..50: mol_type = other DNA
gcttcaaagcctccgacctatcctacacatcacgtgcccagattcaatga

SEQ ID NO: 89 - DNA, 50 bp, synthetic construct
  misc_feature 1..50: note = Synthetic
  source 1..50: mol_type = other DNA
tactttatttcgctccacatcacggcttcgtctcatgcacagcggatttg

SEQ ID NO: 90 - DNA, 50 bp, synthetic construct
  misc_feature 1..50: note = Synthetic
  source 1..50: mol_type = other DNA
catggggtctttccgtcctgtcgcgggtaacctgcatcttcacaggtact

SEQ ID NO: 91 - DNA, 50 bp, synthetic construct
  misc_feature 1..50: note = Synthetic
  source 1..50: mol_type = other DNA
gaccttcctctcagaacccctactgatcgttgccttggtgggccgttacc

SEQ ID NO: 92 - DNA, 50 bp, synthetic construct
  misc_feature 1..50: note = Synthetic
  source 1..50: mol_type = other DNA
atgtttcagttccccgggttcccctccatacgttatggattggcgtatgg

SEQ ID NO: 93 - DNA, 50 bp, synthetic construct
  misc_feature 1..50: note = Synthetic
  source 1..50: mol_type = other DNA
ttaacgctttcgcttggccgcttactgtatatcgcaaacagcgagtattc

SEQ ID NO: 94 - DNA, 50 bp, synthetic construct
  misc_feature 1..50: note = Synthetic
  source 1..50: mol_type = other DNA
ccacggaaaaccacctccgcggccggctcccattccgtgtcacccctgcg

SEQ ID NO: 95 - DNA, 50 bp, synthetic construct
  misc_feature 1..50: note = Synthetic
  source 1..50: mol_type = other DNA
tcgtaactcgccggttcattctacaaaaggcacgctctcacccattaacg

SEQ ID NO: 96 - DNA, 50 bp, synthetic construct
  misc_feature 1..50: note = Synthetic
  source 1..50: mol_type = other DNA
aggatgcgacgagccgacatcgaggtgccaaacctccccgccgatatgga

SEQ ID NO: 97 - DNA, 50 bp, synthetic construct
  misc_feature 1..50: note = Synthetic
  source 1..50: mol_type = other DNA
tccccggagtaccttttatcctttgagcgatgtcccttccatacggaaac

SEQ ID NO: 98 - DNA, 50 bp, synthetic construct
  misc_feature 1..50: note = Synthetic
  source 1..50: mol_type = other DNA
cggcttccctactttaatttcggtcccttacgcccgggtcaaccaacgcc

SEQ ID NO: 99 - DNA, 50 bp, synthetic construct
  misc_feature 1..50: note = Synthetic
  source 1..50: mol_type = other DNA
ctgcttccaagccaacatcctagctgtcttagcagtcagacttcgttagt

SEQ ID NO: 100 - DNA, 50 bp, synthetic construct
  misc_feature 1..50: note = Synthetic
  source 1..50: mol_type = other DNA
gctactcataccggcattctcacttctatgcgttccagcgctcctcacgg

SEQ ID NO: 101 - DNA, 50 bp, synthetic construct
  misc_feature 1..50: note = Synthetic
  source 1..50: mol_type = other DNA
gccttcggtgtctgccttatacccgattattatccatgcccggaccctcg

SEQ ID NO: 102 - DNA, 50 bp, synthetic construct
  misc_feature 1..50: note = Synthetic
  source 1..50: mol_type = other DNA
ccggctttcccaaaaccgttccactaacattgcagaatcttaaatgcagt

SEQ ID NO: 103 - DNA, 50 bp, synthetic construct
  misc_feature 1..50: note = Synthetic
  source 1..50: mol_type = other DNA
tacctgtgtcggtttgcggtacgggcaccttagtatacacataagctttt

SEQ ID NO: 104 - DNA, 50 bp, synthetic construct
  misc_feature 1..50: note = Synthetic
  source 1..50: mol_type = other DNA
tgttacgcactctttcaagggtggctgcttctgagccaacctcctggctg

SEQ ID NO: 105 - DNA, 50 bp, synthetic construct
  misc_feature 1..50: note = Synthetic
  source 1..50: mol_type = other DNA
ctggagaccttggatattcggccacaaggattctcaccttgttctcgcta

SEQ ID NO: 106 - DNA, 50 bp, synthetic construct
  misc_feature 1..50: note = Synthetic
  source 1..50: mol_type = other DNA
cagtaacccgcaaggctgcacctaaatgcatttcggggagtacgagctat

SEQ ID NO: 107 - DNA, 50 bp, synthetic construct
  misc_feature 1..50: note = Synthetic
  source 1..50: mol_type = other DNA
aaaccttggatattcggcctagaggattctcacctctatctcgctactca

SEQ ID NO: 108 - DNA, 50 bp, synthetic construct
  misc_feature 1..50: note = Synthetic
  source 1..50: mol_type = other DNA
cgcttgtgcgggcccccgtcaatttctttgagttttagccttgcgaccgt

SEQ ID NO: 109 - DNA, 50 bp, synthetic construct
  misc_feature 1..50: note = Synthetic
  source 1..50: mol_type = other DNA
accgggacacgtgatcccacaacaccggcaacgcaacccccgacgggtat

SEQ ID NO: 110 - DNA, 50 bp, synthetic construct
  misc_feature 1..50: note = Synthetic
  source 1..50: mol_type = other DNA
gcttttctcgccttcagccaagtgtgcttccctactctaatttcggtccc

SEQ ID NO: 111 - DNA, 50 bp, synthetic construct
  misc_feature 1..50: note = Synthetic
  source 1..50: mol_type = other DNA
cactactcacggagtatcccttcctgcaggtactgagatgtttcacttcc

SEQ ID NO: 112 - DNA, 50 bp, synthetic construct
  misc_feature 1..50: note = Synthetic
  source 1..50: mol_type = other DNA
gattggaatttctccgctacccacagttcatccgctaccatttcaacggg

SEQ ID NO: 113 - DNA, 50 bp, synthetic construct
  misc_feature 1..50: note = Synthetic
  source 1..50: mol_type = other DNA
ttccacgagtcccgcgctactcgggagacaccatccatggtgcacgcgca

SEQ ID NO: 114 - DNA, 50 bp, synthetic construct
  misc_feature 1..50: note = Synthetic
  source 1..50: mol_type = other DNA
gtcttttcgtcccatcgcgggtaatcggcatcttcaccgatactacaatt

SEQ ID NO: 115 - DNA, 50 bp, synthetic construct
  misc_feature 1..50: note = Synthetic
  source 1..50: mol_type = other DNA
ccgtacatcatctcgatggcattcggagtttgatattctttggtaagctt

SEQ ID NO: 116 - DNA, 50 bp, synthetic construct
  misc_feature 1..50: note = Synthetic
  source 1..50: mol_type = other DNA
gggcttggctacccggctatagacttggcagtctaaccggtgcaccagcg

SEQ ID NO: 117 - DNA, 50 bp, synthetic construct
  misc_feature 1..50: note = Synthetic
  source 1..50: mol_type = other DNA
actttcgttactgctcgacccgtcagtctcgcagttaggctcgcttctgc

SEQ ID NO: 118 - DNA, 50 bp, synthetic construct
  misc_feature 1..50: note = Synthetic
  source 1..50: mol_type = other DNA
ctactgtttctccgcgtatacaacgctcccctacccaatccattactgga

SEQ ID NO: 119 - DNA, 50 bp, synthetic construct
  misc_feature 1..50: note = Synthetic
  source 1..50: mol_type = other DNA
acttatagtcagcgccccttctcccgaagttacggggccattttgccgag

SEQ ID NO: 120 - DNA, 50 bp, synthetic construct
  misc_feature 1..50: note = Synthetic
  source 1..50: mol_type = other DNA
cttccaagccaacatcctagctgtcttagcaatctgacttcgttagttca

SEQ ID NO: 121 - DNA, 50 bp, synthetic construct
  misc_feature 1..50: note = Synthetic
  source 1..50: mol_type = other DNA
cctcggcaactggcgttaccgattctcagcctcccacctatcctgtacat

SEQ ID NO: 122 - DNA, 50 bp, synthetic construct
  misc_feature 1..50: note = Synthetic
  source 1..50: mol_type = other DNA
ccataacggctcccatcatcacacctcgccatgcatgccatgcggatttg

SEQ ID NO: 123 - DNA, 50 bp, synthetic construct
  misc_feature 1..50: note = Synthetic
  source 1..50: mol_type = other DNA
cgtgcaggtcggaacttacccgacaaggaatttcgctaccttaggaccgt

SEQ ID NO: 124 - DNA, 50 bp, synthetic construct
  misc_feature 1..50: note = Synthetic
  source 1..50: mol_type = other DNA
catccaaacacttttcaacgtgtcctggttcggtcctccagtgcgtttta

SEQ ID NO: 125 - DNA, 50 bp, synthetic construct
  misc_feature 1..50: note = Synthetic
  source 1..50: mol_type = other DNA
gccctaaagctatttcggggagaaccagctatatccgggttcgattggaa

SEQ ID NO: 126 - DNA, 50 bp, synthetic construct
  misc_feature 1..50: note = Synthetic
  source 1..50: mol_type = other DNA
cagtaaagctctacggggtctctccgtccagtcgcgggtaatgggcatct

SEQ ID NO: 127 - DNA, 50 bp, synthetic construct
  misc_feature 1..50: note = Synthetic
  source 1..50: mol_type = other DNA
ggaacctttgggcgcctccgttacgctttaggaggcgaccgccccagtca

SEQ ID NO: 128 - DNA, 50 bp, synthetic construct
  misc_feature 1..50: note = Synthetic
  source 1..50: mol_type = other DNA
cccgccgtgtgtctcccgtgataacattctccggtattcgcagtttgcat

SEQ ID NO: 129 - DNA, 50 bp, synthetic construct
  misc_feature 1..50: note = Synthetic
  source 1..50: mol_type = other DNA
caggtgtcagcccctatacttcatctttcgatttggcagagacctgtgtt

SEQ ID NO: 130 - DNA, 50 bp, synthetic construct
  misc_feature 1..50: note = Synthetic
  source 1..50: mol_type = other DNA
gactcttcccagagtcttcttctattcccttggctgctttatcgcagtcc

SEQ ID NO: 131 - DNA, 50 bp, synthetic construct
  misc_feature 1..50: note = Synthetic
  source 1..50: mol_type = other DNA
ggcaacccaacaacccacacaccatcatcttcagctacaggactatcacc

SEQ ID NO: 132 - DNA, 50 bp, synthetic construct
  misc_feature 1..50: note = Synthetic
  source 1..50: mol_type = other DNA
agcaccgggcaggtgtcaggctatatacctcatgtttccatttcgcatag

SEQ ID NO: 133 - DNA, 50 bp, synthetic construct
  misc_feature 1..50: note = Synthetic
  source 1..50: mol_type = other DNA
ttgcatactattaagttcagctcggaaggtggatttgcctgccttcctca

SEQ ID NO: 134 - DNA, 50 bp, synthetic construct
  misc_feature 1..50: note = Synthetic
  source 1..50: mol_type = other DNA
ccggcggatttgccaaccggacaccctacacccttggaccaggtcaattc

SEQ ID NO: 135 - DNA, 50 bp, synthetic construct
  misc_feature 1..50: note = Synthetic
  source 1..50: mol_type = other DNA
gccggttataacggttcatatcaccttaccgacgcttatcgcagattagc

SEQ ID NO: 136 - DNA, 50 bp, synthetic construct
  misc_feature 1..50: note = Synthetic
  source 1..50: mol_type = other DNA
ctgatacaaccagtatcgctccgtccatttgcgcagcaccagtaatcatg

SEQ ID NO: 137 - DNA, 50 bp, synthetic construct
  misc_feature 1..50: note = Synthetic
  source 1..50: mol_type = other DNA
tctttgaatgtatggctgcttctgagccaacatcctagttgtcttcgaga

SEQ ID NO: 138 - DNA, 50 bp, synthetic construct
  misc_feature 1..50: note = Synthetic
  source 1..50: mol_type = other DNA
tggattctcgccctcttgtactcatttcgactacgggactgttaccctct

SEQ ID NO: 139 - DNA, 50 bp, synthetic construct
  misc_feature 1..50: note = Synthetic
  source 1..50: mol_type = other DNA
cagtatcaactgcaattttacggttgagccgcaaactttcacaactgact

SEQ ID NO: 140 - DNA, 50 bp, synthetic construct
  misc_feature 1..50: note = Synthetic
  source 1..50: mol_type = other DNA
ttctctgcggcttaccttcgtaagcaccccttctcccgaagttacggggt

SEQ ID NO: 141 - DNA, 50 bp, synthetic construct
  misc_feature 1..50: note = Synthetic
  source 1..50: mol_type = other DNA
attactagcgattccagcttcacgcagtcgagttgcagactgcgatccga

SEQ ID NO: 142 - DNA, 50 bp, synthetic construct; identical to SEQ ID NO: 32
  misc_feature 1..50: note = Synthetic
  source 1..50: mol_type = other DNA

SEQ ID NO: 143 - DNA, 50 bp, synthetic construct
  misc_feature 1..50: note = Synthetic
  source 1..50: mol_type = other DNA
tataagtcgaggctgcacctaaatgcatttcggggagtacgagctatctc

SEQ ID NO: 144 - DNA, 50 bp, synthetic construct
  misc_feature 1..50: note = Synthetic
  source 1..50: mol_type = other DNA
tcaacctgttgtccatcgcctacgcctttcggcctcggcttaggtcccga

SEQ ID NO: 145 - DNA, 50 bp, synthetic construct
  misc_feature 1..50: note = Synthetic
  source 1..50: mol_type = other DNA
ggggtagcttttatccgttgagcgatggcccttccatgcggaaccaccgg

SEQ ID NO: 146 - DNA, 50 bp, synthetic construct
  misc_feature 1..50: note = Synthetic
  source 1..50: mol_type = other DNA
attaacctatggattcagttaatgatagtgtgtcgaaacacactgggttt

SEQ ID NO: 147 - DNA, 50 bp, synthetic construct
  misc_feature 1..50: note = Synthetic
  source 1..50: mol_type = other DNA
cctcttaaccttccagcaccgggcaggcgtcagcccctatacttcgcctt

SEQ ID NO: 148 - DNA, 50 bp, synthetic construct
  misc_feature 1..50: note = Synthetic
  source 1..50: mol_type = other DNA
aaaaagcaagctctctcaagttccgttcgacttgcatgtgttaggcgcgc

SEQ ID NO: 149 - DNA, 50 bp, synthetic construct
  misc_feature 1..50: note = Synthetic
  source 1..50: mol_type = other DNA
gggcccgtgtctcagtgcccatgtgggggaccctcctcaggccggctatc

SEQ ID NO: 150 - DNA, 50 bp, synthetic construct
  misc_feature 1..50: note = Synthetic
  source 1..50: mol_type = other DNA
gacttaacaaaccgcctgcgtgcgctttacgcccagtaattccgattaac

SEQ ID NO: 151 - DNA, 50 bp, synthetic construct
  misc_feature 1..50: note = Synthetic
  source 1..50: mol_type = other DNA
caacctgttgtccatcggctacgcttttcagcctcaccttaggtcccgac

SEQ ID NO: 152 - DNA, 50 bp, synthetic construct
  misc_feature 1..50: note = Synthetic
  source 1..50: mol_type = other DNA
cacacaccaccaccacccgaaagcggaggcggggcgcgggcagattggtt

SEQ ID NO: 153 - DNA, 50 bp, synthetic construct
  misc_feature 1..50: note = Synthetic
  source 1..50: mol_type = other DNA
ccgttcgacttgcatgtgttaagcacgccgccagcgttcatcctgagcca

SEQ ID NO: 154 - DNA, 50 bp, synthetic construct
  misc_feature 1..50: note = Synthetic
  source 1..50: mol_type = other DNA
ggcaccctctacggccaggccttcaagcctgttcccctggcaagccgttt

SEQ ID NO: 155 - DNA, 50 bp, synthetic construct
  misc_feature 1..50: note = Synthetic
  source 1..50: mol_type = other DNA
gcccttcaaaagcgtccctgtgtttaaatcttcggaggttacggaatttc

SEQ ID NO: 156 - DNA, 50 bp, synthetic construct
  misc_feature 1..50: note = Synthetic
  source 1..50: mol_type = other DNA
tcgtggtgtgacgggcggtgtgtacaaggcccgggaacgtattcaccgcg

SEQ ID NO: 157 - DNA, 50 bp, synthetic construct
  misc_feature 1..50: note = Synthetic
  source 1..50: mol_type = other DNA
tcccggggttcttttcaccgttccttcacagtactatgcgctatcggtca

SEQ ID NO: 158 - DNA, 50 bp, synthetic construct
  misc_feature 1..50: note = Synthetic
  source 1..50: mol_type = other DNA
gactgttcgaggttagacatcaaacgagaacagagcggtatttcaccttg

SEQ ID NO: 159 - DNA, 50 bp, synthetic construct
  misc_feature 1..50: note = Synthetic
  source 1..50: mol_type = other DNA
caccttagagtgcccaactgaatgctggcaactaagatcaagggttgcgc

SEQ ID NO: 160 - DNA, 50 bp, synthetic construct
  misc_feature 1..50: note = Synthetic
  source 1..50: mol_type = other DNA
tatggcacttaagccgacacctcacggcacgagctgacgacaaccatgca

SEQ ID NO: 161 - DNA, 50 bp, synthetic construct
  misc_feature 1..50: note = Synthetic
  source 1..50: mol_type = other DNA
tctcgtccattgaccaatattcctcactgctgcctcccgtaggagtttgg

SEQ ID NO: 162 - DNA, 50 bp, synthetic construct
  misc_feature 1..50: note = Synthetic
  source 1..50: mol_type = other DNA
ttttcacctttccctcacggtactggttcgctatcggtctctcgggagta

SEQ ID NO: 163 - DNA, 50 bp, synthetic construct
  misc_feature 1..50: note = Synthetic
  source 1..50: mol_type = other DNA
ttccccattcagagatctccggatcaatggatatttgctcctccccgaag

SEQ ID NO: 164 - DNA, 50 bp, synthetic construct
  misc_feature 1..50: note = Synthetic
  source 1..50: mol_type = other DNA
tgagccaacatcctggttgtctgcgtatcttcacatcgttttccacttaa

SEQ ID NO: 165 - DNA, 50 bp, synthetic construct
  misc_feature 1..50: note = Synthetic
  source 1..50: mol_type = other DNA
tcggagtttgatattcttcggtaggctttgacgccccctaggaaattcag

SEQ ID NO: 166 - DNA, 50 bp, synthetic construct
  misc_feature 1..50: note = Synthetic
  source 1..50: mol_type = other DNA
ccttcggctcccctattcggttaaccttgctacagaatataagtcgctga

SEQ ID NO: 167 - DNA, 50 bp, synthetic construct
  misc_feature 1..50: note = Synthetic
  source 1..50: mol_type = other DNA
gtctggaccgtgtctcagttccagtgtggctggtcatcctctcagaccag

SEQ ID NO: 168 - DNA, 50 bp, synthetic construct
  misc_feature 1..50: note = Synthetic
  source 1..50: mol_type = other DNA
ttatccgttccgtacatagctgcccagccgtgccattggcatgacaactg

SEQ ID NO: 169 - DNA, 50 bp, synthetic construct
  misc_feature 1..50: note = Synthetic
  source 1..50: mol_type = other DNA
ttcacagtactatgcgctatcggtcactaaggagtatttagccttgcggg

SEQ ID NO: 170 - DNA, 50 bp, synthetic construct
  misc_feature 1..50: note = Synthetic
  source 1..50: mol_type = other DNA
gactcacccggggacgacgaacgtggccccggaacccttggtcatccagc

SEQ ID NO: 171 - DNA, 50 bp, synthetic construct
  misc_feature 1..50: note = Synthetic
  source 1..50: mol_type = other DNA
ggcaacttcaacctgcacatggatagatcacccggtttcgggtctacgta

SEQ ID NO: 172 - DNA, 50 bp, synthetic construct
  misc_feature 1..50: note = Synthetic
  source 1..50: mol_type = other DNA
accacgaattccgcctgcctcaactgcactcaagatatccagtatcaact

SEQ ID NO: 173 - DNA, 50 bp, synthetic construct
  misc_feature 1..50: note = Synthetic
  source 1..50: mol_type = other DNA
accacgcattgctgcatcccaagcttcggttacatgcttagccccgttac

SEQ ID NO: 174 - DNA, 50 bp, synthetic construct
  misc_feature 1..50: note = Synthetic
  source 1..50: mol_type = other DNA
ccagagcttttctcgcctccgtccaagcatgcttccctactaaatttcag

SEQ ID NO: 175 - DNA, 50 bp, synthetic construct
  misc_feature 1..50: note = Synthetic
  source 1..50: mol_type = other DNA
gctgcacctaaatgcatttcggagagaaccagctatcacggaatttgatt

SEQ ID NO: 176 - DNA, 50 bp, synthetic construct
  misc_feature 1..50: note = Synthetic
  source 1..50: mol_type = other DNA
cctggttcgggcctccagtgagttttacctcaccttcaccctgctcatgg

SEQ ID NO: 177 - DNA, 50 bp, synthetic construct
  misc_feature 1..50: note = Synthetic
  source 1..50: mol_type = other DNA
actcacccggggacgacgaacgtggccccggaacccttggtcatccagcg

SEQ ID NO: 178 - DNA, 50 bp, synthetic construct
  misc_feature 1..50: note = Synthetic
  source 1..50: mol_type = other DNA
aacatcctggttgtctgtgcaattccacatccttctccacttaacgtgaa

SEQ ID NO: 179 - DNA, 50 bp, synthetic construct
  misc_feature 1..50: note = Synthetic
  source 1..50: mol_type = other DNA
ctacgacttctccccatacagaacgctctcctaccatacattagatgtat

SEQ ID NO: 180 - DNA, 50 bp, synthetic construct
  misc_feature 1..50: note = Synthetic
  source 1..50: mol_type = other DNA
cacacttagccccggacaaccatcaccggggatgagctacctcactgcgt

SEQ ID NO: 181 - DNA, 50 bp, synthetic construct
  misc_feature 1..50: note = Synthetic
  source 1..50: mol_type = other DNA
gggcgaccctccaacagcggcggaacacatttcgactacgggactctcac

SEQ ID NO: 182 - DNA, 50 bp, synthetic construct
  misc_feature 1..50: note = Synthetic
  source 1..50: mol_type = other DNA
ctccggtgcttaaccttgccagtgagcgcaactcgccggaccgttctaca

SEQ ID NO: 183 - DNA, 50 bp, synthetic construct
  misc_feature 1..50: note = Synthetic
  source 1..50: mol_type = other DNA
ttcgcaggcttacagaacgctcccctacccaacaacgcataagcgtcgct

SEQ ID NO: 184 - DNA, 50 bp, synthetic construct
  misc_feature 1..50: note = Synthetic
  source 1..50: mol_type = other DNA
ccgtcaagccatgggagccgggtgtacctaaagtcggtaaccgcaaggag

SEQ ID NO: 185 - DNA, 50 bp, synthetic construct
  misc_feature 1..50: note = Synthetic
  source 1..50: mol_type = other DNA
ttacctacaccatcacctacacgcttacaccaacaatccactaagcggca

SEQ ID NO: 186 - DNA, 50 bp, synthetic construct
  misc_feature 1..50: note = Synthetic
  source 1..50: mol_type = other DNA
gcgtacacctgcagcctatctacctcgtagtcttcaaggggtcttacctg

SEQ ID NO: 187 - DNA, 50 bp, synthetic construct
  misc_feature 1..50: note = Synthetic
  source 1..50: mol_type = other DNA
gccgtcgcccgttagtaccggtcggctccacccctcgcggggcttccacc

SEQ ID NO: 188 - DNA, 50 bp, synthetic construct
  misc_feature 1..50: note = Synthetic
  source 1..50: mol_type = other DNA
cacagtgctgtgtttttaataaacagttgcagccagctggtatcttcgac

SEQ ID NO: 189 - DNA, 50 bp, synthetic construct
  misc_feature 1..50: note = Synthetic
  source 1..50: mol_type = other DNA
ctgttatccccagggtagcttttatccgttgagcgacggcatttccactc

SEQ ID NO: 190 - DNA, 50 bp, synthetic construct
  misc_feature 1..50: note = Synthetic
  source 1..50: mol_type = other DNA
acttagatgctttcagcacttatccaatcccgacttagatacccggcaat

SEQ ID NO: 191 - DNA, 50 bp, synthetic construct
  misc_feature 1..50: note = Synthetic
  source 1..50: mol_type = other DNA
gcttgcgctaacctctcctcttaaccttccagcaccgggcaggcgtcagc

SEQ ID NO: 192 - DNA, 50 bp, synthetic construct
  misc_feature 1..50: note = Synthetic
  source 1..50: mol_type = other DNA
acctatcctgtacatgtggtacagatactcaatatcaaactgcagtaaag

SEQ ID NO: 193 - DNA, 50 bp, synthetic construct
  misc_feature 1..50: note = Synthetic
  source 1..50: mol_type = other DNA
ctccaccagactaaaacgaggctagccctaaagctatttcgaggagaacc

SEQ ID NO: 194 - DNA, 50 bp, synthetic construct
  misc_feature 1..50: note = Synthetic
  source 1..50: mol_type = other DNA
cccggcttaccttgggcggacgaaccttccccaagaaaccttagattttc

SEQ ID NO: 195 - DNA, 50 bp, synthetic construct
  misc_feature 1..50: note = Synthetic
  source 1..50: mol_type = other DNA
gcagaacaactggtacaccagcggtgcgtccatcccggtcctctcgtact

SEQ ID NO: 196 - DNA, 50 bp, synthetic construct
  misc_feature 1..50: note = Synthetic
  source 1..50: mol_type = other DNA
gaccaggtcgattccattgcctggcccggctaccttcctgcgtcacacct

SEQ ID NO: 197 - DNA, 50 bp, synthetic construct
  misc_feature 1..50: note = Synthetic
  source 1..50: mol_type = other DNA
ctctgagacttcaaatgtgtccctgtgcttaactcttttggtggtgacgg

SEQ ID NO: 198 - DNA, 50 bp, synthetic construct
  misc_feature 1..50: note = Synthetic
  source 1..50: mol_type = other DNA
acctcgcggtacgccttcgacgctgactggaatgctcccctaccgatcat

SEQ ID NO: 199 - DNA, 50 bp, synthetic construct
  misc_feature 1..50: note = Synthetic
  source 1..50: mol_type = other DNA
cgtccatcctgagggaacctttgggcgcctccgataccctttcggaggcg

SEQ ID NO: 200 - DNA, 50 bp, synthetic construct
  misc_feature 1..50: note = Synthetic
  source 1..50: mol_type = other DNA
cacctatcggtctctccttaggtcccgactaacccagggcggacgagcct

SEQ ID NO: 201 - DNA, 50 bp, synthetic construct
  misc_feature 1..50: note = Synthetic
  source 1..50: mol_type = other DNA
cgctcgccgctactaaggaaatcgatgtttctttctcttcctccggctac

SEQ ID NO: 202 - DNA, 50 bp, synthetic construct
  misc_feature 1..50: note = Synthetic
  source 1..50: mol_type = other DNA
cgcgagtccatcttcaagcgataaaatctttgatatcaaaaccatgtggt

SEQ ID NO: 203 - DNA, 50 bp, synthetic construct
  misc_feature 1..50: note = Synthetic
  source 1..50: mol_type = other DNA
tgactggagtttgtccagccgggtttccccattcagagatctgcggatca

SEQ ID NO: 204 - DNA, 50 bp, synthetic construct
  misc_feature 1..50: note = Synthetic
  source 1..50: mol_type = other DNA
cctacttagctacccggctatgcccctggcggaacaaccggtgcaccagc

SEQ ID NO: 205 - DNA, 50 bp, synthetic construct
  misc_feature 1..50: note = Synthetic
  source 1..50: mol_type = other DNA
acgcttaaaccgggacaaccgtcgcccggccaacatagccttctccgtcc

SEQ ID NO: 206 - DNA, 50 bp, synthetic construct
  misc_feature 1..50: note = Synthetic
  source 1..50: mol_type = other DNA
gatttgcctgggataatcaacatctacaccctttaacggactattccgtc

SEQ ID NO: 207 - DNA, 50 bp, synthetic construct
  misc_feature 1..50: note = Synthetic
  source 1..50: mol_type = other DNA
ctaatgcgccgcgggtccatctgtaagtggtagccgaagccaccttttat

SEQ ID NO: 208 - DNA, 50 bp, synthetic construct
  misc_feature 1..50: note = Synthetic
  source 1..50: mol_type = other DNA
ggatcttagcactcgcagtctgactgccgaccataaatcaatggcattcg

SEQ ID NO: 209 - DNA, 50 bp, synthetic construct
  misc_feature 1..50: note = Synthetic
  source 1..50: mol_type = other DNA
acctatcctgtacatgtggtacaggtactcaatatcaaactgcagtaaag

SEQ ID NO: 210 - DNA, 50 bp, synthetic construct
  misc_feature 1..50: note = Synthetic
  source 1..50: mol_type = other DNA
tcaccggggatgagctacctcactgcgtccctccgcagcttgcctactac

SEQ ID NO: 211 - DNA, 50 bp, synthetic construct
  misc_feature 1..50: note = Synthetic
  source 1..50: mol_type = other DNA
gccatgcagattctcactgcattcgcgctactcattccggcattctcact

SEQ ID NO: 212 - DNA, 50 bp, synthetic construct
  misc_feature 1..50: note = Synthetic
  source 1..50: mol_type = other DNA
cttcacctcacatacgacgctcccctacccctgacaattacttgtcaagc

SEQ ID NO: 213 - DNA, 50 bp, synthetic construct
  misc_feature 1..50: note = Synthetic
  source 1..50: mol_type = other DNA
ccctactgatcgtcgccttggtgggccgttaccccgccaacaagctaatc

SEQ ID NO: 214 - DNA, 50 bp, synthetic construct
  misc_feature 1..50: note = Synthetic
  source 1..50: mol_type = other DNA
acgcattcggagtttgtcaagacttgataggcggtgaagccctcgcatct

SEQ ID NO: 215 - DNA, 50 bp, synthetic construct
  misc_feature 1..50: note = Synthetic
  source 1..50: mol_type = other DNA
acattttaggaggcgaccgccccagtcaaactgcccgtcagacactgtct

SEQ ID NO: 216 - DNA, 50 bp, synthetic construct
  misc_feature 1..50: note = Synthetic
  source 1..50: mol_type = other DNA
ggtgggtttccccattcggaaatctccggatcaaagcttgcttacagctc

SEQ ID NO: 217 - DNA, 50 bp, synthetic construct
  misc_feature 1..50: note = Synthetic
  source 1..50: mol_type = other DNA
ctcatccccacccttttcaacggatgtgggttcggtcctccattgccttt

SEQ ID NO: 218 - DNA, 50 bp, synthetic construct
  misc_feature 1..50: note = Synthetic
  source 1..50: mol_type = other DNA
aggtcacttggtttcgggtctacatctacgtacttaaccgcccttttcag

SEQ ID NO: 219 - DNA, 50 bp, synthetic construct
  misc_feature 1..50: note = Synthetic
  source 1..50: mol_type = other DNA
acacactcaccacaccaccacaacatcaaagacatcacaatggcaggctc

SEQ ID NO: 220 - DNA, 50 bp, synthetic construct
  misc_feature 1..50: note = Synthetic
  source 1..50: mol_type = other DNA
tgacaactggtgcaccagaggtgcgtccatcccggtcctctcgtactagg

SEQ ID NO: 221 - DNA, 50 bp, synthetic construct
  misc_feature 1..50: note = Synthetic
  source 1..50: mol_type = other DNA
tctgcctctgcacattgctcctctaccgcgcatcttcttcagacgcaccc

SEQ ID NO: 222 - DNA, 50 bp, synthetic construct
  misc_feature 1..50: note = Synthetic
  source 1..50: mol_type = other DNA
cttttctcgacagtacgggatcaccaacttcaccaattaaggctacgcat

SEQ ID NO: 223 - DNA, 50 bp, synthetic construct
  misc_feature 1..50: note = Synthetic
  source 1..50: mol_type = other DNA
ccctcatgtcactatttattcatgacatgatgacacgctgttaacgtgcc

SEQ ID NO: 224 - DNA, 50 bp, synthetic construct
  misc_feature 1..50: note = Synthetic
  source 1..50: mol_type = other DNA
gtacgcagtcacacgcctaagcgtgctcccactgcttgtacgtacacggt

SEQ ID NO: 225 - DNA, 50 bp, synthetic construct
  misc_feature 1..50: note = Synthetic
  source 1..50: mol_type = other DNA
ggcgaccaccccagtcaaactacccaccaagcaatgtccgcgcatagcgc

SEQ ID NO: 226 - DNA, 50 bp, synthetic construct
  misc_feature 1..50: note = Synthetic
  source 1..50: mol_type = other DNA
gacttagtcccaatcacgagcctcaccttagacggctccatcccacaagg

SEQ ID NO: 227 - DNA, 50 bp, synthetic construct
  misc_feature 1..50: note = Synthetic
  source 1..50: mol_type = other DNA
gcgcttatgcggtattagcagtcatttctaactgttatccccctgtataa

SEQ ID NO: 228 - DNA, 50 bp, synthetic construct
  misc_feature 1..50: note = Synthetic
  source 1..50: mol_type = other DNA
cgctttcactgcggctacgtgtctcgtgacactcaacctcgccagtgacg

SEQ ID NO: 229 - DNA, 50 bp, synthetic construct
  misc_feature 1..50: note = Synthetic
  source 1..50: mol_type = other DNA
atgcttttcgcttacaggactataaccttctttggtgtgccttcccatac

SEQ ID NO: 230 - DNA, 50 bp, synthetic construct
  misc_feature 1..50: note = Synthetic
  source 1..50: mol_type = other DNA
cgactaacccagggcggacgagccttcccctggaaaccttagtcttacgg

SEQ ID NO: 231 - DNA, 50 bp, synthetic construct
  misc_feature 1..50: note = Synthetic
  source 1..50: mol_type = other DNA
taggacccgactaaccctgatccgattagcgttgatcaggaaaccttagt

SEQ ID NO: 232 - DNA, 50 bp, synthetic construct
  misc_feature 1..50: note = Synthetic
  source 1..50: mol_type = other DNA
acagcttttctcgtctctttccaaactgacttccgcttacgcgtccctta

SEQ ID NO: 233 - DNA, 50 bp, synthetic construct
  misc_feature 1..50: note = Synthetic
  source 1..50: mol_type = other DNA
taagacttgctctcgctgcggcttcagaccttaagtccttaaccttgcca

SEQ ID NO: 234 - DNA, 50 bp, synthetic construct
  misc_feature 1..50: note = Synthetic
  source 1..50: mol_type = other DNA
ctctcaaaccagctatggatcgtcggcttggtaggccattaccccaccaa

SEQ ID NO: 235 - DNA, 50 bp, synthetic construct
  misc_feature 1..50: note = Synthetic
  source 1..50: mol_type = other DNA
ggaatttctcccctatccacacgtcatctccacccttttcaacggatgtg

SEQ ID NO: 236 - DNA, 50 bp, synthetic construct
  misc_feature 1..50: note = Synthetic
  source 1..50: mol_type = other DNA
ccggtccatggtcggtacgggaatatccacccgttcatccattcgactac

SEQ ID NO: 237 - DNA, 50 bp, synthetic construct
  misc_feature 1..50: note = Synthetic
  source 1..50: mol_type = other DNA
cccccgaccggtttcacggccgcaggttagaattccagaaacctaagggc

SEQ ID NO: 238 - DNA, 50 bp, synthetic construct
  misc_feature 1..50: note = Synthetic
  source 1..50: mol_type = other DNA
aagtttcggtggctacggaatttcaaccgtatgtgcatcgactacgcctc

SEQ ID NO: 239 - DNA, 50 bp, synthetic construct
  misc_feature 1..50: note = Synthetic
  source 1..50: mol_type = other DNA
tgcgctccctttacacccagtaaatccggataacgcttgccccctacgta

SEQ ID NO: 240 - DNA, 50 bp, synthetic construct
  misc_feature 1..50: note = Synthetic
  source 1..50: mol_type = other DNA
atttcgcctacgggactgtcaccctctatggtccacctttccaggtgagt

SEQ ID NO: 241 - DNA, 50 bp, synthetic construct; identical to SEQ ID NO: 87
  misc_feature 1..50: note = Synthetic
  source 1..50: mol_type = other DNA

SEQ ID NO: 242 - DNA, 50 bp, synthetic construct
  misc_feature 1..50: note = Synthetic
  source 1..50: mol_type = other DNA
gacatgtctccacatcattcagttgcaattcaagcccgggtaaggttcct

SEQ ID NO: 243 - DNA, 50 bp, synthetic construct
  misc_feature 1..50: note = Synthetic
  source 1..50: mol_type = other DNA
cgataactggcacaccagaggtgcgtccttcccggtcctctcgtactagg

SEQ ID NO: 244 - DNA, 50 bp, synthetic construct
  misc_feature 1..50: note = Synthetic
  source 1..50: mol_type = other DNA
aacgcttatcggtgcggacctccatcccgtgttaccgggacttcatcctg

SEQ ID NO: 245 - DNA, 50 bp, synthetic construct
  misc_feature 1..50: note = Synthetic
  source 1..50: mol_type = other DNA
ccactccgtcgatgtgaactcttgggagtgataagcctgttatccccagg

SEQ ID NO: 246 - DNA, 50 bp, synthetic construct
  misc_feature 1..50: note = Synthetic
  source 1..50: mol_type = other DNA
gccgccttttcaacggaggtcggttcggccctccatggagttttacctcc

SEQ ID NO: 247 - DNA, 50 bp, synthetic construct
  misc_feature 1..50: note = Synthetic
  source 1..50: mol_type = other DNA
accgttatagttacggccgccgtttactggggcttcaattcgcaccttcg

SEQ ID NO: 248 - DNA, 50 bp, synthetic construct
  misc_feature 1..50: note = Synthetic
  source 1..50: mol_type = other DNA
aggtgttctcatgtgggtttccccattcagagatctgcgggtcaatggat

SEQ ID NO: 249 - DNA, 50 bp, synthetic construct
  misc_feature 1..50: note = Synthetic
  source 1..50: mol_type = other DNA
agcctgttcccctggcaagccgttttatgactcccgcccggtccgtcgga

SEQ ID NO: 250 - DNA, 50 bp, synthetic construct
  misc_feature 1..50: note = Synthetic
  source 1..50: mol_type = other DNA
gctgacctactacgaggggggatcccaacgcgcccgcgccgcgacccccc

SEQ ID NO: 251 - DNA, 50 bp, synthetic construct
  misc_feature 1..50: note = Synthetic
  source 1..50: mol_type = other DNA
gttatccccctgtatgaggcaggttacccacgcgttactcacccgtccgc

SEQ ID NO: 252 - DNA, 50 bp, synthetic construct
  misc_feature 1..50: note = Synthetic
  source 1..50: mol_type = other DNA
cggacatcttcggcgcacaatcactcgaccagtgagctattacgcactct

SEQ ID NO: 253 - DNA, 50 bp, synthetic construct
  misc_feature 1..50: note = Synthetic
  source 1..50: mol_type = other DNA
tgcttgatgcccgattattatccacgccaaactcctcgactagtgagctg

SEQ ID NO: 254 - DNA, 50 bp, synthetic construct
  misc_feature 1..50: note = Synthetic
  source 1..50: mol_type = other DNA
ctccattcggaaatctgcggatcaaagcctacttacggctccccgcagct

SEQ ID NO: 255 - DNA, 50 bp, synthetic construct
  misc_feature 1..50: note = Synthetic
  source 1..50: mol_type = other DNA
gctgttggtccggattgttctcctttaggacatggaccttagcacccatg

SEQ ID NO: 256 - DNA, 50 bp, synthetic construct
  misc_feature 1..50: note = Synthetic
  source 1..50: mol_type = other DNA
tgctggcacggagttagccgtcacttccttgttgagtaccgtcattatct

SEQ ID NO: 257 - DNA, 50 bp, synthetic construct
  misc_feature 1..50: note = Synthetic
  source 1..50: mol_type = other DNA
gctatcggtcagacaggtatgcttagacttacccaacggtctgggctgat

SEQ ID NO: 258 - DNA, 50 bp, synthetic construct
  misc_feature 1..50: note = Synthetic
  source 1..50: mol_type = other DNA
tattcctcactgctgcctcccgtaggagtttggaccgtgtctcagttcca

SEQ ID NO: 259 - DNA, 50 bp, synthetic construct
  misc_feature 1..50: note = Synthetic
  source 1..50: mol_type = other DNA
tcccgctggccttagaattctcttcctgtccacctgtgtcggtttgcggt

SEQ ID NO: 260 - DNA, 50 bp, synthetic construct
  misc_feature 1..50: note = Synthetic
  source 1..50: mol_type = other DNA
cgactattgtcctcggcttaggtcccgacttaccctgagaggacgagcct

SEQ ID NO: 261 - DNA, 50 bp, synthetic construct
  misc_feature 1..50: note = Synthetic
  source 1..50: mol_type = other DNA
ggtccttttcacctttccttcacagtactatgcgctatcggtcactaagt

SEQ ID NO: 262 - DNA, 50 bp, synthetic construct
  misc_feature 1..50: note = Synthetic
  source 1..50: mol_type = other DNA
tcggctactgatcgtcgccttggtaggccgttgccctgccaactagctaa

SEQ ID NO: 263 - DNA, 50 bp, synthetic construct
  misc_feature 1..50: note = Synthetic
  source 1..50: mol_type = other DNA
cttgggagtatgtttacacgcactattaccgttttccgaggaaattggta

SEQ ID NO: 264 - DNA, 50 bp, synthetic construct
  misc_feature 1..50: note = Synthetic
  source 1..50: mol_type = other DNA
cacacaacccctaccaggtatcacatgcacacggtttagcctcatccacg

SEQ ID NO: 265 - DNA, 50 bp, synthetic construct
  misc_feature 1..50: note = Synthetic
  source 1..50: mol_type = other DNA
ccacggcttcggtgttgtgttttagccccggacattttcggcgcagggcc

SEQ ID NO: 266 - DNA, 50 bp, synthetic construct
  misc_feature 1..50: note = Synthetic
  source 1..50: mol_type = other DNA
ccaccttcctccagtttatcactggcagtctcctttgagttcccggccgg

SEQ ID NO: 267 - DNA, 50 bp, synthetic construct
  misc_feature 1..50: note = Synthetic
  source 1..50: mol_type = other DNA
agctttcggggagaaccagctatctcccggtttgattggcctttcacccc

SEQ ID NO: 268 - DNA, 50 bp, synthetic construct
  misc_feature 1..50: note = Synthetic
  source 1..50: mol_type = other DNA
cgagccttcctcaggaaaccttaggcattcggtggaggggattctcaccc

SEQ ID NO: 269 - DNA, 50 bp, synthetic construct
  misc_feature 1..50: note = Synthetic
  source 1..50: mol_type = other DNA
cccagggctagatcatcccgcttcgggtccaggacaagcgactgaaaacg

SEQ ID NO: 270 - DNA, 50 bp, synthetic construct
  misc_feature 1..50: note = Synthetic
  source 1..50: mol_type = other DNA
aaaatcatgggaaatctcatcttgaggggggcttcgcacttagatgcttt

SEQ ID NO: 271 - DNA, 50 bp, synthetic construct
  misc_feature 1..50: note = Synthetic
  source 1..50: mol_type = other DNA
atcctgtacaagctgtaccaacattcaatatcaggctgcagtaaagctcc

SEQ ID NO: 272 - DNA, 50 bp, synthetic construct
  misc_feature 1..50: note = Synthetic
  source 1..50: mol_type = other DNA
ttagcaggtggtccggattcttctcctctcgggcacggaccttagcaccc

SEQ ID NO: 273 - DNA, 50 bp, synthetic construct
  misc_feature 1..50: note = Synthetic
  source 1..50: mol_type = other DNA
gtccgtttacggtacgggtacctcaaggataagtttagcgggttttctag

SEQ ID NO: 274 - DNA, 50 bp, synthetic construct
  misc_feature 1..50: note = Synthetic
  source 1..50: mol_type = other DNA
cactggcgtgctgccttctctgcctcccacctatcctgtacatgaaatac

SEQ ID NO: 275 - DNA, 50 bp, synthetic construct
  misc_feature 1..50: note = Synthetic
  source 1..50: mol_type = other DNA
tgcggtattagcagtcatttctaactgttatccccctgtataaggcaggt

SEQ ID NO: 276 - DNA, 50 bp, synthetic construct
  misc_feature 1..50: note = Synthetic
  source 1..50: mol_type = other DNA
gctatcggtcagacaggtatgcttagacttacaccacggtcggtgcggat

SEQ ID NO: 277 - DNA, 50 bp, synthetic construct
  misc_feature 1..50: note = Synthetic
  source 1..50: mol_type = other DNA
tttactcctttcggatgggatatctcatcttgaggggggcttcacgctta

SEQ ID NO: 278 - DNA, 50 bp, synthetic construct
  misc_feature 1..50: note = Synthetic
  source 1..50: mol_type = other DNA
tggccggtcgccctctcaggccggctacccgtcgaagccttggtgagccg

SEQ ID NO: 279 - DNA, 50 bp, synthetic construct
  misc_feature 1..50: note = Synthetic
  source 1..50: mol_type = other DNA
aagcctgttcccctggcaagccgttttatgactcccgcccggcccgtcgg

SEQ ID NO: 280 - DNA, 50 bp, synthetic construct
  misc_feature 1..50: note = Synthetic
  source 1..50: mol_type = other DNA
aaggttaagcctcacggttcattagtaccggttagctcaacgcatcgctg

SEQ ID NO: 281 - DNA, 50 bp, synthetic construct
  misc_feature 1..50: note = Synthetic
  source 1..50: mol_type = other DNA
gacatcatactaacgcgccctattaagactcggtttccctacggctccgt

SEQ ID NO: 282 - DNA, 50 bp, synthetic construct
  misc_feature 1..50: note = Synthetic
  source 1..50: mol_type = other DNA
tgtgtttttgttaaacagttgcctggaccgattctctgcgcctcaagtcg

SEQ ID NO: 283 - DNA, 50 bp, synthetic construct
  misc_feature 1..50: note = Synthetic
  source 1..50: mol_type = other DNA
gccccagtcaaactacccaccagacactgtccgcaacccggattacgggt

SEQ ID NO: 284 - DNA, 50 bp, synthetic construct
  misc_feature 1..50: note = Synthetic
  source 1..50: mol_type = other DNA
gcgtcacacctgttaatgcgcttgccttaccggttcaggtcccgcgctcc

SEQ ID NO: 285 - DNA, 50 bp, synthetic construct
  misc_feature 1..50: note = Synthetic
  source 1..50: mol_type = other DNA
gcgatggcccttccatgcggaaccaccggatcactaagcccgactttcgt

SEQ ID NO: 286 - DNA, 50 bp, synthetic construct
  misc_feature 1..50: note = Synthetic
  source 1..50: mol_type = other DNA
aagctccatggggtctttccgtctagtcgcgggtaaccggcatcttcacc

SEQ ID NO: 287 - DNA, 50 bp, synthetic construct
  misc_feature 1..50: note = Synthetic
  source 1..50: mol_type = other DNA
cgctagccctaaagctatttcggagagaaccagctatctccaagttcgtt

SEQ ID NO: 288 - DNA, 50 bp, synthetic construct
  misc_feature 1..50: note = Synthetic
  source 1..50: mol_type = other DNA
tcccatccgcacttcgcttccctgctatgccgttggcacgacaacagttg

SEQ ID NO: 289 - DNA, 50 bp, synthetic construct
  misc_feature 1..50: note = Synthetic
  source 1..50: mol_type = other DNA
tttcactcccctcccggggtccttttcacctttccttcacagtactctgc

SEQ ID NO: 290 - DNA, 50 bp, synthetic construct
  misc_feature 1..50: note = Synthetic
  source 1..50: mol_type = other DNA
cgtcctcggcttaggccccgacttaccctgggcggatgaaccttccccag

SEQ ID NO: 291 - DNA, 50 bp, synthetic construct
  misc_feature 1..50: note = Synthetic
  source 1..50: mol_type = other DNA
cgacatcgaggtgccaaacctccccgtcgatgtggactcttgggggagat

SEQ ID NO: 292 - DNA, 50 bp, synthetic construct; identical to SEQ ID NO: 53
  misc_feature 1..50: note = Synthetic
  source 1..50: mol_type = other DNA

SEQ ID NO: 293 - DNA, 50 bp, synthetic construct; identical to SEQ ID NO: 120
  misc_feature 1..50: note = Synthetic
  source 1..50: mol_type = other DNA

SEQ ID NO: 294 - DNA, 50 bp, synthetic construct
  misc_feature 1..50: note = Synthetic
  source 1..50: mol_type = other DNA
acgccttaaccatgtgaagggtagattttctgaccccttcggcctgaacg

SEQ ID NO: 295 - DNA, 50 bp, synthetic construct
  misc_feature 1..50: note = Synthetic
  source 1..50: mol_type = other DNA
ctcaaggattaagtttagcggattttctcgggagtatgtttacacgcact

SEQ ID NO: 296 - DNA, 50 bp, synthetic construct
  misc_feature 1..50: note = Synthetic
  source 1..50: mol_type = other DNA
ccccatccatcaccgataaatctttaatctctttcagatgtcttctagag

SEQ ID NO: 297 - DNA, 50 bp, synthetic construct
  misc_feature 1..50: note = Synthetic
  source 1..50: mol_type = other DNA
atactttgggaccttagctgtgggtctgggctgtttcccttttgacaatg

SEQ ID NO: 298 - DNA, 50 bp, synthetic construct
  misc_feature 1..50: note = Synthetic
  source 1..50: mol_type = other DNA
cgcccataggcggtgccggcccatgacggccggcgggttcccccattcgg

SEQ ID NO: 299 - DNA, 50 bp, synthetic construct
  misc_feature 1..50: note = Synthetic
  source 1..50: mol_type = other DNA
aaaatcatgggaaatctcatcttgaggtgggcttcgcacttagatgcttt

SEQ ID NO: 300 - DNA, 50 bp, synthetic construct
  misc_feature 1..50: note = Synthetic
  source 1..50: mol_type = other DNA
acaacttgatacccgattattatccacgcccgactcctcgactagtgagc

SEQ ID NO: 301 - DNA, 50 bp, synthetic construct
  misc_feature 1..50: note = Synthetic
  source 1..50: mol_type = other DNA
ctgagtttgataagcttcgctaacctctcggccgctaggctattcagtgc

SEQ ID NO: 302 - DNA, 50 bp, synthetic construct
  misc_feature 1..50: note = Synthetic
  source 1..50: mol_type = other DNA
gcccagatcgttgcgcctttcgtgcgggtcggaacttacccgacaaggaa

SEQ ID NO: 303 - DNA, 50 bp, synthetic construct
  misc_feature 1..50: note = Synthetic
  source 1..50: mol_type = other DNA
ttatagttacggccgccgttcactggggcttcggatcactgcttcagatc

SEQ ID NO: 304 - DNA, 50 bp, synthetic construct
  misc_feature 1..50: note = Synthetic
  source 1..50: mol_type = other DNA
ggcattgtcccaccgccgggtcacggcggctggttagaaacccaatactg

SEQ ID NO: 305 - DNA, 50 bp, synthetic construct
  misc_feature 1..50: note = Synthetic
  source 1..50: mol_type = other DNA
gtccacacatttagccccagacaaccatcgctggggttgagctacctcac

SEQ ID NO: 306 - DNA, 50 bp, synthetic construct
  misc_feature 1..50: note = Synthetic
  source 1..50: mol_type = other DNA
tctcacgacgttctgaacccagctcgcgtgccgctttaatgggcgaacag

SEQ ID NO: 307 - DNA, 50 bp, synthetic construct
  misc_feature 1..50: note = Synthetic
  source 1..50: mol_type = other DNA
atgcgacgagccgacatcgaggtgccaaacctccccgtcgatgtgaactc

SEQ ID NO: 308 - DNA, 50 bp, synthetic construct
  misc_feature 1..50: note = Synthetic
  source 1..50: mol_type = other DNA
cctgtgtcggtttagggtacgggcagtttgaacctcgcgccgatgctttt

SEQ ID NO: 309 - DNA, 50 bp, synthetic construct
  misc_feature 1..50: note = Synthetic
  source 1..50: mol_type = other DNA
cgatattgcaagggtggtatcccaacagcgcctcctcagagactggcgtc

SEQ ID NO: 310 - DNA, 50 bp, synthetic construct
  misc_feature 1..50: note = Synthetic
  source 1..50: mol_type = other DNA
cccccgaccggattcacggccgcaggttagaatttcagcacctcaagagt

SEQ ID NO: 311 - DNA, 50 bp, synthetic construct
  misc_feature 1..50: note = Synthetic
  source 1..50: mol_type = other DNA
tcagatggcggcattgtcactactgcgtctccacatcactcctggaggta

SEQ ID NO: 312 - DNA, 50 bp, synthetic construct
  misc_feature 1..50: note = Synthetic
  source 1..50: mol_type = other DNA
cttttcgtcccatcgcgggtaatcggcatcttcaccgatactacaatttc

SEQ ID NO: 313 - DNA, 50 bp, synthetic construct
  misc_feature 1..50: note = Synthetic
  source 1..50: mol_type = other DNA
acaacgaattccgccaacttcccgcgcactcaagccctccagttcgcgct

SEQ ID NO: 314 - DNA, 50 bp, synthetic construct
  misc_feature 1..50: note = Synthetic
  source 1..50: mol_type = other DNA
cccgaagttacggggccaatttgccgagttccttaacaacccttctcccg

SEQ ID NO: 315 - DNA, 50 bp, synthetic construct
  misc_feature 1..50: note = Synthetic
  source 1..50: mol_type = other DNA
tcaagggggtttacttctttcgaatgggatatctcatcttaaggggggct

SEQ ID NO: 316 - DNA, 50 bp, synthetic construct
  misc_feature 1..50: note = Synthetic
  source 1..50: mol_type = other DNA
cttcacagtactatacgctatcggtcactgggtagtatttagggttggag

SEQ ID NO: 317 - DNA, 50 bp, synthetic construct
  misc_feature 1..50: note = Synthetic
  source 1..50: mol_type = other DNA
attccgtcagacggccggactgtcacttctccgtcaccacatcgctctct

SEQ ID NO: 318 - DNA, 50 bp, synthetic construct
  misc_feature 1..50: note = Synthetic
  source 1..50: mol_type = other DNA
cggtactggttcactatcggtcactagggagtatttagggttgggagatg

SEQ ID NO: 319 - DNA, 50 bp, synthetic construct
  misc_feature 1..50: note = Synthetic
  source 1..50: mol_type = other DNA
agctgatggtccggattcttctcctttaggacatggaccttagcacccat

SEQ ID NO: 320 - DNA, 50 bp, synthetic construct
  misc_feature 1..50: note = Synthetic
  source 1..50: mol_type = other DNA
cgtattaccgcggctgctggcacggaattagccggtccttattcataagg

SEQ ID NO: 321 - DNA, 50 bp, synthetic construct
  misc_feature 1..50: note = Synthetic
  source 1..50: mol_type = other DNA
acgggttagcctcgccacgcaccactgactcgcagactcatttttcgata

SEQ ID NO: 322 - DNA, 50 bp, synthetic construct
  misc_feature 1..50: note = Synthetic
  source 1..50: mol_type = other DNA
acggcgtggactaccagggtatctaatcctgttcgctccccacgctttcg

SEQ ID NO: 323 - DNA, 50 bp, synthetic construct
  misc_feature 1..50: note = Synthetic
  source 1..50: mol_type = other DNA
tgcgcattcggagtttatcaagacttgataggcggtgaagccctcgcatc

SEQ ID NO: 324 - DNA, 50 bp, synthetic construct
  misc_feature 1..50: note = Synthetic
  source 1..50: mol_type = other DNA
ctgttgtccatcggctacgactctcgtcctcaccttaggccccgacttac

SEQ ID NO: 325 - DNA, 50 bp, synthetic construct
  misc_feature 1..50: note = Synthetic
  source 1..50: mol_type = other DNA
ggctcacgcctcaccttcgacgcggagtggaatgctcccctaccgatgtt

SEQ ID NO: 326 - DNA, 50 bp, synthetic construct
  misc_feature 1..50: note = Synthetic
  source 1..50: mol_type = other DNA
gatgtttcagttcaggcggttccctcgatatacctatttttaagttcagt

SEQ ID NO: 327 - DNA, 50 bp, synthetic construct
  misc_feature 1..50: note = Synthetic
  source 1..50: mol_type = other DNA
cattgtctaagattccccactgctgcctcccgtaggagtctgggccgtgt

SEQ ID NO: 328 - DNA, 50 bp, synthetic construct
  misc_feature 1..50: note = Synthetic
  source 1..50: mol_type = other DNA
tcacagtactatgcgctatcggtcactaagtggtatttagccttaggggg

SEQ ID NO: 329 - DNA, 50 bp, synthetic construct
  misc_feature 1..50: note = Synthetic
  source 1..50: mol_type = other DNA
gtagtatttaggcttggaggatggtccctcctgcttcccacagggtttca

SEQ ID NO: 330 - DNA, 50 bp, synthetic construct
  misc_feature 1..50: note = Synthetic
  source 1..50: mol_type = other DNA
ttgggaccttagctgcgggtctgggctctttcccttttgactatccaact

SEQ ID NO: 331 - DNA, 50 bp, synthetic construct
  misc_feature 1..50: note = Synthetic
  source 1..50: mol_type = other DNA
cagcttggtggcgcagaactaagcatttgactcagtcctcacctcactgc

SEQ ID NO: 332 - DNA, 50 bp, synthetic construct
  misc_feature 1..50: note = Synthetic
  source 1..50: mol_type = other DNA
accaagtacaggaatattaacctgtttcccatcgactacgcctttcggcc

SEQ ID NO: 333 - DNA, 50 bp, synthetic construct
  misc_feature 1..50: note = Synthetic
  source 1..50: mol_type = other DNA
aagcccgcttgtgcgattacactcgacacccgattgccaaccgggccgag

SEQ ID NO: 334 - DNA, 50 bp, synthetic construct
  misc_feature 1..50: note = Synthetic
  source 1..50: mol_type = other DNA
ccttaaatacgcacaaccatcggcgcactgcagctacctgtctgcgtcac

SEQ ID NO: 335 - DNA, 50 bp, synthetic construct
  misc_feature 1..50: note = Synthetic
  source 1..50: mol_type = other DNA
ctacccagcgatgcctttggcaagacaactggtacaccagcggtaagtcc

SEQ ID NO: 336 - DNA, 50 bp, synthetic construct
  misc_feature 1..50: note = Synthetic
  source 1..50: mol_type = other DNA
cctgtgtcggtttacggtacgggcgcatggcaaacaatagcggcttttct

SEQ ID NO: 337 - DNA, 50 bp, synthetic construct
  misc_feature 1..50: note = Synthetic
  source 1..50: mol_type = other DNA
ccgcgcttaccctatcctcctgcgtccccccattgctcaaatggtgagga

SEQ ID NO: 338 - DNA, 50 bp, synthetic construct
  misc_feature 1..50: note = Synthetic
  source 1..50: mol_type = other DNA
ggctctctgtactgtcaggtttcagcaaggactaactcttaatctgcccc

SEQ ID NO: 339 - DNA, 50 bp, synthetic construct
  misc_feature 1..50: note = Synthetic
  source 1..50: mol_type = other DNA
ggatcaccggattcgggccgtaaggcccccatcatcgcgcctcgccccga

SEQ ID NO: 340 - DNA, 50 bp, synthetic construct
  misc_feature 1..50: note = Synthetic
  source 1..50: mol_type = other DNA
tggtctccgctcgttcagacaaggtttcacgtgtctcgtcctactctgga

SEQ ID NO: 341 - DNA, 50 bp, synthetic construct
  misc_feature 1..50: note = Synthetic
  source 1..50: mol_type = other DNA
caatcccactttatgccaccggatcactaagtcctactttcgtacctgct

SEQ ID NO: 342 - DNA, 50 bp, synthetic construct
  misc_feature 1..50: note = Synthetic
  source 1..50: mol_type = other DNA
gtcaccaagtagtatttagccttggggggtgggccccccgtcttcccacc

SEQ ID NO: 343 - DNA, 50 bp, synthetic construct
  misc_feature 1..50: note = Synthetic
  source 1..50: mol_type = other DNA
atccccggagtaccttttatccgttgagcgatggcccttccattcagaac

SEQ ID NO: 344 - DNA, 50 bp, synthetic construct
  misc_feature 1..50: note = Synthetic
  source 1..50: mol_type = other DNA
tacctctcacggtgaccatccgacgcggcacctaaatgcctttcggggag

SEQ ID NO: 345 - DNA, 50 bp, synthetic construct
  misc_feature 1..50: note = Synthetic
  source 1..50: mol_type = other DNA
ccgtactccccaggcggagtgcttaatgcgttagctgcagcactaagggg

SEQ ID NO: 346 - DNA, 50 bp, synthetic construct
  misc_feature 1..50: note = Synthetic
  source 1..50: mol_type = other DNA
atcaccagttttaccctagggcgctccttgcggttacgcacttcaggtac

SEQ ID NO: 347 - DNA, 50 bp, synthetic construct
  misc_feature 1..50: note = Synthetic
  source 1..50: mol_type = other DNA
ggagggcacctttagaagcctccgttacgcttttggaggcgaccacccca

SEQ ID NO: 348 - DNA, 50 bp, synthetic construct; identical to SEQ ID NO: 105
  misc_feature 1..50: note = Synthetic
  source 1..50: mol_type = other DNA

SEQ ID NO: 349 - DNA, 50 bp, synthetic construct
  misc_feature 1..50: note = Synthetic
  source 1..50: mol_type = other DNA
gggctttcaccctctttggctggctttcccaaaaccattctgctaggatc

SEQ ID NO: 350 - DNA, 50 bp, synthetic construct
  misc_feature 1..50: note = Synthetic
  source 1..50: mol_type = other DNA
gtgggattggcttaacctcgcggtttcgctgccctttgttctgtccattg

SEQ ID NO: 351 - DNA, 50 bp, synthetic construct
  misc_feature 1..50: note = Synthetic
  source 1..50: mol_type = other DNA
atgctacgcagagaagtccggatatcaatgccagactagagtaaagctcc

SEQ ID NO: 352 - DNA, 50 bp, synthetic construct
  misc_feature 1..50: note = Synthetic
  source 1..50: mol_type = other DNA
tccgtatactctcaggttcgactctccccgcggatttgcctacgggaatc

SEQ ID NO: 353 - DNA, 50 bp, synthetic construct
  misc_feature 1..50: note = Synthetic
  source 1..50: mol_type = other DNA
ctggacctattctctgcgcctcacattgctgtgaggaccctttatcccga

SEQ ID NO: 354 - DNA, 50 bp, synthetic construct; identical to SEQ ID NO: 272
  misc_feature 1..50: note = Synthetic
  source 1..50: mol_type = other DNA

SEQ ID NO: 355 - DNA, 50 bp, synthetic construct
  misc_feature 1..50: note = Synthetic
  source 1..50: mol_type = other DNA
gcctgtacacctgcatcctatcaacgtcatagtctttgacgaccctgaga

SEQ ID NO: 356 - DNA, 50 bp, synthetic construct
  misc_feature 1..50: note = Synthetic
  source 1..50: mol_type = other DNA
agactccaatccggactacgacgcactttatgaggtccgcttgctctcgc

SEQ ID NO: 357 - DNA, 50 bp, synthetic construct
  misc_feature 1..50: note = Synthetic
  source 1..50: mol_type = other DNA
ggtttgccctcctgcctcttcgctcgccgctactgaggcaatcgctcttg

SEQ ID NO: 358 - DNA, 50 bp, synthetic construct
  misc_feature 1..50: note = Synthetic
  source 1..50: mol_type = other DNA
acctttccctcacggtactggtacgctatcggtcagacaggtatgcttag

SEQ ID NO: 359 - DNA, 50 bp, synthetic construct
  misc_feature 1..50: note = Synthetic
  source 1..50: mol_type = other DNA
ccggtcctctcgtactagggacagctcccatcaaatatcctgcgcccacg

SEQ ID NO: 360 - DNA, 50 bp, synthetic construct
  misc_feature 1..50: note = Synthetic
  source 1..50: mol_type = other DNA
ccattggcatgacaacccgaacaccagtgatgcgtccactccggtcctct

SEQ ID NO: 361 - DNA, 50 bp, synthetic construct
  misc_feature 1..50: note = Synthetic
  source 1..50: mol_type = other DNA
atgtgcttgtaagcacagagtttcaggttcttttcactcccctcccgggg

SEQ ID NO: 362 - DNA, 50 bp, synthetic construct
  misc_feature 1..50: note = Synthetic
  source 1..50: mol_type = other DNA
cccttctcccgaagttacggggtaattttgccgagttccttaacaaccct

SEQ ID NO: 363 - DNA, 50 bp, synthetic construct
  misc_feature 1..50: note = Synthetic
  source 1..50: mol_type = other DNA
cctgagtcggtttagggtacgggcgcgttatgccctcacgtcgaggcttt

SEQ ID NO: 364 - DNA, 50 bp, synthetic construct
  misc_feature 1..50: note = Synthetic
  source 1..50: mol_type = other DNA
atctgggctgtttccctttcgacaatgaaacttatctcacactgtctgac

SEQ ID NO: 365 - DNA, 50 bp, synthetic construct
  misc_feature 1..50: note = Synthetic
  source 1..50: mol_type = other DNA
cgtatttcaaggatggctccacaaacactggcgtgcctgcttcaaagcct

SEQ ID NO: 366 - DNA, 50 bp, synthetic construct
  misc_feature 1..50: note = Synthetic
  source 1..50: mol_type = other DNA
ggtcattgcctgcttgcggctgaccatggcttatcgcagctgaccacgtc

SEQ ID NO: 367 - DNA, 50 bp, synthetic construct
  misc_feature 1..50: note = Synthetic
  source 1..50: mol_type = other DNA
cctggcgcgggtaaccagcatcttcactggtacttcaatttcaccgggtg

SEQ ID NO: 368 - DNA, 50 bp, synthetic construct
  misc_feature 1..50: note = Synthetic
  source 1..50: mol_type = other DNA
gtaactcacaaggctgcacctaaatgcatttcggggagtacgagctatct

SEQ ID NO: 369 - DNA, 50 bp, synthetic construct
  misc_feature 1..50: note = Synthetic
  source 1..50: mol_type = other DNA
gtcggtttggggtacgggcggccatagccctcacgccgaggcttttctcg

SEQ ID NO: 370 - DNA, 50 bp, synthetic construct
  misc_feature 1..50: note = Synthetic
  source 1..50: mol_type = other DNA
caccgtctatggtcccattttccaaagggttctactcatgaaatgtcttg

SEQ ID NO: 371 - DNA, 50 bp, synthetic construct
  misc_feature 1..50: note = Synthetic
  source 1..50: mol_type = other DNA
ccggcaacgcaacccccgacgggtatcacgcgcaaccggtttggtctgat

SEQ ID NO: 372 - DNA, 50 bp, synthetic construct
  misc_feature 1..50: note = Synthetic
  source 1..50: mol_type = other DNA
ttatccttctgtgtcactgcttcattccatcggtagtgcaggaatctaca

SEQ ID NO: 373 - DNA, 50 bp, synthetic construct
  misc_feature 1..50: note = Synthetic
  source 1..50: mol_type = other DNA
cagagcaccccttctcccgaagttacggggtcattttgccgagttcctta

SEQ ID NO: 374 - DNA, 50 bp, synthetic construct
  misc_feature 1..50: note = Synthetic
  source 1..50: mol_type = other DNA
atactatcaggttcgattctcatggtggatttgcctgccaagatcaacat

SEQ ID NO: 375 - DNA, 50 bp, synthetic construct
  misc_feature 1..50: note = Synthetic
  source 1..50: mol_type = other DNA
cttacggggctttcaccctctctggccggctttcccaaaaccgttctgct

SEQ ID NO: 376 - DNA, 50 bp, synthetic construct
  misc_feature 1..50: note = Synthetic
  source 1..50: mol_type = other DNA
gaccggccttcccatgccgttcggttaacaacttaagtcctaaatgcggt

SEQ ID NO: 377 - DNA, 50 bp, synthetic construct
  misc_feature 1..50: note = Synthetic
  source 1..50: mol_type = other DNA
cgtttatccgatccgtacgtagttgcccagctatgctcctggcggaacaa

SEQ ID NO: 378 - DNA, 50 bp, synthetic construct
  misc_feature 1..50: note = Synthetic
  source 1..50: mol_type = other DNA
gtatctaatcctgtttgatacccacactttcgagcatcagcgtcagttac

SEQ ID NO: 379 - DNA, 50 bp, synthetic construct
  misc_feature 1..50: note = Synthetic
  source 1..50: mol_type = other DNA
ggtgcttgtaaacacaaggtttcaggttctttttcactccccgtcagggg

SEQ ID NO: 380 - DNA, 50 bp, synthetic construct
  misc_feature 1..50: note = Synthetic
  source 1..50: mol_type = other DNA
gtaggcgcacggtttcaggaactctttcactcccctcccggggtgctttt

SEQ ID NO: 381 - DNA, 50 bp, synthetic construct
  misc_feature 1..50: note = Synthetic
  source 1..50: mol_type = other DNA
acttctgagttcggcatggggtcaggtgggaccaccgcgctacggccgcc

SEQ ID NO: 382 - DNA, 50 bp, synthetic construct
  misc_feature 1..50: note = Synthetic
  source 1..50: mol_type = other DNA
ttccgtgttcggtatgggaacgggtgtgacctcttcgctatcgccaccaa

SEQ ID NO: 383 - DNA, 50 bp, synthetic construct
  misc_feature 1..50: note = Synthetic
  source 1..50: mol_type = other DNA
tcgccttaggacccgactcacccggggacgttaaccgtggccccggaacc

SEQ ID NO: 384 - DNA, 50 bp, synthetic construct
  misc_feature 1..50: note = Synthetic
  source 1..50: mol_type = other DNA
cactcacccacaaccatgggctccccatcatgcctcaaccttcacgccca

SEQ ID NO: 385 - DNA, 50 bp, synthetic construct
  misc_feature 1..50: note = Synthetic
  source 1..50: mol_type = other DNA
ctccgagacttcatatgtgtccctgtgtttaactcttttggtggtgacgg

SEQ ID NO: 386 - DNA, 50 bp, synthetic construct
  misc_feature 1..50: note = Synthetic
  source 1..50: mol_type = other DNA
aaaattccctactgctgcctcccgtaggagtttgggccgtgtctcagtcc

SEQ ID NO: 387 - DNA, 50 bp, synthetic construct
  misc_feature 1..50: note = Synthetic
  source 1..50: mol_type = other DNA
gaccaggtaaggttcttcgcgttgcatcgaattaaaccacatgctccacc

SEQ ID NO: 388 - DNA, 50 bp, synthetic construct
  misc_feature 1..50: note = Synthetic
  source 1..50: mol_type = other DNA
cgaagtttgatagggttcggtaagctttgtggccccctagcccattcagt

SEQ ID NO: 389 - DNA, 50 bp, synthetic construct
  misc_feature 1..50: note = Synthetic
  source 1..50: mol_type = other DNA
aggcttgcgccgccgcttcgccccgatggggacgctctcctacccagcgt

SEQ ID NO: 390 - DNA, 50 bp, synthetic construct
  misc_feature 1..50: note = Synthetic
  source 1..50: mol_type = other DNA
cgaacagagcggtatttcaccttacggctccgcgcgatctggcgaccgcg

SEQ ID NO: 391 - DNA, 50 bp, synthetic construct
  misc_feature 1..50: note = Synthetic
  source 1..50: mol_type = other DNA
accgttctacaaaaagtacgcggttgtactcgtatggtacttccacagtt

SEQ ID NO: 392 - DNA, 50 bp, synthetic construct
  misc_feature 1..50: note = Synthetic
  source 1..50: mol_type = other DNA
cgtttcgctcgccgctactcagggaatcgcatttgctttctcttcctccg

SEQ ID NO: 393 - DNA, 50 bp, synthetic construct
  misc_feature 1..50: note = Synthetic
  source 1..50: mol_type = other DNA
gctacttgggacaacacgatcggaagacggctcacgtccaggtacggggc

SEQ ID NO: 394 - DNA, 50 bp, synthetic construct
  misc_feature 1..50: note = Synthetic
  source 1..50: mol_type = other DNA
aaggtccccctctttggtcttgcgacgttatgcggtattagctaccgttt

SEQ ID NO: 395 - DNA, 50 bp, synthetic construct
  misc_feature 1..50: note = Synthetic
  source 1..50: mol_type = other DNA
gttctgaacccagctcgcgtaccactttaatcggcgaacagccgaaccct

SEQ ID NO: 396 - DNA, 50 bp, synthetic construct
  misc_feature 1..50: note = Synthetic
  source 1..50: mol_type = other DNA
tgattcaaagcctccggcctatcctacacatcaatcacccaaattcaatg

SEQ ID NO: 397 - DNA, 50 bp, synthetic construct; identical to SEQ ID NO: 114
  misc_feature 1..50: note = Synthetic
  source 1..50: mol_type = other DNA

SEQ ID NO: 398 - DNA, 50 bp, synthetic construct
  misc_feature 1..50: note = Synthetic
  source 1..50: mol_type = other DNA
ccccccccccccttcccccctctcctcccccttccccctttcgcgccccc

SEQ ID NO: 399 - DNA, 50 bp, synthetic construct
  misc_feature 1..50: note = Synthetic
  source 1..50: mol_type = other DNA
caggtgtcaccccatatacgtcatctttcgatttagcatagagctgtgtt

SEQ ID NO: 400 - DNA, 50 bp, synthetic construct; identical to SEQ ID NO: 193
  misc_feature 1..50: note = Synthetic
  source 1..50: mol_type = other DNA

SEQ ID NO: 401 - DNA, 50 bp, synthetic construct
  misc_feature 1..50: note = Synthetic
  source 1..50: mol_type = other DNA
ttccgtcagccggcaggactgtcacttctccgtctccacgtcactccatg

SEQ ID NO: 402 - DNA, 50 bp, synthetic construct
  misc_feature 1..50: note = Synthetic
  source 1..50: mol_type = other DNA
cgctaatttttcaacattagtcggttcggtcctccagttagtgttaccca

SEQ ID NO: 403 - DNA, 50 bp, synthetic construct
  misc_feature 1..50: note = Synthetic
  source 1..50: mol_type = other DNA
cttggcagtgtgacatcactaacttcgctactaaacttcgctccccatca

SEQ ID NO: 404 - DNA, 50 bp, synthetic construct
  misc_feature 1..50: note = Synthetic
  source 1..50: mol_type = other DNA
cccgttaaattttcggcgcagagtcactcgaccagtgagctattacgcac

SEQ ID NO: 405 - DNA, 50 bp, synthetic construct
  misc_feature 1..50: note = Synthetic
  source 1..50: mol_type = other DNA
cccggagtaccttttatcctttgagcgatgtcccttccatgcggaaacac

SEQ ID NO: 406 - DNA, 50 bp, synthetic construct
  misc_feature 1..50: note = Synthetic
  source 1..50: mol_type = other DNA
ttctctgcggctccatcgctgcagcaccccttctcccgaagttacggggt

SEQ ID NO: 407 - DNA, 50 bp, synthetic construct
  misc_feature 1..50: note = Synthetic
  source 1..50: mol_type = other DNA
aagctacctacttcttttgcaacccactcccatggtgtgacgggcggtgt

SEQ ID NO: 408 - DNA, 50 bp, synthetic construct
  misc_feature 1..50: note = Synthetic
  source 1..50: mol_type = other DNA
gcacagccatgtgtttttgttaaacagttgcctggacctattctctgcgc

SEQ ID NO: 409 - DNA, 50 bp, synthetic construct
  misc_feature 1..50: note = Synthetic
  source 1..50: mol_type = other DNA
gccaacatcctggttgtctgtgcaattccacatccttttccacttaacta

SEQ ID NO: 410 - DNA, 50 bp, synthetic construct
  misc_feature 1..50: note = Synthetic
  source 1..50: mol_type = other DNA
ggtcacccggtttcgggcccattatatgcaacttaacgcccttttcaaac

SEQ ID NO: 411 - DNA, 50 bp, synthetic construct
  misc_feature 1..50: note = Synthetic
  source 1..50: mol_type = other DNA
ttatagttacggccgccgttcactggggcttcgattcaatgcttgcacat

SEQ ID NO: 412 - DNA, 50 bp, synthetic construct
  misc_feature 1..50: note = Synthetic
  source 1..50: mol_type = other DNA
gtttatctgagattggtaatccgggatggacccctcaatcaaacagtgct

SEQ ID NO: 413 - DNA, 50 bp, synthetic construct
  misc_feature 1..50: note = Synthetic
  source 1..50: mol_type = other DNA
cgaagttacggggtcattttgccgagttccttgacaatgcttcttccgcc

SEQ ID NO: 414 - DNA, 50 bp, synthetic construct
  misc_feature 1..50: note = Synthetic
  source 1..50: mol_type = other DNA
gtccacacacgcgtgtgtccctcatcagttctcaccctccatgccccccg

SEQ ID NO: 415 - DNA, 50 bp, synthetic construct
  misc_feature 1..50: note = Synthetic
  source 1..50: mol_type = other DNA
ccggcccgtcggggccgggacacacgctcccgcaaccccggccacgcaac

SEQ ID NO: 416 - DNA, 50 bp, synthetic construct
  misc_feature 1..50: note = Synthetic
  source 1..50: mol_type = other DNA
ccggtacattttcggcgcagggtcactcgactagtgagctattacgcact

SEQ ID NO: 417 - DNA, 50 bp, synthetic construct
  misc_feature 1..50: note = Synthetic
  source 1..50: mol_type = other DNA
ctcgaacttcttgtaagcacacggtttcaggttctctttcactccccttc

SEQ ID NO: 418 - DNA, 50 bp, synthetic construct
  misc_feature 1..50: note = Synthetic
  source 1..50: mol_type = other DNA
tttcagttcaggcggttccccccgtatccctatggattcagaatacggtg

SEQ ID NO: 419 - DNA, 50 bp, synthetic construct
  misc_feature 1..50: note = Synthetic
  source 1..50: mol_type = other DNA
tccgttacattttgggaggcgaccgccccagtcaaactgcctacctgaca

SEQ ID NO: 420 - DNA, 50 bp, synthetic construct
  misc_feature 1..50: note = Synthetic
  source 1..50: mol_type = other DNA
ccgctccttccatcaaggttccacgtgtctcgatgtactctggatcctgc

SEQ ID NO: 421 - DNA, 50 bp, synthetic construct
  misc_feature 1..50: note = Synthetic
  source 1..50: mol_type = other DNA
ccacgtgttactcacccgtccgccgctaacatcagggagcaagctcccat

SEQ ID NO: 422 - DNA, 50 bp, synthetic construct
  misc_feature 1..50: note = Synthetic
  source 1..50: mol_type = other DNA
gactccgtactgtcaggttcggctcaacgggtggatttgcctgcccatct

SEQ ID NO: 423 - DNA, 50 bp, synthetic construct
  misc_feature 1..50: note = Synthetic
  source 1..50: mol_type = other DNA
acgtgtccggcggtactctggatacagatggctgttcaggcttttcgtgt

SEQ ID NO: 424 - DNA, 50 bp, synthetic construct
  misc_feature 1..50: note = Synthetic
  source 1..50: mol_type = other DNA
tgggctgtttccctttggacaatgaaacttatctcccactgtctgactcc

SEQ ID NO: 425 - DNA, 50 bp, synthetic construct
  misc_feature 1..50: note = Synthetic
  source 1..50: mol_type = other DNA
acatagctacccagccatgcccttggcagaacaactggtacaccagcggt

SEQ ID NO: 426 - DNA, 50 bp, synthetic construct
  misc_feature 1..50: note = Synthetic
  source 1..50: mol_type = other DNA
cagaggtcagtccaacacggtcctctcgtactagtgtcagagccacgcaa

SEQ ID NO: 427 - DNA, 50 bp, synthetic construct
  misc_feature 1..50: note = Synthetic
  source 1..50: mol_type = other DNA
gtttgatagggttcagtaacttctcagcccctagcccattcagtgcttta

SEQ ID NO: 428 - DNA, 50 bp, synthetic construct
  misc_feature 1..50: note = Synthetic
  source 1..50: mol_type = other DNA
cggcaccgggcaggcgtcacaccctatacgtccactgttcgtgttggcag

SEQ ID NO: 429 - DNA, 50 bp, synthetic construct
  misc_feature 1..50: note = Synthetic
  source 1..50: mol_type = other DNA
aacccaataaatccggataacgcttgccccctacgtattaccgcggctgc

SEQ ID NO: 430 - DNA, 50 bp, synthetic construct
  misc_feature 1..50: note = Synthetic
  source 1..50: mol_type = other DNA
ccatacatcaattatctggcattctgagtttgatagggttcagtaacctc

SEQ ID NO: 431 - DNA, 50 bp, synthetic construct
  misc_feature 1..50: note = Synthetic
  source 1..50: mol_type = other DNA
cctccgttacactttgggaggcgaccgccccagtcaaactgcccgccaag

SEQ ID NO: 432 - DNA, 50 bp, synthetic construct
  misc_feature 1..50: note = Synthetic
  source 1..50: mol_type = other DNA
ctgttatccccgaggtagcttttatccgttaagcgacggcttttccactc

SEQ ID NO: 433 - DNA, 50 bp, synthetic construct
  misc_feature 1..50: note = Synthetic
  source 1..50: mol_type = other DNA
tagcccattcagtgctttacctccggtaatctaaatcaacgctagcccta

SEQ ID NO: 434 - DNA, 50 bp, synthetic construct
  misc_feature 1..50: note = Synthetic
  source 1..50: mol_type = other DNA
tccacagctccttacggtactgcttcgtcccgcatgcaatgctcctctac

SEQ ID NO: 435 - DNA, 50 bp, synthetic construct
  misc_feature 1..50: note = Synthetic
  source 1..50: mol_type = other DNA
ccatcgcgggtaatcggcatcttcaccgatactacaatttcaccgagctc

SEQ ID NO: 436 - DNA, 50 bp, synthetic construct
  misc_feature 1..50: note = Synthetic
  source 1..50: mol_type = other DNA
ctggacctattctctgcgcccaactctcgttgggaccctttatcccgaag

SEQ ID NO: 437 - DNA, 50 bp, synthetic construct
  misc_feature 1..50: note = Synthetic
  source 1..50: mol_type = other DNA
cttttacctttacactctacgattgatttccaaccaatctgagccaacct

SEQ ID NO: 438 - DNA, 50 bp, synthetic construct
  misc_feature 1..50: note = Synthetic
  source 1..50: mol_type = other DNA
ttatagttacggccgccgtttaccggggcttcaattcaaagcttcatatt

SEQ ID NO: 439 - DNA, 50 bp, synthetic construct
  misc_feature 1..50: note = Synthetic
  source 1..50: mol_type = other DNA
gccattaagattctcacttaattctcgctacttattccggcattctcact

SEQ ID NO: 440 - DNA, 50 bp, synthetic construct
  misc_feature 1..50: note = Synthetic
  source 1..50: mol_type = other DNA
ggccgatcaccctctcaggtcggctacgcatcgtcgccttggtgagccgt

SEQ ID NO: 441 - DNA, 50 bp, synthetic construct
  misc_feature 1..50: note = Synthetic
  source 1..50: mol_type = other DNA
cttctcccgctggccttagaatcttcttcctatctacctgtgtcggtttg

SEQ ID NO: 442 - DNA, 50 bp, synthetic construct
  misc_feature 1..50: note = Synthetic
  source 1..50: mol_type = other DNA
ttccttcacccgagttctctcaagcgccttggtattctctacctgaccac

SEQ ID NO: 443 - DNA, 50 bp, synthetic construct
  misc_feature 1..50: note = Synthetic
  source 1..50: mol_type = other DNA
gctagtcctaaaactatttcggggagaaccagctatctccgggttcgatt

SEQ ID NO: 444 - DNA, 50 bp, synthetic construct
  misc_feature 1..50: note = Synthetic
  source 1..50: mol_type = other DNA
cctccggccggtttcacggccgcaagttagaattccagcactacaagagt

SEQ ID NO: 445 - DNA, 50 bp, synthetic construct
  misc_feature 1..50: note = Synthetic
  source 1..50: mol_type = other DNA
tgttcgtcccgtccttcatcggctcctagtgccaaggcatccaccgtgcg

SEQ ID NO: 446 - DNA, 50 bp, synthetic construct
  misc_feature 1..50: note = Synthetic
  source 1..50: mol_type = other DNA
gccaggccttcaagcctgttcccctggctagccgctttatgactcccgcc

SEQ ID NO: 447 - DNA, 50 bp, synthetic construct
  misc_feature 1..50: note = Synthetic
  source 1..50: mol_type = other DNA
ctttcttttcctccggctacttagatgtttcagttcaccgggttcccttc

SEQ ID NO: 448 - DNA, 50 bp, synthetic construct
  misc_feature 1..50: note = Synthetic
  source 1..50: mol_type = other DNA
atgattctcacataattctcgctactcattccggcattctcactcgtatg

SEQ ID NO: 449 - DNA, 50 bp, synthetic construct
  misc_feature 1..50: note = Synthetic
  source 1..50: mol_type = other DNA
cgggcacggaccttagcacccatgcccttactgccggactgcagaccgtg

SEQ ID NO: 450 - DNA, 50 bp, synthetic construct
  misc_feature 1..50: note = Synthetic
  source 1..50: mol_type = other DNA
gtgagtttcctcattcagagatctccggatcaatgcttatttgcagctcc

SEQ ID NO: 451 - DNA, 50 bp, synthetic construct
  misc_feature 1..50: note = Synthetic
  source 1..50: mol_type = other DNA
taaatgcagtccgaaccccggagtgcacgcactccggtttgggctctttc

SEQ ID NO: 452 - DNA, 50 bp, synthetic construct
  misc_feature 1..50: note = Synthetic
  source 1..50: mol_type = other DNA
gcccaagggtagatcacttggtttcgcgtctactccttccgactatacgc

SEQ ID NO: 453 - DNA, 50 bp, synthetic construct
  misc_feature 1..50: note = Synthetic
  source 1..50: mol_type = other DNA
agcttagcggattttctcgggagtctgattaccggcgctattggattcca

SEQ ID NO: 454 - DNA, 50 bp, synthetic construct
  misc_feature 1..50: note = Synthetic
  source 1..50: mol_type = other DNA
ctcgcagtcaagctcccttctgcctttgcactctccgaatgatttccaac

SEQ ID NO: 455 - DNA, 50 bp, synthetic construct
  misc_feature 1..50: note = Synthetic
  source 1..50: mol_type = other DNA
gtctagtcccacgtacttgtgcgccctgttcagactcgctttcgctccgc

SEQ ID NO: 456 - DNA, 50 bp, synthetic construct
  misc_feature 1..50: note = Synthetic
  source 1..50: mol_type = other DNA
ttctccgctatccacacctcatcgccacccttttcaacggatgtgcgttc

SEQ ID NO: 457 - DNA, 50 bp, synthetic construct
  misc_feature 1..50: note = Synthetic
  source 1..50: mol_type = other DNA
gccggctcccattccgtgtcacccctgcgctcacctaccacggctacgct

SEQ ID NO: 458 - DNA, 50 bp, synthetic construct
  misc_feature 1..50: note = Synthetic
  source 1..50: mol_type = other DNA
tcccggggtccttttcacctttccttcacagtactatgcgctatcggtca

SEQ ID NO: 459 - DNA, 50 bp, synthetic construct
  misc_feature 1..50: note = Synthetic
  source 1..50: mol_type = other DNA
ccaacatcctggttgtctgtgcaattccacatccttttccacttaaatcc

SEQ ID NO: 460 - DNA, 50 bp, synthetic construct
  misc_feature 1..50: note = Synthetic
  source 1..50: mol_type = other DNA
gctggcgccgcggcttcgaagcctcccgcctatgctacacaatccgcacc

SEQ ID NO: 461 - DNA, 50 bp, synthetic construct
  misc_feature 1..50: note = Synthetic
  source 1..50: mol_type = other DNA
acgcccaataattccggacaacgcttgccacctacgtattaccgcggctg

SEQ ID NO: 462 - DNA, 50 bp, synthetic construct
  misc_feature 1..50: note = Synthetic
  source 1..50: mol_type = other DNA
ccctaccaggtatcacatgcacacggtttagcctcatccacgttcgttcg

SEQ ID NO: 463 - DNA, 50 bp, synthetic construct
  misc_feature 1..50: note = Synthetic
  source 1..50: mol_type = other DNA
agcaccgggcaggtgtcaggctgtatacgtgatctttcaatttggcacag

SEQ ID NO: 464 - DNA, 50 bp, synthetic construct
  misc_feature 1..50: note = Synthetic
  source 1..50: mol_type = other DNA
ctccccatcatgcctcaaccttcacgcccagcggatttacctaccagaca

SEQ ID NO: 465 - DNA, 50 bp, synthetic construct
  misc_feature 1..50: note = Synthetic
  source 1..50: mol_type = other DNA
cttcaacttaacctcgcacgtaaacgtaactcgccggttcattctacaaa

SEQ ID NO: 466 - DNA, 50 bp, synthetic construct
  misc_feature 1..50: note = Synthetic
  source 1..50: mol_type = other DNA
agagtagccataacacaagggtagtatcccaacaacgcctcagtcgaaac

SEQ ID NO: 467 - DNA, 50 bp, synthetic construct
  misc_feature 1..50: note = Synthetic
  source 1..50: mol_type = other DNA
gctcgcgtaccactttaaatggcgaacagccatacccttgggacctactt

SEQ ID NO: 468 - DNA, 50 bp, synthetic construct; identical to SEQ ID NO: 32
  misc_feature 1..50: note = Synthetic
  source 1..50: mol_type = other DNA

SEQ ID NO: 469 - DNA, 50 bp, synthetic construct
  misc_feature 1..50: note = Synthetic
  source 1..50: mol_type = other DNA
acacacaacccctaccaagtatcacatgcacacggtttagcctcatccac

SEQ ID NO: 470 - DNA, 50 bp, synthetic construct
  misc_feature 1..50: note = Synthetic
  source 1..50: mol_type = other DNA
tctacgaccacgtactcatgcgccctattcagactcgctttcgctgcggc

SEQ ID NO: 471 - DNA, 50 bp, synthetic construct
  misc_feature 1..50: note = Synthetic
  source 1..50: mol_type = other DNA
cattcggatatctctggatcaaggcttacttacagctccccaaagcatgt

SEQ ID NO: 472 - DNA, 50 bp, synthetic construct
  misc_feature 1..50: note = Synthetic
  source 1..50: mol_type = other DNA
gctctcctaccactgttcgaagaacagtccgcagcttcggtgatacgttt

SEQ ID NO: 473 - DNA, 50 bp, synthetic construct
  misc_feature 1..50: note = Synthetic
  source 1..50: mol_type = other DNA
tcttttcgtcccatcgcgggtaatcggcatcttcaccgatactacaattt

SEQ ID NO: 474 - DNA, 50 bp, synthetic construct
  misc_feature 1..50: note = Synthetic
  source 1..50: mol_type = other DNA
tgtaccccccattgtaacacgtgtgtagccccggacgtaagggccgtgct

SEQ ID NO: 475 - DNA, 50 bp, synthetic construct; identical to SEQ ID NO: 97
  misc_feature 1..50: note = Synthetic
  source 1..50: mol_type = other DNA

SEQ ID NO: 476 - DNA, 50 bp, synthetic construct
  misc_feature 1..50: note = Synthetic
  source 1..50: mol_type = other DNA
cgttgagcgatggcccttcctttcggtaccaccggatcactaagcccgac

SEQ ID NO: 477 - DNA, 50 bp, synthetic construct
  misc_feature 1..50: note = Synthetic
  source 1..50: mol_type = other DNA
ttcaaggggtcttactcgttatacgatgggatatctaatcttggagtcgg

SEQ ID NO: 478 - DNA, 50 bp, synthetic construct
  misc_feature 1..50: note = Synthetic
  source 1..50: mol_type = other DNA
cctcctgatgtccgaccaggattagccaaccttcgtgctcctccgttact

SEQ ID NO: 479 - DNA, 50 bp, synthetic construct
  misc_feature 1..50: note = Synthetic
  source 1..50: mol_type = other DNA
accttggtcttacggcgggagggaatctcaccctccttatcgttacttat

SEQ ID NO: 480 - DNA, 50 bp, synthetic construct
  misc_feature 1..50: note = Synthetic
  source 1..50: mol_type = other DNA
cgtgccccgccctactcaggatactgctagccacgatcaacttttaggta

SEQ ID NO: 481 - DNA, 50 bp, synthetic construct
  misc_feature 1..50: note = Synthetic
  source 1..50: mol_type = other DNA
caccctcagttcatccggaagcttttcaacgcttatcggttcggtcctcc

SEQ ID NO: 482 - DNA, 50 bp, synthetic construct
  misc_feature 1..50: note = Synthetic
  source 1..50: mol_type = other DNA
tctacctccatgagactaatacgaggctagccctaaagctatttcgagga

SEQ ID NO: 483 - DNA, 50 bp, synthetic construct
  misc_feature 1..50: note = Synthetic
  source 1..50: mol_type = other DNA
tacctgtgtcggtttgcggtacgggcaccttagcatacactagaactttt

SEQ ID NO: 484 - DNA, 50 bp, synthetic construct
  misc_feature 1..50: note = Synthetic
  source 1..50: mol_type = other DNA
agcggttccacagcttgtaaacatatggtttcaggttctctttcactccc

SEQ ID NO: 485 - DNA, 50 bp, synthetic construct
  misc_feature 1..50: note = Synthetic
  source 1..50: mol_type = other DNA
ttatagttacggccgccgttcactggggcttcgggtcaaagcttgcactc

SEQ ID NO: 486 - DNA, 50 bp, synthetic construct
  misc_feature 1..50: note = Synthetic
  source 1..50: mol_type = other DNA
ttatagttacggccgccgtttactggggcttcggttcgatgcttcgattg

SEQ ID NO: 487 - DNA, 50 bp, synthetic construct
  misc_feature 1..50: note = Synthetic
  source 1..50: mol_type = other DNA
gccttacggggtggtccccgctcattcccacaaggtttctcgtgtctcgt

SEQ ID NO: 488 - DNA, 50 bp, synthetic construct
  misc_feature 1..50: note = Synthetic
  source 1..50: mol_type = other DNA
ccggagtttttcacactgagccatgcagctctgtgcgcttatgcggtatt

SEQ ID NO: 489 - DNA, 50 bp, synthetic construct
  misc_feature 1..50: note = Synthetic
  source 1..50: mol_type = other DNA
cttctcccgttggccttagaatcttcttcctacctacctgtgtcggtttg

SEQ ID NO: 490 - DNA, 50 bp, synthetic construct
  misc_feature 1..50: note = Synthetic
  source 1..50: mol_type = other DNA
tgccgctttaatgggcgaacagcccaacccttgggaccgactacagcccc

SEQ ID NO: 491 - DNA, 50 bp, synthetic construct
  misc_feature 1..50: note = Synthetic
  source 1..50: mol_type = other DNA
ggagttcttcgtgatatctaagcatttcaccgctacaccacgaattccgc

SEQ ID NO: 492 - DNA, 50 bp, synthetic construct
  misc_feature 1..50: note = Synthetic
  source 1..50: mol_type = other DNA
agtgatgggcaggttggatacgcgttactcacccgtgcgccggtcgacgc

SEQ ID NO: 493 - DNA, 50 bp, synthetic construct
  misc_feature 1..50: note = Synthetic
  source 1..50: mol_type = other DNA
tcacggtactcgtacgctatcggtcagacaggtatactcaggcttacccg

SEQ ID NO: 494 - DNA, 50 bp, synthetic construct; identical to SEQ ID NO: 214
  misc_feature 1..50: note = Synthetic
  source 1..50: mol_type = other DNA

SEQ ID NO: 495 - DNA, 50 bp, synthetic construct
  misc_feature 1..50: note = Synthetic
  source 1..50: mol_type = other DNA
catcatctgtatggcattcggagtttgatatcccttagtaagctttgacg

SEQ ID NO: 496 - DNA, 50 bp, synthetic construct; identical to SEQ ID NO: 456
  misc_feature 1..50: note = Synthetic
  source 1..50: mol_type = other DNA

SEQ ID NO: 497 - DNA, 50 bp, synthetic construct
  misc_feature 1..50: note = Synthetic
  source 1..50: mol_type = other DNA
aagcactttggtttgggctgttccccgttcgctcgccgctacttagggaa

SEQ ID NO: 498 - DNA, 50 bp, synthetic construct
  misc_feature 1..50: note = Synthetic
  source 1..50: mol_type = other DNA
cacttatgcccgattattatccacgccaaactcctcgactagtgagctgt

SEQ ID NO: 499 - DNA, 50 bp, synthetic construct
  misc_feature 1..50: note = Synthetic
  source 1..50: mol_type = other DNA
cttaggacccgactcacccagggcagacaaacttgaccctggaacccttg

SEQ ID NO: 500 - DNA, 50 bp, synthetic construct
  misc_feature 1..50: note = Synthetic
  source 1..50: mol_type = other DNA
ctcatcagttctcacccccaatgtcccccggatttacctgagggacgggc

SEQ ID NO: 501 - DNA, 50 bp, synthetic construct
  misc_feature 1..50: note = Synthetic
  source 1..50: mol_type = other DNA
cccatggtgcacgcaccatggtttgggctcttccgcgttcgctcgccgct

SEQ ID NO: 502 - DNA, 50 bp, synthetic construct; identical to SEQ ID NO: 443
  misc_feature 1..50: note = Synthetic
  source 1..50: mol_type = other DNA

SEQ ID NO: 503 - DNA, 50 bp, synthetic construct
  misc_feature 1..50: note = Synthetic
  source 1..50: mol_type = other DNA
accccatcaattaaccttccggcaccgggcaggcgtcacaccgtatacgt

SEQ ID NO: 504 - DNA, 50 bp, synthetic construct
  misc_feature 1..50: note = Synthetic
  source 1..50: mol_type = other DNA
cattccggcattctcactcgaatacaatccaccgctgcttccgctacgac

SEQ ID NO: 505 - DNA, 50 bp, synthetic construct
  misc_feature 1..50: note = Synthetic
  source 1..50: mol_type = other DNA
gtttcagttcgccgggtacctctcttgcaggccatgtattcacctgcaga

SEQ ID NO: 506 - DNA, 50 bp, synthetic construct
  misc_feature 1..50: note = Synthetic
  source 1..50: mol_type = other DNA
acctgaggctactcgcctcgactacctgtgtcggtttgcggtacgggtag

SEQ ID NO: 507 - DNA, 50 bp, synthetic construct
  misc_feature 1..50: note = Synthetic
  source 1..50: mol_type = other DNA
aaggctagccctaaagctatttcgaggagaaccagctatctccgggttcg

SEQ ID NO: 508 - DNA, 50 bp, synthetic construct
  misc_feature 1..50: note = Synthetic
  source 1..50: mol_type = other DNA
attattattttctcctcctacgggtactgagatgtttcacttccccgcgt

SEQ ID NO: 509 - DNA, 50 bp, synthetic construct; identical to SEQ ID NO: 191
  misc_feature 1..50: note = Synthetic
  source 1..50: mol_type = other DNA

SEQ ID NO: 510 - DNA, 50 bp, synthetic construct
  misc_feature 1..50: note = Synthetic
  source 1..50: mol_type = other DNA
cagaggtctgtccaacacggtcctctcgtactagtgtcagagccacgcaa

SEQ ID NO: 511 - DNA, 50 bp, synthetic construct
  misc_feature 1..50: note = Synthetic
  source 1..50: mol_type = other DNA
atcctctcagaccagttacggatcgtcgccttggtaggcctttaccccac

SEQ ID NO: 512 - DNA, 50 bp, synthetic construct
  misc_feature 1..50: note = Synthetic
  source 1..50: mol_type = other DNA
tcacgcagaattcctcgtgctccgcgctactcaggataccactaggcttc

SEQ ID NO: 513 - DNA, 50 bp, synthetic construct
  misc_feature 1..50: note = Synthetic
  source 1..50: mol_type = other DNA
cgcgtcttcggtggcgtgcttgagccccgctacattgtcggcgcggaacc

SEQ ID NO: 514 - DNA, 50 bp, synthetic construct
  misc_feature 1..50: note = Synthetic
  source 1..50: mol_type = other DNA
tacttatgcccgattattatccacgccaaactcctcgactagtgagctgt

SEQ ID NO: 515 - DNA, 50 bp, synthetic construct
  misc_feature 1..50: note = Synthetic
  source 1..50: mol_type = other DNA
accgtagtgcctcgtcatcacgcctcagccttgattttccggatttgcct

SEQ ID NO: 516 - DNA, 50 bp, synthetic construct
  misc_feature 1..50: note = Synthetic
  source 1..50: mol_type = other DNA
agctgacgcctgtatttcccagtctcccacctatcctgtacatgaaatac

SEQ ID NO: 517 - DNA, 50 bp, synthetic construct
  misc_feature 1..50: note = Synthetic
  source 1..50: mol_type = other DNA
ggcgttgctgatccgcgattactagcgactccgccttcacggagccgggt

SEQ ID NO: 518 - DNA, 50 bp, synthetic construct
  misc_feature 1..50: note = Synthetic
  source 1..50: mol_type = other DNA
gggtgccgcatgggttaagcttagcggattttctcgggagtatggttacc

SEQ ID NO: 519 - DNA, 50 bp, synthetic construct
  misc_feature 1..50: note = Synthetic
  source 1..50: mol_type = other DNA
tcttcagccccaggatgcgatgagccgacatcgaggtgccaaacttcctc

SEQ ID NO: 520 - DNA, 50 bp, synthetic construct
  misc_feature 1..50: note = Synthetic
  source 1..50: mol_type = other DNA
cgccggcaccggatcactatctccgactttcgtccctgctcgatccgtcg

SEQ ID NO: 521 - DNA, 50 bp, synthetic construct
  misc_feature 1..50: note = Synthetic
  source 1..50: mol_type = other DNA
cacactatccgtctccgtcactccttcgctccatatacgggtgcaggaat

SEQ ID NO: 522 - DNA, 50 bp, synthetic construct
  misc_feature 1..50: note = Synthetic
  source 1..50: mol_type = other DNA
actgtcaggttcgactcttcctgcggatttgcctgcaggaatcaacatct

SEQ ID NO: 523 - DNA, 50 bp, synthetic construct
  misc_feature 1..50: note = Synthetic
  source 1..50: mol_type = other DNA
tctttcggcgagggggtttcccaccccctttatcgttacttatacctaca

SEQ ID NO: 524 - DNA, 50 bp, synthetic construct
  misc_feature 1..50: note = Synthetic
  source 1..50: mol_type = other DNA
cttttcagtgctctacaggacacatccatcacctgaggctgtacctcaat

SEQ ID NO: 525 - DNA, 50 bp, synthetic construct
  misc_feature 1..50: note = Synthetic
  source 1..50: mol_type = other DNA
atgaccctccccggttgagccgggggctttcacatcagacttaagaaacc

SEQ ID NO: 526 - DNA, 50 bp, synthetic construct
  misc_feature 1..50: note = Synthetic
  source 1..50: mol_type = other DNA
tttcacaactgacttaaatatccatctacgctccctttaaacccaataaa

SEQ ID NO: 527 - DNA, 50 bp, synthetic construct
  misc_feature 1..50: note = Synthetic
  source 1..50: mol_type = other DNA
ctacttattttcggtcccttacgcccgggtcaaccaacgcccgggtccag

SEQ ID NO: 528 - DNA, 50 bp, synthetic construct
  misc_feature 1..50: note = Synthetic
  source 1..50: mol_type = other DNA
gtatttaggcttaccgggtggtcccggcagattcacagcagattccacga

SEQ ID NO: 529 - DNA, 50 bp, synthetic construct
  misc_feature 1..50: note = Synthetic
  source 1..50: mol_type = other DNA
cttcaacctggacatggataggtcacccggtttcgggtctgcacacactg

SEQ ID NO: 530 - DNA, 50 bp, synthetic construct
  misc_feature 1..50: note = Synthetic
  source 1..50: mol_type = other DNA
tccggaagccacgcctcaagggcacaacctccaagtcgacatcgtttacg

SEQ ID NO: 531 - DNA, 50 bp, synthetic construct
  misc_feature 1..50: note = Synthetic
  source 1..50: mol_type = other DNA
ggtcacccggtttcgggcccattgtatgcaacttaacgcccttttcaaac

SEQ ID NO: 532 - DNA, 50 bp, synthetic construct
  misc_feature 1..50: note = Synthetic
  source 1..50: mol_type = other DNA
ggctacacattttaaaatgcttaaccttgccggaaaaagtaactcgtagg

SEQ ID NO: 533 - DNA, 50 bp, synthetic construct
  misc_feature 1..50: note = Synthetic
  source 1..50: mol_type = other DNA
caaatttcctgcgcccgcgacggatagggaccgaactgtctcacgacgtt

SEQ ID NO: 534 - DNA, 50 bp, synthetic construct
  misc_feature 1..50: note = Synthetic
  source 1..50: mol_type = other DNA
gccagggtagtatcccaccgatgcctccaccgaagctggcgctccggttt

SEQ ID NO: 535 - DNA, 50 bp, synthetic construct
  misc_feature 1..50: note = Synthetic
  source 1..50: mol_type = other DNA
ttcactgaagggtaacaccccataacaggtgccaggtttccccattcgga

SEQ ID NO: 536 - DNA, 50 bp, synthetic construct
  misc_feature 1..50: note = Synthetic
  source 1..50: mol_type = other DNA
tccagctaatcagacgcgggtccatcttataccaccggagtttttcacac

SEQ ID NO: 537 - DNA, 50 bp, synthetic construct
  misc_feature 1..50: note = Synthetic
  source 1..50: mol_type = other DNA
ctttatgaatatgcttagcggattttcttgggagcctgattacgtccatt

SEQ ID NO: 538 - DNA, 50 bp, synthetic construct
  misc_feature 1..50: note = Synthetic
  source 1..50: mol_type = other DNA
catcaggtagtattcaggcttaccaggtggtcctggcagattcacacgaa

SEQ ID NO: 539 - DNA, 50 bp, synthetic construct
  misc_feature 1..50: note = Synthetic
  source 1..50: mol_type = other DNA
catgcaccacggatttgcctatgatgcgcgctgcgtgcttgaccacggaa

SEQ ID NO: 540 - DNA, 50 bp, synthetic construct
  misc_feature 1..50: note = Synthetic
  source 1..50: mol_type = other DNA
gacagcctggccatcattacgccattcgtgcaggtcggaacttacccgac

SEQ ID NO: 541 - DNA, 50 bp, synthetic construct
  misc_feature 1..50: note = Synthetic
  source 1..50: mol_type = other DNA
tcactgctttaagcagctccgaccgcttgtaggcgcacggtttcaggaac

SEQ ID NO: 542 - DNA, 50 bp, synthetic construct
  misc_feature 1..50: note = Synthetic
  source 1..50: mol_type = other DNA
gctcccaacaccacgcggcgataccaacccgaaggaaggaaccaccacga

SEQ ID NO: 543 - DNA, 50 bp, synthetic construct
  misc_feature 1..50: note = Synthetic
  source 1..50: mol_type = other DNA
gacttcccattccattccactaaacctttacaataccgttttctgtccga

SEQ ID NO: 544 - DNA, 50 bp, synthetic construct
  misc_feature 1..50: note = Synthetic
  source 1..50: mol_type = other DNA
acttaacgacccgtctgcgctccctttaaacccaataaatccggataacg

SEQ ID NO: 545 - DNA, 50 bp, synthetic construct
  misc_feature 1..50: note = Synthetic
  source 1..50: mol_type = other DNA
ggggtgggtttcatacttagatgctttcagcagttatccgctccgcactt

SEQ ID NO: 546 - DNA, 50 bp, synthetic construct
  misc_feature 1..50: note = Synthetic
  source 1..50: mol_type = other DNA
gaaatcctcggatcaaagccctgctggcggctccccgaggcatatcgcag

SEQ ID NO: 547 - DNA, 50 bp, synthetic construct
  misc_feature 1..50: note = Synthetic
  source 1..50: mol_type = other DNA
ctttcatggcccctactgatcatcgccttggtaggccattaccctaccaa

SEQ ID NO: 548 - DNA, 50 bp, synthetic construct
  misc_feature 1..50: note = Synthetic
  source 1..50: mol_type = other DNA
ctgttatccccagggtaacttttatccgttgagcgatggcatttccactc

SEQ ID NO: 549 - DNA, 50 bp, synthetic construct
  misc_feature 1..50: note = Synthetic
  source 1..50: mol_type = other DNA
cctaccctcagctcatccagaagcttttcaacgcttattggtgcggtcct

SEQ ID NO: 550 - DNA, 50 bp, synthetic construct
  misc_feature 1..50: note = Synthetic
  source 1..50: mol_type = other DNA
accaagaaggtgctccgaccgcttgtaggcacatggtttcaggaactatt

SEQ ID NO: 551 - DNA, 50 bp, synthetic construct; identical to SEQ ID NO: 489
  misc_feature 1..50: note = Synthetic
  source 1..50: mol_type = other DNA

SEQ ID NO: 552 - DNA, 50 bp, synthetic construct
  misc_feature 1..50: note = Synthetic
  source 1..50: mol_type = other DNA
cctggccaagggtagatcacttggtttcgcgtctgccactgccgactata

SEQ ID NO: 553 - DNA, 50 bp, synthetic construct
  misc_feature 1..50: note = Synthetic
  source 1..50: mol_type = other DNA
gggggtctcccttatgccgaaggcacgggagcaatttgccgagttccttg

SEQ ID NO: 554 - DNA, 50 bp, synthetic construct
  misc_feature 1..50: note = Synthetic
  source 1..50: mol_type = other DNA
catggtttagccccgttacatcttccgcgcaggccgactcgaccagtgag

SEQ ID NO: 555 - DNA, 50 bp, synthetic construct
  misc_feature 1..50: note = Synthetic
  source 1..50: mol_type = other DNA
atccgccgccttttcaacggaggtcggttcggtcctccatggaattttac

SEQ ID NO: 556 - DNA, 50 bp, synthetic construct
  misc_feature 1..50: note = Synthetic
  source 1..50: mol_type = other DNA
ccaaagtcaatgctaagctgtagtaaaggttcacggggtcttttcgtccc

SEQ ID NO: 557 - DNA, 50 bp, synthetic construct
  misc_feature 1..50: note = Synthetic
  source 1..50: mol_type = other DNA
aaagttcggtggttacggaatttctaccgtatgtgcatcgactacgccgt

SEQ ID NO: 558 - DNA, 50 bp, synthetic construct
  misc_feature 1..50: note = Synthetic
  source 1..50: mol_type = other DNA
caggtgtcagcccctatacttcatctttcgatttagcagagacctgtgtt

SEQ ID NO: 559 - DNA, 50 bp, synthetic construct
  misc_feature 1..50: note = Synthetic
  source 1..50: mol_type = other DNA
acttaaagccagcgccccttctcccgaagttacggggccattttgccgag

SEQ ID NO: 560 - DNA, 50 bp, synthetic construct
  misc_feature 1..50: note = Synthetic
  source 1..50: mol_type = other DNA
acttagatgctttcagcacttatccgatccagacttagatacccggcaat

SEQ ID NO: 561 - DNA, 50 bp, synthetic construct
  misc_feature 1..50: note = Synthetic
  source 1..50: mol_type = other DNA
ctacaggatttagtttagcggattttcttggcagcatgattacatgcact

SEQ ID NO: 562 - DNA, 50 bp, synthetic construct
  misc_feature 1..50: note = Synthetic
  source 1..50: mol_type = other DNA
ccttaaccttccggcactgggcaggtgtcagcccgtatacgtcgtatctc

SEQ ID NO: 563 - DNA, 50 bp, synthetic construct
  misc_feature 1..50: note = Synthetic
  source 1..50: mol_type = other DNA
tgagccaacatcctagttgtcttcgaaatcccacatccttttccacttaa

SEQ ID NO: 564 - DNA, 50 bp, synthetic construct
  misc_feature 1..50: note = Synthetic
  source 1..50: mol_type = other DNA
caggatgtgacgagccgacatcgaggtgccaaacccctccgtcgatatga

SEQ ID NO: 565 - DNA, 50 bp, synthetic construct
  misc_feature 1..50: note = Synthetic
  source 1..50: mol_type = other DNA
ggttttgccggtccatggtcggtacgggaatatccacccgttcatccatt

SEQ ID NO: 566 - DNA, 50 bp, synthetic construct
  misc_feature 1..50: note = Synthetic
  source 1..50: mol_type = other DNA
ctttacgctatcggtcattgggtagtatttaggcttggagggtggtcccc

SEQ ID NO: 567 - DNA, 50 bp, synthetic construct
  misc_feature 1..50: note = Synthetic
  source 1..50: mol_type = other DNA
gcatggattaagtttagcggattttctaggaagtatgattacctacgcta

SEQ ID NO: 568 - DNA, 50 bp, synthetic construct
  misc_feature 1..50: note = Synthetic
  source 1..50: mol_type = other DNA
actgtccatcctctggtttcacagagctatgttagaatttcagtaaccga

SEQ ID NO: 569 - DNA, 50 bp, synthetic construct
  misc_feature 1..50: note = Synthetic
  source 1..50: mol_type = other DNA
acctcgcggtacgccttcgacgccgactggaatgctcccctaccgatcat

SEQ ID NO: 570 - DNA, 50 bp, synthetic construct
  misc_feature 1..50: note = Synthetic
  source 1..50: mol_type = other DNA
ctcttgcgatgagctctcctcttaaccttccagcaccgggcaggtgtcag

SEQ ID NO: 571 - DNA, 50 bp, synthetic construct
  misc_feature 1..50: note = Synthetic
  source 1..50: mol_type = other DNA
agctgacgccttggcttcccagtctcccacctatcctgtacatgtaatac

SEQ ID NO: 572 - DNA, 50 bp, synthetic construct
  misc_feature 1..50: note = Synthetic
  source 1..50: mol_type = other DNA
gaatgaatggctgcttccaagccaacatcctagctgtcactgggaccaga

SEQ ID NO: 573 - DNA, 50 bp, synthetic construct
  misc_feature 1..50: note = Synthetic
  source 1..50: mol_type = other DNA
tgagccaacatcctggttgtctacgtatcttcacatcgttttccacttaa

SEQ ID NO: 574 - DNA, 50 bp, synthetic construct
  misc_feature 1..50: note = Synthetic
  source 1..50: mol_type = other DNA
tgagggcacctttagaagcctccgttacgcttttggaggcgaccacccca

SEQ ID NO: 575 - DNA, 50 bp, synthetic construct
  misc_feature 1..50: note = Synthetic
  source 1..50: mol_type = other DNA
ttaaatcgaccgaagtttcaataaagtaattcccgttcgacttgcatgtg

SEQ ID NO: 576 - DNA, 50 bp, synthetic construct
  misc_feature 1..50: note = Synthetic
  source 1..50: mol_type = other DNA
agtcgggttgcagactccaatccgaactgagagaggctttagggattagc

SEQ ID NO: 577 - DNA, 50 bp, synthetic construct
  misc_feature 1..50: note = Synthetic
  source 1..50: mol_type = other DNA
cctgtgtcggtttacggtacgggtatggtatgaacaatagcggcttttct

SEQ ID NO: 578 - DNA, 50 bp, synthetic construct
  misc_feature 1..50: note = Synthetic
  source 1..50: mol_type = other DNA
ctcccggattccgacggaatttcacgtgttccgccgtactcaggatccac

SEQ ID NO: 579 - DNA, 50 bp, synthetic construct
  misc_feature 1..50: note = Synthetic
  source 1..50: mol_type = other DNA
aaacattaaagggtggtatttcaaggtcggctccatgcagactggcgtcc

SEQ ID NO: 580 - DNA, 50 bp, synthetic construct
  misc_feature 1..50: note = Synthetic
  source 1..50: mol_type = other DNA
cctgagtatattcaacccgactacgtgtgtccgtttacggtacgggtacc

SEQ ID NO: 581 - DNA, 50 bp, synthetic construct; identical to SEQ ID NO: 172
  misc_feature 1..50: note = Synthetic
  source 1..50: mol_type = other DNA

SEQ ID NO: 582 - DNA, 50 bp, synthetic construct
  misc_feature 1..50: note = Synthetic
  source 1..50: mol_type = other DNA
agtgagctattacgcactcttttaatgagtggctgcttctaagccaacat

SEQ ID NO: 583 - DNA, 50 bp, synthetic construct
  misc_feature 1..50: note = Synthetic
  source 1..50: mol_type = other DNA
ggctcacgccccgccttcaacgccgagtggaatgctcccctaccgatgat

SEQ ID NO: 584 - DNA, 50 bp, synthetic construct
  misc_feature 1..50: note = Synthetic
  source 1..50: mol_type = other DNA
agggcacctttagaagcctccgttacacttttggaggcgaccaccccagt

SEQ ID NO: 585 - DNA, 50 bp, synthetic construct
  misc_feature 1..50: note = Synthetic
  source 1..50: mol_type = other DNA
ctctgccatcgccatcgccgttcggcttagacttaggacccgactgaccc

SEQ ID NO: 586 - DNA, 50 bp, synthetic construct
  misc_feature 1..50: note = Synthetic
  source 1..50: mol_type = other DNA
gccgagttccttaacaagggttctcccgctcgtcttaggattctctcctc

SEQ ID NO: 587 - DNA, 50 bp, synthetic construct
  misc_feature 1..50: note = Synthetic
  source 1..50: mol_type = other DNA
ctccccccccccccttcccctccgcggccacctttccccccccctcccca

SEQ ID NO: 588 - DNA, 50 bp, synthetic construct
  misc_feature 1..50: note = Synthetic
  source 1..50: mol_type = other DNA
cccatatacacgggttagaatccaaacaaatgaagggtcgtatttcaaca

SEQ ID NO: 589 - DNA, 50 bp, synthetic construct
  misc_feature 1..50: note = Synthetic
  source 1..50: mol_type = other DNA
cccgcatcagcgggttagaactcaaataatcaaagggccgtatttcaaca

SEQ ID NO: 590 - DNA, 50 bp, synthetic construct; identical to SEQ ID NO: 316
  misc_feature 1..50: note = Synthetic
  source 1..50: mol_type = other DNA

SEQ ID NO: 591 - DNA, 50 bp, synthetic construct
  misc_feature 1..50: note = Synthetic
  source 1..50: mol_type = other DNA
cattcccacttaataccaccggatcactaagccctactttcgtacctgct

SEQ ID NO: 592 - DNA, 50 bp, synthetic construct
  misc_feature 1..50: note = Synthetic
  source 1..50: mol_type = other DNA
cttccgtcgccccgcggtggtttcactgctccgtctccacgtcgccccat

SEQ ID NO: 593 - DNA, 50 bp, synthetic construct
  misc_feature 1..50: note = Synthetic
  source 1..50: mol_type = other DNA
gcgggtaacctgcatcttcacaggtactaaaatttcaccgagtctctcgt

SEQ ID NO: 594 - DNA, 50 bp, synthetic construct
  misc_feature 1..50: note = Synthetic
  source 1..50: mol_type = other DNA
aaaagtacgcggttgagctaataatgctcttccacagcttgtaaacacag

SEQ ID NO: 595 - DNA, 50 bp, synthetic construct
  misc_feature 1..50: note = Synthetic
  source 1..50: mol_type = other DNA
cggtacgggaatatcaacccgttcatccattcgactacgcctgtcggcct

SEQ ID NO: 596 - DNA, 50 bp, synthetic construct
  misc_feature 1..50: note = Synthetic
  source 1..50: mol_type = other DNA
cctcatctacctgtgtcggtttgcggtacgggcgccttagtatacctcat

SEQ ID NO: 597 - DNA, 50 bp, synthetic construct
  misc_feature 1..50: note = Synthetic
  source 1..50: mol_type = other DNA
gtagtatttagccttggagggtggtccctcctgcttcccacagggtttca

SEQ ID NO: 598 - DNA, 50 bp, synthetic construct
  misc_feature 1..50: note = Synthetic
  source 1..50: mol_type = other DNA
ttccgtcaggtggcggcacttacgttccttcgtctctccatcgaggtata

SEQ ID NO: 599 - DNA, 50 bp, synthetic construct
  misc_feature 1..50: note = Synthetic
  source 1..50: mol_type = other DNA
cttcaaagtctccggcctatcctacacatcaattacccaaattcaatgtt

SEQ ID NO: 600 - DNA, 50 bp, synthetic construct
  misc_feature 1..50: note = Synthetic
  source 1..50: mol_type = other DNA
ctctcagggctcttactaactgaacgttatgggaaatctcatcttgaggg

SEQ ID NO: 601 - DNA, 50 bp, synthetic construct
  misc_feature 1..50: note = Synthetic
  source 1..50: mol_type = other DNA
aagtcctcgagcgattagtattggtccgcttcacgtctcacaacgcttcc

SEQ ID NO: 602 - DNA, 50 bp, synthetic construct
  misc_feature 1..50: note = Synthetic
  source 1..50: mol_type = other DNA
acgcctttcgtgcaggtcggaacttacccgacaaggaatttcgctacctt

SEQ ID NO: 603 - DNA, 50 bp, synthetic construct
  misc_feature 1..50: note = Synthetic
  source 1..50: mol_type = other DNA
cctgatcgacttgtatgtctcccagtcaagcgcccttatgccattacact

SEQ ID NO: 604 - DNA, 50 bp, synthetic construct
  misc_feature 1..50: note = Synthetic
  source 1..50: mol_type = other DNA
cgttttccacttagcatgtattagggaccttagctgtgggtctgggctgt

SEQ ID NO: 605 - DNA, 50 bp, synthetic construct
  misc_feature 1..50: note = Synthetic
  source 1..50: mol_type = other DNA
tagtcaagtatcgtctctcttcttccttgctgatagacctttacataccg

SEQ ID NO: 606 - DNA, 50 bp, synthetic construct
  misc_feature 1..50: note = Synthetic
  source 1..50: mol_type = other DNA
gacacatggttttctgcaactgccggccggcccgtcggagccggcgcacg

SEQ ID NO: 607 - DNA, 50 bp, synthetic construct
  misc_feature 1..50: note = Synthetic
  source 1..50: mol_type = other DNA
tttctcgtgtctcgtggtactctggatcccgccttgccgctcccggtttc

SEQ ID NO: 608 - DNA, 50 bp, synthetic construct
  misc_feature 1..50: note = Synthetic
  source 1..50: mol_type = other DNA
ctaatgagatgtttcagttcacagcgtttacctccaactagactatgaat

SEQ ID NO: 609 - DNA, 50 bp, synthetic construct
  misc_feature 1..50: note = Synthetic
  source 1..50: mol_type = other DNA
atcctttcccacttagcacgcgcttggggaccttagacgacgatctgggc

SEQ ID NO: 610 - DNA, 50 bp, synthetic construct
  misc_feature 1..50: note = Synthetic
  source 1..50: mol_type = other DNA
gtttcacgtgtctggccgtactctggaactcgctcagctcttgtcgtttt

SEQ ID NO: 611 - DNA, 50 bp, synthetic construct
  misc_feature 1..50: note = Synthetic
  source 1..50: mol_type = other DNA
atggttatagttaccaccgccgtttaccggggcttgaattcaccgcttcg

SEQ ID NO: 612 - DNA, 50 bp, synthetic construct
  misc_feature 1..50: note = Synthetic
  source 1..50: mol_type = other DNA
ccgcacggaatggccgtctcgtctcggggcgggcttcccgcttagatgct

SEQ ID NO: 613 - DNA, 50 bp, synthetic construct
  misc_feature 1..50: note = Synthetic
  source 1..50: mol_type = other DNA
tgctcgacttgtctgtctcgcagtcaagctcccttatacctttacactct

SEQ ID NO: 614 - DNA, 50 bp, synthetic construct
  misc_feature 1..50: note = Synthetic
  source 1..50: mol_type = other DNA
atgcattgccagaagcttttcctggaagccgtcatcatgtgcttcgctac

SEQ ID NO: 615 - DNA, 50 bp, synthetic construct
  misc_feature 1..50: note = Synthetic
  source 1..50: mol_type = other DNA
tcttgcggcgagcaggtttctcacctgctttatcgttacttatacctaca

SEQ ID NO: 616 - DNA, 50 bp, synthetic construct
  misc_feature 1..50: note = Synthetic
  source 1..50: mol_type = other DNA
cgcgcacgcaacccccgacgggtatcacgcgcacgcggtttggtctgatc

SEQ ID NO: 617 - DNA, 50 bp, synthetic construct
  misc_feature 1..50: note = Synthetic
  source 1..50: mol_type = other DNA
cgctttatcgttacttatgtcagcattcgcacttctgatacctccagcat

SEQ ID NO: 618 - DNA, 50 bp, synthetic construct
  misc_feature 1..50: note = Synthetic
  source 1..50: mol_type = other DNA
gacagtgcccaaatcattacgcctttcgtgcgggtcggaacttacccgac

SEQ ID NO: 619 - DNA, 50 bp, synthetic construct
  misc_feature 1..50: note = Synthetic
  source 1..50: mol_type = other DNA
tcccatctatcctgtgcatgcaacaccgaaacccaatattaggctacagt

SEQ ID NO: 620 - DNA, 50 bp, synthetic construct
  misc_feature 1..50: note = Synthetic
  source 1..50: mol_type = other DNA
cccgggtcatgccctttcagagtgtccctctgcttaaaactttcggtggt

SEQ ID NO: 621 - DNA, 50 bp, synthetic construct
  misc_feature 1..50: note = Synthetic
  source 1..50: mol_type = other DNA
gggatcccattcccggcttccgctctctgcacgtgtccccacagttctgt

SEQ ID NO: 622 - DNA, 50 bp, synthetic construct
  misc_feature 1..50: note = Synthetic
  source 1..50: mol_type = other DNA
cacctcgccatacacgccgcacggatttgcctatgcgactggctgcgtgc

SEQ ID NO: 623 - DNA, 50 bp, synthetic construct
  misc_feature 1..50: note = Synthetic
  source 1..50: mol_type = other DNA
tcgctcctcagcgtcagttacagaccagagagtcgccttcgccactggtg

SEQ ID NO: 624 - DNA, 50 bp, synthetic construct
  misc_feature 1..50: note = Synthetic
  source 1..50: mol_type = other DNA
tatcgaaccataacggctcccatcatcacacctcgccatgcatgccatgc

SEQ ID NO: 625 - DNA, 50 bp, synthetic construct
  misc_feature 1..50: note = Synthetic
  source 1..50: mol_type = other DNA
ttcaccggggcttcaattcggagcttgcacccctcctcttgaccttccgg

SEQ ID NO: 626 - DNA, 50 bp, synthetic construct
  misc_feature 1..50: note = Synthetic
  source 1..50: mol_type = other DNA
ctgcaggattaagtttagcggattttctcggcagcatgcttacgcgcact

SEQ ID NO: 627 - DNA, 50 bp, synthetic construct
  misc_feature 1..50: note = Synthetic
  source 1..50: mol_type = other DNA
tctcctaccatacctataaaggtatccacagcttcggtaatatgttttag

SEQ ID NO: 628 - DNA, 50 bp, synthetic construct
  misc_feature 1..50: note = Synthetic
  source 1..50: mol_type = other DNA
gggcgcgtcatgccctcacgtcgaggcttttctcggcagcataggatcac

SEQ ID NO: 629 - DNA, 50 bp, synthetic construct
  misc_feature 1..50: note = Synthetic
  source 1..50: mol_type = other DNA
ctccgacggattgtaggcgcacggtttcaggaactctttcactcccctcc

SEQ ID NO: 630 - DNA, 50 bp, synthetic construct
  misc_feature 1..50: note = Synthetic
  source 1..50: mol_type = other DNA
cactcgactagtgagctattacgcactctttgaatgaatagctgcttcta

SEQ ID NO: 631 - DNA, 50 bp, synthetic construct
  misc_feature 1..50: note = Synthetic
  source 1..50: mol_type = other DNA
actcccctcgccggggttcttttcgcctttccctcacggtactggttcac

SEQ ID NO: 632 - DNA, 50 bp, synthetic construct
  misc_feature 1..50: note = Synthetic
  source 1..50: mol_type = other DNA
ccctcccggggttcttttcacctttccctcacggtactatgcgctatcgg

SEQ ID NO: 633 - DNA, 50 bp, synthetic construct
  misc_feature 1..50: note = Synthetic
  source 1..50: mol_type = other DNA
ctggtcctctcgtactaggagcagatcctctcaaatttccttcgcccgcg

SEQ ID NO: 634 - DNA, 50 bp, synthetic construct
  misc_feature 1..50: note = Synthetic
  source 1..50: mol_type = other DNA
actttcgttactgctcgggccgtcaccctcgcagttaggctagcttttgc

SEQ ID NO: 635 - DNA, 50 bp, synthetic construct
  misc_feature 1..50: note = Synthetic
  source 1..50: mol_type = other DNA
tgtaatagccacgtaatttaaaactgaaattgagagagacttacccagag

SEQ ID NO: 636 - DNA, 50 bp, synthetic construct
  misc_feature 1..50: note = Synthetic
  source 1..50: mol_type = other DNA
ggtggtctaccgggagacttaccctcatgtgaggtgggaatactcatctt

SEQ ID NO: 637 - DNA, 50 bp, synthetic construct
  misc_feature 1..50: note = Synthetic
  source 1..50: mol_type = other DNA
tggcggtctgggctgtttccctttcgactacggatcttatcactcgcagt

SEQ ID NO: 638 - DNA, 50 bp, synthetic construct
  misc_feature 1..50: note = Synthetic
  source 1..50: mol_type = other DNA
tctccacatcactcttataggtagtacaggaatattaacctgttctgcca

SEQ ID NO: 639 - DNA, 50 bp, synthetic construct
  misc_feature 1..50: note = Synthetic
  source 1..50: mol_type = other DNA
ccattctgagggtacctttgggcgcctccgttactctttcggaggcgacc

SEQ ID NO: 640 - DNA, 50 bp, synthetic construct
  misc_feature 1..50: note = Synthetic
  source 1..50: mol_type = other DNA
gatggcaggactgtcacttctccgtctccacatcgctccataaagtagta

SEQ ID NO: 641 - DNA, 50 bp, synthetic construct
  misc_feature 1..50: note = Synthetic
  source 1..50: mol_type = other DNA
tcggcgcagagtcactcgaccagtgagctattacgcactctttaaatggt

SEQ ID NO: 642 - DNA, 50 bp, synthetic construct
  misc_feature 1..50: note = Synthetic
  source 1..50: mol_type = other DNA
cgcggcatggctgcatcaggcttgcgcccattgtgcagtattccccactg

SEQ ID NO: 643 - DNA, 50 bp, synthetic construct
  misc_feature 1..50: note = Synthetic
  source 1..50: mol_type = other DNA
cggacatccttaatgacattcgcagtttgattgtattcagtaccccggga

SEQ ID NO: 644 - DNA, 50 bp, synthetic construct
  misc_feature 1..50: note = Synthetic
  source 1..50: mol_type = other DNA
taccggcattctcacttctaagcgctccaccagtccttccggtctggctt

SEQ ID NO: 645 - DNA, 50 bp, synthetic construct
  misc_feature 1..50: note = Synthetic
  source 1..50: mol_type = other DNA
ttcgggcctccattcagtgttacctgaacttcaccctggacatgggtaga

SEQ ID NO: 646 - DNA, 50 bp, synthetic construct
  misc_feature 1..50: note = Synthetic
  source 1..50: mol_type = other DNA
cggaggcgaccgccccagtcaaactccccgcctggcattgtcccaccgcc

SEQ ID NO: 647 - DNA, 50 bp, synthetic construct
  misc_feature 1..50: note = Synthetic
  source 1..50: mol_type = other DNA
accttttaggaggcgaccgccccagtcaaactgcccgtcagacactgtct

SEQ ID NO: 648 - DNA, 50 bp, synthetic construct
  misc_feature 1..50: note = Synthetic
  source 1..50: mol_type = other DNA
acagcccagccttccgttgtgcgtacttcactacacaacagcctcactgc

SEQ ID NO: 649 - DNA, 50 bp, synthetic construct
  misc_feature 1..50: note = Synthetic
  source 1..50: mol_type = other DNA
tcataccaccggagtttttacccctgcaccatgcggtgctgtggtcttat

SEQ ID NO: 650 - DNA, 50 bp, synthetic construct
  misc_feature 1..50: note = Synthetic
  source 1..50: mol_type = other DNA
cactcacccgaaggcttgctcccaaacaaaagaggtttacaacccgaagg

SEQ ID NO: 651 - DNA, 50 bp, synthetic construct
  misc_feature 1..50: note = Synthetic
  source 1..50: mol_type = other DNA
cgtcaattcatttgagttttaaccttgcggccgtactccccaggcggtcg

SEQ ID NO: 652 - DNA, 50 bp, synthetic construct
  misc_feature 1..50: note = Synthetic
  source 1..50: mol_type = other DNA
actttcgttcctgctcgacttgtcagtctcgcagtcaggctggcttgtgc

SEQ ID NO: 653 - DNA, 50 bp, synthetic construct
  misc_feature 1..50: note = Synthetic
  source 1..50: mol_type = other DNA
ccaccagggaggctccgacggtttgtgggcgcacggtttcaggaactgtt

SEQ ID NO: 654 - DNA, 50 bp, synthetic construct
  misc_feature 1..50: note = Synthetic
  source 1..50: mol_type = other DNA
actggcgtgcacgtctctttgtctcccacctatcctgtacatgtatgacc

SEQ ID NO: 655 - DNA, 50 bp, synthetic construct
  misc_feature 1..50: note = Synthetic
  source 1..50: mol_type = other DNA
tgatagcgtgaggtccgaagatcccccactttctccctcaggacgtatgc

SEQ ID NO: 656 - DNA, 50 bp, synthetic construct
  misc_feature 1..50: note = Synthetic
  source 1..50: mol_type = other DNA
aaatctttaatctctttcagatgtcttctagagacgtcattgggtattag

SEQ ID NO: 657 - DNA, 50 bp, synthetic construct
  misc_feature 1..50: note = Synthetic
  source 1..50: mol_type = other DNA
caccggggccccaagacccacacacaccaacaaacccgaaggcttagtgg

SEQ ID NO: 658 - DNA, 50 bp, synthetic construct
  misc_feature 1..50: note = Synthetic
  source 1..50: mol_type = other DNA
tacttttccaattttttttttttttttttttttttttttcttccaataaa

SEQ ID NO: 659 - DNA, 50 bp, synthetic construct
  misc_feature 1..50: note = Synthetic
  source 1..50: mol_type = other DNA
ctctgcctatccttctgtgtcactgcatccggttgctcggcggtatcgga

SEQ ID NO: 660 - DNA, 50 bp, synthetic construct
  misc_feature 1..50: note = Synthetic
  source 1..50: mol_type = other DNA
atgcctggcagttccctactctcgcatggggagaccccacactaccatcg

SEQ ID NO: 661 - DNA, 50 bp, synthetic construct
  misc_feature 1..50: note = Synthetic
  source 1..50: mol_type = other DNA
aacatcctggttgtctaagcaactccacatccttttccacttaacgtata

SEQ ID NO: 662 - DNA, 50 bp, synthetic construct
  misc_feature 1..50: note = Synthetic
  source 1..50: mol_type = other DNA
ctccggccgggcccgccaggacccggacacacgctccctcaacaccacgc

SEQ ID NO: 663 - DNA, 50 bp, synthetic construct
  misc_feature 1..50: note = Synthetic
  source 1..50: mol_type = other DNA
ttctctgcggctctttcgagcactccttattccgaagttacggagtcaat

SEQ ID NO: 664 - DNA, 50 bp, synthetic construct
  misc_feature 1..50: note = Synthetic
  source 1..50: mol_type = other DNA
ggcacagccctgtgtttttgttaaacagttgcctggaccgattctctgcg

SEQ ID NO: 665 - DNA, 50 bp, synthetic construct
  misc_feature 1..50: note = Synthetic
  source 1..50: mol_type = other DNA
tgctccccacgctttcgagcctcaacgtcagttactgtccagtaagccgc

SEQ ID NO: 666 - DNA, 50 bp, synthetic construct
  misc_feature 1..50: note = Synthetic
  source 1..50: mol_type = other DNA
atgcgtcccacggatttgcctatgggacgggctgcgtgcttgaccacgga

SEQ ID NO: 667 - DNA, 50 bp, synthetic construct
  misc_feature 1..50: note = Synthetic
  source 1..50: mol_type = other DNA
cccagacaaccatcgctggggttgagctacctccctgcgtccctccgcag

SEQ ID NO: 668 - DNA, 50 bp, synthetic construct
  misc_feature 1..50: note = Synthetic
  source 1..50: mol_type = other DNA
acgccgttaggcctcaccttagctcccgactgacctggagcggacgaacc

SEQ ID NO: 669 - DNA, 50 bp, synthetic construct
  misc_feature 1..50: note = Synthetic
  source 1..50: mol_type = other DNA
gcctttagccttaaccttgccagccggcgtaactcgccggaccgttctac

SEQ ID NO: 670 - DNA, 50 bp, synthetic construct
  misc_feature 1..50: note = Synthetic
  source 1..50: mol_type = other DNA
tggccgttcaacctctcagtccggctactgatcgtcgccatggtgagccg

SEQ ID NO: 671 - DNA, 50 bp, synthetic construct
  misc_feature 1..50: note = Synthetic
  source 1..50: mol_type = other DNA
cgctttcgctcgccactactcacggagtatcccttcctgcaggtactgag

SEQ ID NO: 672 - DNA, 50 bp, synthetic construct
  misc_feature 1..50: note = Synthetic
  source 1..50: mol_type = other DNA
aggacccgactcacccggggacgacgaacgtggccccggaacccttggtc

SEQ ID NO: 673 - DNA, 50 bp, synthetic construct
  misc_feature 1..50: note = Synthetic
  source 1..50: mol_type = other DNA
cattgcggaagattccccactgctgcctcccgtaggagtctggaccgtgt

SEQ ID NO: 674 - DNA, 50 bp, synthetic construct
  misc_feature 1..50: note = Synthetic
  source 1..50: mol_type = other DNA
gcatgtattaggcacgccgccagcgttcgtcctgagccaggatcaaactc

SEQ ID NO: 675 - DNA, 50 bp, synthetic construct
  misc_feature 1..50: note = Synthetic
  source 1..50: mol_type = other DNA
cccgttacccatcatcgccatggtaggcctttaccctaccatctagctaa

SEQ ID NO: 676 - DNA, 50 bp, synthetic construct
  misc_feature 1..50: note = Synthetic
  source 1..50: mol_type = other DNA
gccctcacccgattagtaacagtcagctccatgtgttgccacacttccac

SEQ ID NO: 677 - DNA, 50 bp, synthetic construct
  misc_feature 1..50: note = Synthetic
  source 1..50: mol_type = other DNA
accccaagtcatcccccggttttcaacccaggtgggttcggtcctccacg

SEQ ID NO: 678 - DNA, 50 bp, synthetic construct
  misc_feature 1..50: note = Synthetic
  source 1..50: mol_type = other DNA
cgccttaggacccgactaacccagggcggataaacctagccctggaaccc

SEQ ID NO: 679 - DNA, 50 bp, synthetic construct
  misc_feature 1..50: note = Synthetic
  source 1..50: mol_type = other DNA
ttccgtcttgccgcgggtacactgcatcttcacagcgagttcaatttcac

SEQ ID NO: 680 - DNA, 50 bp, synthetic construct
  misc_feature 1..50: note = Synthetic
  source 1..50: mol_type = other DNA
gtacgggtaacacagaaatatgcttagcgggttttcttgggagccggttt

SEQ ID NO: 681 - DNA, 50 bp, synthetic construct
  misc_feature 1..50: note = Synthetic
  source 1..50: mol_type = other DNA
aagctccatggggtctttccgtcttgtcgcgggtaaccggcatcttcacc

SEQ ID NO: 682 - DNA, 50 bp, synthetic construct
  misc_feature 1..50: note = Synthetic
  source 1..50: mol_type = other DNA
aactttattcccttatagaagcagtttacaacccatagggccgtcttcgt

SEQ ID NO: 683 - DNA, 50 bp, synthetic construct
  misc_feature 1..50: note = Synthetic
  source 1..50: mol_type = other DNA
gggcgggattcgcacccgcctctcgctactcatgtctgcattctcactcc

SEQ ID NO: 684 - DNA, 50 bp, synthetic construct
  misc_feature 1..50: note = Synthetic
  source 1..50: mol_type = other DNA
atactatcaggttcggatctcatggtggatttgcctgccatgatcgactc

SEQ ID NO: 685 - DNA, 50 bp, synthetic construct
  misc_feature 1..50: note = Synthetic
  source 1..50: mol_type = other DNA
acgccgtcgggcatataaagccctccgacagtttgtaaacacagggtttc

SEQ ID NO: 686 - DNA, 50 bp, synthetic construct
  misc_feature 1..50: note = Synthetic
  source 1..50: mol_type = other DNA
gcctatcgaccacgtgttctgcatggggtcttcagcggctcggggccgca

SEQ ID NO: 687 - DNA, 50 bp, synthetic construct
  misc_feature 1..50: note = Synthetic
  source 1..50: mol_type = other DNA
ggataagggttgcgctcgttgcgggacttaacccaacatttcacaacacg

SEQ ID NO: 688 - DNA, 50 bp, synthetic construct
  misc_feature 1..50: note = Synthetic
  source 1..50: mol_type = other DNA
gcccccgagccttggcagtgctctacacggcgtgaggttcatccgaggct

SEQ ID NO: 689 - DNA, 50 bp, synthetic construct
  misc_feature 1..50: note = Synthetic
  source 1..50: mol_type = other DNA
ttccttaaccaagaatctctcaacgccttagtatgttctacccgaccacg

SEQ ID NO: 690 - DNA, 50 bp, synthetic construct; identical to SEQ ID NO: 62
  misc_feature 1..50: note = Synthetic
  source 1..50: mol_type = other DNA

SEQ ID NO: 691 - DNA, 50 bp, synthetic construct
  misc_feature 1..50: note = Synthetic
  source 1..50: mol_type = other DNA
tactgtcaggttcgactcttgcaccggatttgcctggcacaatcaacatc

SEQ ID NO: 692 - DNA, 50 bp, synthetic construct
  misc_feature 1..50: note = Synthetic
  source 1..50: mol_type = other DNA
gccttcccatgccattctgctagataccttccataccgtgcgctgtccga

SEQ ID NO: 693 - DNA, 50 bp, synthetic construct
  misc_feature 1..50: note = Synthetic
  source 1..50: mol_type = other DNA
atgagccgacatcgaggtgccaaacaccgccgtcgatatgaactcttggg

SEQ ID NO: 694 - DNA, 50 bp, synthetic construct
  misc_feature 1..50: note = Synthetic
  source 1..50: mol_type = other DNA
ttcggctcaaagtccggatttgcctggacctctcatcacctacactcttc

SEQ ID NO: 695 - DNA, 50 bp, synthetic construct
  misc_feature 1..50: note = Synthetic
  source 1..50: mol_type = other DNA
acgcatttcaccgctacacgtggaattccactctcctcttctgcactcaa

SEQ ID NO: 696 - DNA, 50 bp, synthetic construct
  misc_feature 1..50: note = Synthetic
  source 1..50: mol_type = other DNA
tttccgtttcgcctacggggctctcaccctctctggccggtctttccaga

SEQ ID NO: 697 - DNA, 50 bp, synthetic construct
  misc_feature 1..50: note = Synthetic
  source 1..50: mol_type = other DNA
gccccggacaaccatcgccggggatgagctacctccctgcgtccctccgc

SEQ ID NO: 698 - DNA, 50 bp, synthetic construct
  misc_feature 1..50: note = Synthetic
  source 1..50: mol_type = other DNA
tgtcgcgggtaaccggcatcttcaccggtactacaatttcgccgggcggg

SEQ ID NO: 699 - DNA, 50 bp, synthetic construct
  misc_feature 1..50: note = Synthetic
  source 1..50: mol_type = other DNA
aagccctcgatctattagtacacacttgctgaatggatcgctccacttac

SEQ ID NO: 700 - DNA, 50 bp, synthetic construct
  misc_feature 1..50: note = Synthetic
  source 1..50: mol_type = other DNA
ccttggcaacagttctctcgctcacctcgggatactctccctgcccacct

SEQ ID NO: 701 - DNA, 50 bp, synthetic construct
  misc_feature 1..50: note = Synthetic
  source 1..50: mol_type = other DNA
tctccgccaaagccaaagccttggtttcccagagtcccatctatcctgtg

SEQ ID NO: 702 - DNA, 50 bp, synthetic construct
  misc_feature 1..50: note = Synthetic
  source 1..50: mol_type = other DNA
aggagtattcaggcttaccaggtggtcctggcagattcacacgagatttc

SEQ ID NO: 703 - DNA, 50 bp, synthetic construct
  misc_feature 1..50: note = Synthetic
  source 1..50: mol_type = other DNA
caggatgtgacgagccgacatcgaggtgccaaaccactccgtcgatatga

SEQ ID NO: 704 - DNA, 50 bp, synthetic construct; identical to SEQ ID NO: 151
  misc_feature 1..50: note = Synthetic
  source 1..50: mol_type = other DNA

SEQ ID NO: 705 - DNA, 50 bp, synthetic construct
  misc_feature 1..50: note = Synthetic
  source 1..50: mol_type = other DNA
tcagatggcggcactgccacgactccgtctccacgtcactccccaaggta

SEQ ID NO: 706 - DNA, 50 bp, synthetic construct
  misc_feature 1..50: note = Synthetic
  source 1..50: mol_type = other DNA
ctacggggccatcaccctctgcggcccggcattcaatccggttcgcctca

SEQ ID NO: 707 - DNA, 50 bp, synthetic construct
  misc_feature 1..50: note = Synthetic
  source 1..50: mol_type = other DNA
ccaggtcataaggggcatgatgatttgacgtcatccccaccttcctccgg

SEQ ID NO: 708 - DNA, 50 bp, synthetic construct
  misc_feature 1..50: note = Synthetic
  source 1..50: mol_type = other DNA
cctttaatcatgtgaacatgcggactcatgatgccatcttgtattaatct

SEQ ID NO: 709 - DNA, 50 bp, synthetic construct
  misc_feature 1..50: note = Synthetic
  source 1..50: mol_type = other DNA
ttttcacacctgacttaagatcccgccttaagcttccctttacacccagt

SEQ ID NO: 710 - DNA, 50 bp, synthetic construct; identical to SEQ ID NO: 549
  misc_feature 1..50: note = Synthetic
  source 1..50: mol_type = other DNA

SEQ ID NO: 711 - DNA, 50 bp, synthetic construct
  misc_feature 1..50: note = Synthetic
  source 1..50: mol_type = other DNA
gtcacactgagtatttaggcttaccgggtggtcccggcagattcacagca

SEQ ID NO: 712 - DNA, 50 bp, synthetic construct
  misc_feature 1..50: note = Synthetic
  source 1..50: mol_type = other DNA
ccaggataacttacgtacaccattcgacgccgtgagtatgctcccctacc

SEQ ID NO: 713 - DNA, 50 bp, synthetic construct
  misc_feature 1..50: note = Synthetic
  source 1..50: mol_type = other DNA
agagaaccagctatctccaagttcgtttggaatttctccgctacccacaa

SEQ ID NO: 714 - DNA, 50 bp, synthetic construct
  misc_feature 1..50: note = Synthetic
  source 1..50: mol_type = other DNA
cccgaagttacggggtaattttgccgagttccttaacaacccttctcccg

SEQ ID NO: 715 - DNA, 50 bp, synthetic construct
  misc_feature 1..50: note = Synthetic
  source 1..50: mol_type = other DNA
ggctcacgccccaccttcgacgcggagtggaatgctcccctaccgatgtt

SEQ ID NO: 716 - DNA, 50 bp, synthetic construct
  misc_feature 1..50: note = Synthetic
  source 1..50: mol_type = other DNA
gtatctaatcctgtttgctccccacgctttcgcactgagcgtcagtcttc

SEQ ID NO: 717 - DNA, 50 bp, synthetic construct
  misc_feature 1..50: note = Synthetic
  source 1..50: mol_type = other DNA
cgcgagtccatcctgaagcgaataaatccttttccctcagcaccatgcgg

SEQ ID NO: 718 - DNA, 50 bp, synthetic construct
  misc_feature 1..50: note = Synthetic
  source 1..50: mol_type = other DNA
ttatcgcagcttatcacgtctttcttcggctcttagtgccaaggcatcca

SEQ ID NO: 719 - DNA, 50 bp, synthetic construct
  misc_feature 1..50: note = Synthetic
  source 1..50: mol_type = other DNA
cggcaaagattctcactttgctctcgctactcatgccggcattctctctc

SEQ ID NO: 720 - DNA, 50 bp, synthetic construct
  misc_feature 1..50: note = Synthetic
  source 1..50: mol_type = other DNA
ccggcagaccgatcaagaaaaaacccacaaccccgcacgcgcaacccctg

SEQ ID NO: 721 - DNA, 50 bp, synthetic construct
  misc_feature 1..50: note = Synthetic
  source 1..50: mol_type = other DNA
gggctgtttcccttttgactatgagacttatctcacatagtctgactgct

SEQ ID NO: 722 - DNA, 50 bp, synthetic construct
  misc_feature 1..50: note = Synthetic
  source 1..50: mol_type = other DNA
ccccactgctgcctcccgtaggagtctggaccgtgtctcagttccagtgt

SEQ ID NO: 723 - DNA, 50 bp, synthetic construct
  misc_feature 1..50: note = Synthetic
  source 1..50: mol_type = other DNA
ttgtgactattctctgcggcctgctctcgcaggcaccccttatcccgaag

SEQ ID NO: 724 - DNA, 50 bp, synthetic construct
  misc_feature 1..50: note = Synthetic
  source 1..50: mol_type = other DNA
ttacctccacttcaacctggacatgggtaggtcacccggtttcgggtcga

SEQ ID NO: 725 - DNA, 50 bp, synthetic construct
  misc_feature 1..50: note = Synthetic
  source 1..50: mol_type = other DNA
tcgcaaggttatccccaagtgaagggcaggttggatacgcgttactcacc

SEQ ID NO: 726 - DNA, 50 bp, synthetic construct
  misc_feature 1..50: note = Synthetic
  source 1..50: mol_type = other DNA
cgcgatcggcagaccatgcgcgttcaggtacggggccctcaccctctgcg

SEQ ID NO: 727 - DNA, 50 bp, synthetic construct
  misc_feature 1..50: note = Synthetic
  source 1..50: mol_type = other DNA
gcctttcactcctacactcggctcatccagaagcttttcaacgcttattg

SEQ ID NO: 728 - DNA, 50 bp, synthetic construct
  misc_feature 1..50: note = Synthetic
  source 1..50: mol_type = other DNA
agtttgataaggttcagtaacctctcggcccctagccaattcagtgcttt

SEQ ID NO: 729 - DNA, 50 bp, synthetic construct
  misc_feature 1..50: note = Synthetic
  source 1..50: mol_type = other DNA
ggctgcaacacggtgacgtgaagcgaatcccaaaaaccatctctcagttc

SEQ ID NO: 730 - DNA, 50 bp, synthetic construct
  misc_feature 1..50: note = Synthetic
  source 1..50: mol_type = other DNA
ccggtctctcgactagtgagctgttacgcactctttgaatgaatggctgc

SEQ ID NO: 731 - DNA, 50 bp, synthetic construct
  misc_feature 1..50: note = Synthetic
  source 1..50: mol_type = other DNA
ggatcactaactccaactttcgttactgctcgaactgtcgctctcgcagt

SEQ ID NO: 732 - DNA, 50 bp, synthetic construct
  misc_feature 1..50: note = Synthetic
  source 1..50: mol_type = other DNA
ctcgcgtaccgctttaatgggcgaacagcccaacccttgggaccgactac

SEQ ID NO: 733 - DNA, 50 bp, synthetic construct
  misc_feature 1..50: note = Synthetic
  source 1..50: mol_type = other DNA
cggctacgcctttcggcctcaccttagctcccgactaacttggagcggac

SEQ ID NO: 734 - DNA, 50 bp, synthetic construct
  misc_feature 1..50: note = Synthetic
  source 1..50: mol_type = other DNA
acctttccctcacggtactggttcactatcggtcactagggagtatttag

SEQ ID NO: 735 - DNA, 50 bp, synthetic construct
  misc_feature 1..50: note = Synthetic
  source 1..50: mol_type = other DNA
atactgtcaggttcgactcttgcaccggatttgcctggcgcaatcagcat

SEQ ID NO: 736 - DNA, 50 bp, synthetic construct
  misc_feature 1..50: note = Synthetic
  source 1..50: mol_type = other DNA
tgtcatgctctatggtctttctttccagaaagttcttctccgatgtcttc

SEQ ID NO: 737 - DNA, 50 bp, synthetic construct
  misc_feature 1..50: note = Synthetic
  source 1..50: mol_type = other DNA
atcaccttaggattctctcctcgcctacctgtgtcggtttgcggtacggg

SEQ ID NO: 738 - DNA, 50 bp, synthetic construct
  misc_feature 1..50: note = Synthetic
  source 1..50: mol_type = other DNA
acgtattcaccgtggcattctgatccacgattactagcgattccgacttc

SEQ ID NO: 739 - DNA, 50 bp, synthetic construct
  misc_feature 1..50: note = Synthetic
  source 1..50: mol_type = other DNA
tagagcattttcttggaagcaggattacccacactattggtttactccga

SEQ ID NO: 740 - DNA, 50 bp, synthetic construct
  misc_feature 1..50: note = Synthetic
  source 1..50: mol_type = other DNA
cattgaccaatattcctcactgctgcctcccgtaggagtttgggccgtgt

SEQ ID NO: 741 - DNA, 50 bp, synthetic construct; identical to SEQ ID NO: 555
  misc_feature 1..50: note = Synthetic
  source 1..50: mol_type = other DNA

SEQ ID NO: 742 - DNA, 50 bp, synthetic construct
  misc_feature 1..50: note = Synthetic
  source 1..50: mol_type = other DNA
cctgtgtcggtttacggtacgggcgcatggcaaacgatagcggcttttct

SEQ ID NO: 743 - DNA, 50 bp, synthetic construct; identical to SEQ ID NO: 452
  misc_feature 1..50: note = Synthetic
  source 1..50: mol_type = other DNA

SEQ ID NO: 744 - DNA, 50 bp, synthetic construct
  misc_feature 1..50: note = Synthetic
  source 1..50: mol_type = other DNA
ggcggattttcccaaatccttcgactatcaagttctttggtaactcaaat

SEQ ID NO: 745 - DNA, 50 bp, synthetic construct
  misc_feature 1..50: note = Synthetic
  source 1..50: mol_type = other DNA
ctttcggggagtacgagctatctccgagtttgattggcctttcactccta

SEQ ID NO: 746 - DNA, 50 bp, synthetic construct
  misc_feature 1..50: note = Synthetic
  source 1..50: mol_type = other DNA
ctctagttagcctgctgcgtccctccttcactcaatactctagtacagga

SEQ ID NO: 747 - DNA, 50 bp, synthetic construct
  misc_feature 1..50: note = Synthetic
  source 1..50: mol_type = other DNA
cgccgtcgatgtgaactcttgggcgagatcagcctgttatccccagggta

SEQ ID NO: 748 - DNA, 50 bp, synthetic construct
  misc_feature 1..50: note = Synthetic
  source 1..50: mol_type = other DNA
agtcgtttccaactgttgtcccccactccagggcaggttactcacgcgtt

SEQ ID NO: 749 - DNA, 50 bp, synthetic construct
  misc_feature 1..50: note = Synthetic
  source 1..50: mol_type = other DNA
gcatgcttaaagttcggcggctacggaatttcaaccgtatgtgcatcgac

SEQ ID NO: 750 - DNA, 50 bp, synthetic construct
  misc_feature 1..50: note = Synthetic
  source 1..50: mol_type = other DNA
attaccgcggctgctggcacggaattagccggtccttattcttatggtac

SEQ ID NO: 751 - DNA, 50 bp, synthetic construct
  misc_feature 1..50: note = Synthetic
  source 1..50: mol_type = other DNA
cgcacagccctgtgtttttgttaaacagttgcctggacctattctctgcg

SEQ ID NO: 752 - DNA, 50 bp, synthetic construct
  misc_feature 1..50: note = Synthetic
  source 1..50: mol_type = other DNA
cataattttattttcttctcctacgggtactgagatgtttcacttccccg

SEQ ID NO: 753 - DNA, 50 bp, synthetic construct
  misc_feature 1..50: note = Synthetic
  source 1..50: mol_type = other DNA
accttgggcggacgaaccttccccaagaaaccttagattttcggccatta

SEQ ID NO: 754 - DNA, 50 bp, synthetic construct
  misc_feature 1..50: note = Synthetic
  source 1..50: mol_type = other DNA
tactatcaggttcggctctcaaggtggatttgcctgcctcgatctgcgcc

SEQ ID NO: 755 - DNA, 50 bp, synthetic construct
  misc_feature 1..50: note = Synthetic
  source 1..50: mol_type = other DNA
ctgtacatgcaataccaagctccagtaccaaactggagtaaagctccatg

SEQ ID NO: 756 - DNA, 50 bp, synthetic construct
  misc_feature 1..50: note = Synthetic
  source 1..50: mol_type = other DNA
tgcttgaccacggaaaaccacctccgcggccggctcccattccgtgtcac

SEQ ID NO: 757 - DNA, 50 bp, synthetic construct; identical to SEQ ID NO: 106
  misc_feature 1..50: note = Synthetic
  source 1..50: mol_type = other DNA

SEQ ID NO: 758 - DNA, 50 bp, synthetic construct
  misc_feature 1..50: note = Synthetic
  source 1..50: mol_type = other DNA
aagccaacatcctggttgtctacgcaattgcacatccttttccacttaac

SEQ ID NO: 759 - DNA, 50 bp, synthetic construct
  misc_feature 1..50: note = Synthetic
  source 1..50: mol_type = other DNA
cacatcttacgacggcagtctcgacagagtccccagcatcacctgatggt

SEQ ID NO: 760 - DNA, 50 bp, synthetic construct; identical to SEQ ID NO: 411
  misc_feature 1..50: note = Synthetic
  source 1..50: mol_type = other DNA

SEQ ID NO: 761 - DNA, 50 bp, synthetic construct
  misc_feature 1..50: note = Synthetic
  source 1..50: mol_type = other DNA
catctttactcgtactgcaatttcgccgagctcctggtcgagacagtggg

SEQ ID NO: 762 - DNA, 50 bp, synthetic construct
  misc_feature 1..50: note = Synthetic
  source 1..50: mol_type = other DNA
acaccgagccatgcagctctgtgcgcttatgcggtattagcagtcatttc

SEQ ID NO: 763 - DNA, 50 bp, synthetic construct
  misc_feature 1..50: note = Synthetic
  source 1..50: mol_type = other DNA
aggtcccgcgctccccaccaccgtccccgtcaaagacggggttcgggatg

SEQ ID NO: 764 - DNA, 50 bp, synthetic construct
  misc_feature 1..50: note = Synthetic
  source 1..50: mol_type = other DNA
atcgagctcacagcatgtgcatttttgtgtacggggctgtcaccctgtat

SEQ ID NO: 765 - DNA, 50 bp, synthetic construct
  misc_feature 1..50: note = Synthetic
  source 1..50: mol_type = other DNA
ggaatttctcccctagccacaagtcatccgctaacttttcaacggtagtc

SEQ ID NO: 766 - DNA, 50 bp, synthetic construct
  misc_feature 1..50: note = Synthetic
  source 1..50: mol_type = other DNA
gctctacctccaagactcttaccttgaggctagccctaaagctatttcgg

SEQ ID NO: 767 - DNA, 50 bp, synthetic construct
  misc_feature 1..50: note = Synthetic
  source 1..50: mol_type = other DNA
ttatagttacggccgccgtttactggggcttcaattcaatgcttctcttg

SEQ ID NO: 768 - DNA, 50 bp, synthetic construct; identical to SEQ ID NO: 529
  misc_feature 1..50: note = Synthetic
  source 1..50: mol_type = other DNA

SEQ ID NO: 769 - DNA, 50 bp, synthetic construct
  misc_feature 1..50: note = Synthetic
  source 1..50: mol_type = other DNA
gaggctagccctaaagctatttcgaggagaaccagctatctccgggttcg

SEQ ID NO: 770 - DNA, 50 bp, synthetic construct
  misc_feature 1..50: note = Synthetic
  source 1..50: mol_type = other DNA
tgggctgtttccctttcgactacggatcttagcactcgcagtctgactgc

SEQ ID NO: 771 - DNA, 50 bp, synthetic construct
  misc_feature 1..50: note = Synthetic
  source 1..50: mol_type = other DNA
ctccggcctatcctacacatcgattgcccaaattcaatgtaaagctatag

SEQ ID NO: 772 - DNA, 50 bp, synthetic construct
  misc_feature 1..50: note = Synthetic
  source 1..50: mol_type = other DNA
ccacttcacctaacaacaatgcaaaaagggcgtgccactggtagatgaca

SEQ ID NO: 773 - DNA, 50 bp, synthetic construct
  misc_feature 1..50: note = Synthetic
  source 1..50: mol_type = other DNA
accctcaggtcatccagaagcttttcaacgcttattggttcggtcctcca

SEQ ID NO: 774 - DNA, 50 bp, synthetic construct
  misc_feature 1..50: note = Synthetic
  source 1..50: mol_type = other DNA
agtatcccttcctgcaggtactgagatgtttcacttccctgcgtaccccc

SEQ ID NO: 775 - DNA, 50 bp, synthetic construct
  misc_feature 1..50: note = Synthetic
  source 1..50: mol_type = other DNA
acttggtatcccttcggctccgcaccttaagtgcttaacctcgccagtat

SEQ ID NO: 776 - DNA, 50 bp, synthetic construct
  misc_feature 1..50: note = Synthetic
  source 1..50: mol_type = other DNA
tcggatacgtgtgtcgtcacacttaaccttgccggcaaaggcaactcgta

SEQ ID NO: 777 - DNA, 50 bp, synthetic construct; identical to SEQ ID NO: 731
  misc_feature 1..50: note = Synthetic
  source 1..50: mol_type = other DNA

SEQ ID NO: 778 - DNA, 50 bp, synthetic construct
  misc_feature 1..50: note = Synthetic
  source 1..50: mol_type = other DNA
cgaacgccttagtattttcaacctgactacctgtgtcggtttggggtacg

SEQ ID NO: 779 - DNA, 50 bp, synthetic construct
  misc_feature 1..50: note = Synthetic
  source 1..50: mol_type = other DNA
ttctgcttctgcccgtacacgttgctcccctacccagaagtttccttctg

SEQ ID NO: 780 - DNA, 50 bp, synthetic construct
  misc_feature 1..50: note = Synthetic
  source 1..50: mol_type = other DNA
tcacggtactagttcgctatcggtcagacaggtatatctaggcttacccc

SEQ ID NO: 781 - DNA, 50 bp, synthetic construct
  misc_feature 1..50: note = Synthetic
  source 1..50: mol_type = other DNA
acttcttacaaagctccgaccgcttgtaggcgcatggtttcagggactat

SEQ ID NO: 782 - DNA, 50 bp, synthetic construct
  misc_feature 1..50: note = Synthetic
  source 1..50: mol_type = other DNA
tctttaaaggatggctgcttctgagccaacctcctagttgtctgggcatc

SEQ ID NO: 783 - DNA, 50 bp, synthetic construct
  misc_feature 1..50: note = Synthetic
  source 1..50: mol_type = other DNA
ccccattggggcccacaacaccgcacacacaacccctaccaagtatcaca

SEQ ID NO: 784 - DNA, 50 bp, synthetic construct
  misc_feature 1..50: note = Synthetic
  source 1..50: mol_type = other DNA
ctcaacttcaacctgctcatggctagatcacccggtttcgggtctgcaac

SEQ ID NO: 785 - DNA, 50 bp, synthetic construct
  misc_feature 1..50: note = Synthetic
  source 1..50: mol_type = other DNA
gcatacgccacacggcttatgctcgccacccgccactgactcgcagactc

SEQ ID NO: 786 - DNA, 50 bp, synthetic construct
  misc_feature 1..50: note = Synthetic
  source 1..50: mol_type = other DNA
gttcgtctatatgcccgcacctcactgcgccatgccggcagacatgacca

SEQ ID NO: 787 - DNA, 50 bp, synthetic construct
  misc_feature 1..50: note = Synthetic
  source 1..50: mol_type = other DNA
atctgggctgtttcccttttgacaatgacatttatctgacactgtctgac

SEQ ID NO: 788 - DNA, 50 bp, synthetic construct
  misc_feature 1..50: note = Synthetic
  source 1..50: mol_type = other DNA
ctattagtagcagtcagctccatgtgttaccacacttccacccctgccct

SEQ ID NO: 789 - DNA, 50 bp, synthetic construct
  misc_feature 1..50: note = Synthetic
  source 1..50: mol_type = other DNA
tttcacaactgacttaaacatccatctacgctccctttaaacccaataaa

SEQ ID NO: 790 - DNA, 50 bp, synthetic construct
  misc_feature 1..50: note = Synthetic
  source 1..50: mol_type = other DNA
ccgttgaattttcggcgcagagtcactcgaccagtgagctattacgcact

SEQ ID NO: 791 - DNA, 50 bp, synthetic construct
  misc_feature 1..50: note = Synthetic
  source 1..50: mol_type = other DNA
tccttaacgagagttcgctcgctcacctgaggctactcgcctcgactacc

SEQ ID NO: 792 - DNA, 50 bp, synthetic construct; identical to SEQ ID NO: 245
  misc_feature 1..50: note = Synthetic
  source 1..50: mol_type = other DNA

SEQ ID NO: 793 - DNA, 50 bp, synthetic construct
  misc_feature 1..50: note = Synthetic
  source 1..50: mol_type = other DNA
caacaggatgaagtttagcggattttctcgggagtatgattacatgcgct

SEQ ID NO: 794 - DNA, 50 bp, synthetic construct
  misc_feature 1..50: note = Synthetic
  source 1..50: mol_type = other DNA
gacgggctgcgtgcttgaccacggaaaaccacctccgcggccggctaccc

SEQ ID NO: 795 - DNA, 50 bp, synthetic construct
  misc_feature 1..50: note = Synthetic
  source 1..50: mol_type = other DNA
cggatttgcctatgatgcgcgctgcgtgcttgaccacggaaaaccacctc

SEQ ID NO: 796 - DNA, 50 bp, synthetic construct; identical to SEQ ID NO: 301
  misc_feature 1..50: note = Synthetic
  source 1..50: mol_type = other DNA

SEQ ID NO: 797 - DNA, 50 bp, synthetic construct
  misc_feature 1..50: note = Synthetic
  source 1..50: mol_type = other DNA
tgcagcacctgtctcacggttcccgaaggcacattctcatctctgaaaac

SEQ ID NO: 798 - DNA, 50 bp, synthetic construct
  misc_feature 1..50: note = Synthetic
  source 1..50: mol_type = other DNA
aggctagccctaaagctatttcggggagaaccagctatctccgagttcga

SEQ ID NO: 799 - DNA, 50 bp, synthetic construct
  misc_feature 1..50: note = Synthetic
  source 1..50: mol_type = other DNA
gacgtcctatctctaggattgtcagaggatgtcaagacctggtaaggttc

SEQ ID NO: 800 - DNA, 50 bp, synthetic construct
  misc_feature 1..50: note = Synthetic
  source 1..50: mol_type = other DNA
gttttgactacagggctgttacctcctatggcgggcctttccagacctct

SEQ ID NO: 801 - DNA, 50 bp, synthetic construct
  misc_feature 1..50: note = Synthetic
  source 1..50: mol_type = other DNA
ctggggcttcaattcagatcttcgctaacgctaaaccctcctcttaacct

SEQ ID NO: 802 - DNA, 50 bp, synthetic construct
  misc_feature 1..50: note = Synthetic
  source 1..50: mol_type = other DNA
ccttagtatattcaacccgactacgtgtgtccgtttacggtacgggtacc

SEQ ID NO: 803 - DNA, 50 bp, synthetic construct
  misc_feature 1..50: note = Synthetic
  source 1..50: mol_type = other DNA
ctatacatcatcttacgatttagcagagagctgtgtttttgataaacagt

SEQ ID NO: 804 - DNA, 50 bp, synthetic construct
  misc_feature 1..50: note = Synthetic
  source 1..50: mol_type = other DNA
ctaacaatgtcccccgactcgattcagagccgcaggttagaattccaata

SEQ ID NO: 805 - DNA, 50 bp, synthetic construct
  misc_feature 1..50: note = Synthetic
  source 1..50: mol_type = other DNA
tttggcctcttccgcgttcgctcgccactacttacggaatctcagttgat

SEQ ID NO: 806 - DNA, 50 bp, synthetic construct
  misc_feature 1..50: note = Synthetic
  source 1..50: mol_type = other DNA
cccgccaactggctaatcagacgcgggtccatcttataccaccggagttt

SEQ ID NO: 807 - DNA, 50 bp, synthetic construct
  misc_feature 1..50: note = Synthetic
  source 1..50: mol_type = other DNA
gctacttgggacacgcgatcggaagacggcaagcgtccaggtacggggct

SEQ ID NO: 808 - DNA, 50 bp, synthetic construct
  misc_feature 1..50: note = Synthetic
  source 1..50: mol_type = other DNA
catcaccggggatgagctacctcactgcgtccctccgcagcttgcctact

SEQ ID NO: 809 - DNA, 50 bp, synthetic construct
  misc_feature 1..50: note = Synthetic
  source 1..50: mol_type = other DNA
acaacttaatacccgattattatccacgccagactcctcgactagtgagc

SEQ ID NO: 810 - DNA, 50 bp, synthetic construct
  misc_feature 1..50: note = Synthetic
  source 1..50: mol_type = other DNA
ctctcagaccagttacggatcgtcgccttggtaggcctttaccccaccaa

SEQ ID NO: 811 - DNA, 50 bp, synthetic construct
  misc_feature 1..50: note = Synthetic
  source 1..50: mol_type = other DNA
tcacgtagtctgactgctgatcatcaattagccggcattcagagtttgat

SEQ ID NO: 812 - DNA, 50 bp, synthetic construct
  misc_feature 1..50: note = Synthetic
  source 1..50: mol_type = other DNA
taggtcacccggtttcgggtgtactgcatgcaactttacgcccttttcag

SEQ ID NO: 813 - DNA, 50 bp, synthetic construct
  misc_feature 1..50: note = Synthetic
  source 1..50: mol_type = other DNA
tactttagttcgctccacatcacggcttcgtctcatgcacagcggatttg

SEQ ID NO: 814 - DNA, 50 bp, synthetic construct
  misc_feature 1..50: note = Synthetic
  source 1..50: mol_type = other DNA
cttacggggctttcaccctctctggcaggctttcccaaaaacctttctgc

SEQ ID NO: 815 - DNA, 50 bp, synthetic construct
  misc_feature 1..50: note = Synthetic
  source 1..50: mol_type = other DNA
ggccgggctttcgatcccgttcttctatcctctctcttgccatatcatgg

SEQ ID NO: 816 - DNA, 50 bp, synthetic construct
  misc_feature 1..50: note = Synthetic
  source 1..50: mol_type = other DNA
acggcttctactcgtatacaacgctcccctaccactatagtttcctacaa

SEQ ID NO: 817 - DNA, 50 bp, synthetic construct
  misc_feature 1..50: note = Synthetic
  source 1..50: mol_type = other DNA
atcgagttttctttctcttcctccggctacttagatgtttcagttcaccg

SEQ ID NO: 818 - DNA, 50 bp, synthetic construct
  misc_feature 1..50: note = Synthetic
  source 1..50: mol_type = other DNA
gctttacataccgaaatacttcttcactcacgcggcgtcgctgcatcagg

SEQ ID NO: 819 - DNA, 50 bp, synthetic construct
  misc_feature 1..50: note = Synthetic
  source 1..50: mol_type = other DNA
tcccttctgcctttgcactcttctaatggtttccgaccattatgagggaa

SEQ ID NO: 820 - DNA, 50 bp, synthetic construct
  misc_feature 1..50: note = Synthetic
  source 1..50: mol_type = other DNA
ctccatcaggcagtttcccagacattactcacccgtccgccactcgtcag

SEQ ID NO: 821 - DNA, 50 bp, synthetic construct
  misc_feature 1..50: note = Synthetic
  source 1..50: mol_type = other DNA
tgccaaacctccccgtcgatgtgaactcttgggggagataagcctgttat

SEQ ID NO: 822 - DNA, 50 bp, synthetic construct
  misc_feature 1..50: note = Synthetic
  source 1..50: mol_type = other DNA
gcctggacctattctctgcgcctcacattactgtgaggaccctttatccc

SEQ ID NO: 823 - DNA, 50 bp, synthetic construct
  misc_feature 1..50: note = Synthetic
  source 1..50: mol_type = other DNA
acctttacacctgcatcctatcaacgtcgtagtctacaacgaccctcaga

SEQ ID NO: 824 - DNA, 50 bp, synthetic construct
  misc_feature 1..50: note = Synthetic
  source 1..50: mol_type = other DNA
gtattcattaacgctagaagcttttcttggcagagtgacatcactagctt

SEQ ID NO: 825 - DNA, 50 bp, synthetic construct; identical to SEQ ID NO: 255
  misc_feature 1..50: note = Synthetic
  source 1..50: mol_type = other DNA

SEQ ID NO: 826 - DNA, 50 bp, synthetic construct
  misc_feature 1..50: note = Synthetic
  source 1..50: mol_type = other DNA
aaaaaccctccccccccccccttcccctccgcggccacctttcccccccc

SEQ ID NO: 827 - DNA, 50 bp, synthetic construct
  misc_feature 1..50: note = Synthetic
  source 1..50: mol_type = other DNA
ctgtcggtacccgatacgggccctcaagcatccagtagctctaccccccg

SEQ ID NO: 828 - DNA, 50 bp, synthetic construct
  misc_feature 1..50: note = Synthetic
  source 1..50: mol_type = other DNA
atctacgcatttcaccgctacactaggaattccgcttacctctgttgcac

SEQ ID NO: 829 - DNA, 50 bp, synthetic construct
  misc_feature 1..50: note = Synthetic
  source 1..50: mol_type = other DNA
tctgtcccaccttcggcggctggctcctaaaaggttacctcaccgacttc

SEQ ID NO: 830 - DNA, 50 bp, synthetic construct
  misc_feature 1..50: note = Synthetic
  source 1..50: mol_type = other DNA
tgaccaagggtagatcacttggtttcgcgtctactccttccgactaatcg

SEQ ID NO: 831 - DNA, 50 bp, synthetic construct
  misc_feature 1..50: note = Synthetic
  source 1..50: mol_type = other DNA
tgtgcacttgcactcgccacccgattgccaaccgggctgagcggaccttt

SEQ ID NO: 832 - DNA, 50 bp, synthetic construct
  misc_feature 1..50: note = Synthetic
  source 1..50: mol_type = other DNA
cagcctcactcccaggctgtaaaatatgccccttcggagtttgataaggt

SEQ ID NO: 833 - DNA, 50 bp, synthetic construct
  misc_feature 1..50: note = Synthetic
  source 1..50: mol_type = other DNA
acgcttccactaacacacacactgattcaggctctgggctgctccccgtt

SEQ ID NO: 834 - DNA, 50 bp, synthetic construct
  misc_feature 1..50: note = Synthetic
  source 1..50: mol_type = other DNA
ctgtcaaggtcgactctccctgcggatttgcctacaggaatctacatcta

SEQ ID NO: 835 - DNA, 50 bp, synthetic construct
  misc_feature 1..50: note = Synthetic
  source 1..50: mol_type = other DNA
cctgtgtttttggtaaacagtcgctaccccctggcctgtgccaccccccg

SEQ ID NO: 836 - DNA, 50 bp, synthetic construct
  misc_feature 1..50: note = Synthetic
  source 1..50: mol_type = other DNA
atctgatagcgtgaggtccgaagatcccccactttctccctcaggacgta

SEQ ID NO: 837 - DNA, 50 bp, synthetic construct
  misc_feature 1..50: note = Synthetic
  source 1..50: mol_type = other DNA
acactttgggaccttagccggtggtctgggctctttcccttttgactacc

SEQ ID NO: 838 - DNA, 50 bp, synthetic construct
  misc_feature 1..50: note = Synthetic
  source 1..50: mol_type = other DNA
ctacaagggatcttacctgattgaatcagtgggatatcttatctttgggt

SEQ ID NO: 839 - DNA, 50 bp, synthetic construct
  misc_feature 1..50: note = Synthetic
  source 1..50: mol_type = other DNA
ctgaagggtaaccccacataaccagggccaggtttccccattcggacatc

SEQ ID NO: 840 - DNA, 50 bp, synthetic construct
  misc_feature 1..50: note = Synthetic
  source 1..50: mol_type = other DNA
tcagtccgcggcgctgtcacgcctccgtctccacgtcactccttaaggta

SEQ ID NO: 841 - DNA, 50 bp, synthetic construct
  misc_feature 1..50: note = Synthetic
  source 1..50: mol_type = other DNA
ttaacaagggttctcccgttcgtctcaggattctctcctcgcccacctgc

SEQ ID NO: 842 - DNA, 50 bp, synthetic construct
  misc_feature 1..50: note = Synthetic
  source 1..50: mol_type = other DNA
ctaacatcctagttgtctgtgcaaccccacatccttttccacttaacaat

SEQ ID NO: 843 - DNA, 50 bp, synthetic construct
  misc_feature 1..50: note = Synthetic
  source 1..50: mol_type = other DNA
gataaatctttcccccgtagggcacattcggtattactcccagtttcccg

SEQ ID NO: 844 - DNA, 50 bp, synthetic construct
  misc_feature 1..50: note = Synthetic
  source 1..50: mol_type = other DNA
gtttacaatccgaagaccttcttcccacacgcggcgttgctgcatcaggg

SEQ ID NO: 845 - DNA, 50 bp, synthetic construct
  misc_feature 1..50: note = Synthetic
  source 1..50: mol_type = other DNA
cggcgcactgcagctacctgtctgcgtcacccctgttaacacgcttgcct

SEQ ID NO: 846 - DNA, 50 bp, synthetic construct
  misc_feature 1..50: note = Synthetic
  source 1..50: mol_type = other DNA
atgaagctggaatcgctagtaatcgtatatcagcaatgatacggtgaata

SEQ ID NO: 847 - DNA, 50 bp, synthetic construct
  misc_feature 1..50: note = Synthetic
  source 1..50: mol_type = other DNA
cggatttgcctatgggacgggctgcgtgcttgaccacggaaaaccacctc

SEQ ID NO: 848 - DNA, 50 bp, synthetic construct
  misc_feature 1..50: note = Synthetic
  source 1..50: mol_type = other DNA
ggatgacccccttgccgaaacagtgctctacccccggagatgaattcacg

SEQ ID NO: 849 - DNA, 50 bp, synthetic construct
  misc_feature 1..50: note = Synthetic
  source 1..50: mol_type = other DNA
ggtacgggtaacatatactataacttagaagattttctcggaagtcgact

SEQ ID NO: 850 - DNA, 50 bp, synthetic construct
  misc_feature 1..50: note = Synthetic
  source 1..50: mol_type = other DNA
ctttgtaactccgtacagagtgtcctacaaccccaagaggcaagcctctt

SEQ ID NO: 851 - DNA, 50 bp, synthetic construct
  misc_feature 1..50: note = Synthetic
  source 1..50: mol_type = other DNA
tcttacttcttgcgaatgggagatctcatcttggagtaggcttcgtgctt

SEQ ID NO: 852 - DNA, 50 bp, synthetic construct
  misc_feature 1..50: note = Synthetic
  source 1..50: mol_type = other DNA
gtcaagctcccttatacctttacactctgcgattgatttccaaccaatct

SEQ ID NO: 853 - DNA, 50 bp, synthetic construct
  misc_feature 1..50: note = Synthetic
  source 1..50: mol_type = other DNA
ccacctatcctacacatcaaggctcaatgttcagtgtcaagctatagtaa

SEQ ID NO: 854 - DNA, 50 bp, synthetic construct
  misc_feature 1..50: note = Synthetic
  source 1..50: mol_type = other DNA
aaaagcagtttacaacccatagggccgtcatcctgcacgctacttggctg

SEQ ID NO: 855 - DNA, 50 bp, synthetic construct
  misc_feature 1..50: note = Synthetic
  source 1..50: mol_type = other DNA
tgagggcacctttagaagcctccgttacacttttggaggcgaccacccca

SEQ ID NO: 856 - DNA, 50 bp, synthetic construct
  misc_feature 1..50: note = Synthetic
  source 1..50: mol_type = other DNA
acgctctaaccttatggtaaccggatttgcctggtaaccagccgcttcgc

SEQ ID NO: 857 - DNA, 50 bp, synthetic construct
  misc_feature 1..50: note = Synthetic
  source 1..50: mol_type = other DNA
gcttccaagccaacatcctagctgtcttagcaatctgacttcgttagttc

SEQ ID NO: 858 - DNA, 50 bp, synthetic construct
  misc_feature 1..50: note = Synthetic
  source 1..50: mol_type = other DNA
tggccgttcaccctctcaggccggctatggatcgtcgccttggtaggccg

SEQ ID NO: 859 - DNA, 50 bp, synthetic construct
  misc_feature 1..50: note = Synthetic
  source 1..50: mol_type = other DNA
tgagccaacatcctggttgtcttcgaaatcccacatccttttccacttaa

SEQ ID NO: 860 - DNA, 50 bp, synthetic construct
  misc_feature 1..50: note = Synthetic
  source 1..50: mol_type = other DNA
ctagagagtatttagggttaggagatggtcctcccagattccgacgagat

SEQ ID NO: 861 - DNA, 50 bp, synthetic construct
  misc_feature 1..50: note = Synthetic
  source 1..50: mol_type = other DNA
gcctttcggcctcgcgttaggtcccgacttacccagggcggacgaacctt

SEQ ID NO: 862 - DNA, 50 bp, synthetic construct
  misc_feature 1..50: note = Synthetic
  source 1..50: mol_type = other DNA
gtcaaactgcccacctgacactgtctccccgcccgataagggcggcgggt

SEQ ID NO: 863 - DNA, 50 bp, synthetic construct
  misc_feature 1..50: note = Synthetic
  source 1..50: mol_type = other DNA
tggagtaaagctccatggggtctttccgtcctggcgcaggtaaccagcat

SEQ ID NO: 864 - DNA, 50 bp, synthetic construct
  misc_feature 1..50: note = Synthetic
  source 1..50: mol_type = other DNA
tttcttctcctacgggtactgagatgtttcacttccccgcgtaaccccca

SEQ ID NO: 865 - DNA, 50 bp, synthetic construct
  misc_feature 1..50: note = Synthetic
  source 1..50: mol_type = other DNA
accagctatggatcgtcggcttggtaggccattaccccaccaactaccta

SEQ ID NO: 866 - DNA, 50 bp, synthetic construct
  misc_feature 1..50: note = Synthetic
  source 1..50: mol_type = other DNA
ggggcaagtttcgtgcttagatgctttcagcacttatctcttccgcattt

SEQ ID NO: 867 - DNA, 50 bp, synthetic construct
  misc_feature 1..50: note = Synthetic
  source 1..50: mol_type = other DNA
caccagtgtcggtttggggtacgggcggccatagccctcacgccgaggct

SEQ ID NO: 868 - DNA, 50 bp, synthetic construct
  misc_feature 1..50: note = Synthetic
  source 1..50: mol_type = other DNA
gacgttctgaacccagctcgcgtgccgctttaatgggcgaacagcccaac

SEQ ID NO: 869 - DNA, 50 bp, synthetic construct
  misc_feature 1..50: note = Synthetic
  source 1..50: mol_type = other DNA
ggttagaattccaatatcgcaaggatggtatcccaacggcctctccgcca

SEQ ID NO: 870 - DNA, 50 bp, synthetic construct
  misc_feature 1..50: note = Synthetic
  source 1..50: mol_type = other DNA
aggttacccacgcgttactcacccgtccgccactagaaacaatctaaatc

SEQ ID NO: 871 - DNA, 50 bp, synthetic construct; identical to SEQ ID NO: 399
  misc_feature 1..50: note = Synthetic
  source 1..50: mol_type = other DNA

SEQ ID NO: 872 - DNA, 50 bp, synthetic construct; identical to SEQ ID NO: 523
  misc_feature 1..50: note = Synthetic
  source 1..50: mol_type = other DNA

SEQ ID NO: 873 - DNA, 50 bp, synthetic construct
  misc_feature 1..50: note = Synthetic
  source 1..50: mol_type = other DNA
cttaggaccgttatagttacggccgccgtttaccggggcttcgatcaaga

SEQ ID NO: 874 - DNA, 50 bp, synthetic construct
  misc_feature 1..50: note = Synthetic
  source 1..50: mol_type = other DNA
ccacttagtgatgatttggggaccttagctggcggtctgggttgtttccc

SEQ ID NO: 875 - DNA, 50 bp, synthetic construct
  misc_feature 1..50: note = Synthetic
  source 1..50: mol_type = other DNA
tcccccattcggacacctccgcttcttcgcttccttacagcttcacggag

SEQ ID NO: 876 - DNA, 50 bp, synthetic construct
  misc_feature 1..50: note = Synthetic
  source 1..50: mol_type = other DNA
atagatcacccggtttcgggtctgcccccactgactctggccctcttaag

SEQ ID NO: 877 - DNA, 50 bp, synthetic construct
  misc_feature 1..50: note = Synthetic
  source 1..50: mol_type = other DNA
gcctatcaaacacgtgttccacatgcgggcttcaggaccccgaagggccc

SEQ ID NO: 878 - DNA, 50 bp, synthetic construct
  misc_feature 1..50: note = Synthetic
  source 1..50: mol_type = other DNA
ccatttctgactgttatccccctgtataaggcaggttgcccacgcgttac

SEQ ID NO: 879 - DNA, 50 bp, synthetic construct; identical to SEQ ID NO: 495
  misc_feature 1..50: note = Synthetic
  source 1..50: mol_type = other DNA

SEQ ID NO: 880 - DNA, 50 bp, synthetic construct
  misc_feature 1..50: note = Synthetic
  source 1..50: mol_type = other DNA
gtttggggtacgggcggctaaaacctcgcgccgatgcttttctaggcagc

SEQ ID NO: 881 - DNA, 50 bp, synthetic construct
  misc_feature 1..50: note = Synthetic
  source 1..50: mol_type = other DNA
gcgatggcccttccatacggtaccaccggatcactaagcccgactttcgt

SEQ ID NO: 882 - DNA, 50 bp, synthetic construct
  misc_feature 1..50: note = Synthetic
  source 1..50: mol_type = other DNA
gagttaaccccggcggtcccccgtgagttcccaccataacgtgctggcaa

SEQ ID NO: 883 - DNA, 50 bp, synthetic construct
  misc_feature 1..50: note = Synthetic
  source 1..50: mol_type = other DNA
ggataatcggcggacgggattcccacccgtcacacgctactcatgcctgc

SEQ ID NO: 884 - DNA, 50 bp, synthetic construct
  misc_feature 1..50: note = Synthetic
  source 1..50: mol_type = other DNA
tacctcttcgttatgatatgtccgcaaccccaataaagaaaactttattg

SEQ ID NO: 885 - DNA, 50 bp, synthetic construct
  misc_feature 1..50: note = Synthetic
  source 1..50: mol_type = other DNA
acgtgtccggcggtactctggattcagctggcggatcttctctttcgcat

SEQ ID NO: 886 - DNA, 50 bp, synthetic construct
  misc_feature 1..50: note = Synthetic
  source 1..50: mol_type = other DNA
tcgagaccagacttcgttagactaactcagacaggattccgggaccttag

SEQ ID NO: 887 - DNA, 50 bp, synthetic construct
  misc_feature 1..50: note = Synthetic
  source 1..50: mol_type = other DNA
tggccgttcaacctctcagtccggctaccaatcgtcgccttggtgggccg

SEQ ID NO: 888 - DNA, 50 bp, synthetic construct
  misc_feature 1..50: note = Synthetic
  source 1..50: mol_type = other DNA
tataagtcaaggctgcacctaaatgcatttcggggagtacgagctatctc

SEQ ID NO: 889 - DNA, 50 bp, synthetic construct
  misc_feature 1..50: note = Synthetic
  source 1..50: mol_type = other DNA
ctactgtttcaccgcgtatacaacgctcccctacccagcatgtaaacatg

SEQ ID NO: 890 - DNA, 50 bp, synthetic construct
  misc_feature 1..50: note = Synthetic
  source 1..50: mol_type = other DNA
ttatagttacggccgccgtttactggggcttcaattcacaccttcgacaa

SEQ ID NO: 891 - DNA, 50 bp, synthetic construct
  misc_feature 1..50: note = Synthetic
  source 1..50: mol_type = other DNA
ggatggacccctcacccaaacagtgctctacctccatgattcttaatgtc

SEQ ID NO: 892 - DNA, 50 bp, synthetic construct; identical to SEQ ID NO: 330
  misc_feature 1..50: note = Synthetic
  source 1..50: mol_type = other DNA

SEQ ID NO: 893 - DNA, 50 bp, synthetic construct
  misc_feature 1..50: note = Synthetic
  source 1..50: mol_type = other DNA
ggctctgactacttgtaggcacacggtttcaggatctctttcactcccct

SEQ ID NO: 894 - DNA, 50 bp, synthetic construct
  misc_feature 1..50: note = Synthetic
  source 1..50: mol_type = other DNA
tcgctactcattccggcattctcactcgtgtacagtccaccgctgctttc

SEQ ID NO: 895 - DNA, 50 bp, synthetic construct
  misc_feature 1..50: note = Synthetic
  source 1..50: mol_type = other DNA
cctcccccccccccccccccccccccccttcccccctctcctcccccttc

SEQ ID NO: 896 - DNA, 50 bp, synthetic construct
  misc_feature 1..50: note = Synthetic
  source 1..50: mol_type = other DNA
taacaccccataacaggtgccaggtttccccattcggacatcctcggatc

SEQ ID NO: 897 - DNA, 50 bp, synthetic construct
  misc_feature 1..50: note = Synthetic
  source 1..50: mol_type = other DNA
acctcgacacggacggtgacaagccggtaccagaatatcaactggttacc

SEQ ID NO: 898 - DNA, 50 bp, synthetic construct
  misc_feature 1..50: note = Synthetic
  source 1..50: mol_type = other DNA
atagatcacccggtttcgggtctactccggctgactcgctcgccctattc

SEQ ID NO: 899 - DNA, 50 bp, synthetic construct
  misc_feature 1..50: note = Synthetic
  source 1..50: mol_type = other DNA
taaatgatggctgcttctaagccaacatcctggctgtctgggccttccca

SEQ ID NO: 900 - DNA, 50 bp, synthetic construct
  misc_feature 1..50: note = Synthetic
  source 1..50: mol_type = other DNA
cagcttatagggttgcgtacttcactacaacccaaccttgatgcttgcac

SEQ ID NO: 901 - DNA, 50 bp, synthetic construct
  misc_feature 1..50: note = Synthetic
  source 1..50: mol_type = other DNA
gcttgggccttttcactgcggctgacttatcgccagcgccccttctcccg

SEQ ID NO: 902 - DNA, 50 bp, synthetic construct
  misc_feature 1..50: note = Synthetic
  source 1..50: mol_type = other DNA
tgaggtcggcttcacgcttagatgctttcagcgtttatccgttccgcact

SEQ ID NO: 903 - DNA, 50 bp, synthetic construct
  misc_feature 1..50: note = Synthetic
  source 1..50: mol_type = other DNA
ctccgggtactgtcaggttcgactctcagggcggatttgcctaccccgat

SEQ ID NO: 904 - DNA, 50 bp, synthetic construct
  misc_feature 1..50: note = Synthetic
  source 1..50: mol_type = other DNA
gcttgggcctcttcactgcggcttaattgcttaagcactccttctcgcta

SEQ ID NO: 905 - DNA, 50 bp, synthetic construct
  misc_feature 1..50: note = Synthetic
  source 1..50: mol_type = other DNA
tttatcccgaagttacagggtcagtttgcctagttccttaaccgtgaatc

SEQ ID NO: 906 - DNA, 50 bp, synthetic construct
  misc_feature 1..50: note = Synthetic
  source 1..50: mol_type = other DNA
gtagttagccggagcttcctcctaaagtaccgtcattatcgtcctttaag

SEQ ID NO: 907 - DNA, 50 bp, synthetic construct
  misc_feature 1..50: note = Synthetic
  source 1..50: mol_type = other DNA
tctttcggcgagggggtttcccgccccctttatcgttacttatacctaca

SEQ ID NO: 908 - DNA, 50 bp, synthetic construct
  misc_feature 1..50: note = Synthetic
  source 1..50: mol_type = other DNA
ggatgtactagcagcttttctcgccagcgtgaactcactcgcttccctac

SEQ ID NO: 909 - DNA, 50 bp, synthetic construct
  misc_feature 1..50: note = Synthetic
  source 1..50: mol_type = other DNA
ttagtatcagtgctttatcaggggcgcatatactcgggtaccagaatatc

SEQ ID NO: 910 - DNA, 50 bp, synthetic construct
  misc_feature 1..50: note = Synthetic
  source 1..50: mol_type = other DNA
gcttggcggcgtcctactctcacagggggaaacccccgactaccatcggc

SEQ ID NO: 911 - DNA, 50 bp, synthetic construct
  misc_feature 1..50: note = Synthetic
  source 1..50: mol_type = other DNA
agattcacgcagaattcctcgtgctccgcgctactcaggatactactatg

SEQ ID NO: 912 - DNA, 50 bp, synthetic construct
  misc_feature 1..50: note = Synthetic
  source 1..50: mol_type = other DNA
tatcaacctgatcatctttcagggatcttacttccttgcggaatgggaaa

SEQ ID NO: 913 - DNA, 50 bp, synthetic construct
  misc_feature 1..50: note = Synthetic
  source 1..50: mol_type = other DNA
tcaataggcacgccaccacactcttatggagcggtgactgcttgtaagtc

SEQ ID NO: 914 - DNA, 50 bp, synthetic construct
  misc_feature 1..50: note = Synthetic
  source 1..50: mol_type = other DNA
ctactatatttcggtcccttacgcccggggcaaccatcgcccgggataac

SEQ ID NO: 915 - DNA, 50 bp, synthetic construct
  misc_feature 1..50: note = Synthetic
  source 1..50: mol_type = other DNA
tgccatgactgcttgtaagtccacggtttcaggttctctttcactcccct

SEQ ID NO: 916 - DNA, 50 bp, synthetic construct
  misc_feature 1..50: note = Synthetic
  source 1..50: mol_type = other DNA
tccatttgcgcagcaccagtaatcatgttcttaacatagtcagcatgtcc

SEQ ID NO: 917 - DNA, 50 bp, synthetic construct
  misc_feature 1..50: note = Synthetic
  source 1..50: mol_type = other DNA
tctcagtcccaatgtggccggtcaccctctcaggtcggctactgatcgtc

SEQ ID NO: 918 - DNA, 50 bp, synthetic construct
  misc_feature 1..50: note = Synthetic
  source 1..50: mol_type = other DNA
tggccgttcaacctctcagtccggctactgatcgtcgccttggtgggcct

SEQ ID NO: 919 - DNA, 50 bp, synthetic construct
  misc_feature 1..50: note = Synthetic
  source 1..50: mol_type = other DNA
ttatagttacggccgccgtttaccggggcttcaattcggagctctcactc

SEQ ID NO: 920 - DNA, 50 bp, synthetic construct
  misc_feature 1..50: note = Synthetic
  source 1..50: mol_type = other DNA
tagtgaaaggtagattttctgaccctttcgacctgaacgtaccaaccagc

SEQ ID NO: 921 - DNA, 50 bp, synthetic construct
  misc_feature 1..50: note = Synthetic
  source 1..50: mol_type = other DNA
tcttggcagtgtgacatcactaacttcgctactaaacttcgctccccatc

SEQ ID NO: 922 - DNA, 50 bp, synthetic construct
  misc_feature 1..50: note = Synthetic
  source 1..50: mol_type = other DNA
acctgctttcgcacctgctcgcgccgtcacgctcgcagtcaagctggctt

SEQ ID NO: 923 - DNA, 50 bp, synthetic construct
  misc_feature 1..50: note = Synthetic
  source 1..50: mol_type = other DNA
tcggagtttgatattcttcggtaagctttgacgccccctaggaaattcag

SEQ ID NO: 924 - DNA, 50 bp, synthetic construct
  misc_feature 1..50: note = Synthetic
  source 1..50: mol_type = other DNA
acccaccgagtgggcgcccatcaggtctcaagcacatagccggcggattt

SEQ ID NO: 925 - DNA, 50 bp, synthetic construct
  misc_feature 1..50: note = Synthetic
  source 1..50: mol_type = other DNA
tacgggtgccgcatggataagtttagcggattttctcgggagcatggtta

SEQ ID NO: 926 - DNA, 50 bp, synthetic construct
  misc_feature 1..50: note = Synthetic
  source 1..50: mol_type = other DNA
ttcaaacaaccatccggtattagccccggtttcccggagttatcccagtc

SEQ ID NO: 927 - DNA, 50 bp, synthetic construct
  misc_feature 1..50: note = Synthetic
  source 1..50: mol_type = other DNA
tccttaaccacgctgcataccataactcgccggaccattctacaaaaggt

SEQ ID NO: 928 - DNA, 50 bp, synthetic construct
  misc_feature 1..50: note = Synthetic
  source 1..50: mol_type = other DNA
ccggcaccgggcaggtgtcaggctgtatacgtcatctttcgagtttgcac

SEQ ID NO: 929 - DNA, 50 bp, synthetic construct
  misc_feature 1..50: note = Synthetic
  source 1..50: mol_type = other DNA
caggaatattcaggcttacccaacggtctgggcggattcgcacggggttc

SEQ ID NO: 930 - DNA, 50 bp, synthetic construct; identical to SEQ ID NO: 905
  misc_feature 1..50: note = Synthetic
  source 1..50: mol_type = other DNA

SEQ ID NO: 931 - DNA, 50 bp, synthetic construct
  misc_feature 1..50: note = Synthetic
  source 1..50: mol_type = other DNA
cttctgcaattgcactcgtcgattggtttccatccaatctgagcgtacct

SEQ ID NO: 932 - DNA, 50 bp, synthetic construct
  misc_feature 1..50: note = Synthetic
  source 1..50: mol_type = other DNA
tcggtttgccctcttccgcgttcgctcgccactacttacggaatctcgtt

SEQ ID NO: 933 - DNA, 50 bp, synthetic construct; identical to SEQ ID NO: 681
  misc_feature 1..50: note = Synthetic
  source 1..50: mol_type = other DNA

SEQ ID NO: 934 - DNA, 50 bp, synthetic construct
  misc_feature 1..50: note = Synthetic
  source 1..50: mol_type = other DNA
catcggcctcaccgttcggctgagccttaggacccgactaaccctgatcc

SEQ ID NO: 935 - DNA, 50 bp, synthetic construct
  misc_feature 1..50: note = Synthetic
  source 1..50: mol_type = other DNA
cctcgccatacacgccgcacggatttgcctatgcgactggctgcgtgctt

SEQ ID NO: 936 - DNA, 50 bp, synthetic construct
  misc_feature 1..50: note = Synthetic
  source 1..50: mol_type = other DNA
cctgtcgcgggtaacctgcatcttcacaggtactataatttcaccgagtc

SEQ ID NO: 937 - DNA, 50 bp, synthetic construct
  misc_feature 1..50: note = Synthetic
  source 1..50: mol_type = other DNA
tcagccttatgggaaacggatttgcctatttcccagcctaactgcttgga

SEQ ID NO: 938 - DNA, 50 bp, synthetic construct
  misc_feature 1..50: note = Synthetic
  source 1..50: mol_type = other DNA
tttcacaacacgcttaaaaggcggcctacgctccctttaaacccaataaa

SEQ ID NO: 939 - DNA, 50 bp, synthetic construct
  misc_feature 1..50: note = Synthetic
  source 1..50: mol_type = other DNA
ccccgcggtactctggatcctgctagctctcgctccttttcgtctacgtg

SEQ ID NO: 940 - DNA, 50 bp, synthetic construct
  misc_feature 1..50: note = Synthetic
  source 1..50: mol_type = other DNA
atcggttcacacactcacccaccccagaagcatcaaaaacactcccaaga

SEQ ID NO: 941 - DNA, 50 bp, synthetic construct
  misc_feature 1..50: note = Synthetic
  source 1..50: mol_type = other DNA
tagaaaggaggtgatccagccgcaccttccgatacggctaccttgttacg

SEQ ID NO: 942 - DNA, 50 bp, synthetic construct
  misc_feature 1..50: note = Synthetic
  source 1..50: mol_type = other DNA
gcccattgtccaatattccccactgctgcctcccgtaggagtctggaccg

SEQ ID NO: 943 - DNA, 50 bp, synthetic construct
  misc_feature 1..50: note = Synthetic
  source 1..50: mol_type = other DNA
tcacctttccctcacggtactggttcgctatcggtctctcgggagtattt

SEQ ID NO: 944 - DNA, 50 bp, synthetic construct
  misc_feature 1..50: note = Synthetic
  source 1..50: mol_type = other DNA
cgaagttacggggtcattttgccgagttccttgacaatgcttctcccgcc

SEQ ID NO: 945 - DNA, 50 bp, synthetic construct
  misc_feature 1..50: note = Synthetic
  source 1..50: mol_type = other DNA
agatcctctcaaatttcctacgcccgcgacggatagggaccgaactgtct

SEQ ID NO: 946 - DNA, 50 bp, synthetic construct; identical to SEQ ID NO: 917
  misc_feature 1..50: note = Synthetic
  source 1..50: mol_type = other DNA

SEQ ID NO: 947 - DNA, 50 bp, synthetic construct
  misc_feature 1..50: note = Synthetic
  source 1..50: mol_type = other DNA
ggcaacccaacaacccacacatcatcatcttcagctacaggactctcacc

SEQ ID NO: 948 - DNA, 50 bp, synthetic construct
  misc_feature 1..50: note = Synthetic
  source 1..50: mol_type = other DNA
gcactattgccttgtcccggaggacgcggcatactgtcaggttcgaatca

SEQ ID NO: 949 - DNA, 50 bp, synthetic construct
  misc_feature 1..50: note = Synthetic
  source 1..50: mol_type = other DNA
ccgtggctttctggttaggtaccgtcaaggtaccgccctattcgaacggt

SEQ ID NO: 950 - DNA, 50 bp, synthetic construct
  misc_feature 1..50: note = Synthetic
  source 1..50: mol_type = other DNA
atactatcaggttcgactcttatcccggatttgcctgggataatcaacat

SEQ ID NO: 951 - DNA, 50 bp, synthetic construct
  misc_feature 1..50: note = Synthetic
  source 1..50: mol_type = other DNA
taagtccttaaccttgctgcatacaatcgctcgccggaccgttctacaaa

SEQ ID NO: 952 - DNA, 50 bp, synthetic construct; identical to SEQ ID NO: 787
  misc_feature 1..50: note = Synthetic
  source 1..50: mol_type = other DNA

SEQ ID NO: 953 - DNA, 50 bp, synthetic construct
  misc_feature 1..50: note = Synthetic
  source 1..50: mol_type = other DNA
agagtaaccataacacaagggtagtatcccaacaacgcctcctccgaaac

SEQ ID NO: 954 - DNA, 50 bp, synthetic construct
  misc_feature 1..50: note = Synthetic
  source 1..50: mol_type = other DNA
tggacaggattctcacctgtcttacgctactcataccggcattctcactt

SEQ ID NO: 955 - DNA, 50 bp, synthetic construct
  misc_feature 1..50: note = Synthetic
  source 1..50: mol_type = other DNA
gcccggctaccttcctgcgtcacacctgttaatacgcttggctccccagt

SEQ ID NO: 956 - DNA, 50 bp, synthetic construct
  misc_feature 1..50: note = Synthetic
  source 1..50: mol_type = other DNA
gtcaagctcccttatacctttacactctgcgaatgatttccaaccattct

SEQ ID NO: 957 - DNA, 50 bp, synthetic construct
  misc_feature 1..50: note = Synthetic
  source 1..50: mol_type = other DNA
cccaacccttggaacatactacagccccaggtggcgaagagccgacatcg

SEQ ID NO: 958 - DNA, 50 bp, synthetic construct; identical to SEQ ID NO: 523
  misc_feature 1..50: note = Synthetic
  source 1..50: mol_type = other DNA

SEQ ID NO: 959 - DNA, 50 bp, synthetic construct
  misc_feature 1..50: note = Synthetic
  source 1..50: mol_type = other DNA
gggtgttccccttttgcccgcggaacttatctctcgcggactgactccca

SEQ ID NO: 960 - DNA, 50 bp, synthetic construct
  misc_feature 1..50: note = Synthetic
  source 1..50: mol_type = other DNA
acccggtttcgggtctatggcatacaacttctcgcccttgtcagactcgc

SEQ ID NO: 961 - DNA, 50 bp, synthetic construct
  misc_feature 1..50: note = Synthetic
  source 1..50: mol_type = other DNA
ctgcctggcttacgcctacggggctttcaccctctccggcgccggcattc

SEQ ID NO: 962 - DNA, 50 bp, synthetic construct
  misc_feature 1..50: note = Synthetic
  source 1..50: mol_type = other DNA
gctgcggggctgagccccttaacctcgccggaaaaagtaactcgtaggtt

SEQ ID NO: 963 - DNA, 50 bp, synthetic construct
  misc_feature 1..50: note = Synthetic
  source 1..50: mol_type = other DNA
aaggatggctctcttcaaatctcctgcgcccgcgacggatagggaccgaa

SEQ ID NO: 964 - DNA, 50 bp, synthetic construct
  misc_feature 1..50: note = Synthetic
  source 1..50: mol_type = other DNA
caggccccacaacaccgcacacacaacccccgccgggtatcacatgcaca

SEQ ID NO: 965 - DNA, 50 bp, synthetic construct
  misc_feature 1..50: note = Synthetic
  source 1..50: mol_type = other DNA
cccctacggatccatgccttggtgggccattaccccaccaactagctaat

SEQ ID NO: 966 - DNA, 50 bp, synthetic construct
  misc_feature 1..50: note = Synthetic
  source 1..50: mol_type = other DNA
acttagcactcatcgtttacggcgtggactaccagggtatctaatcctgt

SEQ ID NO: 967 - DNA, 50 bp, synthetic construct
  misc_feature 1..50: note = Synthetic
  source 1..50: mol_type = other DNA
tatccatcgaagactaggtgggccgttaccccgcctactatctaatggaa

SEQ ID NO: 968 - DNA, 50 bp, synthetic construct; identical to SEQ ID NO: 10
  misc_feature 1..50: note = Synthetic
  source 1..50: mol_type = other DNA

SEQ ID NO: 969 - DNA, 50 bp, synthetic construct
  misc_feature 1..50: note = Synthetic
  source 1..50: mol_type = other DNA
tggccgttcaacctctcagtccggctaccgatcgcggtcttggtgagccg

SEQ ID NO: 970 - DNA, 50 bp, synthetic construct
  misc_feature 1..50: note = Synthetic
  source 1..50: mol_type = other DNA
cctgtgtttttgctaaacagtcgcctgggcctattcactgcggctctctc

SEQ ID NO: 971 - DNA, 50 bp, synthetic construct
  misc_feature 1..50: note = Synthetic
  source 1..50: mol_type = other DNA
acgcctttcggcctgaccttagctcccgacttacttggagcggacgaacc

SEQ ID NO: 972 - DNA, 50 bp, synthetic construct
  misc_feature 1..50: note = Synthetic
  source 1..50: mol_type = other DNA
ggtctgggctctttcccttttgactgcccaacttatctcgtgcagtctga

SEQ ID NO: 973 - DNA, 50 bp, synthetic construct
  misc_feature 1..50: note = Synthetic
  source 1..50: mol_type = other DNA
gaatgaatggctgcttctgagccaacatcctagttgtcttagagatccca

SEQ ID NO: 974 - DNA, 50 bp, synthetic construct
  misc_feature 1..50: note = Synthetic
  source 1..50: mol_type = other DNA
ccccatcatgcctcaaccttcacgcccagcggatttacctaccagacagt

SEQ ID NO: 975 - DNA, 50 bp, synthetic construct
  misc_feature 1..50: note = Synthetic
  source 1..50: mol_type = other DNA
aaaagtacgcggttcatcatataaagatgttccacagcttgtaaacacag

SEQ ID NO: 976 - DNA, 50 bp, synthetic construct
  misc_feature 1..50: note = Synthetic
  source 1..50: mol_type = other DNA
atctgaagtcttctcgtttaacatacaggactattaccttctgtggtgag

SEQ ID NO: 977 - DNA, 50 bp, synthetic construct
  misc_feature 1..50: note = Synthetic
  source 1..50: mol_type = other DNA
ggtcacacccttttgaagtgtccctttgcttaaattacagatggttacgg

SEQ ID NO: 978 - DNA, 50 bp, synthetic construct
  misc_feature 1..50: note = Synthetic
  source 1..50: mol_type = other DNA
cagcttatcacgtctttcatcggctcttagtgccaaggcatccaccctgc

SEQ ID NO: 979 - DNA, 50 bp, synthetic construct
  misc_feature 1..50: note = Synthetic
  source 1..50: mol_type = other DNA
ttccattcggcaccgccggatcactattcccgactttcgtccctgttcga

SEQ ID NO: 980 - DNA, 50 bp, synthetic construct
  misc_feature 1..50: note = Synthetic
  source 1..50: mol_type = other DNA
tccaggttcgattggcatttcacccctacccacacctcatccccgcactt

SEQ ID NO: 981 - DNA, 50 bp, synthetic construct
  misc_feature 1..50: note = Synthetic
  source 1..50: mol_type = other DNA
tacaccttctgcgtacatagaacgctctcctaccatcccctaagggatcc

SEQ ID NO: 982 - DNA, 50 bp, synthetic construct; identical to SEQ ID NO: 191
  misc_feature 1..50: note = Synthetic
  source 1..50: mol_type = other DNA

SEQ ID NO: 983 - DNA, 50 bp, synthetic construct
  misc_feature 1..50: note = Synthetic
  source 1..50: mol_type = other DNA
cgcccgttagtaccggtcggctccacccctcgcggggcttccacctccgg

SEQ ID NO: 984 - DNA, 50 bp, synthetic construct
  misc_feature 1..50: note = Synthetic
  source 1..50: mol_type = other DNA
ctccgggaccttagacggcggtctggattcttctcctctcggggacggac

SEQ ID NO: 985 - DNA, 50 bp, synthetic construct
  misc_feature 1..50: note = Synthetic
  source 1..50: mol_type = other DNA
tggttaagtcctcgatcgattagtatctgtcagctccatgtgtcgccaca

SEQ ID NO: 986 - DNA, 50 bp, synthetic construct; identical to SEQ ID NO: 951
  misc_feature 1..50: note = Synthetic
  source 1..50: mol_type = other DNA

SEQ ID NO: 987 - DNA, 50 bp, synthetic construct
  misc_feature 1..50: note = Synthetic
  source 1..50: mol_type = other DNA
accggactttccatttccggcccatgtttccctcccgtgtccccacagtt

SEQ ID NO: 988 - DNA, 50 bp, synthetic construct
  misc_feature 1..50: note = Synthetic
  source 1..50: mol_type = other DNA
cggctcccacctatgctacgcagaagaatccggatatcaatgccagacta

SEQ ID NO: 989 - DNA, 50 bp, synthetic construct
  misc_feature 1..50: note = Synthetic
  source 1..50: mol_type = other DNA
accccacatccttttccacttaacatatatttggggaccttagctggtgg

SEQ ID NO: 990 - DNA, 50 bp, synthetic construct
  misc_feature 1..50: note = Synthetic
  source 1..50: mol_type = other DNA
ccacaccacttcacctaacaacaacacacaagcacgatgatggtagtcac

SEQ ID NO: 991 - DNA, 50 bp, synthetic construct
  misc_feature 1..50: note = Synthetic
  source 1..50: mol_type = other DNA
tcatccccgcacttttcacgtacgtgtggttcggacctccacgacgtctt

SEQ ID NO: 992 - DNA, 50 bp, synthetic construct
  misc_feature 1..50: note = Synthetic
  source 1..50: mol_type = other DNA
cccttcaaagcctccgacctatcctacacatcacgtgcccagattcaatg

SEQ ID NO: 993 - DNA, 50 bp, synthetic construct
  misc_feature 1..50: note = Synthetic
  source 1..50: mol_type = other DNA
cttcacctaacaacaatgcgcaagcaggacgtcagtagccatcctcatca

SEQ ID NO: 994 - DNA, 50 bp, synthetic construct
  misc_feature 1..50: note = Synthetic
  source 1..50: mol_type = other DNA
ggggtacgggcggcaacgcgcctgacgccgaagcttttctcggcaccacg

SEQ ID NO: 995 - DNA, 50 bp, synthetic construct
  misc_feature 1..50: note = Synthetic
  source 1..50: mol_type = other DNA
atggctagatcaccgggtttcgggtctataccctgcaacttaacgcccag

SEQ ID NO: 996 - DNA, 50 bp, synthetic construct
  misc_feature 1..50: note = Synthetic
  source 1..50: mol_type = other DNA
attaaaccacatgctccaccgcttgtgcgggcccccgtcaattcctttga

SEQ ID NO: 997 - DNA, 50 bp, synthetic construct
  misc_feature 1..50: note = Synthetic
  source 1..50: mol_type = other DNA
gccggctttcccaaagccgttctgctacctctcgcggatcaattatgcgg

SEQ ID NO: 998 - DNA, 50 bp, synthetic construct
  misc_feature 1..50: note = Synthetic
  source 1..50: mol_type = other DNA
acgccttccggcctcaccttagctcccgactaacttggagcggacgaacc

SEQ ID NO: 999 - DNA, 50 bp, synthetic construct
  misc_feature 1..50: note = Synthetic
  source 1..50: mol_type = other DNA
acaccacgcggcgataccaacccgaaggaaggaaccaccacgaggcggag

SEQ ID NO: 1000 - DNA, 50 bp, synthetic construct
  misc_feature 1..50: note = Synthetic
  source 1..50: mol_type = other DNA
ccgaaccccgagatgcacgcatctcggtttggcctctttcgcgttcgctc

SEQ ID NO: 1001 - DNA, 50 bp, synthetic construct
  misc_feature 1..50: note = Synthetic
  source 1..50: mol_type = other DNA
gggacttcatcctggccaagtgtagatcacttggtttcgcgtctaccccc

SEQ ID NO: 1002 - DNA, 50 bp, synthetic construct
  misc_feature 1..50: note = Synthetic
  source 1..50: mol_type = other DNA
agccctcgacctattagtactgccaagctgaatgcctcacggcacttaca

SEQ ID NO: 1003 - DNA, 50 bp, synthetic construct
  misc_feature 1..50: note = Synthetic
  source 1..50: mol_type = other DNA
gggagcgggattaccttcactatcaatccacccgaaggtttcatgtacta

SEQ ID NO: 1004 - DNA, 50 bp, synthetic construct
  misc_feature 1..50: note = Synthetic
  source 1..50: mol_type = other DNA
cacgcgggattccacgaggcccgcgctacttgggacaacacgatcggaag

SEQ ID NO: 1005 - DNA, 50 bp, synthetic construct
  misc_feature 1..50: note = Synthetic
  source 1..50: mol_type = other DNA
cctacacccttcaaccatctattccgtcagatggcggcactgtcactact

SEQ ID NO: 1006 - DNA, 50 bp, synthetic construct
  misc_feature 1..50: note = Synthetic
  source 1..50: mol_type = other DNA
ccccgtacctgttctcgataccaggttagaaccccggtcacacaagagtg

SEQ ID NO: 1007 - DNA, 50 bp, synthetic construct
  misc_feature 1..50: note = Synthetic
  source 1..50: mol_type = other DNA
gtttcacgtgtctggccgtactctggatcctgcgcagctctctccgtttt

SEQ ID NO: 1008 - DNA, 50 bp, synthetic construct
  misc_feature 1..50: note = Synthetic
  source 1..50: mol_type = other DNA
ttcccgcttagatgctttcagcggttatccctcccgaacgtagccaaccg

SEQ ID NO: 1009 - DNA, 50 bp, synthetic construct
  misc_feature 1..50: note = Synthetic
  source 1..50: mol_type = other DNA
gcactcccacagcttgtagacacagggtttcaggttctctttcactcccc

SEQ ID NO: 1010 - DNA, 50 bp, synthetic construct; identical to SEQ ID NO: 552
  misc_feature 1..50: note = Synthetic
  source 1..50: mol_type = other DNA

SEQ ID NO: 1011 - DNA, 50 bp, synthetic construct
  misc_feature 1..50: note = Synthetic
  source 1..50: mol_type = other DNA
ccgcgagggacctcacctacatatcagcgtgccttctcccgaagttacgg

SEQ ID NO: 1012 - DNA, 50 bp, synthetic construct
  misc_feature 1..50: note = Synthetic
  source 1..50: mol_type = other DNA
aagctccatggggtctttccgtcttgccgcaggtaaccggcatcttcacc

SEQ ID NO: 1013 - DNA, 50 bp, synthetic construct
  misc_feature 1..50: note = Synthetic
  source 1..50: mol_type = other DNA
cgtcggcttggtgggccgttacctcaccaactacctaatccaacgcgggt

SEQ ID NO: 1014 - DNA, 50 bp, synthetic construct
  misc_feature 1..50: note = Synthetic
  source 1..50: mol_type = other DNA
gctcccacctatcctgtacatgcaataccaagctccagtaccaaactgga

SEQ ID NO: 1015 - DNA, 50 bp, synthetic construct; identical to SEQ ID NO: 987
  misc_feature 1..50: note = Synthetic
  source 1..50: mol_type = other DNA

SEQ ID NO: 1016 - DNA, 50 bp, synthetic construct
  misc_feature 1..50: note = Synthetic
  source 1..50: mol_type = other DNA
cagttccccgggtctgccttctcatatcctatgaattcagatatggatac

SEQ ID NO: 1017 - DNA, 50 bp, synthetic construct
  misc_feature 1..50: note = Synthetic
  source 1..50: mol_type = other DNA
ggtcccggcagattcgcgcaggattcctcgtgtcccgcgttactcaggat

SEQ ID NO: 1018 - DNA, 50 bp, synthetic construct
  misc_feature 1..50: note = Synthetic
  source 1..50: mol_type = other DNA
gtattaactttactcccttcctccccgctgaaagtactttacaacccgaa

SEQ ID NO: 1019 - DNA, 50 bp, synthetic construct
  misc_feature 1..50: note = Synthetic
  source 1..50: mol_type = other DNA
gggggcggggagcggggcgtgggcgggaggaggggaggaggcgtgggggg

SEQ ID NO: 1020 - DNA, 50 bp, synthetic construct
  misc_feature 1..50: note = Synthetic
  source 1..50: mol_type = other DNA
cacgaggcccgcgctacttgggacacgcgatcgggagacggcaagcgtcc

SEQ ID NO: 1021 - DNA, 50 bp, synthetic construct
  misc_feature 1..50: note = Synthetic
  source 1..50: mol_type = other DNA
cgtttatcccctccctacttagctacccagcgatgctcttggcagaacaa

SEQ ID NO: 1022 - DNA, 50 bp, synthetic construct
  misc_feature 1..50: note = Synthetic
  source 1..50: mol_type = other DNA
cctcttaaccttccggcaccgggcaggcgtcagagcgtatacagcggctt

SEQ ID NO: 1023 - DNA, 50 bp, synthetic construct; identical to SEQ ID NO: 753
  misc_feature 1..50: note = Synthetic
  source 1..50: mol_type = other DNA

SEQ ID NO: 1024 - DNA, 50 bp, synthetic construct
  misc_feature 1..50: note = Synthetic
  source 1..50: mol_type = other DNA
ttcgttcgccactactagcagaatcataattttattttcttctcctacgg

SEQ ID NO: 1025 - DNA, 50 bp, synthetic construct
  misc_feature 1..50: note = Synthetic
  source 1..50: mol_type = other DNA
gtttctcgcatgcctctcgctactcataccggcattctctcttgtgcagt

SEQ ID NO: 1026 - DNA, 50 bp, synthetic construct
  misc_feature 1..50: note = Synthetic
  source 1..50: mol_type = other DNA
cctatcaacgtcgtcgtcttcaacgttccttcaggacccttaaagggtca

SEQ ID NO: 1027 - DNA, 50 bp, synthetic construct
  misc_feature 1..50: note = Synthetic
  source 1..50: mol_type = other DNA
ctgttatccccagggtagcttttatccgttgagcgacggcatttccattc

SEQ ID NO: 1028 - DNA, 50 bp, synthetic construct
  misc_feature 1..50: note = Synthetic
  source 1..50: mol_type = other DNA
caacaatatatggaacacctacctggcgagacaatagaatgtgttccctc

SEQ ID NO: 1029 - DNA, 50 bp, synthetic construct; identical to SEQ ID NO: 767
  misc_feature 1..50: note = Synthetic
  source 1..50: mol_type = other DNA

SEQ ID NO: 1030 - DNA, 50 bp, synthetic construct
  misc_feature 1..50: note = Synthetic
  source 1..50: mol_type = other DNA
acaacagagctttacgatccgaaaaccttcatcactcacgcggcgttgct

SEQ ID NO: 1031 - DNA, 50 bp, synthetic construct
  misc_feature 1..50: note = Synthetic
  source 1..50: mol_type = other DNA
cccgttccacgggttagaatccaaacaaataaagggtcgtatttcaacag

SEQ ID NO: 1032 - DNA, 50 bp, synthetic construct
  misc_feature 1..50: note = Synthetic
  source 1..50: mol_type = other DNA
cccccttcccccctctcctcccccttccccctttcgcgcccccttttccc

SEQ ID NO: 1033 - DNA, 50 bp, synthetic construct
  misc_feature 1..50: note = Synthetic
  source 1..50: mol_type = other DNA
tggtgttccaaccaattcggcttggggggatggatcttaaaaactggtcc

SEQ ID NO: 1034 - DNA, 50 bp, synthetic construct
  misc_feature 1..50: note = Synthetic
  source 1..50: mol_type = other DNA
ctcgtgtcccgccgtactcaggatcctgcttggcatcaagtgaatttcaa

SEQ ID NO: 1035 - DNA, 50 bp, synthetic construct
  misc_feature 1..50: note = Synthetic
  source 1..50: mol_type = other DNA
agcttctacacccttcaaccatctattccgtcagatggcggcactgtcac

SEQ ID NO: 1036 - DNA, 50 bp, synthetic construct
  misc_feature 1..50: note = Synthetic
  source 1..50: mol_type = other DNA
ccgattagtaccagtcaactccgtacatcactgcacttccatccctggcc

SEQ ID NO: 1037 - DNA, 50 bp, synthetic construct
  misc_feature 1..50: note = Synthetic
  source 1..50: mol_type = other DNA
cgcttgaaccacacatcaggccccacggcttgccaccatgttaacccgaa

SEQ ID NO: 1038 - DNA, 50 bp, synthetic construct
  misc_feature 1..50: note = Synthetic
  source 1..50: mol_type = other DNA
tggcgagacaatagaatgtgttccctcgtttgtggcataggaccatcagc

SEQ ID NO: 1039 - DNA, 50 bp, synthetic construct; identical to SEQ ID NO: 28
  misc_feature 1..50: note = Synthetic
  source 1..50: mol_type = other DNA

SEQ ID NO: 1040 - DNA, 50 bp, synthetic construct
  misc_feature 1..50: note = Synthetic
  source 1..50: mol_type = other DNA
tcgaggtgccaaacctccccgtcgatgtgaactcttgggggagataagcc

SEQ ID NO: 1041 - DNA, 50 bp, synthetic construct
  misc_feature 1..50: note = Synthetic
  source 1..50: mol_type = other DNA
cttaacaacttaacctcgctgcacacagtaactcgccggcccgttctaca

SEQ ID NO: 1042 - DNA, 50 bp, synthetic construct
  misc_feature 1..50: note = Synthetic
  source 1..50: mol_type = other DNA
gtcaacaggtagtattcaggcttaccaggtggtcctggcagattcacacg

SEQ ID NO: 1043 - DNA, 50 bp, synthetic construct
  misc_feature 1..50: note = Synthetic
  source 1..50: mol_type = other DNA
aggcacgccgtcacacattgctgtgctccgaccgcttgtaggcgtatggt

SEQ ID NO: 1044 - DNA, 50 bp, synthetic construct
  misc_feature 1..50: note = Synthetic
  source 1..50: mol_type = other DNA
tccctttcccccttccccccccccccccccccccccccctttcccccccc

SEQ ID NO: 1045 - DNA, 50 bp, synthetic construct
  misc_feature 1..50: note = Synthetic
  source 1..50: mol_type = other DNA
aaccatgactttgggaccttagctggcggtctgggttgtttccctcttca

SEQ ID NO: 1046 - DNA, 50 bp, synthetic construct
  misc_feature 1..50: note = Synthetic
  source 1..50: mol_type = other DNA
tgccattacactctatgagaccggttaccaatcggtccgaagggcacctt

SEQ ID NO: 1047 - DNA, 50 bp, synthetic construct
  misc_feature 1..50: note = Synthetic
  source 1..50: mol_type = other DNA
gattggaatttctccgctacccacacctcatccgctaccatttcaacggg

SEQ ID NO: 1048 - DNA, 50 bp, synthetic construct
  misc_feature 1..50: note = Synthetic
  source 1..50: mol_type = other DNA
ttctcgtgtcccgcggtactctggatcctgctcagtctgctctgttttcg

SEQ ID NO: 1049 - DNA, 50 bp, synthetic construct
  misc_feature 1..50: note = Synthetic
  source 1..50: mol_type = other DNA
gtaaacccccacaacagctatgaattcactgaagggtaacaccccataac

SEQ ID NO: 1050 - DNA, 50 bp, synthetic construct
  misc_feature 1..50: note = Synthetic
  source 1..50: mol_type = other DNA
tcccgaagttacagggtcaatttgcctagttccttaaccgtgaatcactc

SEQ ID NO: 1051 - DNA, 50 bp, synthetic construct
  misc_feature 1..50: note = Synthetic
  source 1..50: mol_type = other DNA
cccccgacgggtatcacacgcgcaaggtttggccatcatccgctttcgct

SEQ ID NO: 1052 - DNA, 50 bp, synthetic construct
  misc_feature 1..50: note = Synthetic
  source 1..50: mol_type = other DNA
cccttgtctcagtgcccatctccgggctcctccttccagagcccgtaccc

SEQ ID NO: 1053 - DNA, 50 bp, synthetic construct
  misc_feature 1..50: note = Synthetic
  source 1..50: mol_type = other DNA
tcagacttgctctcgctgcggcttcacaccttaagtgcttaacctcgccg

SEQ ID NO: 1054 - DNA, 50 bp, synthetic construct
  misc_feature 1..50: note = Synthetic
  source 1..50: mol_type = other DNA
ctccattcggaaatccacggatcaatgcctacttacggctccccgtggct

SEQ ID NO: 1055 - DNA, 50 bp, synthetic construct
  misc_feature 1..50: note = Synthetic
  source 1..50: mol_type = other DNA
ttttacggttgagccgcaaactttcacaactgacttaacaacccgcctac

SEQ ID NO: 1056 - DNA, 50 bp, synthetic construct
  misc_feature 1..50: note = Synthetic
  source 1..50: mol_type = other DNA
cggtttaggctcttccgcgttcgctcgccgctacttacggaatcgagttt

SEQ ID NO: 1057 - DNA, 50 bp, synthetic construct
  misc_feature 1..50: note = Synthetic
  source 1..50: mol_type = other DNA
cttcactatatactctagtacaggaatatcaacctgttggccatcggata

SEQ ID NO: 1058 - DNA, 50 bp, synthetic construct
  misc_feature 1..50: note = Synthetic
  source 1..50: mol_type = other DNA
tgtttcagttcactgcgtcttccttctcataaccttaacagttatggata

SEQ ID NO: 1059 - DNA, 50 bp, synthetic construct
  misc_feature 1..50: note = Synthetic
  source 1..50: mol_type = other DNA
gacggagcttatcccccgccgactcactgccgggatacgcgtcacgggta

SEQ ID NO: 1060 - DNA, 50 bp, synthetic construct
  misc_feature 1..50: note = Synthetic
  source 1..50: mol_type = other DNA
ccgaactgtctcacgacgttctgaacccagctcgcgtaccgctttaatgg

SEQ ID NO: 1061 - DNA, 50 bp, synthetic construct
  misc_feature 1..50: note = Synthetic
  source 1..50: mol_type = other DNA
gacggtgacaagccggtaccagaatatcaactggttacccatcgactacg

SEQ ID NO: 1062 - DNA, 50 bp, synthetic construct
  misc_feature 1..50: note = Synthetic
  source 1..50: mol_type = other DNA
gatgcgcattcggagtttgtcaagacttgataggcggtgaagccctcgca

SEQ ID NO: 1063 - DNA, 50 bp, synthetic construct
  misc_feature 1..50: note = Synthetic
  source 1..50: mol_type = other DNA
taggtgagccgttaccccacctactagctaatcccatctgggcacatccg

SEQ ID NO: 1064 - DNA, 50 bp, synthetic construct
  misc_feature 1..50: note = Synthetic
  source 1..50: mol_type = other DNA
tggtccccgctcattccatcaaggtttctcgtgtctcgatgtactctgga

SEQ ID NO: 1065 - DNA, 50 bp, synthetic construct
  misc_feature 1..50: note = Synthetic
  source 1..50: mol_type = other DNA
atgctcccctaccgatactttttaatgctatcccgcgccttcggtacctg

SEQ ID NO: 1066 - DNA, 50 bp, synthetic construct
  misc_feature 1..50: note = Synthetic
  source 1..50: mol_type = other DNA
ttacctttacttcaacctgaccatgggtaggtcacccggtttcgggtcga

SEQ ID NO: 1067 - DNA, 50 bp, synthetic construct
  misc_feature 1..50: note = Synthetic
  source 1..50: mol_type = other DNA
gtagtatttagccttggaggatggtccctcctgcttcccacagggtttca

SEQ ID NO: 1068 - DNA, 50 bp, synthetic construct
  misc_feature 1..50: note = Synthetic
  source 1..50: mol_type = other DNA
gatttccaaccattctgagggaacctttgggcgcctccgttaccttttag

SEQ ID NO: 1069 - DNA, 50 bp, synthetic construct
  misc_feature 1..50: note = Synthetic
  source 1..50: mol_type = other DNA
atcccttccgggcttggctactcggccgtagacttggcagtctaaccgat

SEQ ID NO: 1070 - DNA, 50 bp, synthetic construct; identical to SEQ ID NO: 1062
  misc_feature 1..50: note = Synthetic
  source 1..50: mol_type = other DNA

SEQ ID NO: 1071 - DNA, 50 bp, synthetic construct
  misc_feature 1..50: note = Synthetic
  source 1..50: mol_type = other DNA
gtaatcgccttggtgggccattaccccaccaacaagctgataggccgcag

SEQ ID NO: 1072 - DNA, 50 bp, synthetic construct; identical to SEQ ID NO: 773
  misc_feature 1..50: note = Synthetic
  source 1..50: mol_type = other DNA

SEQ ID NO: 1073 - DNA, 50 bp, synthetic construct
  misc_feature 1..50: note = Synthetic
  source 1..50: mol_type = other DNA
agctccatggggtctttccgtctagttgcgggtaacctgcattttcacag

SEQ ID NO: 1074 - DNA, 50 bp, synthetic construct
  misc_feature 1..50: note = Synthetic
  source 1..50: mol_type = other DNA
cgtggggattaagtttagcggattttctcgggagtatgattacgtgcgct

SEQ ID NO: 1075 - DNA, 50 bp, synthetic construct
  misc_feature 1..50: note = Synthetic
  source 1..50: mol_type = other DNA
tattttgggaccttaactggcggtctgggctgtttccctcttgaccatgg

SEQ ID NO: 1076 - DNA, 50 bp, synthetic construct
  misc_feature 1..50: note = Synthetic
  source 1..50: mol_type = other DNA
taaccttgcacgggatcgtaactcgccggttcattctacaaaaggcacgc

SEQ ID NO: 1077 - DNA, 50 bp, synthetic construct
  misc_feature 1..50: note = Synthetic
  source 1..50: mol_type = other DNA
gacggcccagagacctgccttcgccatcggtgttcttcccgatatctaca

SEQ ID NO: 1078 - DNA, 50 bp, synthetic construct
  misc_feature 1..50: note = Synthetic
  source 1..50: mol_type = other DNA
tcacacgggattccacgagtcccgcgctacttgggagacacgatccggag

SEQ ID NO: 1079 - DNA, 50 bp, synthetic construct
  misc_feature 1..50: note = Synthetic
  source 1..50: mol_type = other DNA
agtatttagccttggaggatggtccccccatattcagacaggataccacg

SEQ ID NO: 1080 - DNA, 50 bp, synthetic construct
  misc_feature 1..50: note = Synthetic
  source 1..50: mol_type = other DNA
tttggcctcttccgcgttcgctcgccactactagcggaatctcggttgat

SEQ ID NO: 1081 - DNA, 50 bp, synthetic construct; identical to SEQ ID NO: 99
  misc_feature 1..50: note = Synthetic
  source 1..50: mol_type = other DNA

SEQ ID NO: 1082 - DNA, 50 bp, synthetic construct
  misc_feature 1..50: note = Synthetic
  source 1..50: mol_type = other DNA
ctggggcttcaattcacaccttcgcttacgctaagcgctcctcttaacct

SEQ ID NO: 1083 - DNA, 50 bp, synthetic construct
  misc_feature 1..50: note = Synthetic
  source 1..50: mol_type = other DNA
gtttgggcttctcccctttcgctcgccgctactcagggaatcactgttgt

SEQ ID NO: 1084 - DNA, 50 bp, synthetic construct
  misc_feature 1..50: note = Synthetic
  source 1..50: mol_type = other DNA
acaatccacaccgaatgccaataccaaggtatagtaaaggtcccggggtc

SEQ ID NO: 1085 - DNA, 50 bp, synthetic construct
  misc_feature 1..50: note = Synthetic
  source 1..50: mol_type = other DNA
cagggtagcttttatccgttgagcgatggcccttccatacggtaccaccg

SEQ ID NO: 1086 - DNA, 50 bp, synthetic construct
  misc_feature 1..50: note = Synthetic
  source 1..50: mol_type = other DNA
ataggcggtgaagccctcttgacctatcggtcgctctacctctcacggtg

SEQ ID NO: 1087 - DNA, 50 bp, synthetic construct; identical to SEQ ID NO: 211
  misc_feature 1..50: note = Synthetic
  source 1..50: mol_type = other DNA

SEQ ID NO: 1088 - DNA, 50 bp, synthetic construct
  misc_feature 1..50: note = Synthetic
  source 1..50: mol_type = other DNA
cggtacgccgccggtacgggaatatccacccgttcatccattcgactacg

SEQ ID NO: 1089 - DNA, 50 bp, synthetic construct; identical to SEQ ID NO: 58
  misc_feature 1..50: note = Synthetic
  source 1..50: mol_type = other DNA

SEQ ID NO: 1090 - DNA, 50 bp, synthetic construct
  misc_feature 1..50: note = Synthetic
  source 1..50: mol_type = other DNA
cgttcactcttccttggctcctacctatcctgtacatgtgtaacagatac

SEQ ID NO: 1091 - DNA, 50 bp, synthetic construct
  misc_feature 1..50: note = Synthetic
  source 1..50: mol_type = other DNA
cccctgacctgattcaaggccacaggttagaatttcagcacttcaagagt

SEQ ID NO: 1092 - DNA, 50 bp, synthetic construct
  misc_feature 1..50: note = Synthetic
  source 1..50: mol_type = other DNA
ctacccagcaatgcctttggcaagacaactggtacaccagcggtaagtcc

SEQ ID NO: 1093 - DNA, 50 bp, synthetic construct
  misc_feature 1..50: note = Synthetic
  source 1..50: mol_type = other DNA
ccagcaccgggcaggcgtcaccccctatacttcatcttacgatttcgcag

SEQ ID NO: 1094 - DNA, 50 bp, synthetic construct
  misc_feature 1..50: note = Synthetic
  source 1..50: mol_type = other DNA
attcctcactgctgcctcccgtaggagtttggaccgtgtctcagtcccaa

SEQ ID NO: 1095 - DNA, 50 bp, synthetic construct
  misc_feature 1..50: note = Synthetic
  source 1..50: mol_type = other DNA
ctacgagactcaagcttgccagtatcagatgcagttcccaggttgagccc

SEQ ID NO: 1096 - DNA, 50 bp, synthetic construct
  misc_feature 1..50: note = Synthetic
  source 1..50: mol_type = other DNA
ctctcaacgatgacgtctcctcttaaccttccagcaccgggcaggtgtca

SEQ ID NO: 1097 - DNA, 50 bp, synthetic construct
  misc_feature 1..50: note = Synthetic
  source 1..50: mol_type = other DNA
attaccgcggctgctggcacggagttagccggtgcttcttctgcgggtaa

SEQ ID NO: 1098 - DNA, 50 bp, synthetic construct
  misc_feature 1..50: note = Synthetic
  source 1..50: mol_type = other DNA
gcgatggactttcacaccggacgcgacgagccgcctacgagccctttacg

SEQ ID NO: 1099 - DNA, 50 bp, synthetic construct
  misc_feature 1..50: note = Synthetic
  source 1..50: mol_type = other DNA
cccacaccggatatggaccgaactgtctcacgacgttctgaacccagctc

SEQ ID NO: 1100 - DNA, 50 bp, synthetic construct; identical to SEQ ID NO: 973
  misc_feature 1..50: note = Synthetic
  source 1..50: mol_type = other DNA

SEQ ID NO: 1101 - DNA, 50 bp, synthetic construct; identical to SEQ ID NO: 97
  misc_feature 1..50: note = Synthetic
  source 1..50: mol_type = other DNA

SEQ ID NO: 1102 - DNA, 50 bp, synthetic construct
  misc_feature 1..50: note = Synthetic
  source 1..50: mol_type = other DNA
gtaaagccaccttatacccttgcattctacaggagatttctgacctcctt

SEQ ID NO: 1103 - DNA, 50 bp, synthetic construct
  misc_feature 1..50: note = Synthetic
  source 1..50: mol_type = other DNA
tccgcctgcgcaccctttaaacccaataaatccggataacgctcgtatcc

SEQ ID NO: 1104 - DNA, 50 bp, synthetic construct
  misc_feature 1..50: note = Synthetic
  source 1..50: mol_type = other DNA
aggaagtattcaggcttaccaggtggtcctggcagattcacacacgattc

SEQ ID NO: 1105 - DNA, 50 bp, synthetic construct
  misc_feature 1..50: note = Synthetic
  source 1..50: mol_type = other DNA
gtgtaggattctcacctacatctcgctactcacaccggcattctcacttc

SEQ ID NO: 1106 - DNA, 50 bp, synthetic construct
  misc_feature 1..50: note = Synthetic
  source 1..50: mol_type = other DNA
gaactgagaccggttttcagggatccgctccatgtcgccatgtcgcatcc

SEQ ID NO: 1107 - DNA, 50 bp, synthetic construct
  misc_feature 1..50: note = Synthetic
  source 1..50: mol_type = other DNA
ttcctgaagttgattcttcgggttagacagccaaacttctcagggtggta

SEQ ID NO: 1108 - DNA, 50 bp, synthetic construct
  misc_feature 1..50: note = Synthetic
  source 1..50: mol_type = other DNA
cggtactggtacgctatcggtcagacaggtatgcttagacttacgccacg

SEQ ID NO: 1109 - DNA, 50 bp, synthetic construct
  misc_feature 1..50: note = Synthetic
  source 1..50: mol_type = other DNA
gtttcccctcgacttgcatgtgttaagcctgtagctagcgttcatcctga

SEQ ID NO: 1110 - DNA, 50 bp, synthetic construct; identical to SEQ ID NO: 944
  misc_feature 1..50: note = Synthetic
  source 1..50: mol_type = other DNA

SEQ ID NO: 1111 - DNA, 50 bp, synthetic construct
  misc_feature 1..50: note = Synthetic
  source 1..50: mol_type = other DNA
cttgggaatgatcagcctgttatccccggggtaccttttatccgttgagc

SEQ ID NO: 1112 - DNA, 50 bp, synthetic construct
  misc_feature 1..50: note = Synthetic
  source 1..50: mol_type = other DNA
gtctataagtacttcgatttttgcaagtccgaaccccgaacgtccgtaga

SEQ ID NO: 1113 - DNA, 50 bp, synthetic construct
  misc_feature 1..50: note = Synthetic
  source 1..50: mol_type = other DNA
cacctttccttcacagtactggttcactatcggtctctcgggagtattta

SEQ ID NO: 1114 - DNA, 50 bp, synthetic construct
  misc_feature 1..50: note = Synthetic
  source 1..50: mol_type = other DNA
ccgggaattccagtctcccctaccgcactccagcccgcccgtacccggcg

SEQ ID NO: 1115 - DNA, 50 bp, synthetic construct
  misc_feature 1..50: note = Synthetic
  source 1..50: mol_type = other DNA
acagcttttctcgccatcttccatctcggacttcggtactaatttccctc

SEQ ID NO: 1116 - DNA, 50 bp, synthetic construct
  misc_feature 1..50: note = Synthetic
  source 1..50: mol_type = other DNA
tctttcggcgaggggggttcccgccccctttatcgttacttatacctaca

SEQ ID NO: 1117 - DNA, 50 bp, synthetic construct
  misc_feature 1..50: note = Synthetic
  source 1..50: mol_type = other DNA
tgtatgcgccattgtagcacgtgtgtagccctggtcgtaagggccatgat

SEQ ID NO: 1118 - DNA, 50 bp, synthetic construct
  misc_feature 1..50: note = Synthetic
  source 1..50: mol_type = other DNA
ctttcgtctctgatcgagttgtcactctcgcagtcaggcacccttctgcc

SEQ ID NO: 1119 - DNA, 50 bp, synthetic construct
  misc_feature 1..50: note = Synthetic
  source 1..50: mol_type = other DNA
gatactacaatttcactgagctcttggttgagacagcgtccggatcatta

SEQ ID NO: 1120 - DNA, 50 bp, synthetic construct
  misc_feature 1..50: note = Synthetic
  source 1..50: mol_type = other DNA
gatgtttcagttcaggcggttccctcaatacacctattttaaatttcagt

SEQ ID NO: 1121 - DNA, 50 bp, synthetic construct
  misc_feature 1..50: note = Synthetic
  source 1..50: mol_type = other DNA
aaaaaaaaacaaaaaaaaaaaccctccccccccccccttcccctccgcgg

SEQ ID NO: 1122 - DNA, 50 bp, synthetic construct
  misc_feature 1..50: note = Synthetic
  source 1..50: mol_type = other DNA
gccctgttaagacttggtatcccttcggctccgcaccttaagtgcttaac

SEQ ID NO: 1123 - DNA, 50 bp, synthetic construct; identical to SEQ ID NO: 172
  misc_feature 1..50: note = Synthetic
  source 1..50: mol_type = other DNA

SEQ ID NO: 1124 - DNA, 50 bp, synthetic construct
  misc_feature 1..50: note = Synthetic
  source 1..50: mol_type = other DNA
gagtttttcacactgtgccatgcagcactgtgcgcttatgcggtattagc

SEQ ID NO: 1125 - DNA, 50 bp, synthetic construct
  misc_feature 1..50: note = Synthetic
  source 1..50: mol_type = other DNA
tgcctagttccttaaccatgaatctctcaacgcctcagtatgttctaccc

SEQ ID NO: 1126 - DNA, 50 bp, synthetic construct
  misc_feature 1..50: note = Synthetic
  source 1..50: mol_type = other DNA
ggtgtgtacaaggcccgggaacgtattcaccgcgccgtggctgatgcgcg

SEQ ID NO: 1127 - DNA, 50 bp, synthetic construct
  misc_feature 1..50: note = Synthetic
  source 1..50: mol_type = other DNA
ttcgccaccggtattcctccagatctctacgcatttcaccgctacacctg

SEQ ID NO: 1128 - DNA, 50 bp, synthetic construct
  misc_feature 1..50: note = Synthetic
  source 1..50: mol_type = other DNA
cgcttaacgcgttagctccgacacggaacacgtggaacgtgccccacatc

SEQ ID NO: 1129 - DNA, 50 bp, synthetic construct
  misc_feature 1..50: note = Synthetic
  source 1..50: mol_type = other DNA
acacgagccgaaacccgtgtctctcagactcccacctatcctgtgcatca

SEQ ID NO: 1130 - DNA, 50 bp, synthetic construct
  misc_feature 1..50: note = Synthetic
  source 1..50: mol_type = other DNA
actcgatttctcttcggctccacaccttaagtgcttaaccttgccggcac

SEQ ID NO: 1131 - DNA, 50 bp, synthetic construct
  misc_feature 1..50: note = Synthetic
  source 1..50: mol_type = other DNA
tgaacccgccccgaagggaaacgccatctctggcgtcgtcgggaacatgt

SEQ ID NO: 1132 - RNA, 29 nt, synthetic construct
  modified_base 3: mod_base = OTHER; note = thymine
  modified_base 6: mod_base = OTHER; note = thymine
  modified_base 25: mod_base = OTHER; note = thymine
  misc_feature 1..29: note = Synthetic
  source 1..29: mol_type = other RNA
aatgatacggcgaccaccgagatctacac

SEQ ID NO: 1133 - DNA, 24 bp, synthetic construct
  misc_feature 1..24: note = Synthetic
  source 1..24: mol_type = other DNA
caagcagaagacggcatacgagat

SEQ ID NO: 1134 - DNA, 14 bp, synthetic construct
  misc_feature 1..14: note = Synthetic
  source 1..14: mol_type = other DNA
tcgtcggcagcgtc

SEQ ID NO: 1135 - DNA, 15 bp, synthetic construct
  misc_feature 1..15: note = Synthetic
  source 1..15: mol_type = other DNA
gtctcgtgggctcgg